(19) United States
(12) Patent Application Publication
(10) Pub. No.: US 2009/0054398 A1
(43) Pub. Date: Feb. 26, 2009

(54) CHEMICAL COMPOUNDS

(75) Inventors: Kevin Hudson, Cheshire (GB); Naomi Laing, Waltham, MA (US); Paula Lewis, Waltham, MA (US)

Correspondence Address: ASTRAZENECA R&D BOSTON, 35 GATEHOUSE DRIVE, WALTHAM, MA, 02451-1215, US

(73) Assignee: ASTRAZENECA AB, Sodertalje (SE)

(21) Appl. No.: 12/282,211

(22) PCT Filed: Mar. 9, 2007
(86) PCT No.: PCT/GB2007/000813

§ 371 Date: Sep. 9, 2008

Related U.S. Application Data
(60) Provisional application No. 60/781,081, filed on Mar. 10, 2006.

(51) Int. Cl. A61K 31/553; A61K 31/554; A61K 31/551; A61K 31/55; A61P 35/04

(52) U.S. Cl. 514/211.03; 514/212.03; 514/218; 514/212.08

(57) ABSTRACT

The invention provides a new method for treating disorders associated with activation of the Notch signal transduction pathway comprising administering an effective amount of a compound of Formula (I), in free form or in a pharmaceutically acceptable salt form or in the form of a pharmaceutically acceptable solvate of the compound or the salt, to a human or animal patient in need thereof.

### TECHNICAL FIELD

The present invention relates to a new use of a recently synthesized family of lactams which inhibit gamma secretase for the treatment of disorders associated with activation of the Notch signal transduction pathway.

### BACKGROUND OF THE INVENTION

Notch is a transmembrane receptor signaling molecule that functions in cell development and differentiation. Notch is a heterodimer comprised of two noncovalently associated extracellular and transmembrane subunits. Ligand binding to the extracellular subunit triggers proteolytic cleavages in the transmembrane subunit. One of these cleavages is catalyzed by gamma-secretase and results in the creation of intracellular Notch (also referred to herein as “Notch 1 intracellular domain”). The intracellular Notch protein enters the cell nucleus and binds to transcription factors which ultimately results in the activation of downstream target genes. Aberrations in the normal Notch signaling pathway has been linked to pathological conditions including neoplastic growth of a variety of tissues, including cervical carcinomas, lung cancer, breast cancer, pancreatic cancer, endometrial carcinomas, colorectal neoplasms, medulloblastomas, mucoepidermoid carcinomas, ovarian cancers and T-cell leukemias.

WO 2004/031154, the contents of which are incorporated herein by reference, discloses certain novel lactams and methods for their use for the treatment of neurological disorders related to amyloid beta protein production and neurological disorders such as Alzheimer's Disease. This patent application, however, does not teach or suggest the use of the novel lactams as Notch inhibitors or to treat disorders associated with activation of the Notch signal transduction pathway, such as cancer.

### SUMMARY OF THE INVENTION

The invention provides a new method for treating disorders associated with activation of the Notch signal transduction pathway comprising administering an effective amount of a compound as specifically provided below, in free form or in a pharmaceutically acceptable salt form or in the form of a pharmaceutically acceptable solvate of the compound or the salt, to a human or animal patient in need thereof.

### DETAILED DESCRIPTION OF THE INVENTION

It is contemplated herein that the compounds of the present invention have utility for the treatment of disorders associated with activation of the Notch signal transduction pathway by inhibiting the activation of the Notch signaling pathway. Specifically, it is contemplated that the compounds of the present invention inhibit activation of the Notch signal transduction pathway by inhibiting gamma secretase. Gamma secretase normally cleaves the transmembrane subunit of the Notch protein, creating the intracellular Notch protein, which then enters the cell nucleus and binds to transcription factors which in turn cause activation of downstream target genes. Thus, compounds that inhibit gamma secretase activity, either directly or indirectly, may be used to control the production of the intracellular Notch protein and can thus be used to treat disorders associated with activation of the Notch signal transduction pathway.

As used in this application, the term “disorders associated with the activation of the Notch signal transduction pathway” includes, but is not limited to, cancer. For example, such disorder may also include Down's syndrome (Fischer et al., FASEB Journal, 2005; 19:1451-1458 or other inherited disease syndromes (Gridley, T. Human Molelcular Genetics, 2003, Apr. 12 (Suppl 1): R9-R13). As used herein, the term “cancer” includes, but is not limited to, adult/childhood haematological cancers, including leukaemias (including but not limited to T cell acute lymphocytic leukaemia (T-ALL)), lymphomas and myelomas, genitourinary, gynecologic, digestive, gastrointestinal, neurologic, breast, lung and mucoepidermoid cancers, for example, multiple myeloma, extramedullary plasmacytoma, renal cell carcinoma, ovarian, endometrial, cervical, colon, prostate or pancreatic malignancies; for example, cancers mediated in whole or in part, by the Notch signal transduction pathway.

An effective amount of a compound as described herein for use in the methods disclosed herein refers to that amount of active ingredient useful to treat, prevent and/or ameliorate the pathological effects of a disorder associated with activation of the Notch signal transduction pathway.

As used in this application, the term “substituted,” means that any number of hydrogens on the designated atom is replaced with a selection from the indicated group, provided that the designated atom's normal valency is not exceeded, and that the substitution results in a stable compound. For example, when a substituent is keto (i.e., ═O), then 2 hydrogens on the atom are replaced.

When any variable (e.g., R¹, R⁷, R^{a}, R^{e} etc.) occurs more than one time in any constituent or formula for a compound, its definition at each occurrence is independent of its definition at every other occurrence. Thus, for example, if a group is shown to be substituted with 0-3 R¹, then said group may optionally be substituted with 0, 1, 2 or 3 R¹ groups and R¹ at each occurrence is selected independently from the definition of R¹. Also, combinations of substituents and/or variables are permissible only if such combinations result in stable compounds.

The compounds herein described may have asymmetric centers. Compounds of the present invention containing an asymmetrically substituted atom may be isolated in optically active or racemic forms. It is well known in the art how to prepare optically active forms, such as by resolution of racemic forms or by synthesis from optically active starting materials. When required, separation of the racemic material can be achieved by methods known in the art. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. All chiral, diastereomeric, racemic forms and all geometric isomeric forms of a structure are intended, unless the specific stereochemistry or isomeric form is specifically indicated.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent. Combinations of substituents and/or variables are permissible only if such combinations result in stable compounds.

As used herein “acyl” refers to radicals of the of the general Formula —C(═O)—R, wherein R is hydrogen, hydrocarbyl radical, amino or alkoxy. Acyl groups include, for example, acetyl, propionyl, benzoyl, phenyl acetyl, carboethoxy, and dimethylcarbamoyl.

As used herein “aromatic” refers to hydrocarbyl radicals having one or more polyunsaturated carbon rings having aromatic character, (e.g., 4n+2 delocalized electrons) and comprising up to about 14 carbon atoms.

As used herein, “alkyl” or “alkylene” is intended to include both branched and straight-chain saturated aliphatic hydrocarbon groups having the specified number of carbon atoms. For example, “C₁₋₆ alkyl” denotes alkyl having 1, 2, 3, 4, 5 or 6 carbon atoms. Examples of alkyl include, but are not limited to, methyl, ethyl, n-propyl, i-propyl, n-butyl, i-butyl, sec-butyl, t-butyl, pentyl, and hexyl. As used herein, “C₁₋₃ alkyl”, whether a terminal substituent or an alkylene group linking two substituents, is understood to specifically include both branched and straight-chain methyl, ethyl, and propyl.

As used herein, “alkylcycloalkyl is intended to include both an alkyl portion as defined herein and a cycloalkyl portion. For example C₁₋₃alkylC₃₋₆cycloalkyl would include —CH₂—CH₂—CH₂-cyclopropyl.

As used herein, “alkenyl” or “alkenylene” is intended to include hydrocarbon chains of either a straight or branched configuration with one or more unsaturated carbon-carbon bonds that may occur at any stable point along the chain. Examples of “C₃₋₆alkenyl” include, but are not limited to, 1-propenyl, 2-propenyl, 1-butenyl, 2-butenyl, 3-butenyl, 3-methyl-2-butenyl, 2-pentenyl, 3-pentenyl, hexenyl, and the like.

As used herein, “alkynyl” or “alkynylene” is intended to include hydrocarbon chains of either a straight or branched configuration with one or more carbon-carbon triple bonds that may occur at any stable point along the chain, such as ethynyl, 1-propynyl, 2-propynyl, 1-butynyl, 2-butynyl, 3-butynyl, and the like.

As used herein, “alkoxy” or “alkyloxy” represents an alkyl group as defined above with the indicated number of carbon atoms attached through an oxygen bridge. Examples of alkoxy include, but are not limited to, methoxy, ethoxy, n-propoxy, i-propoxy, n-butoxy, s-butoxy, t-butoxy, n-pentoxy, and s-pentoxy. Preferred alkoxy groups are methoxy, ethoxy, n-propoxy, i-propoxy, n-butoxy, s-butoxy, t-butoxy. Similarly, “alkylthio” or “thioalkoxy” represent an alkyl group as defined above with the indicated number of carbon atoms attached through a sulphur bridge.

As used herein, the term “aryl” is intended to mean aromatic radicals including both monocyclic aromatic radicals comprising 6 carbon atoms and polycyclic aromatic radicals comprising up to about 14 carbon atoms.

As used herein, the term “heteroaryl” is intended to mean aromatic radicals including both monocyclic and bicyclic aromatic radicals comprising 5 to 14 atoms having one or more heteroatoms, independently selected from N, O and S, as part of the ring structure.

As used herein, “carbocycle” is intended to mean any stable 3- to 7-membered monocyclic or bicyclic or 7- to 13-membered bicyclic or tricyclic, any of which may be saturated, partially unsaturated, or aromatic. Examples of such carbocycles include, but are not limited to, cyclopropyl, cyclobutyl, cyclopentyl, cyclohexyl, cycloheptyl, adamantyl, cyclooctyl, bicyclooctane, bicyclononane, bicyclodecane (decalin), bicyclooctane, fluorenyl, phenyl, naphthyl, indanyl, adamantyl, or tetrahydronaphthyl (tetralin).

As used herein “cycloalkyl” is intended to include saturated ring groups, having the specified number of carbon atoms. For example, “C₃₋₆ cycloalkyl” denotes such groups as cyclopropyl, cyclobutyl, cyclopentyl, or cyclohexyl.

As used herein “cycloalkenyl” refers to ring-containing radicals having at least one carbon-carbon double bond in the ring, and having in the range about 3 up to 12 carbons atoms.

As used herein “cycloalkynyl” refers to ring-containing radicals having at least one carbon-carbon triple bond in the ring, and having in the range about 3 up to 12 carbons atoms.

As used herein, “halo” or “halogen” refers to fluoro, chloro, bromo, and iodo. “Counterion” is used to represent a small, negatively charged species such as chloride, bromide, hydroxide, acetate, sulfate, and the like. “Haloalkyl” is intended to include both branched and straight-chain saturated aliphatic hydrocarbon groups having the specified number of carbon atoms, substituted with 1 or more halogen (for example —CᵥF_{w} where v=1 to 3 and w=1 to (2v+1)). Examples of haloalkyl include, but are not limited to, trifluoromethyl, trichloromethyl, pentafluoroethyl, pentachloroethyl, 2,2,2-trifluoroethyl, 2,2-difluoroethyl, heptafluoropropyl, and heptachloropropyl. “Haloalkoxy” is intended to mean a haloalkyl group as defined above with the indicated number of carbon atoms attached through an oxygen bridge; for example trifluoromethoxy, pentafluoroethoxy, 2,2,2-trifluoroethoxy, and the like. “Halothioalkoxy” is intended to mean a haloalkyl group as defined above with the indicated number of carbon atoms attached through a sulphur bridge.

As used herein, the term “heterocycle” or “heterocyclic” refers to a ring-containing monovalent and divalent radicals having one or more heteroatoms, independently selected from N, O and S, as part of the ring structure and comprising at least 3 and up to about 20 atoms, unless otherwise specified, in the rings. Heterocyclic groups may be saturated or unsaturated, containing one or more double bonds, and heterocyclic groups may contain more that one ring. The heterocyclic rings described herein may be substituted on carbon or on a nitrogen atom if the resulting compound is stable. If specifically noted, nitrogen in the heterocycle may optionally be quaternized. It is understood that when the total number of S and O atoms in the heterocycle exceeds 1, then these heteroatoms are not adjacent to one another.

As used herein, “R^{a} and R^{b} and the N to which they are attached in combination form a 5 or 6-membered N-linked heterocycle having 2 nitrogen atoms, wherein the non-linlced nitrogen is substituted with R^{c} or 1 nitrogen and 1 oxygen, ring atoms wherein there is no non-linked nitrogen” refers to a 5- or 6-membered monocyclic ring which may be saturated or unsaturated, containing one or more double bonds.

Examples of heterocycles include, but are not limited to, 1H-indazole, 2-pyrrolidonyl, 2H, 6H-1, 5,2-dithiazinyl, 2H-pyrrolyl, 3H-indolyl, 4-piperidonyl, 4aH-carbazole, 4H-quinolizinyl, 6H-1, 2,5-thiadiazinyl, acridinyl, azetidine, aziridine, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazalonyl, carbazolyl, 4aH-carbazolyl, b-carbolinyl, chromanyl, chromenyl, cinnolinyl, decahydroquinolinyl, 2H,6H-1,5,2-dithiazinyl, dioxolane, furyl, 2,3-dihydrofuran, 2,5-dihydrofuran, dihydrofuro[2,3-b]THF, furanyl, furazanyl, homopiperidinyl, imidazolidine, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxirane, oxazolidinylperimidinyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, piperidonyl, 4-piperidonyl, purinyl, pyranyl, pyrrolidine, pyrroline, pyrrolidine, pyrazinyl, pyrazolidinyl, pyrazolinyl, pyrazolyl, pyridazinyl, pyridooxazole, pyridoimidazole, pyridothiazole, pyridinyl, N-oxide-pyridinyl, pyridyl, pyrimidinyl, pyrrolidinyl, pyrrolinyl, pyrrolyl, quinazolinyl, quinolinyl, 4H-quinolizinyl, quinoxalinyl, quinuclidinyl, carbolinyl, THFyl, tetrahydroisoquinolinyl, thiophane, thiotetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, thiirane, triazinyl, 1,2,3-triazolyl, 1,2,4-triazolyl, 1,2,5-triazolyl, 1,3,4-triazolyl, xanthenyl.

As used herein, the term 5- or 6-membered ring means a 6-membered aromatic ring, or a 5- or 6-membered heterocyclic ring.

As used herein, the term 6-membered ring means a 6-membered aromatic ring, or 6-membered heterocyclic ring.

Compounds of the invention may exist in free or salt form, e.g., as acid addition salts. In this specification, unless otherwise indicated, it is understood that the compounds disclosed herein include the compounds in any form, e.g., free or acid addition salt form, or where the compounds contain acidic substituents, in base addition salt form. As the compounds disclosed herein are intended for use as pharmaceuticals, pharmaceutically acceptable salts are preferred.

As used herein, “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like EtOAc, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, the disclosure of which is hereby incorporated by reference.

“Prodrugs” are intended to include any covalently bonded carriers that release the active parent drug according to formula (I) in vivo when such prodrug is administered to a mammalian subject. Prodrugs of a compound of formula (I) are prepared by modifying functional groups present in the compound in such a way that the modifications are cleaved, either in routine manipulation or in vivo, to the parent compound. Prodrugs include compounds of Formula (I) wherein a hydroxy, amino, or sulfhydryl group is bonded to any group that, when the prodrug or compound of Formula (I) is administered to a mammalian subject, cleaves to form a free hydroxyl, free amino, or free sulfhydryl group, respectively. Examples of prodrugs include, but are not limited to, acetate, formate and benzoate derivatives of alcohol and amine functional groups in the compounds of Formula (I), and the like.

“Stable compound” and “stable structure” are meant to indicate a compound that is sufficiently robust to survive isolation to a useful degree of purity from a reaction mixture, and formulation into an efficacious therapeutic agent.

The words “treat”, “treatment” and “treating” are to be understood as embracing prophylaxis and treatment or amelioration of symptoms of disease as well as treatment of the cause of the disease.

The term “substantially free” refers to less than 10% of the other sterioisomers, more particularly less than 5%, in particular less than 2%, more particularly less than 1%, particularly less then 0.5%, in particular less than 0.2%.

As used herein, by “free form” refers to the non-ionized compound at neutral pH.

### Compounds for Use in the Methods of the Invention

Preferably, the compounds of the invention for use in the methods of treatment described herein are selected from the compounds disclosed herein, in free form or in the form of a pharmaceutically acceptable salt of the compound or in the form of a pharmaceutically acceptable solvate of the compound or salt:

The compounds for use in the invention include compounds of Formula IA:

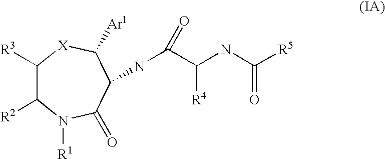

wherein:

X is CH₂, O, NR¹, SO₂ or S;

Ar¹ is a 5- or 6-membered aromatic or heterocyclic ring optionally substituted with 0, 1, 2, or 3 R^{e} moieties, said ring having 0, 1, 2 or 3 nitrogen, oxygen or sulfur atoms, but no more than 2 oxygen atoms or 2 sulfur atoms or 1 oxygen and 1 sulfur atom;

R¹ is H, —C₁₋₃alkylC₃₋₆cycloalkyl, C₁₋₆alkyl, C₃₋₆alkenyl, C₃₋₆alkynyl, C₃₋₆cycloalkyl, C₂₋₄alkylNR^{a}R^{b}, C₁₋₄alkylC(═O)R^{d}; or C₁₋₃alkylphenyl substituted with 0, 1, 2 or 3 R^{e};

R^{a} and R^{b} are at each occurrence independently selected from H, C₁₋₄alkyl or C₃₋₆cycloalkyl, or R^{a} and R^{b} and the N to which they are attached in combination form a 5 or 6-membered N-linked heterocycle having 2 nitrogen atoms, wherein the non-linked nitrogen is substituted with R^{c} or 1 nitrogen and 1 oxygen, ring atoms wherein there is no non-linked nitrogen;

R^{c} is, at each occurrence independently selected from H, C₁₋₃alkyl, or substituted phenyl with 0, 1, 2, or 3 R^{e};

R^{d} is, at each occurrence independently selected from C₁₋₃alkyl, hydroxy, C₁₋₃alkoxy, or NR^{a}R^{b};

R^{e} is, at each occurrence independently selected from H, OH, F, Cl, Br, I, CN, NO₂, CF₃, C₁₋₆alkyl, or C₁₋₆alkoxy;

R² and R³ are at each occurrence independently selected from H, C₁₋₆alkyl, C₄₋₆ cycloalkyl, aryl, or heteroaryl, or R² and R³ in combination form a fused phenyl or cyclohexyl moiety that may be substituted with 0, 1 or 2 R^{f} moieties,

R^{f} is NO₂, F, Cl, Br, I, CF₃, CN, C₁₋₆alkyl, or C₁₋₆alkoxy;

R⁴ is H, CHR⁷R⁸, 5- or 6-membered cycloalkyl, 5- or 6-membered heterocyclic, 5 or 6-membered aromatic ring optionally substituted with 0, 1, or 2 R^{f} moities, said heterocyclic ring having 0, 1, 2 or 3 nitrogen, oxygen or sulfur atoms, but no more than 2 oxygen atoms or 2 sulfur atoms or 1 oxygen and 1 sulfur atom;

R⁵ is C₁₋₃alkylR⁹ or CH(OH)R¹⁰;

R⁷ and R⁸ are, at each occurrence are independently selected from H, C₁₋₄alkyl, OH, SH, CH₂SCH₃, CONH₂, CH₂CONH₂, CO₂H, CH₂CO₂H, (CH₂)₃NHCH(NH₂)₂, C₁₋₄alkylamine, indole, imidazole, phenyl or hydroxyphenyl or R⁷ and R⁸ in combination form a 6-membered aromatic or heterocyclic ring optionally substituted with 0, 1 or 2 R^{f} moieties said heterocyclic ring having 0, 1, 2 or 3 nitrogen, oxygen or sulfur atoms, but no more than 2 oxygen atoms or 2 sulfur atoms or 1 oxygen and 1 sulfur atom;

R⁹ is phenyl substituted with 0, 1, 2 or 3 R^{e};

R¹⁰ is alkyl or R⁹.

Particular values of variable groups are as follows. Such values may be used where appropriate with any of the definitions, claims or embodiments defined hereinbefore or hereinafter.

In addition, compounds of the invention include a compound of Formula (IA) as indicated by the following formulae:

Of Formula (I) wherein:
X is CH₂, O, NR¹, SO₂ or S;
Ar¹ is a 5- or 6-membered aromatic or heterocyclic ring optionally substituted with 0, 1, 2, or 3 R^{e} moieties, said ring having 0, 1, or 2 nitrogen, oxygen or sulfur atoms, but no more than 2 oxygen atoms or 1 oxygen and 1 sulfur atom;
R¹ is H, —C₁₋₃alkylC₃₋₆cycloalkyl, C₁₋₆alkyl, C₃₋₆alkenyl, C₃₋₆alkynyl, C₃₋₆cycloalkyl, C₂₋₄alkylNR^{a}R^{b}, C₁₋₄alkylC(═O)R^{d}; or C₁₋₃alkylphenyl substituted with 0, 1, or 2 R^{e};

R^{a} and R^{b} are, at each occurrence independently selected from H, C₁₋₄alkyl or C₃₋₆cycloalkyl, or R^{a} and R^{b} and the N to which they are attached in combination form a 6-membered N-linked heterocycle having 2 nitrogen atoms, wherein the non-linked nitrogen is substituted with R^{c} or 1 nitrogen and 1 oxygen, ring atoms wherein there is no non-linlked nitrogen;

R^{c} is, at each occurrence independently selected from H, C₁₋₃alkyl, or phenyl;

R^{d} is, at each occurrence independently selected from C₁₋₃alkyl, or NR^{a}R^{b};

R^{e} is, at each occurrence independently selected from OH, F, Cl, Br, I, CN, NO₂, CF₃, C₁₋₃alkyl, or C₁₋₃alkoxy;

R² and R³ are at each occurrence independently selected from H, C₁₋₆alkyl, C₄₋₆ cycloalkyl, or aryl, or R² and R³ in combination form a fused phenyl moiety that may be substituted with 0, 1 or 2 R^{f} moieties,

R^{f} is NO₂, F, Cl, Br, I, CF₃, CN, C₁₋₃alkyl, or C₁₋₃alkoxy;

R⁴ is H, CHR⁷R⁸, 6-membered cycloalkyl, or 6-membered heterocyclic, or 6-membered aromatic ring optionally substituted with 0, 1, or 2 R^{f} moities, said heterocyclic ring having 0, 1, 2 or 3 nitrogen, oxygen or sulfur atoms, but no more than 2 oxygen atoms or 2 sulfur atoms or 1 oxygen and 1 sulfur atom;

R⁵ is C₁₋₃alkylR⁹ or CH(OH)R¹⁰;

R⁷ and R⁸ are, at each occurrence independently selected from H, C₁₋₄alkyl, OH, CONH₂, CH₂CONH₂, CO₂H, CH₂CO₂H, (CH₂)₃NHCH(NH₂)₂, C₁₋₄alkylamine, indole, imidazole, phenyl or hydroxyphenyl or R⁷ and R⁸ in combination form a 6-membered aromatic or heterocyclic ring optionally substituted with 0, 1 or 2 R^{f} moieties said heterocyclic ring having 0, 1, 2 or 3 nitrogen, oxygen or sulfur atoms, but no more than 2 oxygen atoms or 2 sulfur atoms or 1 oxygen and 1 sulfur atom;

R⁹ is phenyl substituted with 0, 1, or 2 R^{e};

R¹⁰ is alkyl or R⁹;

Of Formula (IA) wherein:

X is CH₂, O, NR¹, SO₂ or S;

Ar¹ is a 5- or 6-membered aromatic or heterocyclic ring optionally substituted with 0, 1, 2, or 3 R^{e} moieties, said ring having 0, 1, or 2 nitrogen, oxygen or sulfur atoms, but no more than 2 oxygen atoms or 1 oxygen and 1 sulfur atom;

R¹ is H, —C₁₋₃alkylC₃₋₆cycloalkyl, C₁₋₆alkyl, C₃₋₆alkenyl, C₃₋₆alkynyl C₃₋₆cycloalkyl, C₂₋₄alkylNR^{a}R^{b}, C₁₋₄alkylC(═O)R^{d}; or C₁₋₃alkylphenyl substituted with 0, 1, or 2 R^{e};

R^{a} and R^{b} are, at each occurrence independently selected from H, C₁₋₄alkyl or C₃₋₆cycloalkyl, or R^{a} and R^{b} and the N to which they are attached in combination form a 5-membered N-linked heterocycle having 2 nitrogen atoms, wherein the non-linked nitrogen is substituted with R^{c} or 1 nitrogen and 1 oxygen, ring atoms wherein there is no non-linked nitrogen;

R^{c} is, at each occurrence independently selected from H, C₁₋₃alkyl, phenyl;

R^{d} is, at each occurrence independently selected from C₁₋₃alkyl or NR^{a}R^{b};

R^{e} is, at each occurrence independently selected from H, OH, F, Cl, Br, I, CN, NO₂, CF₃, C₁₋₆alkyl, or C₁₋₆alkoxy;

R² and R³ are at each occurrence independently selected from H, C₁₋₆alkyl, C₄₋₆ cycloalkyl or aryl or R² and R³ in combination form a fused phenyl moiety that may be substituted with 0, 1 or 2 R^{f} moieties,

R^{f} is H, NO₂, F, Cl, Br, I, CF₃, C₁₋₆alkyl, or C₁₋₆alkoxy;

R⁴ is H, CHR⁷R⁸, or 6-membered heterocyclic, or 6-membered aromatic ring optionally substituted with 0, 1, or 2 R^{f} moities, said heterocyclic ring having 0, 1, 2 or 3 nitrogen, oxygen or sulfur atoms, but no more than 2 oxygen atoms or 2 sulfur atoms or 1 oxygen and 1 sulfur atom;

R⁴ is H or CHR⁷R⁸;

R⁵ is C₁₋₃alkylR⁹ or CH(OH)R¹⁰;

n is 0, 1 or 2;

R⁷ and R⁸ are, at each occurrence independently selected from H, C₁₋₄alkyl, OH, CONH₂, CH₂CONH₂, CO₂H, CH₂CO₂H, (CH₂)₃NHCH(NH₂)₂, C₁₋₄alkylamine, indole, imidazole, phenyl or hydroxyphenyl or R⁷ and R⁸ in combination form a 6-membered aromatic or heterocyclic ring optionally substituted with 0, 1 or 2 R^{f} moieties said heterocyclic ring having 0, 1, or 2 nitrogen, oxygen or sulfur atoms;

R⁹ is phenyl substituted with 1, or 2 R^{e};

R¹⁰ is alkyl or phenyl substituted with 1, or 2 R^{e};

Of Formula (IA) wherein:

X is CH₂, O, NR¹, SO₂ or S;

Ar¹ is a 5- or 6-membered aromatic or heterocyclic ring optionally substituted with 0, 1, 2, or 3 R^{e} moieties, said ring having 0, 1, or 2 nitrogen, oxygen or sulfur atoms, but no more than 1 oxygen and 1 sulfur atom;

R¹ is H, —C₁₋₃alkylC₃₋₆cycloalkyl, C₁₋₆alkyl, C₃₋₆alkenyl, C₃₋₆alkynyl C₃₋₆cycloalkyl, C₂₋₄alkylNR^{a}R^{b}, C₁₋₄alkylC(═O)R^{d}; or C₁₋₃alkylphenyl substituted with 0, or 1 R^{e};

R^{a} and R^{b} are, at each occurrence independently selected from H, C₁₋₄alkyl or C₅₋₆cycloalkyl, or R^{a} and R^{b} and the N to which they are attached in combination form a 6-membered N-linked heterocycle having 2 nitrogen atoms, wherein the non-linked nitrogen is substituted with R^{c} or 1 nitrogen and 1 oxygen, ring atoms wherein there is no non-linked nitrogen;

R^{c} is, at each occurrence independently selected from H, C₁₋₃alkyl;

R^{d} is, at each occurrence independently selected from C₁₋₃alkyl;

R^{e} is, at each occurrence independently selected from H, OH, F, Cl, Br, I, CN, NO₂, CF₃, C₁₋₆alkyl;

R² and R³ are at each occurrence independently selected from H, C₁₋₆alkyl, or R² and R³ in combination form a fused phenyl moiety that may be substituted with 0, 1 or 2 R^{f} moieties,

R^{f} is H, F, Cl, Br, I, CF₃, C₁₋₆alkyl;

R⁴ is H, CHR⁷R⁸, or 6-membered heterocyclic, or 6-membered aromatic ring optionally substituted with 0, 1, or 2 R^{f} moieties, said heterocyclic ring having 0, 1, or 2 nitrogen, oxygen or sulfur atoms;

R⁵ is C₁₋₃alkylR⁹ or CH(OH)R¹⁰;

n is 0, 1 or 2;

R⁷ and R⁸ are, at each occurrence independently selected from H, C₁₋₄alkyl, OH, CONH₂, CH₂CONH₂, CO₂H, CH₂CO₂H, (CH₂)₃NHCH(NH₂)₂, C₁₋₄alkylamine, indole, imidazole, phenyl or hydroxyphenyl or R⁷ and R⁸ in combination form a 6-membered aromatic or heterocyclic ring optionally substituted with 0, 1 or 2 R^{f} moieties said heterocyclic ring having 0, 1, or 2 nitrogen, or oxygen atoms;

R⁹ is phenyl substituted with 1, or 2 R^{e};

R¹⁰ is alkyl or R⁹;

Of Formula (IA) wherein:

X is CH₂, O, SO₂ or S;

Ar¹ is a 5- or 6-membered aromatic or heterocyclic ring optionally substituted with 0, 1, or 2 R^{e} moieties, said ring having 0, 1, or 2 nitrogen, oxygen or sulfur atoms;

R¹ is H, —C₁₋₃alkylC₃₋₆cycloalkyl, C₁₋₆alkyl, C₃₋₆alkenyl, C₃₋₆alkynyl C₃₋₆cycloalkyl, C₂₋₄alkylNR^{a}R^{b}, C₁₋₄alkylC(═O)R^{d};

R^{a} and R^{b} are, at each occurrence independently selected from H, C₁₋₄alkyl or C₅₋₆cycloalkyl, or R^{a} and R^{b} and the N to which they are attached in combination form a 6-membered N-linked heterocycle having 2 nitrogen atoms, wherein the non-linked nitrogen is substituted with R^{c} or 1 nitrogen and 1 oxygen, ring atoms wherein there is no non-linked nitrogen;

R^{d} is, at each occurrence independently selected from C₁₋₃alkyl;

R^{e} is, at each occurrence independently selected from H, OH, F, Cl, Br, I, NO₂, CF₃, or C₁₋₆alkyl;

R² and R³ are at each occurrence independently selected from C₁₋₆alkyl or R² and R³ in combination form a fused phenyl moiety that may be substituted with 0, 1 or 2 R^{f} moieties,

R^{f} is H, F, Cl, Br, I, CF₃;

R⁴ is H, CHR⁷R⁸, or 6-membered heterocyclic, or 6-membered aromatic ring optionally substituted with 0, 1, or 2 R^{f} moieties, said heterocyclic ring having 0, 1, or 2 nitrogen, or oxygen atoms;

R⁵ is C₁₋₃alkylR⁹ or CH(OH)R¹⁰;

R⁷ and R⁸ are, at each occurrence independently selected from H, C₁₋₄alkyl, OH, CONH₂, CH₂CONH₂, CO₂H, C₁₋₄alkylamine, phenyl or hydroxyphenyl or R⁷ and R⁸ in combination form a 6-membered aromatic or heterocyclic ring optionally substituted with 0, 1 or 2 R^{f} moieties said heterocyclic ring having 0, 1, or 2 nitrogen, or oxygen atoms;

R⁹ is phenyl substituted with 1 or 2 R^{e};

R¹⁰ is alkyl or R⁹;

Of Formula (IA) wherein:

X is CH₂, O, SO₂ or S;

Ar¹ is a 6-membered aromatic or heterocyclic ring optionally substituted with 0, 1, or 2 R^{e} moieties, said ring having 0, or 1 nitrogen, oxygen or sulfur atoms;

R¹ is H, —C₁₋₃alkylC₃₋₆cycloalkyl, C₁₋₆alkyl, C₃₋₆alkenyl, C₃₋₆alkynyl, C₃₋₆cycloalkyl, C₂₋₄alkylNR^{a}R^{b}, C₁₋₄alkylC(═O)R^{d};

R^{a} and R^{b} are, at each occurrence independently selected from H, C₁₋₄alkyl or C₅₋₆cycloalkyl or R^{a} and R^{b} and the N to which they are attached in combination form a 6-membered N-linked heterocycle having 1 nitrogen and 1 oxygen, ring atom, wherein there is no non-linked nitrogen;

R^{d} is, at each occurrence independently selected from C₁₋₃alkyl;

R^{e} is, at each occurrence independently selected from H, OH, F, Cl, Br, I, CF₃;

R² and R³ are combined to form a fused phenyl moiety substituted with 0, 1 or 2 R^{f} moieties,

R^{f} is H, F, Cl, Br, I, or CF₃;

R⁴ is H, CHR⁷R⁸, or 6-membered heterocyclic, or 6-membered aromatic ring optionally substituted with 0, 1, or 2 R^{f} moieties, said heterocyclic ring having 0, or 1, nitrogen, or oxygen atoms;

R⁵ is C₁₋₃alkylR⁹ or CH(OH)R¹⁰;

R⁷ and R⁸ are, at each occurrence independently selected from H, OH, or R⁷ and R⁸ in combination form a 6-membered aromatic or heterocyclic ring optionally substituted with 0, 1 or 2 R^{f} moieties said heterocyclic ring having 0, or 1, nitrogen, or oxygen atoms;

R⁹ is phenyl substituted with 2 R^{e};

R¹⁰ is phenyl substituted with 2 R^{e};

Of Formula (IA) wherein:

X is CH₂, O, or S;

Ar¹ is a 6-membered aromatic or heterocyclic ring optionally substituted with 0, 1, or 2 R^{e} moieties, said ring having 0, or 1 nitrogen, or oxygen atoms;

R¹ is H, —C₁₋₃alkylC₃₋₆cycloalkyl, C₁₋₆alkyl, C₃₋₆alkenyl, C₃₋₆alkynyl, C₃₋₆cycloalkyl, C₂₋₄alkylNR^{a}R^{b};

R^{a} and R^{b} are, at each occurrence independently selected from H, C₁₋₄alkyl or C₅₋₆cycloalkyl or R^{a} and R^{b} and the N to which they are attached in combination form a 6-membered N-linked heterocycle having 1 nitrogen and 1 oxygen, ring atom, wherein there is no non-linlked nitrogen;

R² and R³ are combined to form a fused phenyl moiety substituted with 0, 1 or 2 R^{f};

R^{e} is, at each occurrence independently selected from H, OH, F, Cl, Br, I, CF₃;

R^{f} is F or Cl;

R⁴ is H, CHR⁷R⁸, or 6-membered aromatic ring optionally substituted with 0, 1, or 2 R^{f} moieties;

R⁵ is C₁₋₃alkylR⁹ or CH(OH)R¹⁰;

R⁷ and R⁸ are, at each occurrence independently selected from H, OH, or R⁷ and R⁸ in combination form a 6-membered aromatic ring optionally substituted with 0, 1 or 2 R^{f} moieties;

R⁷ and R⁸ are, at each occurrence independently selected from H or OH;

R⁹ is phenyl substituted with 2 R^{e};

R¹⁰ is phenyl substituted with 2 R^{e};

Of Formula (IA) wherein:

X is O or CH₂ or S;

Ar¹ is a 6-membered aromatic or heterocyclic ring optionally substituted with 0, 1, or 2 R^{e} moieties, said ring having 0, or 1 nitrogen atom;

R¹ is H, —C₁₋₃alkylC₃₋₆cycloalkyl, C₁₋₆alkyl, C₃₋₆alkenyl, C₃₋₆alkynyl, C₃₋₆cycloalkyl, C₂₋₄alkylNR¹R^{b};

R^{a} and R^{b} are, at each occurrence independently selected from H, C₁₋₄alkyl or C₅₋₆cycloalkyl or R^{a} and R^{b} and the N to which they are attached in combination form a 6-membered N-linked heterocycle having 1 nitrogen and 1 oxygen, ring atom, wherein there is no non-linked nitrogen;

R² and R³ are combined to form a fused phenyl moiety substituted with 0, 1 or 2 R^{f} wherein R^{f} is F or Cl;

R⁴ is H, CH₃, or a 6-membered aromatic ring optionally substituted with 0, 1, or 2 R^{f} moieties;

R⁵ is C₁₋₃alkylR⁹;

R^{e} is, at each occurrence independently selected from H, OH, F, Cl, Br, I, CF₃;

R⁹ is phenyl substituted with 2 R^{e};

Of Formula (IA) wherein:

X is O or CH₂;

Ar¹ is a 6-membered aromatic ring optionally substituted with 0, 1, or 2 R^{e} moieties;

R¹ is H, —C₁₋₃alkylC₃₋₆cycloalkyl, C₁₋₆alkyl, C₃₋₆alkenyl, C₃₋₆alkynyl, C₃₋₆cycloalkyl, C₂₋₄alkylNR^{a}R^{b};

R^{a} and R^{b} are, at each occurrence independently selected from H, C₁₋₄alkyl or C₅₋₆cycloalkyl or R^{a} and R^{b} and the N to which they are attached in combination form a 6-membered N-linked heterocycle having 1 nitrogen and 1 oxygen, ring atom, wherein there is no non-linked nitrogen;

R² and R³ are combined to form a fused phenyl moiety substituted with 0, 1 or 2 R^{f} wherein R^{f} is F or Cl;

R⁴ is H, CH₃, or a 6-membered aromatic ring optionally substituted with 0, 1, or 2 R^{f} moieties;

R⁵ is C₁₋₃alkylR⁹;

R^{e} is, at each occurrence independently selected from H, OH, F, Cl, Br, I, CF₃;

R⁹ is phenyl substituted with 2 R^{e}.

Of Formula (IA) wherein:

X is O;

Ar¹ is a 6-membered aromatic ring optionally substituted with 0, 1, or 2 R^{e} moieties;

R¹ is H, —C₁₋₃alkylC₃₋₆cycloalkyl, C₁₋₆alkyl, C₃₋₆alkenyl, C₃₋₆alkynyl;

R² and R³ are combined to form a fused phenyl moiety substituted with 0, 1 or 2 R^{f} wherein R^{f} is F or Cl;

R⁴ is H, CH₃, or a 6-membered aromatic ring optionally substituted with 0, 1, or 2 R^{f} moieties;

R⁵ is C₁₋₃alkylR⁹;

R^{e} is, at each occurrence independently selected from H, OH, F, Cl, Br, I, CF₃;

R⁹ is phenyl substituted with 2 R^{e}.

Of Formula (IA) wherein X is CH₂, O, SO₂ or S.

Of Formula (IA) wherein Ar¹ is a 5- or 6-membered aromatic or heterocyclic ring optionally substituted with 0 or 1 R^{e}.

Of Formula (IA) wherein R¹ is H, —C₁₋₃alkylC₃₋₆cycloalkyl, C₁₋₆alkyl, C₃₋₆alkenyl, C₃₋₆alkynyl.

Of Formula (IA) wherein R^{a} and R^{b} are, at each occurrence independently selected from H, C₁₋₄alkyl or C₅₋₆cycloalkyl or R^{a} and R^{b} and the N to which they are attached in combination form a 6-membered N-linked heterocycle having 1 nitrogen and 1 oxygen, ring atom, wherein there is no non-linked nitrogen.

Of Formula (I A) wherein R² and R³ are combined to form a fused phenyl moiety substituted with 0, 1 or 2 R^{f}.

Of Formula (IA) wherein R^{e} is, at each occurrence independently selected from F or Cl.

Of Formula (IA) wherein R^{f} is F or Cl.

Of Formula (IA) wherein R⁴ is H or CHR⁷R⁸ or a 6-membered aromatic ring optionally substituted with 0, 1, or 2 R^{f} moieties wherein R⁷ and R⁸ are, at each occurrence independently selected from H or OH.

Of Formula (IA) wherein R⁴ is a 6-membered aromatic ring optionally substituted with 0, 1, or 2 R^{f} moieties wherein R^{f} is halo.

Of Formula (IA) wherein R⁵ is C₁₋₃alkylR⁹ or CH(OH)R¹⁰.

Of Formula (IA) wherein R⁷ and R⁸ are, at each occurrence independently selected from Hor OH.

Of Formula (IA) wherein R⁹ is phenyl substituted with 2 R^{e}.

Of Formula (IA) wherein R¹⁰ is phenyl substituted with 2 R^{e}.

The invention further includes a compound which is selected from the group:
N²-[(3,5-difluorophenyl)acetyl]-N¹-[(2R,3R)-2-(2,5-difluorophenyl)-4-oxo-2,3,4,5-tetrahydro-1,5-benzothiazepin-3-yl]-L-alaninamide;
N¹-[(2R,3R)-5-cyclohexyl-2-(2,5-difluorophenyl)-4-oxo-2,3,4,5-tetrahydro-1,5-benzothiazepin-3-yl]-N²-[(3,5-difluorophenyl)acetyl]-L-alaninamide;
N²-[(3,5-difluorophenyl)acetyl]-N¹-{(2R,3R)-2-(2,5-difluorophenyl)-5-[2-(dimethylamino)ethyl]-4-oxo-2,3,4,5-tetrahydro-1,5-benzothiazepin-3-yl}-L-alaninamide;
N²-[(3,5-difluorophenyl)acetyl]-N¹-[(2R,3R)-2-(2,5-difluorophenyl)-4-oxo-2,3,4,5-tetrahydro-1,5-benzothiazepin-3-yl]-L-serinamide;
N²-[(3,5-difluorophenyl)acetyl]-N¹-[(2R,3R)-2-(2,5-difluorophenyl)-5-methyl-4-oxo-2,3,4,5-tetrahydro-1,5-benzothiazepin-3-yl]-L-alaninamide;
N²-[(3,5-difluorophenyl)acetyl]-N¹-[(2R,3S)-4-oxo-2-phenyl-2,3,4,5-tetrahydro-1,5-benzoxazepin-3-yl]-L-alaninamide;
N²-[(3,5-difluorophenyl)acetyl]-N¹-[(6R,7R)-5-oxo-7-phenyl-1,4-thiazepan-6-yl]-L-alaninamide;
N²-[(3,5-difluorophenyl)acetyl]-N¹-[(3S,4R)-8-fluoro-2-oxo-4-phenyl-2,3,4,5-tetrahydro-1H-1-benzazepin-3-yl]-L-alaninamide;
N²-[(3,5-difluorophenyl)acetyl]-N¹-[(2R,3R)-4-oxo-2-phenyl-2,3,4,5-tetrahydro-1,5-benzothiazepin-3-yl]-L-alaninamide;
N¹-[(2R,3R)-2-(3,4-dichlorophenyl)-4-oxo-2,3,4,5-tetrahydro-1,5-benzothiazepin-3-yl]-N²-[(3,5-difluorophenyl)acetyl]-L-alaninamide;
N¹-[(2R,3R)-2-(4-chlorophenyl)-4-oxo-2,3,4,5-tetrahydro-1,5-benzothiazepin-3-yl]-N¹[(3,5-difluorophenyl)acetyl]-L-alaninamide;
N²-[(3,5-difluorophenyl)acetyl]-N¹-[(2R,3R)-2-(4-methylphenyl)-4-oxo-2,3,4,5-tetrahydro-1,5-benzothiazepin-3-yl]-L-alaninamide;
N¹-[(2R,3R)-7-chloro-4-oxo-2-phenyl-2,3,4,5-tetrahydro-1,5-benzothiazepin-3-yl]-N²-[(3,5-difluorophenyl)acetyl]-L-alaninamide;
N¹-{(2R,3R)-7-chloro-5-[2-(dimethylamino)ethyl]-4-oxo-2-phenyl-2,3,4,5-tetrahydro-1,5-benzothiazepin-3-yl}-N²-[(3,5-difluorophenyl)acetyl]-L-alaninamide;
N¹-[(2R,3R)-2-(3-chlorophenyl)-4-oxo-2,3,4,5-tetrahydro-1,5-benzothiazepin-3-yl]-N²-[(3,5-difluorophenyl)acetyl]-L-alaninamide;
N²-[(3,5-difluorophenyl)acetyl]-N¹-[(2R,3R)-2-(3,5-difluorophenyl)-4-oxo-2,3,4,5-tetrahydro-1,5-benzothiazepin-3-yl]-L-alaninamide;
N²-[(3,5-difluorophenyl)acetyl]-N¹-[(2R,3R)-2-(3,5-difluorophenyl)-5-methyl-4-oxo-2,3,4,5-tetrahydro-1,5-benzothiazepin-3-yl]-L-alaninamide;
N²-[(3,5-difluorophenyl)acetyl]-N¹-[(2R,3R)-2-(2-fluorophenyl)-5-methyl-4-oxo-2,3,4,5-tetrahydro-1,5-benzothiazepin-3-yl]-L-alaninamide;
N¹-{(2R,3R)-2-(3-chlorophenyl)-5-[2-(dimethylamino)ethyl]-4-oxo-2,3,4,5-tetrahydro-1,5-benzothiazepin-3-yl}-N²-[(3,5-difluorophenyl)acetyl]-L-alaninamide;
N²-[(3,5-difluorophenyl)acetyl]-N¹-[(2R,3R)-2-(2,5-difluorophenyl)-4-oxo-2,3,4,5-tetrahydro-1,5-benzothiazepin-3-yl]-D-serinamide;
N¹-[(2R,3R)-2-(3-chlorophenyl)-5-methyl-4-oxo-2,3,4,5-tetrahydro-1,5-benzothiazepin-3-yl]-N²-[(3,5-difluorophenyl)acetyl]-L-alaninamide;
N²-[(3,5-difluorophenyl)acetyl]-N¹-[(2R,3R)-5-methyl-4-oxo-2-phenyl-2,3,4,5-tetrahydro-1,5-benzothiazepin-3-yl]-L-alaninamide;
N¹-[(2R,3R)-5-cyclohexyl-4-oxo-2-phenyl-2,3,4,5-tetrahydro-1,5-benzothiazepin-3-yl]-N²-[(3,5-difluorophenyl)acetyl]-L-alaninamide;
N¹-[(2R,3R)-7-chloro-5-cyclohexyl-4-oxo-2-phenyl-2,3,4,5-tetrahydro-1,5-benzothiazepin-3-yl]-N²-[(3,5-difluorophenyl)acetyl]-L-alaninamide;
N²-[(3,5-difluorophenyl)acetyl]-N¹-[(6R,7R)-7-(1-naphthyl)-5-oxo-1,4-thiazepan-6-yl]-L-alaninamide;
(2S)-2-{[(3,5-difluorophenyl)acetyl]amino}-N-[(6R,7R)-7-(1-naphthyl)-5-oxo-1,4-thiazepan-6-yl]-2-phenylacetamide;
(2S)-2-hydroxy-4-methyl-N-((1S)-2-{[(6R,7R)-7-(1-naphthyl)-5-oxo-1,4-thiazepan-6-yl]amino}-2-oxo-1-phenylethyl)pentanamide;
(2S)-2-hydroxy-4-methyl-N-((1S)-2-oxo-2-{[(2R,3S)-4-oxo-2-phenyl-2,3,4,5-tetrahydro-1,5-benzoxazepin-3-yl]amino}-1-phenylethyl)pentanamide;
N²-[(2S)-2-hydroxy-4-methylpentanoyl]-N¹-[(2R,3S)-4-oxo-2-phenyl-2,3,4,5-tetrahydco-1,5-benzoxazepin-3-yl]-L-leucinamide;
N²-[(3,5-difluorophenyl)acetyl]-N¹-[(6S,7R)-4-methyl-5-oxo-7-phenyl-1,4-oxazepan-6-yl]-L-alaninamide;
N²-[(3,5-difluorophenyl)acetyl]-N¹-[(2S,6S,7R)-4-methyl-5-oxo-2,7-diphenyl-1,4-oxazepan-6-yl]-L-alaninamide;
N²-[(3,5-difluorophenyl)acetyl]-N¹-[(6R,7R)-4-methyl-5-oxo-7-phenyl-1,4-thiazepan-6-yl]-L-alaninamide;
N²-[(3,5-difluorophenyl)acetyl]-N¹-[(3R,6S,7R)-4-methyl-5-oxo-3,7-diphenyl-1,4-oxazepan-6-yl]-L-alaninamide;
(2S)-2-hydroxy-4-methyl-N-((1S)-2-{[(6S,7R)-4-methyl-5-oxo-7-phenyl-1,4-oxazepan-6-yl]amino}-2-oxo-1-phenylethyl)pentanamide;
(2S)-2-{[(3,5-difluorophenyl)acetyl]amino}-N-[(6S,7R)-4-methyl-5-oxo-7-phenyl-1,4-oxazepan-6-yl]-2-phenylacetamide;
(2S)-2-cyclohexyl-2-{[(3,5-difluorophenyl)acetyl]amino}-N-[(3R,6S,7R)-4-methyl-5-oxo-3,7-diphenyl-1,4-oxazepan-6-yl]acetamide;
(2S)-2-{[(3,5-difluorophenyl)acetyl]amino}-N-[(3R,6S,7R)-4-methyl-5-oxo-3,7-diphenyl-1,4-oxazepan-6-yl]-2-phenylacetamide;
N²-[(3,5-difluorophenyl)acetyl]-N¹-[(6S,7R)-4-(4-methoxybenzyl)-5-oxo-7-phenyl-1,4-oxazepan-6-yl]-L-alaninamide;
N²-[(3,5-difluorophenyl)acetyl]-N¹-[(2R,3S,5aR,9aR)-5-methyl-4-oxo-2-phenyldecahydro-1,5-benzoxazepin-3-yl]-L-alaninamide;
(2S)-2-{[(3,5-difluorophenyl)acetyl]amino}-N-[(6S,7R)-4-(4-methoxybenzyl)-5-oxo-7-phenyl-1,4-oxazepan-6-yl]-2-phenylacetamide;
N²-[(3,5-difluorophenyl)acetyl]-N¹-[(2R,3R)-2-(4-methoxyphenyl)-4-oxo-2,3,4,5-tetrahydro-1,5-benzothiazepin-3-yl]-L-alaninamide;
N¹-[(2R,3R)-7-chloro-2-(2,5-difluorophenyl)-4-oxo-2,3,4,5-tetrahydro-1,5-benzothiazepin-3-yl]-N²-[(2S)-2-(3,5-difluorophenyl)-2-hydroxyacetyl]-L-alaninamide;

1.66 A compound which is
N²-[(2S)-2-hydroxy-4-methyl-1-oxopentyl]-N¹-[(2R,3S)-4-oxo-2-phenyl-2,3,4,5-tetrahydro-1,5-benzoxazepin-3-yl]-L-alaninamide;
N²-[(3,5-difluorophenyl)acetyl]-N¹-[(2R,3S)-5-methyl-4-oxo-2-phenyl-2,3,4,5-tetrahydro-1,5-benzoxazepin-3-yl]-L-alaninamide;
N¹-[(2R,3R)-7-chloro-2-(2,5-difluorophenyl)-5-methyl-4-oxo-2,3,4,5-tetrahydro-1,5-benzothiazepin-3-yl]-N²-[(3,5-difluorophenyl)acetyl]-L-alaninamide;
N²-[(3,5-difluorophenyl)acetyl]-N¹-{(2R,35)-5-[2-(dimethylamino)ethyl]-4-oxo-2-phenyl-2,3,4,5-tetrahydro-1,5-benzoxazepin-3-yl}-L-alaninamide;
N¹-[(2R,3R)-7-chloro-2-(2,5-difluorophenyl)-4-oxo-5-phenyl-2,3,4,5-tetrahydro-1,5-benzothiazepin-3-yl]-N²-[(3,5-difluorophenyl)acetyl]-L-alaninamide;
N²-[(3,5-difluorophenyl)acetyl]-N¹-[(6R,7R)-5-oxo-7-phenyl-1,4-thiazepan-6-yl]-L-phenylalaninamide;
N²-[(2S)-2-hydroxy-4-methylpentanoyl]-N¹-[(6R,7R)-5-oxo-7-phenyl-1,4-thiazepan-6-yl]-L-phenylalaninamide;
(2S)-2-{[(3,5-difluorophenyl)acetyl]amino}-N-[(6R,7R)-5-oxo-7-phenyl-1,4-thiazepan-6-yl]-2-phenylacetamide;
(2S)-2-hydroxy-4-methyl-N-((1S)-2-oxo-2-{[(6R,7R)-5-oxo-7-phenyl-1,4-thiazepan-6-yl]amino}-1-phenylethyl)pentanamide;
N²-[(3,5-difluorophenyl)acetyl]-N¹-[(6R,7R)-5-oxo-7-phenyl-1,4-thiazepan-6-yl]-L-leucinamide;
N²-[(2S)-2-hydroxy-4-methylpentanoyl]-N¹-[(6R,7R)-5-oxo-7-phenyl-1,4-thiazepan-6-yl]-L-leucinamide;
N²-[(3,5-difluorophenyl)acetyl]-N¹-[(6R,7R)-5-oxo-7-phenyl-1,4-thiazepan-6-yl]-L-valinamide;
N²-[(2S)-2-hydroxy-4-methylpentanoyl]-N¹-[(6R,7R)-5-oxo-7-phenyl-1,4-thiazepan-6-yl]-L-valinamide;
N¹-[(2R,3S)-7-chloro-4-oxo-2-phenyl-2,3,4,5-tetrahydro-1,5-benzoxazepin-3-yl]-N²-[(3,5-difluorophenyl)acetyl]-L-alaninamide;
(2S)—N-((1S)-2-{[(2R,3S)-7-chloro-4-oxo-2-phenyl-2,3,4,5-tetrahydro-1,5-benzoxazepin-3-yl]amino}-2-oxo-1-phenylethyl)-2-hydroxy-4-methylpentanamide;
(2S)-2-{[(3,5-difluorophenyl)acetyl]amino}-N-[(2R,3S)-4-oxo-2-phenyl-2,3,4,5-tetrahydro-1,5-benzoxazepin-3-yl]-2-phenylacetamide;
N²-[(3,5-difluorophenyl)acetyl]-N¹-[(2R,3S)-4-oxo-2-phenyl-2,3,4,5-tetrahydro-1,5-benzoxazepin-3-yl]-L-serinamide;
(2S)-2-cyclohexyl-2-{[(3,5-difluorophenyl)acetyl]amino}-N-[(2R,3S)-4-oxo-2-phenyl-2,3,4,5-tetrahydro-1,5-benzoxazepin-3-yl]acetamide;
(2S)—N-((1S)-1-cyclohexyl-2-oxo-2-{[(2R,3S)-4-oxo-2-phenyl-2,3,4,5-tetrahydro-1,5-benzoxazepin-3-yl]amino}ethyl)-2-hydroxy-4-methylpentanamide;
3-cyclohexyl-N²-[(3,5-difluorophenyl)acetyl]-N¹-[(6R,7R)-5-oxo-7-phenyl-1,4-thiazepan-6-yl]-L-alaninamide;
N²-[(3,5-difluorophenyl)acetyl]-N¹-[(2R,3S)-5-(2-morpholin-4-ylethyl)-4-oxo-2-phenyl-2,3,4,5-tetrahydro-1,5-benzoxazepin-3-yl]-L-alaninamide;
N²-[(3,5-difluorophenyl)acetyl]-N¹-[(2R,3S)-4-oxo-2-phenyl-2,3,4,5-tetrahydro-1,5-benzoxazepin-3-yl]-L-leucinamide;
(2S)-2-{[(3,5-difluorophenyl)acetyl]amino}-2-(4-fluorophenyl)-N-[(2R,3S)-4-oxo-2-phenyl-2,3,4,5-tetrahydro-1,5-benzoxazepin-3-yl]acetamide;
(2S)-2-[(cyclohexylacetyl)amino]-2-(4-fluorophenyl)-N-[(2R,3S)-4-oxo-2-phenyl-2,3,4,5-tetrahydro-1,5-benzoxazepin-3-yl]acetamide;
N²-[(3,5-difluorophenyl)acetyl]-N¹-[(2R,3S)-4-oxo-2-phenyl-5-prop-2-yn-1-yl-2,3,4,5-tetrahydro-1,5-benzoxazepin-3-yl]-L-alaninamide;
N²-[(3,5-difluorophenyl)acetyl]-N¹-[(2R,3S)-7-methoxy-4-oxo-2-phenyl-2,3,4,5-tetrahydro-1,5-benzoxazepin-3-yl]-L-alaninamide;
N²-[(3,5-difluorophenyl)acetyl]-N¹-[(2R,3S)-5-isopropyl-4-oxo-2-phenyl-2,3,4,5-tetrahydro-1,5-benzoxazepin-3-yl]-L-alaninamide;
methyl [(2R,3S)-3-({N-[(3,5-difluorophenyl)acetyl]-L-alanyl}amino)-4-oxo-2-phenyl-3,4-dihydro-1,5-benzoxazepin-5(2H)-yl]acetate;
[(2R,3S)-3-({N-[(3,5-difluorophenyl)acetyl]-L-alanyl}amino)-4-oxo-2-phenyl-3,4-dihydro-1,5-benzoxazepin-5(2H)-yl]acetic acid;
N¹-[(2R,3S)-5-(cyclopropylmethyl)-4-oxo-2-phenyl-2,3,4,5-tetrahydro-1,5-benzoxazepin-3-yl]-N²-[(3,5-difluorophenyl)acetyl]-L-alaninamide;
N¹-[(2R,3S)-5-(cyclopropylmethyl)-7-methoxy-4-oxo-2-phenyl-2,3,4,5-tetrahydro-1,5-benzoxazepin-3-yl]-N²-[(3,5-difluorophenyl)acetyl]-L-alaninamide;
N¹-[(2R,3S)-5-(2-azetidin-1-yl-2-oxoethyl)-4-oxo-2-phenyl-2,3,4,5-tetrahydro-1,5-benzoxazepin-3-yl]-N²-[(3,5-difluorophenyl)acetyl]-L-alaninamide;
N²-[(3,5-difluorophenyl)acetyl]-N¹-[(2R,3S)-7-fluoro-4-oxo-2-phenyl-2,3,4,5-tetrahydro-1,5-benzoxazepin-3-yl]-L-alaninamide;
(2S)—N-((1S)-2-{[(2R,3S)-7-fluoro-4-oxo-2-phenyl-2,3,4,5-tetrahydro-1,5-benzoxazepin-3-yl]amino}-2-oxo-1-phenylethyl)-2-hydroxy-4-methylpentanamide;
N²-[(2R)-2-(3,5-difluorophenyl)-2-hydroxyacetyl]-N¹-[(2R,3S)-4-oxo-2-phenyl-2,3,4,5-tetrahydro-1,5-benzoxazepin-3-yl]-L-alaninamide;
N²-[(2S)-2-(3,5-difluorophenyl)-2-hydroxyacetyl]-N¹-[(2R,3S)-4-oxo-2-phenyl-2,3,4,5-tetrahydro-1,5-benzoxazepin-3-yl]-L-alaninamide;
N²-[(3,5-difluorophenyl)acetyl]-N¹-[(3S,4R)-2-oxo-4-phenyl-2,3,4,5-tetrahydro-1H-1-benzazepin-3-yl]-L-alaninamide;
N²-[(3,5-difluorophenyl)acetyl]-N¹-[(3S,4R)-8-fluoro-1-methyl-2-oxo-4-phenyl-2,3,4,5-tetrahydro-1H-1-benzazepin-3-yl]-L-alaninamide;
(2S)—N-((1S)-2-{[(3S,4R)-8-fluoro-2-oxo-4-phenyl-2,3,4,5-tetrahydro-1H-1-benzazepin-3-yl]amino}-2-oxo-1-phenylethyl)-2-hydroxy-4-methylpentanamide;
(2S)-2-hydroxy-4-methyl-N-((1S)-2-oxo-2-{[(3S,4R)-2-oxo-4-phenyl-2,3,4,5-tetrahydro-1H-1-benzazepin-3-yl]amino}-1-phenylethyl)pentanamide;
N²-[(3,5-difluorophenyl)acetyl]-N¹-[(3S,4R)-2-oxo-4-phenyl-1-prop-2-yn-1-yl-2,3,4,5-tetrahydro-1H-1-benzazepin-3-yl]-L-alaninamide;
N¹-[(3S,4R)-1-(cyclopropylmethyl)-2-oxo-4-phenyl-2,3,4,5-tetrahydro-1H-1-benzazepin-3-yl]-N²-[(3,5-difluorophenyl)acetyl]-L-alaninamide;
N²-[(3,5-difluorophenyl)acetyl]-N¹-[(3S,4R)-1-isopropyl-2-oxo-4-phenyl-2,3,4,5-tetrahydro-1H-1-benzazepin-3-yl]-L-alaninamide;
N²-[(2S)-2-hydroxy-4-methyl-1-oxopentyl]-N¹-[(2R,3R)-2-(4-methoxyphenyl)-4-oxo-2,3,4,5-tetrahydro-1,5-benzothiazepin-3-yl]-L-alaninamide;
N¹-[(2R,3R)-2-(2-chlorophenyl)-4-oxo-2,3,4,5-tetrahydro-1,5-benzothiazepin-3-yl]-N²-[(2S)-2-hydroxy-4-methyl-1-oxopentyl]-L-alaninamide;
N¹-[(2R,3R)-2-(2-chlorophenyl)-4-oxo-2,3,4,5-tetrahydro-1,5-benzothiazepin-3-yl]-N²-[(3,5-difluorophenyl)acetyl]-L-alaninamide;
N¹-[(2R,3R)-7-chloro-5-methyl-4-oxo-2-phenyl-2,3,4,5-tetrahydro-1,5-benzothiazepin-3-yl]-N-[(3,5-difluorophenyl)acetyl]-L-alaninamide;
N²-[(3,5-difluorophenyl)acetyl]-N¹-[(2R,3R)-2-(2-fluorophenyl)-4-oxo-2,3,4,5-tetrahydro-1,5-benzothiazepin-3-yl]-L-alaninamide;
N²-[(3,5-difluorophenyl)acetyl]-N¹-[(2R,3R)-2-(4-fluorophenyl)-4-oxo-2,3,4,5-tetrahydro-1,5-benzothiazepin-3-yl]-L-alaninamide;
N¹-[(2R,3R)-7-chloro-2-(2,5-difluorophenyl)-4-oxo-2,3,4,5-tetrahydro-1,5-benzothiazepin-3-yl]-N-[(3,5-difluorophenyl)acetyl]-L-alaninamide;
N²-[(2S)-2-hydroxy-4-methyl-1-oxopentyl]-N¹-[(6R,7R)-5-oxo-7-phenyl-1,4-thiazepan-6-yl]-L-alaninamide;
N²-[(3,5-difluorophenyl)acetyl]-N¹-[(2S,3R)-2-(3-methyl-2-thienyl)-4-oxo-2,3,4,5-tetrahydro-1,5-benzothiazepin-3-yl]-L-alaninamide;
N²-[(3,5-difluorophenyl)acetyl]-N¹-[(2S,3R)-2-(4-methyl-2-thienyl)-4-oxo-2,3,4,5-tetrahydro-1,5-benzothiazepin-3-yl]-L-alaninamide;
Methyl 5-[(2S,3R)-3-({N-[(3,5-difluorophenyl)acetyl]-L-alanyl}amino)-4-oxo-2,3,4,5-tetrahydro-1,5-benzothiazepin-2-yl]thiophene-3-carboxylate;
N¹-[(2R,3R)-4-oxo-2-phenyl-2,3,4,5-tetrahydro-1,5-benzothiazepin-3-yl]-N²-(phenylacetyl)-L-alaninamide;
N¹-[(2R,3R)-4-oxo-2-phenyl-2,3,4,5-tetrahydro-1,5-benzothiazepin-3-yl]-N²-(2-phenylethyl)-L-alaninamide;
N²-[(3,5-difluorophenyl)acetyl]-N¹-[(2S,3R)-4-oxo-2-(2-thienyl)-2,3,4,5-tetrahydro-1,5-benzothiazepin-3-yl]-L-alaninamide;
N²-[(3,5-difluorophenyl)acetyl]-N¹-[(2R,3R)-4-oxo-2-(3-thienyl)-2,3,4,5-tetrahydro-1,5-benzothiazepin-3-yl]-L-alaninamide;
N²-[(3,5-difluorophenyl)acetyl]-N¹-[(2S,3R)-2-(2-furyl)-4-oxo-2,3,4,5-tetrahydro-1,5-benzothiazepin-3-yl]-L-alaninamide;
N²-[(3,5-difluorophenyl)acetyl]-N¹-[(2R,3R)-2-(3-furyl)-4-oxo-2,3,4,5-tetrahydro-1,5-benzothiazepin-3-yl]-L-alaninamide;
N¹-[(2S,3R)-2-(5-bromo-2-thienyl)-4-oxo-2,3,4,5-tetrahydro-1,5-benzothiazepin-3-yl]-N²-[(3,5-difluorophenyl)acetyl]-L-alaninamide;
N¹-[(2S,3R)-2-(4-bromo-2-thienyl)-4-oxo-2,3,4,5-tetrahydro-1,5-benzothiazepin-3-yl]-N²-[(3,5-difluorophenyl)acetyl]-L-alaninamide;
N-[(3,5-difluorophenyl)acetyl]-N-[(2R,3R)-4-oxo-2-phenyl-2,3,4,5-tetrahydro-1,5-benzothiazepin-3-yl]-L-phenylalaninamide;
N²-[(3,5-difluorophenyl)acetyl]-N¹-[(2R,3R)-4-oxo-2-phenyl-2,3,4,5-tetrahydro-1,5-benzothiazepin-3-yl]glycinamide;
N²-[(3,5-difluorophenyl)acetyl]-N¹-[(2R,3R)-4-oxo-2-phenyl-2,3,4,5-tetrahydro-1,5-benzothiazepin-3-yl]-L-valinamide;
N²-[(3,5-difluorophenyl)acetyl]-N¹-[(2R,3R)-4-oxo-2-phenyl-2,3,4,5-tetrahydro-1,5-benzothiazepin-3-yl]-L-leucinamide;
N²-[(3,5-difluorophenyl)acetyl]-N¹-[(2R,3R)-4-oxo-2-phenyl-2,3,4,5-tetrahydro-1,5-benzothiazepin-3-yl]-L-methioninamide;
N²-[(3,5-difluorophenyl)acetyl]-3-(1H-indol-2-yl)-N¹-[(2R,3R)-4-oxo-2-phenyl-2,3,4,5-tetrahydro-1,5-benzothiazepin-3-yl]-L-alaninamide;
N²-[(3,5-difluorophenyl)acetyl]-N¹-[(2R,3R)-4-oxo-2-phenyl-2,3,4,5-tetrahydro-1,5-benzothiazepin-3-yl]-L-a-asparagine;
N²-[(3,5-difluorophenyl)acetyl]-N¹-[(2R,3R)-4-oxo-2-phenyl-2,3,4,5-tetrahydro-1,5-benzothiazepin-3-yl]-L-a-glutamine;
N¹-[(2R,3S)-4-oxo-2-phenyl-2,3,4,5-tetrahydro-1,5-benzoxazepin-3-yl]-N²-(phenylacetyl)-L-alaninamide;
N²-[(2-fluorophenyl)acetyl]-N¹-[(2R,3S)-4-oxo-2-phenyl-2,3,4,5-tetrahydro-1,5-benzoxazepin-3-yl]-L-alaninamide;
N²-[(3-fluorophenyl)acetyl]N¹-[(2R,3S)-4-oxo-2-phenyl-2,3,4,5-tetrahydro-1,5-benzoxazepin-3-yl]-L-alaninamide;
N²-[(4-fluorophenyl)acetyl]-N¹-[(2R,3S)-4-oxo-2-phenyl-2,3,4,5-tetrahydro-1,5-benzoxazepin-3-yl]-L-alaninamide;
N¹-[(2R,3S,5aS,9aS)-5-(cyclopropylmethyl)-4-oxo-2-phenyldecahydro-1,5-benzoxazepin-3-yl]-N²-[(3,5-difluorophenyl)acetyl]-L-alaninamide.

The invention further includes a method for the treatment of disorders associated with activation of the Notch signal transduction pathway comprising administering a therapeutically effective amount of a compound of Formula (I):

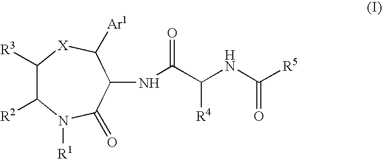

wherein:

X is CH₂, O, NR¹, SO₂ or S;

Ar¹ is a 5- or 6-membered ring optionally substituted with 0, 1, 2, or 3 R^{e} moieties, said ring having 0, 1, 2 or 3 nitrogen, oxygen or sulfur atoms, but no more than 2 oxygen atoms or 2 sulfur atoms or 1 oxygen and 1 sulfur atom;

R¹ is H, —C₁₋₃alkylC₃₋₆cycloalkyl, C₁₋₆alkyl, C₃₋₆alkenyl, C₃₋₆alkynyl, C₃₋₆cycloalkyl, C₂₋₄alkylNR^{a}R^{b}, C₁₋₄alkylC(═O)R^{d}; or C₁₋₃alkylphenyl substituted with 0, 1, 2 or 3 R^{e};

R^{a} and R^{b} are at each occurrence independently selected from H, C₁₋₄alkyl or C₃₋₆cycloalkyl, or R^{a} and R^{b} and the N to which they are attached in combination form a 5 or 6-membered N-linked heterocycle having 2 nitrogen atoms, wherein the non-linked nitrogen is substituted with R^{c} or 1 nitrogen and 1 oxygen, ring atoms wherein there is no non-linked nitrogen;

R^{c} is, at each occurrence independently selected from H, C₁₋₃alkyl, or phenyl substituted with 0, 1, 2, or 3 R^{e};

R^{d} is, at each occurrence independently selected from C₁₋₃alkyl, hydroxy, C₁₋₃alkoxy, or NR^{a}R^{b};

R^{e} is, at each occurrence independently selected from H, OH, F, Cl, Br, I, CN, NO₂, CF₃, C₁₋₆alkyl, or C₁₋₆alkoxy;

R² and R³ are at each occurrence independently selected from H, C₁₋₆alkyl, C₄₋₆cycloalkyl, aryl, or heteroaryl, or R² and R³ in combination form a fused phenyl or cyclohexyl moiety that may be substituted with 0, 1 or 2 R^{f} moieties,

R^{f} is NO₂, F, Cl, Br, I, CF₃, CN, C₁₋₆alkyl, or C₁₋₆alkoxy;

R⁴ is H, CHR⁷R⁸, 5- or 6-membered cycloalkyl, 5- or 6-membered ring optionally substituted with 0, 1, or 2 R^{f} moieties, said ring having 0, 1, 2 or 3 nitrogen, oxygen or sulfur atoms, but no more than 2 oxygen atoms or 2 sulfur atoms or 1 oxygen and 1 sulfur atom;

R⁵ is C₁₋₃alkylR⁹ or CH(OH)R¹⁰;

R⁷ and R⁸ are, at each occurrence are independently selected from H, C₁₋₄alkyl, OH, SH, CH₂SCH₃, CONH₂, CH₂CONH₂, CO₂H, CH₂CO₂H, (CH₂)₃NHCH(NH₂)₂, C₁₋₄alkylamino, indolyl, imidazolyl, phenyl or hydroxyphenyl or R⁷ and R⁸ in combination form a 6-membered ring optionally substituted with 0, 1 or 2 R^{f} moieties said heterocyclic ring having 0, 1, 2 or 3 nitrogen, oxygen or sulfur atoms, but no more than 2 oxygen atoms or 2 sulfur atoms or 1 oxygen and 1 sulfur atom;

R⁹ is phenyl substituted with 0, 1, 2 or 3 R^{e};

R¹⁰ is C₁₋₆alkyl or R⁹;

in free form or in a pharmaceutically acceptable salt form or in the form of a pharmaceutically acceptable solvate of the compound or the salt, to a human or animal patient in need thereof.

X is CH₂, O, SO₂ or S.

X is S, O, or CH₂.

Ar¹ is a 5- or 6-membered ring optionally substituted with 0 or 1 R^{e}.

Ar¹ is a 6-membered aromatic ring optionally substituted with 1, 2 R^{e} moieties wherein R^{e} is F or Cl, C₁₋₆alkyl, or C₁₋₆alkoxy, or Ar¹ is a 5-membered heterocyclic ring optionally substituted with 1 R^{e} moiety wherein R^{e} is F, Cl, Br, C₁₋₆alkyl.

Ar¹ is a 6-membered aromatic ring optionally substituted with 1, 2 R^{e} moieties wherein R^{e} is F or Cl, methyl, or methoxy, or Ar¹ is a 5-membered heterocyclic ring optionally substituted with 1 R^{e} moiety wherein R^{e} is F, Cl, Br, methyl.

Ar¹ is a phenyl optionally substituted with 1, 2 R^{e} moieties wherein R^{e} is F or Cl, methyl, or methoxy, or Ar¹ is a furyl, thienyl optionally substituted with 1 R^{e} moiety wherein R^{e} is F, Cl, Br, methyl.

R¹ is H, —C₁₋₃alkylC₃₋₆cycloalkyl, C₁₋₆alkyl, C₃₋₆alkenyl, C₃₋₆alkynyl.

R¹ is H, C₂₋₄alkyl b, C₁₋₆alkyl, C₃₋₆cycloalkyl, C₃₋₆alkynyl, —C₄alkylC(═O)C₁₋₃alkoxy, —C₁₋₃alkylC₃₋₆cycloalkyl, or C₁₋₃alkylphenyl substituted with C₁₋₆alkoxy.

R¹ is H, dimethylaminoethyl, 2-morpholinoethyl, methyl or isopropyl, cyclohexyl, 2-propyn-1-yl, methoxycarbonylmethyl, carboxymethyl, cyclopropylmethyl, or 4-methoxybenzyl.

R² and R³ are each H, or combined to form a fused phenyl moiety substituted with F, Cl, or C₁₋₆alkoxy, or combined to form a cyclohexyl.

R² and R³ are each H, or combined to form a fused phenyl moiety substituted with F, Cl, or methoxy, or combined to form a cyclohexyl.

R⁴ is H, CHR⁷R⁸ wherein R⁷ and R⁸ is H, C₁₋₄alkyl, CH₂CH₂SCH₃, CO₂H, or CH₂CO₂H, OH, or a 6-membered aromatic ring optionally substituted with 1 F, or a 6-membered cycloalkyl.

R⁴ is H, methyl, benzyl, isopropyl, isopropylmethyl, indol-2ylmethyl, CH₂CH₂SCH₃, or CH₂CO₂H, CH₂CH₂CO₂H, CH₂OH, or phenyl optionally substituted with 1 F, or cyclohexyl.

R⁵ is C₁₋₃alkylR⁹ wherein R⁹ is a phenyl substituted with 2 F or is CH(OH)R¹⁰ wherein R¹⁰ is C₄alkyl or R⁹ wherein R⁹ is phenyl optionally substituted with 0, 1, or 2 F.

R⁵ is benzyl, 1-hydroxy-3-methylbutyl, α-hydroxy-3,5-difluorobenzyl, 3,5-difluorobenzyl or 3-5-difluorophenyl, 2-fluorobenzyl, 3-fluorobenzyl, or 4-fluorobenzyl,

In a further aspect of the invention, there is provided a compound of Formula (I) in free form or a pharmaceutically acceptable salt thereof, wherein:

X is CH₂, O, or S;

Ar¹ is a 6-membered aromatic ring optionally substituted with 1, 2 R^{e} moieties wherein R^{e} is F or Cl, C₁₋₆alkyl, or C₁₋₆alkoxy, or Ar¹ is a 5-membered heterocyclic ring optionally substituted with 1 R^{e} moiety wherein R^{e} is F, Cl, Br, C₁₋₆alkyl;

R¹ is H, C₂₋₄alkylNR^{a}R^{b}, C₁₋₆alkyl, C₃₋₆cycloalkyl, C₃₋₆alkynyl, —C₁₋₄alkylC(═O)C₁₋₃alkoxy, —C₁₋₃alkylC₃₋₆cycloalkyl, or C₁₋₃alkylphenyl substituted with C₁₋₆alkoxy;

R² and R³ are each H, or combined to form a fused phenyl moiety substituted with F, Cl, or C₁₋₆alkoxy, or combined to form a cyclohexyl;

R⁴ is H, CHR⁷R⁸ wherein R⁷ and R⁸ is H, C₁₋₄alkyl, CH₂CH₂SCH₃, CO₂H, or CH₂CO₂H, OH, or a 6-membered aromatic ring optionally substituted with 1 F, or a 6-membered cycloalkyl; and

R⁵ is C₁₋₃alkylR⁹ wherein R⁹ is a phenyl substituted with 2 F or is CH(OH)R¹⁰ wherein R¹⁰ is C₄alkyl or R⁹ wherein R⁹ is phenyl optionally substituted with 0, 1, or 2 F.

In a further aspect of the invention, there is provided a compound of Formula (I) in free form or a pharmaceutically acceptable salt thereof, wherein:

X is S, O, or CH₂;

Ar¹ is a phenyl optionally substituted with 1, 2 R^{e} moieties wherein R^{e} is F or Cl, methyl, or methoxy, or Ar¹ is a furyl, thienyl optionally substituted with 1 R^{e} moiety wherein R^{e} is F, Cl, Br, methyl;

R¹ is H, dimethylaminoethyl, 2-morpholinoethyl, methyl or isopropyl, cyclohexyl, 2-propyn-1-yl, methoxycarbonylmethyl, carboxymethyl, cyclopropylmethyl, or 4-methoxybenzyl;

R² and R³ are each H, or combined to form a fused phenyl moiety substituted with F, Cl, or methoxy, or combined to form a cyclohexyl;

R⁴ is H, methyl, benzyl, isopropyl, isopropylmethyl, indol-2ylmethyl, CH₂CH₂SCH₃, or CH₂CO₂H, CH₂CH₂CO₂H, CH₂OH, or phenyl optionally substituted with 1 F, or cyclohexyl; and

R⁵ is benzyl, 1-hydroxy-3-methylbutyl, α-hydroxy-3,5-difluorobenzyl, 3,5-difluorobenzyl or 3-5-difluorophenyl, 2-fluorobenzyl, 3-fluorobenzyl, or 4-fluorobenzyl.

A compound of Formula I which is a compound of Formula (1A) in free or pharmaceutically acceptable salt form:

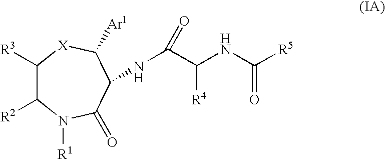

A compound of Formula 1 which is a compound of Formula (1B) in free or pharmaceutically acceptable salt form:

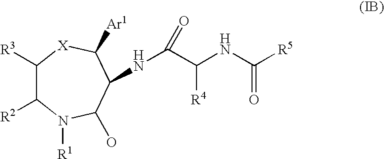

A compound of Formula 1 which is a compound of Formula (1C) in free or pharmaceutically acceptable salt form:

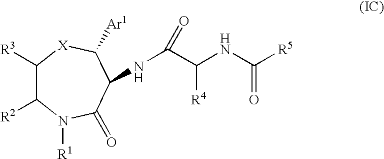

A compound of Formula 1 which is a compound of Formula (1D) in free or pharmaceutically acceptable salt form:

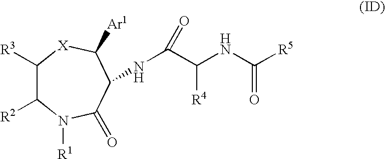

A compound of Formula 1 which is a compound of Formula (1E) in free or pharmaceutically acceptable salt form:

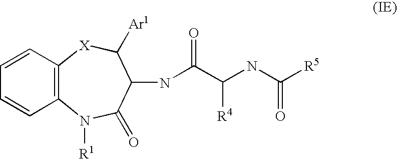

A compound of Formula 1 which is a compound of Formula (1F) in free or pharmaceutically acceptable salt form:

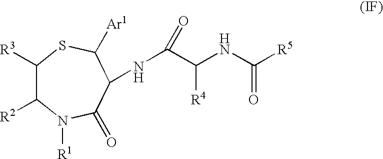

A compound of Formula 1 which is a compound of Formula (1G) in free or pharmaceutically acceptable salt form:

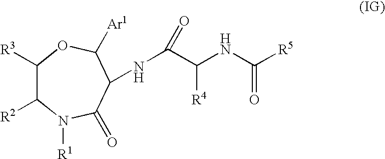

A compound of Formula 1 which is a compound of Formula (1H) in free or pharmaceutically acceptable salt form:

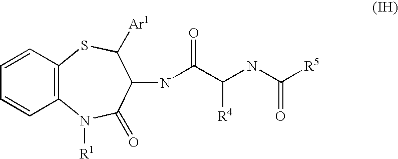

A compound of Formula 1 which is a compound of Formula (1I) in free or pharmaceutically acceptable salt form:

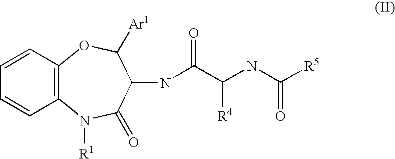

A compound of Formula 1 which is a compound of Formula (1J) in free or pharmaceutically acceptable salt form:

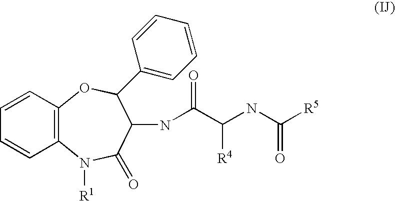

A compound of Formula 1 which is a compound of Formula (1K) in free or pharmaceutically acceptable salt form:

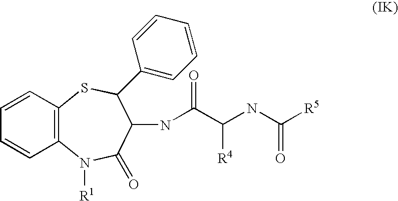

A compound of Formula 1 which is a compound of Formula (1L) in free or pharmaceutically acceptable salt form:

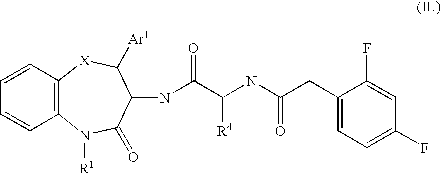

The invention further includes the use of a compound selected from:
N²-[(3,5-difluorophenyl)acetyl]-N¹-[(2R,3R)-2-(2,5-difluorophenyl)-4-oxo-2,3,4,5-tetrahydro-1,5-benzothiazepin-3-yl]-L-alaninamide;
N¹-[(2R,3R)-5-cyclohexyl-2-(2,5-difluorophenyl)-4-oxo-2,3,4,5-tetrahydro-1,5-benzothiazepin-3-yl]-N²-[(3,5-difluorophenyl)acetyl]-L-alaninamide;
N²-[(3,5-difluorophenyl)acetyl]-N¹-{(2R,3R)-2-(2,5-difluorophenyl)-5-[2-(dimethylamino)ethyl]-4-oxo-2,3,4,5-tetrahydro-1,5-benzothiazepin-3-yl}-L-alaninamide;
N²-[(3,5-difluoroplenyl)acetyl]-N-[(2R,3R)-2-(2,5-difluorophenyl)-4-oxo-2,3,4,5-tetrahydro-1,5-benzothiazepin-3-yl]-L-serinamide;

N²-[(3,5-difluorophenyl)acetyl]-N¹-[(2R,3R)-2-(2,5-difluorophenyl)-5-methyl-4-oxo-2,3,4,5-tetrahydro-1,5-benzothiazepin-3-yl]-L-alaninamide;
N²-[(3,5-difluorophenyl)acetyl]-N¹-[(2R,3S)-4-oxo-2-phenyl-2,3,4,5-tetrahydro-1,5-benzoxazepin-3-yl]-L-alaninamide;
N¹-[(2R,3R)-5-cyclohexyl-4-oxo-2-phenyl-2,3,4,5-tetrahydro-1,5-benzothiazepin-3-yl]-N²-[(3,5-difluorophenyl)acetyl]-L-alaninamide;
N²-[(3,5-difluorophenyl)acetyl]-N¹-[(6R,7R)-5-oxo-7-phenyl-1,4-thiazepan-6-yl]-L-alaninamide;
N²-[(3,5-difluorophenyl)acetyl]-N¹-[(3S,4R)-8-fluoro-2-oxo-4-phenyl-2,3,4,5-tetrahydro-1H-1-benzazepin-3-yl]-L-alaninamide;
N²-[(3,5-difluorophenyl)acetyl]-N¹-[(2R,3R)-4-oxo-2-phenyl-2,3,4,5-tetrahydro-1,5-benzothiazepin-3-yl]-L-alaninamide;
N¹-[(2R,3R)-2-(3,4-dichlorophenyl)-4-oxo-2,3,4,5-tetrahydro-1,5-benzothiazepin-3-yl]-N²-[(3,5-difluorophenyl)acetyl]-L-alaninamide;
N¹-[(2R,3R)-2-(4-chlorophenyl)-4-oxo-2,3,4,5-tetrahydro-1,5-benzothiazepin-3-yl]-N²-[(3,5-difluorophenyl)acetyl]-L-alaninamide;
N²-[(3,5-difluorophenyl)acetyl]-N¹-[(2R,3R)-2-(4-methylphenyl)-4-oxo-2,3,4,5-tetrahydro-1,5-benzothiazepin-3-yl]-L-alaninamide;
N¹-[(2R,3R)-7-chloro-4-oxo-2-phenyl-2,3,4,5-tetrahydro-1,5-benzothiazepin-3-yl]-N²-[(3,5-difluorophenyl)acetyl]-L-alaninamide;
N¹-{(2R,3R)-7-chloro-5-[2-(dimethylamino)ethyl]-4-oxo-2-phenyl-2,3,4,5-tetrahydro-1,5-benzothiazepin-3-yl}-N²-[(3,5-difluorophenyl)acetyl]-L-alaninamide;
N¹-[(2R,3R)-2-(3-chlorophenyl)-4-oxo-2,3,4,5-tetrahydro-1,5-benzothiazepin-3-yl]-N²-[(3,5-difluorophenyl)acetyl]-L-alaninamide;
N²-[(3,5-difluorophenyl)acetyl]-N¹-[(2R,3R)-2-(3,5-difluorophenyl)-4-oxo-2,3,4,5-tetrahydro-1,5-benzothiazepin-3-yl]-L-alaninamide;
N²-[(3,5-difluorophenyl)acetyl]-N¹-[(2R,3R)-2-(3,5-difluorophenyl)-5-methyl-4-oxo-2,3,4,5-tetrahydro-1,5-benzothiazepin-3-yl]-L-alaninamide;
N²-[(3,5-difluorophenyl)acetyl]-N¹-[(2R,3R)-2-(2-fluorophenyl)-5-methyl-4-oxo-2,3,4,5-tetrahydro-1,5-benzothiazepin-3-yl]-L-alaninamide;
N¹-{(2R,3R)-2-(3-chlorophenyl)-5-[2-(dimethylamino)ethyl]-4-oxo-2,3,4,5-tetrahydro-1,5-benzothiazepin-3-yl}-N²-[(3,5-difluorophenyl)acetyl]-L-alaninamide;
N²-[(3,5-difluorophenyl)acetyl]-N¹-[(2R,3R)-2-(2,5-difluorophenyl)-4-oxo-2,3,4,5-tetrahydro-1,5-benzothiazepin-3-yl]-D-serinamde;
N¹-[(2R,3R)-2-(3-chlorophenyl)-5-methyl-4-oxo-2,3,4,5-tetrahydro-1,5-benzothiazepin-3-yl]-N²-[(3,5-difluorophenyl)acetyl]-L-alaninamide;
N²-[(3,5-difluorophenyl)acetyl]-N¹-[(2R,3R)-5-methyl-4-oxo-2-phenyl-2,3,4,5-tetrahydro-1,5-benzothiazepin-3-yl]-L-alaninamide;
N¹-[(2R,3R)-7-chloro-5-cyclohexyl-4-oxo-2-phenyl-2,3,4,5-tetrahydro-1,5-benzothiazepin-3-yl]-N²-[(3,5-difluorophenyl)acetyl]-L-alaninamide;
(2S)-2-hydroxy-4-methyl-N-((1S)-2-oxo-2-{[(2R,3S)-4-oxo-2-phenyl-2,3,4,5-tetrahydro-1,5-benzoxazepin-3-yl]amino}-1-phenylethyl)pentanamide;
N²-[(2S)-2-hydroxy-4-methylpentanoyl]-N¹-[(2R,3S)-4-oxo-2-phenyl-2,3,4,5-tetrahydro-1,5-benzoxazepin-3-yl]-L-leucinamide;
N²-[(3,5-difluorophenyl)acetyl]-N¹-[(6S,7R)-4-methyl-5-oxo-7-phenyl-1,4-oxazepan-6-yl]-L-alaninamide;
N²-[(3,5-difluorophenyl)acetyl]-N¹-[(2S,6S,7R)-4-methyl-5-oxo-2,7-diphenyl-1,4-oxazepan-6-yl]-L-alaninamide;
N²-[(3,5-difluorophenyl)acetyl]-N¹-[(6R,7R)-4-methyl-5-oxo-7-phenyl-1,4-thiazepan-6-yl]-L-alaninamide;
N²-[(3,5-difluorophenyl)acetyl]-N¹-[(3R,6S,7R)-4-methyl-5-oxo-3,7-diphenyl-1,4-oxazepan-6-yl]-L-alaninamide;
(2S)-2-hydroxy-4-methyl-N-((1S)-2-{[(6S,7R)-4-methyl-5-oxo-7-phenyl-1,4-oxazepan-6-yl]amino}-2-oxo-1-phenylethyl)pentanamide;
(2S)-2-{[(3,5-difluorophenyl)acetyl]amino}-N-[(6S,7R)-4-methyl-5-oxo-7-phenyl-1,4-oxazepan-6-yl]-2-phenylacetamide;
(2S)-2-cyclohexyl-2-{[(3,5-difluorophenyl)acetyl]amino}-N-[(3R,6S,7R)-4-methyl-5-oxo-3,7-diphenyl-1,4-oxazepan-6-yl]acetamide;
(2S)-2-{[(3,5-difluorophenyl)acetyl]amino}-N-[(3R,6S,7R)-4-methyl-5-oxo-3,7-diphenyl-1,4-oxazepan-6-yl]-2-phenylacetamide;
N²-[(3,5-difluorophenyl)acetyl]-N¹-[(6S,7R)-4-(4-methoxybenzyl)-5-oxo-7-phenyl-1,4-oxazepan-6-yl]-L-alaninamide;
N²-[(3,5-difluorophenyl)acetyl]-N-[(2R,3S,5aR,9aR)-5-methyl-4-oxo-2-phenyldecahydro-1,5-benzoxazepin-3-yl]-L-alaninamide;
(2S)-2-{[(3,5-difluorophenyl)acetyl]amino}-N-[(6S,7R)-4-(4-methoxybenzyl)-5-oxo-7-phenyl-1,4-oxazepan-6-yl]-2-phenylacetamide;
N²-[(3,5-difluorophenyl)acetyl]-N¹-[(2R,3R)-2-(4-methoxyphenyl)-4-oxo-2,3,4,5-tetrahydro-1,5-benzothiazepin-3-yl]-L-alaninamide;
N¹-[(2R,3R)-7-chloro-2-(2,5-difluorophenyl)-4-oxo-2,3,4,5-tetrahydro-1,5-benzothiazepin-3-yl]-N²-[(2S)-2-(3,5-difluorophenyl)-2-hydroxyacetyl]-L-alaninamide;
N²-[(2S)-2-hydroxy-4-methyl-1-oxopentyl]-N¹-[(2R,3S)-4-oxo-2-phenyl-2,3,4,5-tetrahydro-1,5-benzoxazepin-3-yl]-L-alaninamide;
N²-[(3,5-difluorophenyl)acetyl]-N¹-[(2R,3S)-5-methyl-4-oxo-2-phenyl-2,3,4,5-tetrahydro-1,5-benzoxazepin-3-yl]-L-alaninamide;
N¹-[(2R,3R)-7-chloro-2-(2,5-difluorophenyl)-5-methyl-4-oxo-2,3,4,5-tetrahydro-1,5-benzothiazepin-3-yl]-N-[(3,5-difluorophenyl)acetyl]-L-alaninamide;
N²-[(3,5-difluorophenyl)acetyl]-N¹-{(2R,3S)-5-[2-(dimethylamino)ethyl]-4-oxo-2-phenyl-2,3,4,5-tetrahydro-1,5-benzoxazepin-3-yl}-L-alaninamide;
N²-[(3,5-difluorophenyl)acetyl]-N¹-[(6R,7R)-5-oxo-7-phenyl-1,4-thiazepan-6-yl]-L-phenylalaninamide;
N²-[(2S)-2-hydroxy-4-methylpentanoyl]-N¹-[(6R,7R)-5-oxo-7-phenyl-1,4-thiazepan-6-yl]-L-phenylalaninamide;
(2S)-2-{[(3,5-difluorophenyl)acetyl]amino}-N-[(6R,7R)-5-oxo-7-phenyl-1,4-thiazepan-6-yl]-2-phenylacetamide;
(2S)-2-hydroxy-4-methyl-N-((1S)-2-oxo-2-{[(6R,7R)-5-oxo-7-phenyl-1,4-thiazepan-6-yl]amino}-1-phenylethyl)pentanamide;
N²-[(3,5-difluorophenyl)acetyl]-N¹-[(6R,7R)-5-oxo-7-phenyl-1,4-thiazepan-6-yl]-L-leucinamide;
N²-[(2S)-2-hydroxy-4-methylpentanoyl]-N¹-[(6R,7R)-5-oxo-7-phenyl-1,4-thiazepan-6-yl]-L-leucinamide;
N²-[(3,5-difluorophenyl)acetyl]-N¹-[(6R,7R)-5-oxo-7-phenyl-1,4-thiazepan-6-yl]-L-valinamide;
N²-[(2S)-2-hydroxy-4-methylpentanoyl]-N¹-[(6R,7R)-5-oxo-7-phenyl-1,4-thiazepan-6-yl]-L-valinamide;
N¹-[(2R,3S)-7-chloro-4-oxo-2-phenyl-2,3,4,5-tetrahydro-1,5-benzoxazepin-3-yl]-N²-[(3,5-difluorophenyl)acetyl]-L-alaninamide;
(2S)—N-((1S)-2-{[(2R,3S)-7-chloro-4-oxo-2-phenyl-2,3,4,5-tetrahydro-1,5-benzoxazepin-3-yl]amino}-2-oxo-1-phenylethyl)-2-hydroxy-4-methylpentanamide;
(2S)-2-{[(3,5-difluorophenyl)acetyl]amino}-N-[(2R,3S)-4-oxo-2-phenyl-2,3,4,5-tetrahydro-1,5-benzoxazepin-3-yl]-2-phenylacetamide;
N²-[(3,5-difluorophenyl)acetyl]-N¹-[(2R,3S)-4-oxo-2-phenyl-2,3,4,5-tetrahydro-1,5-benzoxazepin-3-yl]-L-serinamide;
(2S)-2-cyclohexyl-2-{[(3,5-difluorophenyl)acetyl]amino}-N-[(2R,3S)-4-oxo-2-phenyl-2,3,4,5-tetrahydro-1,5-benzoxazepin-3-yl]acetamide;
(2S)—N-((1S)-1-cyclohexyl-2-oxo-2-{[(2R,3S)-4-oxo-2-phenyl-2,3,4,5-tetrahydro-1,5-benzoxazepin-3-yl]amino}ethyl)-2-hydroxy-4-methylpentanamide;
3-cyclohexyl-N²-[(3,5-difluorophenyl)acetyl]-N¹-[(6R,7R)-5-oxo-7-phenyl-1,4-thiazepan-6-yl]-L-alaninamide;
N²-[(3,5-difluorophenyl)acetyl]-N⁴-[(2R,3S)-5-(2-morpholin-4-ylethyl)-4-oxo-2-phenyl-2,3,4,5-tetrahydro-1,5-benzoxazepin-3-yl]-L-alaninamide;
N²-[(3,5-difluorophenyl)acetyl]-N¹-[(2R,3S)-4-oxo-2-phenyl-2,3,4,5-tetrahydro-1,5-benzoxazepin-3-yl]-L-leucinamide;
(2S)-2-{[(3,5-difluorophenyl)acetyl]amino}-2-(4-fluorophenyl)-N-[(2R,3S)-4-oxo-2-phenyl-2,3,4,5-tetrahydro-1,5-benzoxazepin-3-yl]acetamide;
N²-[(3,5-difluorophenyl)acetyl]-N¹-[(2R,3S)-4-oxo-2-phenyl-5-prop-2-yl-1-yl-2,3,4,5-tetrahydro-1,5-benzoxazepin-3-yl]-L-alaninamide;
N²-[(3,5-difluorophenyl)acetyl]-N¹-[(2R,3S)-7-methoxy-4-oxo-2-phenyl-2,3,4,5-tetrahydro-1,5-benzoxazepin-3-yl]-L-alaninamide;
N²-[(3,5-difluorophenyl)acetyl]-N¹-[(2R,3S)-5-isopropyl-4-oxo-2-phenyl-2,3,4,5-tetrahydro-1,5-benzoxazepin-3-yl]-L-alaninamide;
methyl [(2R,3S)-3-({N-[(3,5-difluorophenyl)acetyl]-L-alanyl}amino)-4-oxo-2-phenyl-3,4-dihydro-1,5-benzoxazepin-5(2H)-yl]acetate;
[(2R,3S)-3-({N-[(3,5-difluorophenyl)acetyl]-L-alanyl}amino)-4-oxo-2-phenyl-3,4-dihydro-1,5-benzoxazepin-5(2H)-yl]acetic acid;
N¹-[(2R,3S)-5-(cyclopropylmethyl)-4-oxo-2-phenyl-2,3,4,5-tetrahydro-1,5-benzoxazepin-3-yl]-N²-[(3,5-difluorophenyl)acetyl]-L-alaninamide;
N¹-[(2R,3S)-5-(cyclopropylmethyl)-7-methoxy-4-oxo-2-phenyl-2,3,4,5-tetrahydro-1,5-benzoxazepin-3-yl]-N²-[(3,5-difluorophenyl)acetyl]-L-alaninamide;
N²-[(3,5-difluorophenyl)acetyl]-N¹-[(2R,3S)-7-fluoro-4-oxo-2-phenyl-2,3,4,5-tetrahydro-1,5-benzoxazepin-3-yl]-L-alaninamide;
(2S)—N-((1S)-2-{[(2R,3S)-7-fluoro-4-oxo-2-phenyl-2,3,4,5-tetrahydro-1,5-benzoxazepin-3-yl]amino}-2-oxo-1-phenylethyl)-2-hydroxy-4-methylpentanamide;
N²-[(2R)-2-(3,5-difluorophenyl)-2-hydroxyacetyl]-N¹-[(2R,3S)-4-oxo-2-phenyl-2,3,4,5-tetrahydro-1,5-benzoxazepin-3-yl]-L-alaninamide;
N²-[(2S)-2-(3,5-difluorophenyl)-2-hydroxyacetyl]-N¹-[(2R,3S)-4-oxo-2-phenyl-2,3,4,5-tetrahydro-1,5-benzoxazepin-3-yl]-L-alaninamide;
N²-[(3,5-difluorophenyl)acetyl]-N¹-[(3S,4R)-2-oxo-4-phenyl-2,3,4,5-tetrahydro-1H-1-benzazepin-3-yl]-L-alaninamide;
N²-[(3,5-difluorophenyl)acetyl]-N¹-[(3S,4R)-8-fluoro-1-methyl-2-oxo-4-phenyl-2,3,4,5-tetrahydro-1H-1-benzazepin-3-yl]-L-alaninamide;
(2S)—N-((1S)-2-{[(3S,4R)-8-fluoro-2-oxo-4-phenyl-2,3,4,5-tetrahydro-1H-1-benzazepin-3-yl]amino}-2-oxo-1-phenylethyl)-2-hydroxy-4-methylpentanamide;
(2S)-2-hydroxy-4-methyl-N-((1S)-2-oxo-2-{[(3S,4R)-2-oxo-4-phenyl-2,3,4,5-tetrahydro-1H-1-benzazepin-3-yl]amino}-1-phenylethyl)pentanamide;
N²-[(3,5-difluorophenyl)acetyl]-N-[(3S,4R)-2-oxo-4-phenyl-1-prop-2-yn-1-yl-2,3,4,5-tetrahydro-1H-1-benzazepin-3-yl]-L-alaninamide;
N¹-[(3S,4R)-1-(cyclopropylmethyl)-2-oxo-4-phenyl-2,3,4,5-tetrahydro-1H-1-benzazepin-3-yl]-N²-[(3,5-difluorophenyl)acetyl]-L-alaninamide;
N²-[(3,5-difluorophenyl)acetyl]-N¹-[(3S,4R)-1-isopropyl-2-oxo-4-phenyl-2,3,4,5-tetrahydro-1H-1-benzazepin-3-yl]-L-alaninamide;
N²-[(2S)-2-hydroxy-4-methyl-1-oxopentyl]-N¹-[(2R,3R)-2-(4-methoxyphenyl)-4-oxo-2,3,4,5-tetrahydro-1,5-benzothiazepin-3-yl]-L-alaninamide;
N¹-[(2R,3R)-2-(2-chlorophenyl)-4-oxo-2,3,4,5-tetrahydro-1,5-benzothiazepin-3-yl]-N²-[(2S)-2-hydroxy-4-methyl-1-oxopentyl]-L-alaninamide;
N¹-[(2R,3R)-2-(2-chlorophenyl)-4-oxo-2,3,4,5-tetrahydro-1,5-benzothiazepin-3-yl]-N-[(3,5-difluorophenyl)acetyl]-L-alaninamide;
N¹-[(2R,3R)-7-chloro-5-methyl-4-oxo-2-phenyl-2,3,4,5-tetrahydro-1,5-benzothiazepin-3-yl]-N²-[(3,5-difluorophenyl)acetyl]-L-alaninamide;
N²-[(3,5-difluorophenyl)acetyl]-N¹-[(2R,3R)-2-(2-fluorophenyl)-4-oxo-2,3,4,5-tetrahydro-1,5-benzothiazepin-3-yl]-L-alaninamide;
N²-[(3,5-difluorophenyl)acetyl]-N¹-[(2R,3R)-2-(4-fluorophenyl)-4-oxo-2,3,4,5-tetrahydro-1,5-benzothiazepin-3-yl]-L-alaninamide;
N¹-[(2R,3R)-7-chloro-2-(2,5-difluorophenyl)-4-oxo-2,3,4,5-tetrahydro-1,5-benzothiazepin-3-yl]-N²-[(3,5-difluorophenyl)acetyl]-L-alaninamide;
N²-[(2S)-2-hydroxy-4-methyl-1-oxopentyl]-N¹-[(6R,7R)-5-oxo-7-phenyl-1,4-thiazepan-6-yl]-L-alaninamide;
N²-[(3,5-difluorophenyl)acetyl]-N¹-[(2S,3R)-2-(3-methyl-2-thienyl)-4-oxo-2,3,4,5-tetrahydro-1,5-benzothiazepin-3-yl]-L-alaninamide;
N²-[(3,5-difluorophenyl)acetyl]-N¹-[(2S,3R)-2-(4-methyl-2-thienyl)-4-oxo-2,3,4,5-tetrahydro-1,5-benzothiazepin-3-yl]-L-alaninamide;
N¹-[(2R,3R)-4-oxo-2-phenyl-2,3,4,5-tetrahydro-1,5-benzothiazepin-3-yl]-N-(phenylacetyl)-L-alaninamide;
N²-[(3,5-difluorophenyl)acetyl]-N¹-[(2S,3R)-4-oxo-2-(2-thienyl)-2,3,4,5-tetrahydro-1,5-benzothiazepin-3-yl]-L-alaninamide;
N²-[(3,5-difluorophenyl)acetyl]-N¹-[(2R,3R)-4-oxo-2-(3-thienyl)-2,3,4,5-tetrahydro-1,5-benzothiazepin-3-yl]-L-alaninamide;
N²-[(3,5-difluorophenyl)acetyl]-N¹-[(2S,3R)-2-(2-furyl)-4-oxo-2,3,4,5-tetrahydro-1,5-benzothiazepin-3-yl]-L-alaninamide;
N²-[(3,5-difluorophenyl)acetyl]-N¹-[(2R,3R)-2-(3-furyl)-4-oxo-2,3,4,5-tetrahydro-1,5-benzothiazepin-3-yl]-L-alaninamide;
N¹-[(2S,3R)-2-(5-bromo-2-thienyl)-4-oxo-2,3,4,5-tetrahydro-1,5-benzothiazepin-3-yl]-N²-[(3,5-difluorophenyl)acetyl]-L-alaninamide;
N¹-[(2S,3R)-2-(4-bromo-2-thienyl)-4-oxo-2,3,4,5-tetrahydro-1,5-benzothiazepin-3-yl]-N²-[(3,5-difluorophenyl)acetyl]-L-alaninamide;
N-[(3,5-difluorophenyl)acetyl]-N-[(2R,3R)-4-oxo-2-phenyl-2,3,4,5-tetrahydro-1,5-benzothiazepin-3-yl]-L-phenylalaninamide;
N²-[(3,5-difluorophenyl)acetyl]-N¹-[(2R,3R)-4-oxo-2-phenyl-2,3,4,5-tetrahydro-1,5-benzothiazepin-3-yl]glycinamide;
N²-[(3,5-difluorophenyl)acetyl]-N¹-[(2R,3R)-4-oxo-2-phenyl-2,3,4,5-tetrahydro-1,5-benzothiazepin-3-yl]-L-valinamide;
N²-[(3,5-difluorophenyl)acetyl]-N-[(2R,3R)-4-oxo-2-phenyl-2,3,4,5-tetrahydro-1,5-benzothiazepin-3-yl]-L-leucinamide;
N²-[(3,5-difluorophenyl)acetyl]-N¹-[(2R,3R)-4-oxo-2-phenyl-2,3,4,5-tetrahydro-1,5-benzothiazepin-3-yl]-L-methioninamide;
N²-[(3,5-difluorophenyl)acetyl]-3-(1H-indol-2-yl)-N¹-[(2R,3R)-4-oxo-2-phenyl-2,3,4,5-tetrahydro-1,5-benzothiazepin-3-yl]-L-alaninamide;
N²-[(3,5-difluorophenyl)acetyl]-N¹-[(2R,3R)-4-oxo-2-phenyl-2,3,4,5-tetrahydro-1,5-benzothiazepin-3-yl]-L-a-asparagine;
N²-[(3,5-difluorophenyl)acetyl]-N¹-[(2R,3R)-4-oxo-2-phenyl-2,3,4,5-tetrahydro-1,5-benzothiazepin-3-yl]-L-a-glutamine;
N¹-[(2R,3S)-4-oxo-2-phenyl-2,3,4,5-tetrahydro-1,5-benzoxazepin-3-yl]-N²-(phenylacetyl)-L-alaninamide;
N²-[(2-fluorophenyl)acetyl]-N¹-[(2R,3S)-4-oxo-2-phenyl-2,3,4,5-tetrahydro-1,5-benzoxazepin-3-yl]-L-alaninamide;
N²-[(3-fluorophenyl)acetyl]-N¹-[(2R,3S)-4-oxo-2-phenyl-2,3,4,5-tetrahydro-1,5-benzoxazepin-3-yl]-L-alaninamide;
N²-[(4-fluorophenyl)acetyl]-N¹-[(2R,3S)-4-oxo-2-phenyl-2,3,4,5-tetrahydro-1,5-benzoxazepin-3-yl]-L-alaninamide;
N¹-[(2R,3S,5aS,9aS)-5-(cyclopropylmethyl)-4-oxo-2-phenyldecahydro-1,5-benzoxazepin-3-yl]-N²-[(3,5-difluorophenyl)acetyl]-L-alaninamide;
N¹-[(2R,3R)-7-chlro-2-(2,5-difluorophenyl)-4-oxo-5-phenyl-2,3,4,5-tetrahydro-1,5-benzothiazepin-3-yl]-N²-[(3,5-difluorophenyl)acetyl]-L-alaninamide;
   in free or pharmaceutically acceptable salt form.

In another embodiment, the invention further includes a compound selected from:
N²-[(3,5-difluorophenyl)acetyl]-N¹-[(6R,7R)-7-(1-naphthyl)-5-oxo-1,4-thiazepan-6-yl]-L-alaninamide;
(2S)-2-{[(3,5-difluorophenyl)acetyl]amino}-N-[(6R,7R)-7-(1-naphthyl)-5-oxo-1,4-thiazepan-6-yl]-2-phenylacetamide;
(2S)-2-hydroxy-4-methyl-N-((1S)-2-{[(6R,7R)-7-(1-naphthyl)-5-oxo-1,4-thiazepan-6-yl]amino}-2-oxo-1-phenylethyl)pentanamide;
N¹-[(2R,3R)-7-chloro-2-(2,5-difluorophenyl)-4-oxo-5-phenyl-2,3,4,5-tetrahydro-1,5-benzothiazepin-3-yl]-N-[(3,5-difluorophenyl)acetyl]-L-alaninamide;
3-cyclohexyl-N²-[(3,5-difluorophenyl)acetyl]-N¹-[(6R,7R)-5-oxo-7-phenyl-1,4-thiazepan-6-yl]-L-alaninamide;
(2S)-2-[(cyclohexylacetyl)amino]-2-(4-fluorophenyl)-N-[(2R,3S)-4-oxo-2-phenyl-2,3,4,5-tetrahydro-1,5-benzoxazepin-3-yl]acetamide;
N¹-[(2R,3S)-5-(2-azetidin-1-yl-2-oxoethyl)-4-oxo-2-phenyl-2,3,4,5-tetrahydro-1,5-benzoxazepin-3-yl]-N²-[(3,5-difluorophenyl)acetyl]-L-alaninamide;
Methyl 5-[(2S,3R)-3-({N-[(3,5-difluorophenyl)acetyl]-L-alanyl}amino)-4-oxo-2,3,4,5-tetrahydro-1,5-benzothiazepin-2-yl]thiophene-3-carboxylate;
N¹-[(2R,3R)-4-oxo-2-phenyl-2,3,4,5-tetrahydro-1,5-benzothiazepin-3-yl]-N²-(2-phenylethyl)-L-alaninamide.

In another aspect, the invention includes also compounds of Formula (IC):

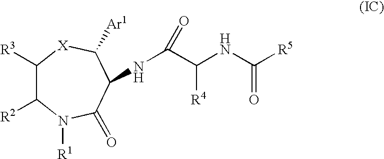

or a pharmaceutically acceptable salt thereof.

In yet another aspect, the invention includes compounds of Formula (ID):

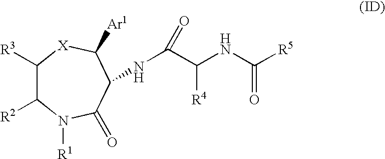

or a pharmaceutically acceptable salt thereof.

Compounds (IC) and (ID) represent particular stereoisomers of Formula (1). In one embodiment of the invention, the compound of (IC) is substantially free of all other stereoisomers. In another embodiment, the compound of (ID) is substantially free of all other stereoisomers.

The method according to any of the proceeding claims wherein the disorder to be treated is cancer.

The method according to any of the proceeding claims wherein the disorder is selected from the group consisting of hematologic, genitourinary, gynecologic, digestive, gastrointestinal, neurologic, breast, lung and mucoepidermoid cancers.

The method according to any of the proceeding claims wherein the disorder is selected from the group consisting of leukemia, multiple myeloma, extramedullary plasmacytoma, renal cell carcinoma and ovarian, endometrial, cervical, colon, prostate, or pancreatic cancer.

The method according to any of the proceeding claims wherein the disorder is T cell acute lymphocytic leukemia.

Use of a compound according to any of the proceeding claims, in free form or in a pharmaceutically acceptable salt form or in the form of a pharmaceutically acceptable solvate of the compound or salt in the manufacture of a medicament for the treatment of disorders associated with activation of the Notch signal transduction pathway.

A pharmaceutical composition of any of the compounds disclosed herein for use in the treatment of disorders associated with activation of the Notch signal transduction pathway.

Use of a compound according to any of the proceeding claims in free form or in a pharmaceutically acceptable salt form or in the form of a pharmaceutically acceptable solvate of the compound or salt for the treatment of disorders associated with activation of the Notch signal transduction pathway.

A method of inhibiting activation of the Notch signal transduction pathway comprising administering an effective amount of a compound of any of the proceeding claims in free form or in a pharmaceutically acceptable salt form or in the form of a pharmaceutically acceptable solvate of the compound or salt, to a human or animal patient in need thereof.

Use of a compound of any of the proceeding claims, in free form or in a pharmaceutically acceptable salt form or in the form of a pharmaceutically acceptable solvate of the compound or salt for the manufacture of a medicament for inhibiting activation of the Notch signal transduction pathway.

Use of a compound of any of the proceeding claims in free form or in a pharmaceutically acceptable salt form or in the form of a pharmaceutically acceptable solvate of the compound or salt for inhibiting activation of the Notch signal transduction pathway.

A pharmaceutical composition of any of the compounds disclosed herein for use in inhibiting activation of the Notch signal transduction pathway.

It is contemplated that use of the pharmaceutically acceptable salts of the compounds described herein, as well as pharmaceutically acceptable solvates of the compounds or the salts are also included within the scope of the present invention.

Method of making and formulating the compounds disclosed herein, as well as intermediates useful for making the compounds are disclosed in WO 2004/031154, the entire contents of which is incorporated by reference herein.

The invention provides methods of treatment of any one or more of the following conditions:

Disorders associated with the activation of the Notch signal transduction pathway (Method 1);

Cancers, particularly those mediated, in whole or in part, by the Notch signal transduction pathway (Method 2);

Disorders which include, but are not limited to, hematologic, genitourinary, gynecologic, digestive, gastrointestinal, neurologic, breast, lung and mucoepidermoid cancers (Method 3);

Disorders which include, but are not limited to, leukemia, multiple myeloma, extramedullary plasmacytoma, renal cell carcinoma, ovarian, endometrial, cervical, colon, prostate or pancreatic cancer. (Method 4);

T cell acute lymphocytic leukemia (T-ALL). (Method 5);
comprising administering a therapeutically effective amount of a compound of the invention, for example, a compound of Formula (I) as described herein, in free form or in a pharmaceutically acceptable salt form or in the form of a pharmaceutically acceptable solvate of the compound or salt, to a human or animal patient in need thereof.

It is also contemplated herein that the compounds disclosed herein may be used in the foregoing methods of treatment as a sole therapeutic agent, but may also be used in combination or for coadministration with, conventional surgery or radiotherapy or chemotherapy. Such chemotherapy may include one or more of the following categories of anti-tumour agents:

(i) other antiproliferative/antineoplastic drugs and combinations thereof, as used in medical oncology, such as alkylating agents (for example cis-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);

(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5a-reductase such as finasteride;

(iii) anti-invasion agents (for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341) and N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661), and metalloproteinase inhibitors like marimastat, inhibitors of urokinase plasminogen activator receptor function or antibodies to Heparanase);

(iv) inhibitors of growth factor function: for example such inhibitors include growth factor antibodies and growth factor receptor antibodies (for example the anti-erbB2 antibody trastuzumab [Herceptin™], the anti-EGFR antibody panitumumab, the anti-erbB1 antibody cetuximab [Erbitux, C225] and any growth factor or growth factor receptor antibodies disclosed by Stem et al. Critical reviews in oncology/haematology, 2005, Vol. 54, ppl 1-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib, inhibitors of the hepatocyte growth factor family, inhibitors of the platelet-derived growth factor family such as imatinib, inhibitors of serine/threonine kinases (for example Ras/Raf signalling inhibitors such as farnesyl transferase inhibitors, for example sorafenib (BAY 43-9006)), inhibitors of cell signalling through MEK and/or AKT kinases, inhibitors of the hepatocyte growth factor family, c-kit inhibitors, abl kinase inhibitors, IGF receptor (insulin-like growth factor) kinase inhibitors; aurora kinase inhibitors (for example AZD1152, PH739358, VX-680, MLN8054, R763, MP235, MP529, VX-528 AND AX39459) and cyclin dependent kinase inhibitors such as CDK2 and/or CDK4 inhibitors;

(v) antiangiogenic agents such as those which inhibit the effects of vascular endothelial growth factor, [for example the anti-vascular endothelial cell growth factor antibody bevacizumab (Avastin™) and VEGF receptor tyrosine kinase inhibitors such as 4-(4-bromo-2-fluoroanilino)-6-methoxy-7-(1-methylpiperidin-4-ylmethoxy)quinazoline (ZD6474; Example 2 within WO 01/32651), 4-(4-fluoro-2-methylindol-5-yloxy)-6-methoxy-7-(3-pyrrolidin-1-ylpropoxy)quinazoline (AZD2171; Example 240 within WO 00/47212), vatalanib (PTK787; WO 98/35985) and SU11248 (sunitinib; WO 01/60814), compounds such as those disclosed in International Patent Applications WO97/22596, WO 97/30035, WO 97/32856 and WO 98/13354 and compounds that work by other mechanisms (for example linomide, inhibitors of integrin αvβ3 function and angiostatin)];

(vi) vascular damaging agents such as Combretastatin A4 and compounds disclosed in International Patent Applications WO 99/02166, WO 00/40529, WO 00/41669, WO 01/92224, WO 02/04434 and WO 02/08213;

(vii) antisense therapies, for example those which are directed to the targets listed above, such as ISIS 2503, an anti-ras antisense;

(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and

(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

Pharmaceutical compositions comprising the compounds of the present invention in combination or association with a pharmaceutically acceptable carrier or diluent for use in the methods of treatment or other methods or uses described herein are also contemplated.

Compounds of this invention may also be used as standards and reagents in methods for determining the ability of a potential pharmaceutical to inhibit activation of the Notch signal transduction pathway. Kits comprising a compound or compounds of this invention to be used in said methods are further contemplated.

The compounds of the invention can be prepared in a number of ways well known to one skilled in the art of organic synthesis, including, but not limited to, as described in detail in WO 2004/031154, the entire contents of which are hereby incorporated by reference.

Compounds according to the present invention may be administered orally, sublingually, intramuscularly, subcutaneously, topically, intranasally, intraperitoneally, intrathoracially, intravenously, epidurally, intrathecally, intracerebroventricularly and by injection into the joints. Preferred routes of administration are orally, intravenously or intramuscularly.

The dosage will depend on the route of administration, the severity of the disease, age and weight of the patient and other factors normally considered by the attending physician, when determining the individual regimen and dosage level as the most appropriate for a particular patient.

For preparing pharmaceutical compositions from the compounds of this invention, inert, pharmaceutically acceptable carriers can be either solid or liquid. Solid form preparations include powders, tablets, dispersible granules, capsules, cachets, and suppositories.

A solid carrier can be one or more substances which may also act as diluents, flavoring agents, solubilizers, lubricants, suspending agents, binders, or tablet disintegrating agents; it can also be an encapsulating material.

In powders, the carrier may be a finely divided solid which is in a mixture with the finely divided active component. In tablets, the active component may be mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture is then poured into convenient sized molds and allowed to cool and solidify.

Suitable carriers include magnesium carbonate, magnesium stearate, talc, lactose, sugar, pectin, dextrin, starch, tragacanth, methyl cellulose, sodium carboxymethyl cellulose, a low-melting wax, cocoa butter, and the like.

Salts include, but are not limited to, pharmaceutically acceptable salts. Examples of pharmaceutically acceptable salts of compounds of the present invention include: acetate, bicarbonate, carbonate, hydrobromide, hydrochloride, phosphate/diphosphate, sulfate, choline, diethanolamine, ethylenediamine, meglumine, aluminum, calcium, magnesium, potassium and sodium.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine

The term “composition” is intended to include the formulation of the active component with encapsulating material as a carrier providing a capsule in which the active component (with or without other carriers) is surrounded by a carrier which is thus in association with it. Similarly, cachets are included. Tablets, powders, cachets, and capsules can be used as solid dosage forms suitable for oral administration.

Liquid form compositions include solutions, suspensions, and emulsions. Sterile water or water-propylene glycol solutions of the active compounds may be mentioned as an example of liquid preparations suitable for parenteral administration. Liquid compositions can also be formulated in solution in aqueous polyethylene glycol solution. Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methylcellulose, sodium carboxymethyl cellulose, and other suspending agents known to the pharmaceutical formulation art.

The pharmaceutical compositions can be in unit dosage form. In such form, the composition is divided into unit doses containing appropriate quantities of the active component. The unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms. In general, satisfactory results, e.g. for the treatment of diseases as hereinbefore set forth are indicated to be obtained on oral administration at dosages of the order from about 0.01 to 2.0 mg/kg. In larger mammals, for example humans, an indicated daily dosage for oral administration will accordingly be in the range of from about 0.75 to 1000 mg, conveniently administered once, or in divided doses 2 to 4 times, daily or in sustained release form. Unit dosage forms for oral administration thus for example may comprise from about 0.2 to 250 mg, e.g. from about 0.2 or 2.0 to 50, 100 or 250 mg of a compound disclosed herein, together with a pharmaceutically acceptable diluent or carrier therefor.

It is contemplated that the invention described herein is not limited to the particular methodology, protocols, and reagents described as these may vary. It is also to be understood that the terminology used herein is for the purpose of describing particular embodiments only, and is not intended to limit the scope of the present invention in any way.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices and materials are herein described. All publications mentioned herein are incorporated by reference for the purpose of describing and disclosing the materials and methodologies that are reported in the publication which might be used in connection with the invention.

### EXAMPLES

Chemical abbreviations used in the Examples are defined as follows: “BOC” denotes N-tert-butoxycarbonyl, “CBZ” denotes carbobenzyloxy; “DBU” denotes 1,8-diazabicyclo[5.4.0]undec-7-ene; “DCC” denotes N,N′-dicylcohexylcarbodiimde; “DCM” denotes dichloromethane; “DIEA” denotes N,N-diisopropylethylamine, “DMF” denotes N,N-dimethylformamide; “EDAC-HCl” denotes 1-Ethyl-3-(dimethylaminopropyl)carbodiimide hydrochloride; “EDC” denotes N-(3-dimethylaminopropyl)-N′-ethylcarbodiimide; “EtOAc” denotes ethyl acetate; “HOBt” denotes hydroxybenzotriazole; “NMM” denotes N-methylmorpholine; “p-TSA” denotes p-toluenesulfonic acid “TBAB” denotes tetrabutylaimnonium bromide; “THF” denotes tetrahydrofuran, “ether” denotes ethyl ether, Tos-Cl denotes p-toluenesulfonyl chloride, “min.” denotes minutes; “h” denotes hours. Unless otherwise noted, organic solutions were “dried” over anhydrous sodium sulfate.

HPLC Method A: Phenomenex Luna 3μ C18(2), 4.6×75 mm column. Solvents: A=H₂O with 0.1% TFA, B=Acetonitrile with 0.1% TFA. Flow rate 2.0 mL/min. 20% B until 0.5 min then a linear gradient to 95% B at 3 min. Maintain at 95% B until 6 min
HPLC Method B: Phenomenex Luna 3μ C18(2), 4.6×75 mm column. Solvents: A=H₂O with 0.1% TFA, B=Acetonitrile with 0.1% TFA. Flow rate 2.0 mL/min. Linear gradient from 10% to 95% B at 5 min. Maintain at 95% B until 7 min.
HPLC Method C: 5μ SB—C8 column 2.1 mm×5 cm. Solvents: A=H₂O with 0.05% TFA, B=10% H₂O, 90% Acetonitrile, 0.05% TFA. Flow rate 1.4 mL/min. Gradient: (5-90% B over 5 min., 90% B hold for 2 min.).
HPLC Method D: Agilent Zorbax 5μ SB—C8 column 2.1 mm×5 cm. Solvents: A=H2O with 0.1% TFA, B=Acetonitrile with 0.1% TFA. Flow rate 1.4 mL/min. Linear gradient from 9% to 81% B at 3 min. then linear gradient to 95% B at 4 min. Maintain 95% B until 4.5 min.
HPLC Method E: Agilent Zorbax 5μ SB—C8 column 2.1 mm×5 cm. Solvents: A=H2O with 0.05% TFA, B=90% Acetonitrile, 10% water, 0.05% TFA. Flow rate 1.4 mL/min. Linear gradient from 15% to 90% B in 12 min.
LC/MS:HPLC method: Agilent Zorbax 5μ SB—C8 column 2.1 mm×5 cm. Solvents: A=H₂O with 0.05% TFA, B=10% H₂O, 90% Acetonitrile, 0.05% TFA. Gradient: 10 to 90% B over 3 min., 90% B hold thru 4 min., 10% B at 5 min. and hold at 10% B until 6 min).
“ISCO” refers to normal phase flash column chromatography using pre-packed silica gel cartridges used according to the manufacturers instruction obtained from ISCO, Inc, 4700 superior street Lincoln, Nebr., USA.

### Example 1

### N²-[(3,5-Difluorophenyl)acetyl]-N¹-[(2R,3R)-2-(2,5-difluorophenyl)-4-oxo-2,3,4,5-tetrahydro-1,5-benzothiazepin-3-yl]-L-alaninamide (1)

To a solution of racemic 2,3-cis-3-amino-2-(2,5-difluorophenyl)-2,3-dihydro-1,5-benzothiazepin-4(5H)-one (1d) (300 mg) in DCM (40 mL) at 0° C. under nitrogen was added N-[(3,5-difluorophenyl)acetyl]-L-alanine (1e) (238 mg), HOBt-hydrate (330 mg), EDAC-HCl (282 mg) and NMM (165 mg). The reaction mixture was stirred 1 h at 0° C., concentrated in vacuo and partitioned between water (100 mL) and EtOAc (125 mL). The organic phase was collected and consecutively washed with water, saturated aqueous sodium bicarbonate, and brine, dried, filtered and evaporated to yield a mixture of the title compound and N²-[(3,5-difluorophenyl)acetyl]-N¹-[(2S,3S)-2-(2,5-difluorophenyl)-4-oxo-2,3,4,5-tetrahydro-1,5-benzothiazepin-3-yl]-L-alaninamide. The crude product (500 mg) was purified by flash chromatography (50% EtOAc-hexanes) to afford the title compound (180 mg, 69%) as an off-white solid. ¹H NMR (300 MHz, CDCl₃) δ1.22 (d, 3H), 3.48 (s, 2H), 4.29 (m, 1H), 4.93 (t, 1H), 5.68 (d, 1H), 6.01 (d, 1H), 6.50 (d, 1H), 6.73-6.80 (m, 3H), 6.93-7.02 (m, 2H), 7.15 (d, 1H), 7.30 (t, 1H), 7.43 (t, 1H), 7.5-7.6 (m, 1H), 7.73 (d, 1H), 7.74 (s, 1H). MS APCI, m/z=532 (M+1). LC/MS: 2.53 min.

The starting amine, racemic 2,3-cis-3-amino-2-(2,5-difluorophenyl)-2,3-dihydro-1,5-benzothiazepin-4(5H)-one (1d), was prepared in the following manner:

### a. Methyl (2Z)-2-{[(benzyloxy)carbonyl]amino}-3-(2,5-difluorophenyl)prop-2-enoate (1a)

A stirred solution of N-(benzyloxycarbonyl)-α-phosphonoglycine trimethyl ester (6.1 g) and 2,5-difluorobenzaldehyde (2.0 g) in dry DCM (60 mL), was treated dropwise with a solution of DBU (2.5 mL) in DCM (20 mL). The mixture was stirred at 25° C. for 2 h, then was concentrated to approximately 20 mL and partitioned between EtOAc (150 mL) and 1N hydrochloric acid (50 mL). The organic extract was collected, consecutively washed with 1N hydrochloric acid, water, saturated aqueous sodium bicarbonate, and brine, dried (sodium sulfate), filtered and evaporated. The crude product (6.5 g) was purified by flash chromatography (20% EtOAc-hexanes) to yield the title compound (4.0 g, 82%). ¹H NMR (300 MHz, CDCl₃) δ 3.85 (s, 3H), 5.10, (s, 2H), 6.60 (bs, 1H), 6.9-7.1 (m, 2H), 7.21 (m, 1H), 7.2-7.3 (m, 6H). MS APCI, m/z=348 (M+1). LC/MS: 2.53 min.

### b. Methyl β-[(2-aminophenyl)thio]-N-[(benzyloxy)carbonyl]-2,5-difluorophenylalaninate (1b)

### Method A

To an ice-cooled solution of sodium methoxide (760 mg) in anhydrous methanol (20 mL) under nitrogen (vacuum degassed 3× with nitrogen) was added 2-aminothiophenol (1.7 g). The reaction mixture stirred at 0° C. for 10 min and then a solution of methyl (2Z)-2-{[(benzyloxy)carbonyl]amino}-3-(2,5-difluorophenyl)prop-2-enoate (2.32 g) in methanol (10 mL) was added. The reaction mixture was heated to reflux for 2 h and then was cooled to 25° C. and stirred for ˜12 h. The reaction mixture was concentrated to ca. 10 mL, then was partitioned between cold 1N hydrochloric acid (75 mL) and EtOAc (125 mL). The organic phase was separated and consecutively washed with 1N hydrochloric acid, dilute aqueous sodium bicarbonate and brine, dried, filtered and evaporated. The title compound was isolated as the hydrochloride salt. (3.0 g, 88%, 2:1 Z:E). ¹H NMR (300 MHz, d6-DMSO) δ3.4 (s, 2H), 3.7 (s, 1H), 4.6-5.1 (m, 7H), 6.3 (t, 0.67H), 6.4 (t, 0.33H), 6.7-7.4 (m, 10H), 8.1 (d, 0.33H), 8.4 (d, 0.67H). MS APCI, m/z=473 (M+1). LC/MS: 2.78 min.

### Method B

To an ice-cooled solution of 2-aminothiophenol (8.7 g) in anhydrous methanol under nitrogen (vacuum degassed 3× with nitrogen) was added methyl (2Z)-2-{[(benzyloxy)carbonyl]amino}-3-(2,5-difluorophenyl)prop-2-enoate (3.46 g) followed by triethylamine (975 uL). The reaction mixture was stirred at 25° C. for 4 d, then was reduced in vacuo to near dryness. The mixture was partitioned between cold 1N hydrochloric acid (75 mL) and EtOAc (125 mL). The organic phase was separated and consecutively washed with 1N hydrochloric acid, dilute aqueous sodium bicarbonate and brine, dried, filtered and evaporated to yield 5.8 g of an oil. Purification by flash chromatography (25% EtOAc-hexanes) afforded the title compound (4.3 g, 65%) Z:E ratio of 82:18. ¹H NMR (300 MHz, CDCl₃) δ3.48 (s, 2.4H), 3.71 (s, 0.6H), 4.28 (s, 1.6H), 4.72 (s, 0.4H), 4.8-5.1 (m, 4H), 5.43 (d, 0.2H), 5.86 (d, 0.8H), 6.58 (t, 0.8H), 6.68 (d, 0.8H), 6.9-7.4 (m, 8H). MS APCI, m/z=473 (M+1). LC/MS: 2.78 min.

### c. Benzyl cis-2-(2,5-difluorophenyl)-4-oxo-2,3,4,5-tetrahydro-1,5-benzothiazepin-3-ylcarbamate (1c)

A suspension of methyl β-[(2-aminophenyl)thio]-N-[(benzyloxy)carbonyl]-2,5-difluorophenylalaninate (4:1, Z:E) (4.3 g) and p-toluenesulfonic acid (catalytic) in xylenes (100 mL) was heated to reflux for 2 h, using a Dean-Stark apparatus to remove water. The mixture was then cooled, resulting in precipitation of the crude product as a white solid (3.3 g, 4:1, cis:trans). This was recrystallized from EtOAc-ether to afford the title compound (2.4 g, 60%). ¹H NMR (300 MHz, d6-DMSO) δ 4.63 (t, 1H), 4.96 (s, 2H), 5.47 (d, 1H), 7.00 (d, 1H), 7.23-7.34 (m, 9H), 7.49-7.53 (m, 2H), 7.70 (d, 1H), 10.57 (s, 1H). MS APCI, m/z=441 (M+1). LC/MS: 2.74 min.

### d. cis-3-Amino-2-(2,5-difluorophenyl)-2,3-dihydro-5-benzothiazepin-4(5H)-one (1d)

### Method C

A mixture of benzyl cis-2-(2,5-difluorophenyl)-4-oxo-2,3,4,5-tetrahydro-1,5-benzothiazepin-3-ylcarbamate (1.7 g) and 10% palladium on carbon (1.7 g, DeGussa type 50% wt water) in glacial acetic acid (80 mL) was hydrogenated at 50 psi H₂ for 3 h. The reaction mixture was filtered through diatomaceous earth and concentrated in vacuo. The crude oil was triturated with ether to yield a white solid (1.3 g). The solid was partitioned between EtOAc and dilute ammonium hydroxide. The organic phase was separated and consecutively washed with dilute ammonium hydroxide and brine, dried and evaporated. The residue was treated with saturated HCl (g) in ethyl aceate-ether to provide the hydrochloride salt of the title compound as a white solid (1.1 g, 90%). ¹H NMR (300 MHz, d6-DMSO) δ 4.33 (d, 1H, J=7 Hz), 5.60 (d, 1H, J=7 Hz), 7.13-7.38 (m, 4H), 7.48-7.60 (m, 2H), 7.72, (d, 1H), 8.4 (bs, 3H), 11.0 (s, 1H). MS APCI, m/z=307 (M+1). LC/MS: 1.65 min.

### Method D

To benzyl cis-2-(2,5-difluorophenyl)-4-oxo-2,3,4,5-tetrahydro-1,5-benzothiazepin-3-ylcarbamate (0.9 g) was added 30% HBr/HOAc (5 mL). The stirred suspension became a homogeneous solution over 20 min. The reaction stirred at 25° C. for an additional 50 min, then was diluted with ether to afford the hydrobromide salt of the title compound (0.75 g, 95%). The solid was partitioned between EtOAc and saturated aqueous sodium bicarbonate. The organic phase was separated and consecutively washed with dilute aqueous sodium bicarbonate and brine, dried, filtered and evaporated. The resulting oil was treated with saturated HCl (g) in ethyl aceate-ether to provide the hydrochloride salt of the title compound as a white solid (0.60 g, 85%). This material was indistinguishable from that obtained by Method C.

### e. N-[(3,5-Difluorophenyl)acetyl]-L-alanine (1e)

To a stirred solution of 3,5-difluorophenylacetic acid (6.02 g, 34.97 mmol), L-alanine methyl ester hydrochloride (4.88 g, 34.96 mmol) and HOBt (5.20 g, 38.48 mmol) in DCM (200 mL) under nitrogen at 0° C. was added NMM (8.84 g, 87.39 mmol) and EDAC-HCl (7.38 g, 38.49 mmol). The mixture was allowed to warm to 25° C. and to stir for ˜12 h. The reaction was diluted with EtOAc and extracted sequentially with aqueous sodium bicarbonate, 1N aqueous HCl and brine. The organic phase was dried, filtered and evaporated. The residue was purified by flash chromatography on silica gel eluting with 1:1 (v/v) hexanes-EtOAc to afford N-[(3,5-difluorophenyl)acetyl]-L-alanine methyl ester (7.91 g, 88% yield) as a white solid. ¹H NMR (300 MHz, CDCl₃) δ 1.39 (d, 3H, J=7.0 Hz), 3.54 (s, 2H), 3.75 (s, 3H), 4.59 (m, 1H), 6.02 (br 1H), 6.67-6.87 (m, 3H). MS APCI, m/z=258 (M+1). LC/MS: 1.68 min. Lithium hydroxide (1.40 g, 33.33 mmol) in water (60 mL) was added dropwise to a solution of N-[(3,5-difluorophenyl)acetyl]-L-alanine methyl ester (7.79 g, 30.28 mmol) in 1,4-dioxane (150 mL). After 2 h the solvent was evaporated. The residue was dissolved in water and the solution extracted with diethyl ether. The aqueous phase was acidified with 1N aqueous HCl and extracted with EtOAc. The combined EtOAc extracts were dried, filtered and evaporated to afford the title compound (7.16 g, 97% yield) as a white solid. ¹H NMR (300 MHz, d6-DMSO) δ 1.28 (d, 3H, J=7.4 Hz), 3.51 (s, 2H), 4.20 (m, 1H), 6.93-7.12 (m, 3H), 8.44 (d, 1H, J=7.0 Hz), 12.46 (br, 1H). HPLC Method A: 2.12 min.

### Example 2

### N¹-[(2,3-cis)-5-Cyclohexyl-2-(2,5-difluorophenyl)-4-oxo-2,3,4,5-tetrahydro-1,5-benzothiazepin-3-yl]-N²-[(3,5-difluorophenyl)acetyl]-L-alaninamide (2)

Using a procedure similar to that described in Example 1, except using (2,3-cis)-3-amino-5-cyclohexyl-2-(2,5-difluorophenyl)-2,3-dihydro-1,5-benzothiazepin-4(5H)-one (2b) (85 mg) as the amine component, the title compound (2) was obtained as a white solid (20 mg, 30%). ¹H NMR (300 MHz, CDCl₃) δ1.0-2.1 (m, 13H), 3.47 (s, 2H), 4.20 (m, 1H), 4.45 (m, 1H), 4.64 (t, 1H), 5.44 (d, 1H), 5.95 (d, 1H), 6.40 (d, 1H), 6.73-6.80 (m, 3H), 6.85-6.95 (m, 2H), 7.35-7.49 (m, 4H), 7.75 (d, 1H). MS APCI, m/z=614 (M+1). LC/MS: 3.44 min.

The amine component, (2,3-cis)-3-amino-5-cyclohexyl-2-(2,5-difluorophenyl)-2,3-dihydro-1,5-benzothiazepin-4(5H)-one (2b) was prepared in the following manner:

### a. Benzyl (2,3-cis)-5-(2-cyclohexen-1-yl)-2-(2,5-difluorophenyl)-4-oxo-2,3,4,5-tetrahydro-1,5-benzothiazepin-3-ylcarbamate (2a)

To a solution of benzyl cis-2-(2,5-difluorophenyl)-4-oxo-2,3,4,5-tetrahydro-1,5-benzothiazepin-3-ylcarbamate (1c) (150 mg,) in THF (10 mL) under nitrogen was added powdered potassium hydroxide (25 mg), tetrabutylammonium bromide (11 mg) and 1-bromo-2-cyclohexene (40 μl). The reaction mixture was stirred at 25° C. for ˜12 h, then was partitioned between water and EtOAc. The organic phase was separated and consecutively washed with water and brine, dried, filtered and evaporated to yield the title compound 2a (175 mg, 98%). This material was used without further purification. ¹H NMR (300 MHz, CDCl₃) δ1.5-2.3 (m, 6H), 4.6 (t, 1H), 5.0 (d, 2H), 5.2-5.5 (m, 3H), 5.7 (m, 1H), 5.9 (m, 1H), 6.9 (m, 2H), 7.2-7.3 (m, 6H), 7.4 (m, 3H), 7.73 (d, 1H). MS APCI, m/z=521 (M+1). LC/MS: 3.63 min

### b. (2,3-cis)-3-Amino-5-cyclohexyl-2-(2,5-difluorophenyl)-2,3-dihydro-1,5-benzothiazepin-4(5H)-one (2b)

Using a method similar to that described in Example 1, part d (Method C), benzyl (2,3-cis)-5-(2-cyclohexen-1-yl)-2-(2,5-difluorophenyl)-4-oxo-2,3,4,5-tetrahydro-1,5-benzothiazepin-3-ylcarbamate 2a (90 mg) was converted to crude 2b. The crude product purified by flash chromatography (2% Methanol, 1% NH₄OH/CHCl₃) to yield 2b (45 mg, 67%), converted to HCl salt (EtOH-ether-HCl). ¹H NMR (300 MHz, d6-DMSO) δ1.0-2.1 (m, 10H), 4.11 (d, 1H), 4.35 (m, 1H), 5.38 (d, 1H), 7.35 (t, 2H), 7.4-7.5 (m, 2H), 7.6-7.7 (m, 2H), 7.81, (d, 1H), 8.29 (bs, 3H). MS APCI, m/z=389 (M+1). LC/MS: 2.57 min.

### Example 3

### N²-[(3,5-Difluorophenyl)acetyl]-N¹-{(2R,3R)-2-(2,5-difluorophenyl)-5-[2-(dimethylamino)ethyl]-4-oxo-2,3,4,5-tetrahydro-1,5-benzothiazepin-3-yl}-L-alaninamide (3)

Using a procedure similar to that described in Example 1, except using racemic (2,3-cis)-3-amino-2-(2,5-difluorophenyl)-5-(2-dimethylamino)ethyl)-2,3-dihydro-1,5-benzothiazepin-4(5H)-one (3b) (100 mg) as the amine component, the title compound (23 was obtained as a white solid (37 mg, 46%). ¹H NMR (300 MHz, d6-DMSO) δ1.2 (3H), 2.3 (s, 6H), 2.35 (m, 1H), 2.6, (m, 1H), 3.48 (s, 2H),), 3.55 (m, 1H),), 4.22 (m, 1H),), 4.65 (m, 1H), 4.77 (t, 1H),), 5.35 (d, 1H), 5.95 (d, 1H), 6.36 (d, 1H), 6.7-7.0 (m, 5H), 7.27-7.35 (t, 1H), 7.38 (d, 1H), 7.48, (t, 1H), 7.71 (d, 1H), 7.9 (m, 1H), MS APCI, m/z=603 (M+1). LC/MS: 2.13 min.

The amine component, (2,3-cis)-3-amino-5-(2-dimethylamino)ethyl-2-(2,5-difluorophenyl)-2,3-dihydro-1,5-benzothiazepin-4(5H)-one (3b) was prepared in the following manner:

### a. Benzyl (2,3-cis)-5-(2-dimethylaminoethyl)-2-(2(5-difluorophenyl)-4-oxo-2,3,4,5-tetrahydro-1,5-benzothiazepin-3-ylcarbamate (3a)

To a solution of benzyl cis-2-(2,5-difluorophenyl)-4-oxo-2,3,4,5-tetrahydro-1,5-benzothiazepin-3-ylcarbamate 1c (530 mg), prepared as described in Example 1, part c, in methyl isobutyl ketone (14 mL) was added 10N NaOH (0.6 mL) followed by water (2.3 mL) and N,N-dimethylaminoethylchloride hydrochloride (260 mg). The reaction mixture was heated to 95° C. for 4H. (HPLC indicated 3:1 3a:1c), allowed to cool to 25° C. and diluted with EtOAc. The organic phase was collected and consecutively washed with water, brine, dried, filtered and the solvent removed in vacuo to yield crude oil. The crude oil was purified by flash chromatography (5% Methanol-CHCl₃) to afford pure title compound (400 mg, 60%). ¹H NMR (300 MHz, d6-DMSO) δ2.2 (d, 6H), 2.3 (m, 1H), 2.4 (m, 1H), 3.27 (m, 1H), 3.6 (d of t, 1H), 4.4 (t, 1H), 4.5 (t, 1H), 4.9 (s, 2H), 5.3 (d, 2H), 6.8 (d, 1H), 7.2-7.3 (m, 6H), 7.4 (t, 1H), 7.6-7.7 (m, 2H), 7.76, (d, 1H), 7.86 (m, 1H). MS APCI, m/z=512 (M+1). LC/MS: 2.23 min.

### b. (2,3-cis)-3-Amino-5-(2-dimethylaminoethyl)-2-(2,5-difluorophenyl)-2,3-dihydro-1,5 benzothiazepin-4(5H)-one (3b)

Using a method similar to that described in Example 1, part d (Method C), benzyl (2,3-cis)-5-(2-dimethylamino)ethyl)-2-(2,5-difluorophenyl)-4-oxo-2,3,4,5-tetrahydro-1,5-benzothiazepin-3-ylcarbamate, 3a (90 mg) was converted overnight to crude 3b. The crude product was purified by flash chromatography (5% Methanol, 1% NH₄OH—CHCl₃) to afford pure title compound (125 mg, 57%). ¹H NMR (300 MHz, d6-DMSO) δ2.29 (s, 6H), 2.39, (m, 1H), 2.64, (m, 1H), 3.62 (m, 1H), 3.79 (d, 1H), 4.56 (dt, 1H), 5.27 (d, 2H), 6.95-7.05 (m, 2H), 7.28 (m, 1H), 7.40 (d, 1H), 7.72, (d, 1H), 7.78 (m, 1H). MS APCI, m/z=378 (M+1). LC/MS: 1.23 min.

### Example 4

### (2S)-2-{[(3,5-Difluorophenyl)acetyl]amino}-N-[(2R,3R)-2-(2,5-difluorophenyl)-4-oxo-2,3,4,5-tetrahydro-1,5-benzothiazepin-3-yl]-3-hydroxypropanamide (4)

To a solution N-[(3,5-difluorophenyl)acetyl]-L-serine (4b) (75 mg) in DCM (15 mL) at 0° C. under N₂, was added racemic 2,3-cis-3-amino-2-(2,5-difluorophenyl)-2,3-dihydro-1,5-benzothiazepin-4(5H)-one-HCl (1d) (100 mg) followed by the HOBt-hydrate (97 mg) and NMM (32 μL). Reaction stirred for 5 min. and then added EDAC-HCl (84 mg) and NMM (50 μL). The reaction mixture was stirred 2 h at 0° C. under N₂, concentrated in vacuo and partitioned between water (100 mL) and EtOAc (125 mL). The organic phase was collected and consecutively washed with water, saturated aqueous sodium bicarbonate, brine, dried, filtered and evaporated to yield a mixture of the title compound and (2S)-2-{[(3,5-difluorophenyl)acetyl]amino}-N-[(2S,3S)-2-(2,5-difluorophenyl)-4-oxo-2,3,4,5-tetrahydro-1,5-benzothiazepin-3-yl]-3-hydroxypropanamide. The crude product (165 mg) was purified by flash chromatography (80% EtOAc-hexanes) to afford the title compound (60 mg, 73%) as a white solid. ¹H NMR (300 MHz, d6-DMSO) δ(d, 3H), 3.48 (s, 2H), 4.21 (q, 1H), 4.74, (t, 1H), 4.85 (bs, 1H), 5.50 (d, 1H), 6.93 (d, 2H), 7.09 (m, 1H), 7.18-7.35 (m, 4H), 7.45-7.55 (m, 2H), 7.71 (t, 2H), 8.17 (d, 1H), 10.68 (s, 1H). MS APCI, m/z=548 (M+1). LC/MS: 2.34 min.

The starting acid, N-[(3,5-difluorophenyl)acetyl]-L-serine (4b), was prepared in the following manner:

### a. N-[(3,5-Difluorophenyl)acetyl]-L-serine methyl ester (4a)

To an ice cooled solution of 3,5-difluorophenylacetic acid (2.16 g) in anhydrous DCM (100 mL) under nitrogen was added HOBt-hydrate (4.23 g), EDAC-HCl (3.6 g), and NMM (2.2 mL). The reaction mixture was stirred at 0° C. under nitrogen for 15 min and L-serine methyl ester-HCl (1.96 g) was added followed by NMM (1.38 mL). The reaction was stirred at 0° C. for 1H and 25° C. for 2 h. The reaction mixture was concentrated in vacuo and partitioned between water (100 mL) and EtOAc (125 mL). The organic phase was collected and consecutively washed with water, dilute aqueous sodium bicarbonate, brine, dried, filtered and the solvent removed in vacuo to yield a white solid. Trituration with CHCl₃ afforded pure title compound (1.8 g). The impure filtrate was subjected to flash chromatography (20% acetone-CHCl₃) to afford additional title compound (800 mg, total yield 76%). ¹H NMR (300 MHz, d6-DMSO) δ3.57 (d, 2H), 3.62 (s, 3H), 3.7 (m, 1H), 4.35, (m, 1H), 5.1 (bs, 1H), 7.00 (d, 2H), 7.09 (t, 1H), 8.53, (d, 1H). MS APCI, m/z=274 (M+1). LC/MS: 1.34 min.

### b. N-[(3,5-Difluorophenyl)acetyl]-L-serine (4b)

To a stirred solution of N-[(3,5-difluorophenyl)acetyl]-L-serine methyl ester (4a) in THF (13 mL) was added 1M aqueous lithium hydroxide (13.2 mL) and the mixture stirred at 25° C. for 40H. Brine (50 mL) was added, the aqueous layer made acidic to pH 1 with 1N hydrochloric acid (˜15 mL), and the aqueous layer extracted with 10% Methanol-CHCl₃. The organic phase was collected, dried, filtered and the solvent removed in vacuo to afford the title compound (112 mg, 54%). This material was used without further purification. ¹H NMR (300 MHz, d6-DMSO) δ3.57 (d, 2H), 3.7 (m, 1H), 4.27 (m, 1H), 5.03 (bs, 1H), 7.00 (d, 2H), 7.09 (t, 1H), 8.38, (d, 1H). 12.6, (bs, 1H). MS APCI, m/z=274 (M+1). LC/MS: 1.0 min.

### Example 5

### N²-[(3,5-Difluorophenyl)acetyl]-N¹-[(2R,3R)-2-(2,5-difluorophenyl)-5-methyl-4-oxo-2,3,4,5-tetrahydro-1,5-benzothiazepin-3-yl]-L-alaninamide (5)

Using a procedure similar to that described in Example 1, except using racemic (2,3-cis)-3-amino-5-methyl-2-(2,5-difluorophenyl)-2,3-dihydro-1,5-benzothiazepin-4(5H)-one (5b) (170 mg) as the amine component, the title compound (5) was obtained as a white solid (85 mg, 59%) ¹H NMR (300 MHz, CDCl₃) δ1.19 (d, 3H), 3.48 (s, 2H), 3.50 (s, 3H), 4.22 (m, 1H), 4.83, (t, 1H), 5.56 (d, 1H), 5.92 (d, 1H), 6.37 (d, 1H), 6.80-6.90 (m, 3H), 6.90-7.00 (m, 2H), 7.32 (d, 1H), 7.40-7.50 (m, 2H), 7.72 (d, 1H). MS APCI, m/z=546 (M+1). LC/MS: 2.67 min.

The amine component (2,3-cis)-3-amino-5-methyl-2-(2,5-difluorophenyl)-2,3-dihydro-1,5-benzothiazepin-4(5H)-one (5b) was prepared in the following manner:

### a. Benzyl (2,3-cis)-5-(methyl)-2-(2,5-difluorophenyl)-4-oxo-2,3,4,5-tetrahydro-1,5-benzothiazepin-3-ylcarbamate (5a)

To a round bottom flask charged with powdered KOH (182 mg) under nitrogen was added a solution of benzyl cis-2-(2,5-difluorophenyl)-4-oxo-2,3,4,5-tetrahydro-1,5-benzothiazepin-3-ylcarbamate (1c) (1.1 g) prepared as described in Example 1, part c, in THF (15 mL). To the suspension was added tetrabutylammonium bromide (80 mg) followed by addition of methyl iodide (156 μl) via syringe. The mixture stirred at 25° C. for ˜48 h. The reaction mixture was partitioned between water and EtOAc. The organic phase was collected and consecutively washed with water and brine, dried, filtered and the solvent removed in vacuo to afford the crude product (1.15 g). Recrystallization from EtOAc (10 mL) yielded pure title compound 5a (660 mg, 58%). ¹H NMR (300 MHz, d6-DMSO) δ3.42 (s, 3H), 4.60 (t, 1H), 4.93 (s, 2H), 5.34 (d, 1H), 7.01 (d, 1H), 7.22-7.34 (m, 7H), 7.42 (q, 2H), 7.62 (s, 1H), 7.63 (d, 1H), 7.76 (d, 1H). MS APCI, m/z=455 (M+1). LC/MS: 2.93 min.

### b. (2,3-cis)-3-Amino-5-methyl-2-(2,5-difluorophenyl)-2,3-dihydro-1,5-benzothiazepin-4(5H)-one (5b)

Using a method similar to that described in Example 1, part d (Method D), benzyl (2,3-cis)-5-(methyl)-2-(2,5-difluorophenyl)-4-oxo-2,3,4,5-tetrahydro-1,5-benzothiazepin-3-ylcarbamate 5a (600 mg) was converted to nearly pure 5b (350 mg, 83%). Recrystallization (ether-hexanes) afforded the pure title compound (162 mg). ¹H NMR (300 MHz, d6DMSO) δ1.6-2.5 (bs, 2H), 3.41 (s, 3H), 3.50 (s, 3H), 3.76 (d, 1H), 5.17 (d, 1H), 7.25-7.38 (m, 4H), 7.58 (s, 1H), 7.59 (d, 1H), 7.72 (d, 1H). MS APCI, m/z=321 (M+1). LC/MS: 1.76 min.

### Example 6

### N²-[(3,5-Difluorophenyl)acetyl]-N¹-[(2R,3S)-4-oxo-2-phenyl-2,3,4,5-tetrahydro-1,5-benzoxazepin-3-yl]-L-alaninamide (6)

To a stirred solution of N¹-[(2R,3S)-4-oxo-2-phenyl-2,3,4,5-tetrahydro-1,5-benzoxazepin-3-yl]-L-alaninamide (6) (765 mg, 2.351 mmol) in DCM (10 mL) was added 3,5-difluorophenylacetic acid (450 mg, 2.614 mmol), HOBt (441 mg, 3.265 mmol), NMM (330 mg, 3.267 mmol) and EDAC-HCl (626 mg, 3.265 mmol). The mixture was stirred at ambient temperature under nitrogen for ˜12 h. The solvent was evaporated and the residue partitioned between EtOAc and saturated aqueous sodium bicarbonate. The organic phase was separated and then washed in succession with 1N aqueous HCl and brine. The organic solution was dried, filtered and evaporated. The residue was purified by flash chromatography on silica gel eluting with 2:1 (v/v) EtOAc-hexanes to afford the title compound (968 mg, 86%) as a white solid. TLC R_{f}=0.30 (2:1 EtOAc-hexanes). ¹H NMR (300 MHz, DMSO-d6) δ1.11 (d, 3H, J=7.0 Hz), 3.46 (q AB, 2H, J=14.4 Hz), 4.24 (m, 1H), 4.95 (m, 1H), 5.61 (d, 1H, J=6.6 Hz), 6.94 (m, 2H), 7.02-7.29 (m, 5H), 7.32-7.40, (m, 6H), 8.32 (m, 1H), 10.29 (br, 1H). MS APCI, m/z=480 (M+1). LC/MS: 2.31 min.

The precursor N¹-[(2R,3S)-4-oxo-2-phenyl-2,3,4,5-tetrahydro-1,5-benzoxazepin-3-yl]-L-alaninamide (6) was prepared as follows:

### a. erythro Ethyl 2-hydroxy-3-(2-nitrophenoxy)-3-phenylpropanoate (6a)

The title compound was prepared according to the published procedure of Carlo Banzatti, Franco Heidempergher, and Piero Melloni; J. Heterocyclic Chem. 20, 259 (1983).

### b. erythro Ethyl 3-(2-aminophenoxy)-2-hydroxy-3-phenylpropanoate (6b)

To a solution of erythro ethyl 2-hydroxy-3-(2-nitrophenoxy)-3-phenylpropanoate (6a) (3.956 g, 11.940 mmol) in ethanol (150 mL) was added 5% palladium on carbon (500 mg) and the mixture was hydrogenated at 35 psi on a Parr shaker for 30 min. The reaction mixture was filtered through diatomaceous earth and the resulting solution concentrated in-vacuo. The residue was purified by flash chromatography on silica gel eluting with EtOAc to afford the title compound (3.585 g, 99%) as a red oil. ¹H NMR (300 MHz, CDCl₃) δ1.18 (t, 3H), 3.34 (m, 1H), 3.99 (br, 2H), 4.17 (m, 2H), 4.60 (m, 1H), 5.42 (d, 1H, J=3 Hz), 6.51-6.65 (m, 2H), 6.69-6.82 (m, 2H), 7.20-7.40 (m, 5H). MS APCI, m/z=324 (M+Na). LC/MS: 1.70 min.

### c. (2,3-trans)-3-Hydroxy-2-phenyl-2,3-dihydro-1,5-benzoxazepin-4(5H)-one (6c)

To a stirred solution of erythro ethyl 3-(2-aminophenoxy)-2-hydroxy-3-phenylpropanoate (6b) (3.585 g, 11.896 mmol) in THF (100 mL) cooled to 0° C. was added a solution of lithium hydroxide monohydrate (600 mg, 14.299 mmol) in water (25 mL) and methanol (2 mL). After 15 min the cooling bath was removed and the mixture stirred an additional 45 min warming to ambient temperature. The reaction was re-cooled to 0° C. and 1N aqueous hydrochloric acid (14.3 mL) was added. Solvent was then removed in-vacuo. The residue was dissolved in DMF (25 mL), HOBt (1.94 g, 14.40 mmol), NMM (3.34 g, 33.00 mmol), and EDAC (2.76 g, 14.40 mmol) were added and the mixture stirred for ˜12 h under nitrogen at ambient temperature. The reaction was diluted with water and extracted with EtOAc. The organic extracts were combined, dried, filtered and evaporated. The residue was purified by flash chromatography (2:1 (v/v) hexane-EtOAc) to afford the title compound (1.05 g, 34%) as a white solid. ¹H NMR (300 MHz, CDCl₃) δ3.70 (d, 1H, J=5 Hz), 4.63 (m, 1H), 5.28 (d, 1H, J=10 Hz), 6.89 (m, 1H), 7.02-7.16 (m, 3H), 7.35-7.47 (m, 5H), 7.78 (br, 1H). MS APCI, m/z=256 (M+1). LC/MS: 1.84 min.

### d. (2,3-cis)-3-Azido-2-phenyl-2,3-dihydro-1,5-benzoxazepin-4(5H)-one (6d)

Trifluoromethanesulfonyl chloride (770 mg, 4.57 mmol) was added via syringe to a stirred solution of (2,3-trans)-3-hydroxy-2-phenyl-2,3-dihydro-1,5-benzoxazepin-4(5H)-one (6c) (765 mg, 3.00 mmol) and triethylamine (508 mg, 5.00 mmol) in DCM (20 mL) under nitrogen at 0° C. The mixture was kept at 0° C. for ˜12 h. Additional trifluoromethanesulfonyl chloride (770 mg, 4.57 mmol) and triethylamine (508 mg, 5.00 mmol) was added and the mixture kept at 0° C. for an additional 4 h. Additional trifluoromethanesulfonyl chloride (1540 mg, 9.14 mmol) and triethylamine (1016 mg, 10.00 mmol) was added and the mixture kept at 0° C. for an additional 3 h. The reaction was concentrated in vacuo without heating, and the resulting residue immediately dissolved in DMF (5 mL) at 0° C. under nitrogen. Sodium azide (650 mg, 10.00 mmol) was added to the solution and the mixture allowed to warm to ambient temperature over 30 min. After an additional 30 min. the reaction was diluted with water and extracted with EtOAc. The organic extracts were dried, filtered and evaporated. The residue was purified by flash chromatography on silica gel eluting with 3:1 (v/v) hexanes:EtOAc to afford the title compound (778 mg, 2.77 mmol, 92%) as a foamy white solid. TLC R_{f}=0.38 (3:1 hexanes:EtOAc). ¹H NMR (300 MHz, CDCl₃) δ4.45 (d, 1H, J=6 Hz), 5.56 (d, 1H, J=6 Hz), 7.00-7.07 (m, 1H), 7.10-7.26 (m, 3H), 7.40-7.46 (m, 3H), 7.51-7.61 (m, 3H). MS APCI, m/z=253 (M+1−N₂). LC/MS: 2.25 min.

### e. (2,3-cis)-3-Amino-2-phenyl-2,3-dihydro-1,5-benzoxazepin-4(5H)-one hydrochloride (6e)

To a solution of (2,3-cis)-3-azido-2-phenyl-2,3-dihydro-1,5-benzoxazepin-4(5H)-one (6d) (610 mg, 2.176 mmol) in ethanol (40 mL) was added 5% palladium on carbon (65 mg) and 1N hydrochloric acid (2.4 mL). The mixture was stirred for 3 h under 1 atmosphere of hydrogen. The mixture was filtered through diatomaceous earth and the resulting solution was evaporated to afford the title compound (550 mg, 99%) as a tan solid. ¹H NMR (300 MHz, DMSO-d6) δ4.55 (d, 1H, J=7 Hz), 5.85 (d, 1H, J=7 Hz), 7.06-7.62 (m, 9H), 8.29 (br, 3H), 10.64 (s, 1H). MS APCI, m/z=255 (M+1). LC/MS: 1.29 min.

### f. N²-[tert-Butoxycarbonyl]-N¹-[(2,3-cis)-4-oxo-2-phenyl-2,3,4,5-tetrahydro-1,5-benzoxazepin-3-yl]-L-alaninamide (6f)

To a stirred solution of (2,3-cis)-3-amino-2-phenyl-2,3-dihydro-1,5-benzoxazepin-4(5H)-one hydrochloride (6e) (1.500 g, 5.90 mmol) in dry DCM (50 mL) under nitrogen was added BOC-L-alanine (1.172 g, 6.190 mmol), HOBt (0.957 g, 7.083 mmol) and EDAC-HCl (1.357 g, 7.078 mmol). The mixture was stirred for ˜12 h at 25° C., then diluted with water and extracted with EtOAc. The combined organic extracts were washed in succession with saturated aqueous sodium bicarbonate, 1N aqueous HCl, and brine. The organic solution was dried, filtered and evaporated. The residue was purified by flash chromatography on silica gel eluting with diethyl ether to afford the title compound as a yellow solid (1.880 g, 75%). R_{f}=0.46 (Et₂O). ¹H NMR (300 MHz, CDCl₃) δ1.14 (d, 1.5H, J=7.0 Hz), 1.21 (d, 1.5H, J=7.0 Hz), 1.38 (s, 4.5H), 1.41 (s, 4.5H), 2.86 (s, 1H), 2.92 (s, 1H), 4.05 (m, 1H), 4.63 (m, 0.5H), 4.75 (m, 0.5H), 5.17 (m, 1H), 5.79 (m, 1H), 6.36 (m, 1H), 6.99-7.49 (m, 9H). MS APCI, m/z=448 (M+Na). LC/MS: 2.13 min.

### g. N¹-[(2R,3S)-4-Oxo-2-phenyl-2,3,4,5-tetrahydro-1,5-benzoxazepin-3-yl]-L-alaninamide (6g)

N²-[tert-butoxycarbonyl]-N¹-[(2,3-cis)-4-oxo-2-phenyl-2,3,4,5-tetrahydro-1,5-benzoxazepin-3-yl]-L-alaninamide (6g) (2.405 g, 5.652 mmol) was dissolved in 5:1 (v/v) DCM-trifluoroacetic acid (50 mL) and kept at ambient temperature under nitrogen for 90 min. The solution was evaporated, and the residue was dissolved in EtOAc and extracted with saturated aqueous sodium bicarbonate. The organic solution was dried and evaporated. The residue was purified by flash chromatography on silica gel (260 g) eluting with 100:5 (v/v) CHCl₃-methanol. The title compound (2R,3S diastereomer) eluted first (TLC R_{f}=0.28 in 100:5 CHCl₃:methanol) then the solvent was changed to 100:15 (v/v) CHCl₃-methanol to complete the elution of the 2S,3R diastereomer (TLC R_{f}=0.16 in 100:5 CHCl₃-methanol). Fractions containing the early eluting isomer were combined and evaporated to afford the title compound (765 mg, 2.351 mmol, 42%). ¹H NMR (300 MHz, CDCl₃) δ1.20 (d, 3H, J=7.0 Hz), 1.38 (br, 2H), 3.36 (q, 1H, J=7.0 Hz), 5.20 (t, 1H, J=7.3 Hz), 5.79 (d, 1H, J=7.0 Hz), 7.06 (m, 1H), 7.15-7.31 (m, 3H), 7.34-7.56 (m, 7H). MS APCI, m/z=326 (M+1). LC/MS: 1.46 min.

### Example 7

### N²-[(3,5-Difluorophenyl)acetyl]-N¹-[(6R,7R)-5-oxo-7-phenyl-1,4-thiazepan-6-yl]-L-alaninamide (7)

Using a procedure similar to that described in Example 1, except using (6,7-cis)-6-amino-7-phenyl-1,4-thiazepan-5-one (7d) (84.3 mg) as the amine component and isolation of title compound by Et₂O trituration of the crude material, the white solid title compound (7) was obtained as a 1:1 mixture with the 6S,7S diastereomer (99.8 mg, 59%), m.p. 128-133°. ¹H NMR (300 MHz, CDCl₃) δ 1.11 (d, 1.5H, J=7.0 Hz), 1.28 (d, 1.5H, J=6.6 Hz), 2.80 (m, 1H), 3.06 (m, 1H), 3.48 (s, 1H), 3.49 (s, 1H), 3.76 (m, 2H), 4.32 (m, 1H), 4.47 (m, 1H), 5.25 (t, 0.5H), 5.33 (t, 0.5H), 6.09 (d, 0.5, exchangeable) 6.25 (m, 1H, exchangeable), 6.38 (t, 0.5H, exchangeable), 6.79 (m, 3H), 7.00 (d, 0.5H, exchangeable), 7.08 (d, 0.5H, exchangeable) 7.24 (m, 5H). MS APCI, m/z=448(M+1). HPLC Method A: 2.62 min.

The starting amine (6,7-cis)-6-amino-7-phenyl-1,4-thiazepan-5-one (7d) was prepared in the following manner:

### b. Methyl 2-{[(benzyloxy)carbonyl]amino}-3-({2-[(tert-butoxycarbonyl)amino]ethyl}thio)-3-phenylpropanoate (7a)

Using a method similar to that described in Example 1, part b (Method B) a solution of tert-butyl N-(2-mercaptoethyl)carbamate (2.4 mL), methyl (2Z)-2-{[(benzyloxy)carbonyl]amino}-3-phenylprop-2-enoate (0.87 g), triethylamine (0.39 mL) and methanol (15 mL) was stirred at 25° C. for 3 d. Removal of the solvent and chromatograpy of the resultant crude oil on silica gel (10% to 25% EtOAc-hexanes) returned the title compound as a viscous oil (1.22 g, 90%). The proton NMR displayed a mixture of diastereomers of approximate ratio 7:3; the major diastereomer is reported. ¹H NMR (300 MHz, CDCl₃) δ 1.41 (s, 9H.), 2.48 (t, 2H), 3.19 (br, 2H), 3.65 (s, 3H), 4.12 (q, 1H), 4.35 (t, 1H), 4.73 (t, 2H), 5.09 (d, 2H), 7.27-7.34 (m, 10H). MS APCI, m/z=389(M-t-BuOCO)⁺. HPLC Method A: 3.47 min.

### b. Methyl 3-[(2-aminoethyl)thio]-2-{[(benzyloxy)carbonyl]amino}-3-phenylpropanoate (7b)

To a stirred cooled (ice-bath) solution of methyl 2-{[(benzyloxy)carbonyl]amino}-3-({2-[(tert-butoxycarbonyl)amino]ethyl}thio)-3-phenylpropanoate (7a) (1.20 g) and methanol (0.284 mL) in EtOAc (2 mL) was added dropwise from a syringe acetyl chloride (0.43 mL) and the mixture stirred in the ice bath for an additional 10 min and then at 25° C. for 50 min. Excess Et₂O was added and the white solid collected, dissolved in water, treated with an excess of K₂CO₃(aq) and extracted with DCM and twice with Et₂O. The dried organics (MgSO₄) were filtered and the solvent removed to yield the title compound as an oil (0.86 g, 90%). The proton NMR displayed a mixture of diastereomers of approximate ratio 7:3; the major diastereomer is reported. ¹H NMR (300 MHz, CDCl₃) [symbol] 1.46 (s, 2H, exchangeable), 2.47 (t, 2H), 2.76 (t, 2H), 3.66 (s, 3H), 3.35 (d, 1H), 4.75 (t, 1H), 5.09 (m, 2H), 5.76 (d, 1H, exchangeable), 7.28-7.34 (m, 10H). MS APCI, m/z=389(M+1). HPLC Method A: 2.27 min.

### c. Benzyl (6,7 cis)-5-oxo-7-phenyl-1,4-thiazepan-6-ylcarbamate (7c)

To a stirred solution of methyl 3-[(2-aminoethyl)thio]-2-{[(benzyloxy)carbonyl]amino}-3-phenylpropanoate (7b) (0.85 g) in DCM (10 mL) was added 2.0 M (CH₃)₃Al in toluene (2.2 mL) and the mixture stirred for ˜12 h at 25° C. The reaction mixture was treated with 0.5N hydrochloric acid (20 mL) and extracted with DCM. The dried extracts (MgSO₄) yielded the title compound as a mixture with the 6,7-trans diastereomer; HPLC Method A: 2.96 and 3.22 min. Trituration with Et₂O returned pure title compound as a white solid (0.32 g, 41%). ¹H NMR (300 MHz, CDCl₃) [symbol] 2.75-2.81 (m, 1H), 3.02-3.08 (m, 1H), 3.68-3.93 (M, 2H), 4.35 (d, 1H, J_{6,7}=3.8 Hz), 5.11 (2H), 5.23 (q, 1H, J_{6,7}=3.8 Hz), 6.02 (d, 1H, NH), 6.13 (br s, 1H, NH) 7.26-7.38 (m, 10H). MS APCI, m/z=357(M+1). HPLC Method A: 2.96 min.

### d. (6,7 cis)-6-Amino-7-phenyl-1,4-thiazepan-5-one (7d)

To benzyl (6,7 cis)-5-oxo-7-phenyl-1,4-thiazepan-6-ylcarbamate (1c) (0.30 g) was added 30% HBr/HOAc (3 mL) and magnetic stirring initiated. After a few minutes the evolution of CO₂ was evident. After 45 min, the mixture was treated with excess Et₂O and the white solid collected by filtration, dissolved in water (˜65 mL), treated with excess saturated aqueous sodium bicarbonate and extracted with DCM (25 mL portions). The dried DCM extracts (MgSO₄) yielded the title compound as a white solid (0.17 g, 91%). ¹H NMR (300 MHz, CDCl₃) [symbol] 1.70 (br s, 2H, NH₂), 2.75-2.82 (m, 1H), 2.95-3.02 (m, 1H), 3.68-3.87 (m, 2H), 4.14 (d, 1H, J_{6,7}=3.1 Hz), 4.33 (d, 1H, J_{6,7}=3.1 Hz), 6.13 (br s, 1H, NH), 7.29-7.42 (m, 5H). MS APCI, m/z=223(M+1). HPLC Method A: 0.63 min.

### Example 8

### N²-[(3,5-Difluorophenyl)acetyl]-N¹-[(3S,4R)-8-fluoro-2-oxo-4-phenyl-2,3,4,5-tetrahydro-1H-1-benzazepin-3-yl]-L-alaninamide (8)

To a solution of (3,4-cis)-3-amino-8-fluoro-4-phenyl-1,3,4,5-tetrahydro-2H-1-benzazepin-2-one (8k) (15 mg) in DCM (6 mL) at 0° C. under nitrogen was added N-[3,5-difluorophenyl)acetyl]-L-alanine (8e) (14 mg), HOBt-hydrate (15 mg), EDAC-HCl (16 mg) and NMM (14 μL). The reaction mixture was stirred for 1 h at 0° C. and then for 2 h at 25° C. The mixture was concentrated in vacuo and then partitioned between water (10 mL) and EtOAc (12 mL). The organic phase was collected and consecutively washed with water, saturated aqueous sodium bicarbonate, and brine, dried, filtered and evaporated to yield the off-white solid title compound (14 mg, 52%) as a 1:1 mixture with the 3R,4S diastereomer. ¹H NMR (400 MHz, CD₃OD) δ 0.86 (d 1.5H), 0.97 (d, 1.5H), 2.8 (m, 2H), 3.76 (s, 2H), 3.86 (m, 1H), 4.02 (m, 1H), 4.53 (t, 1H), 6.68-6.78 (m, 3H), 6.99 (m, 2H), 7.17-7.39 (m, 5H). MS APCI, m/z=496 (M+1), 518 (M+Na). LC/MS: 2.41 min.

The starting amine, (3,4-cis)-3-amino-8-fluoro-4-phenyl-1,3,4,5-tetrahydro-2H-1-benzazepin-2-one (8k), was prepared in the following manner:

### a. Dimethyl [2-(4-fluoro-2-nitrophenyl)-1-phenylethyl]malonate (8a)

A solution of benzylidene malonate (14.2 g) in DMF (280 mL) was treated with sodium hydride (2.57 g, 95%). A solution of 4-fluoro-2-nitrotoluene in DMF (10 mL) was added over 1 h, and the reaction mixture was stirred at 25° C. for ˜12 h and then quenched by the addition of glacial acetic acid (175 mL) at 0° C. A total of 500 mL of 70:30 water-methanol was added with stirring, and the organics were extracted with EtOAc. The organic extracts were combined and washed with saturated aqueous potassium carbonate solution and brine, dried over sodium sulfate, filtered, and concentrated in vacuo to give a dark brown oil. Flash chromatography on silica gel (75:25 hexane-EtOAc, then 50:50 hexane-EtOAc) provided 7.0 g (30%) of the title compound as a red-brown solid. ¹H NMR (300 MHz, CDCl₃) δ 3.72 (d, 2H), 3.73 (d, 1H), 4.32 (m, 1H), 7.09-7.29 (m, 7H), 7.71 (m, 1H). MS APCI, m/z=398 (M+Na). LC/MS: 2.66 min.

### b. Dimethyl [2-(2-amino-4-fluorophenyl)-1-phenylethyl]malonate (8b)

To a solution of dimethyl [2-(4-fluoro-2-nitrophenyl)-1-phenylethyl]malonate (8a) (5.0 g) in methanol (20 mL) was added ammonium chloride (1.5 g) and zinc dust (11.0 g). The reaction mixture was then heated to reflux for 1 h. The reaction mixture was filtered through a diatomaceous earth pad, and the organic solvents were removed in vacuo. The resulting yellow oil was dissolved in EtOAc (100 mL) and washed with saturated aqueous potassium carbonate solution, the organic layer was dried, filtered, and concentrated in vacuo to afford the title compound as a tan gum (4.1 g, 90%). MS APCI, m/z=346 (M+1). LC/MS: 2.51 min.

### c. Methyl (3,4-trans)-8-fluoro-2-oxo-4-phenyl-2,3,4,5-tetrahydro-1H-1-benzazepine-3-carboxylate (8c)

A solution of dimethyl [2-(2-amino-4-fluorophenyl)-1-phenylethyl]malonate (8b) (4.3 g) in methanol (130 mL) was treated with sodium methoxide (1.73 g). The reaction mixture was heated to reflux for 5 h, cooled to 25° C., and acidified with 1N hydrochloric acid. The methanol was evaporated in vacuo, the residue was extracted with EtOAc, and the extract was washed with brine, 1N hydrochloric acid, and brine, dried, filtered, and concentrated in vacuo. The resulting crude product (4.0 g) was triturated with 1:1 diethyl ether-EtOAc to give the title compound as a colorless solid (3.87 g, 93%). ¹H NMR (300 MHz, CDCl₃) δ 2.4 (m, 2H), 3.23 (m, 1H), 3.72 (s, 3H), 3.77 (m, 1H), 6.71-7.50 (m, 7H), 8.95 (s, 1H). MS APCI, m/z=336 (M+Na). LC/MS: 2.28 min.

### d. Methyl (3,4-cis)-8-fluoro-3-hydroxyl-2-oxo-4-phenyl-2,3,4,5-tetrahydro-1H-1-benzazepine-3-carboxylate (8d)

A solution of methyl (3,4-trans)-8-fluoro-2-oxo-4-phenyl-2,3,4,5-tetrahydro-1H-1-benzazepine-3-carboxylate (8c) (156 mg) in THF (10 mL) was cooled to −78° C. under nitrogen. Potassium hexamethyldisilazide (0.5 M in toluene, 4.0 mL, 4 equiv) was added, and the reaction mixture was stirred for 1 h at −78° C. Trimethyl phosphite (0.24 mL, 4 equiv) was added, and bubbling with oxygen gas through the solution was started. Bubbling with oxygen gas was continued while the temperature was allowed to warm to 0° C. over approximately 30 min. The reaction was quenched with acetic acid (7 mL), the solvents were partially removed in vacuo, EtOAc was added, and the organic layer was washed with 1N hydrochloric acid, saturated potassium carbonate, and brine, dried, filtered, and concentrated in vacuo to provide the title compound as a light yellow solid (130 mg, 80%). MS APCI, m/z=330 (M+1). LC/MS: 2.22 min.

### e. (3,4-cis)-8-Fluoro-3-hydroxy-4-phenyl-1,3,4,5-tetrahydro-2H-1-benzazepin-2-one (8e)

A solution of methyl (3,4-trans)-8-fluoro-3-hydroxyl-2-oxo-4-phenyl-2,3,4,5-tetrahydro-1H-1-benzazepine-3-carboxylate (18d (1.4 g) and LiI (2.3 g, 4 equiv) in pyridine (35 ml) and water (0.35 mL) was heated to reflux for 3 h. Pyridine was removed in vacuo, EtOAc was added, and the EtOAc solution was washed with 1N hydrochloric acid, saturated aqueous potassium carbonate solution, and brine, dried, and filtered. A small amount of insoluble material was collected from the separatory funnel. This material was washed several times with water and ether, and then used to seed the EtOAc solution. Refrigeration and filtration provided the title compound as an off-white solid (700 mg, 60%). ¹H NMR (300 MHz, CDCl₃) δ 1.78-2.05 (m, 2H), 2.87 (m, 1H), 4.20 (m, 1H), 4.43 (d, 1H), 6.71-7.75 (m, 7H), 8.35 (s, 1H). MS APCI, m/z=272 (M+1). LC/MS: 1.95 min.

### f. (3,4-cis)-Fluoro-2-oxo-4-phenyl-2,3,4,5-tetrahydro-1H-1-benzazepin-3-yl-methanesulfonate (8f)

To a solution of (3,4-cis)-8-fluoro-3-hydroxyl-4-phenyl-1,3,4,5-tetrahydro-2H-1-benzazepin-2-one (8e) (400 mg) in pyridine (3 mL) was added methylsulfonyl chloride (0.17 mL, 1.5 equiv) at 0° C. The reaction mixture was stirred for 3 h at 0° C., and then diluted with diethyl ether (50 mL), washed several times with water, 1N hydrochloric acid, saturated aqueous sodium bicarbonate solution, and brine, dried, filtered and concentrated in vacuo to afford the title compound as a white solid (512 mg, 90.9%). MS APCI, m/z=350 (M+1). LC/MS: 2.58 min.

### g. (3,4-trans)-8-Fluoro-2-oxo-4-phenyl-2,3,4,5-tetrahydro-1H-1-benzazepin-3-yl acetate (8g)

To a solution of (3,4-cis)-8-fluoro-2-oxo-4-phenyl-2,3,4,5-tetrahydro-1H-1-benzazepin-3-yl methanesulfonate (8f) (512 mg) in toluene (10 mL) was added 18-crown-6 (391.2 mg) and cesium acetate (2.8 g) at 25° C. under N². The reaction mixture was refluxed for ˜12 h and then washed consecutively with water, brine, 1N hydrochloric acid, saturated aqueous sodium bicarbonate solution, and brine, dried, filtered and concentrated in vacuo to afford light yellow solid (394 mg, 85%) as trans racemic. MS APCI, m/z=314 (M+1). LC/MS: 2.56 min.

### h. (3,4-trans)-8-Fluoro-3-hydroxy-4-phenyl-1,3,4,5-tetrahydro-2H-1-benzazepin-2-one (8h)

To a solution of (3,4-trans)-8-fluoro-2-oxo-4-phenyl-2,3,4,5-tetrahydro-1H-1-benzazepin-3-yl acetate (8g) (360 mg) in methanol (10 mL) with water (5 mL) was added lithium hydroxide (46 mg, 2 equiv). The reaction mixture was stirred at 25° C. for 5 h, and it was then acidified to ˜pH 1 with 1N hydrochloric acid. The mixture was extracted with EtOAc twice and the combined EtOAc layers were washed with water, and brine, dried, filtered and concentrated in vacuo. Flash chromatography (4:1 hexane-EtOAc) provided the title compound (218.20 mg, 70%). ¹H NMR (300 MHz, CDCl₃) δ 1.8-2.15 (m, 2H), 2.9 (m, 1H), 4.30 (m, 1H), 4.53 (d, 1H), 6.71-7.5 (m, 7H), 7.95 (s, 1H). MS APCI, m/z=272 (M+1). LC/MS: 1.94 min.

### i. (3,4-trans)-8-Fluoro-2-oxo-4-phenyl-2,3,4,5-tetrahydro-1H-1-benzazepin-3-yl 4-methylbenzensulfonate (8i)

To a solution of (3,4-trans)-8-fluoro-3-hydroxy-4-phenyl-1,3,4,5-tetrahydro-2H-1-benzazepin-2-one (8h) (140.0 mg) in pyridine (4 mL) was added p-toluenesulfonyl chloride (170.0 mg, 2.3 equiv) at 0° C. The reaction mixture was stirred for 3 h at 0° C., and then for 24 h at 25° C. The reaction mixture was then diluted with DCM (50 mL), and washed several times with water, saturated aqueous copper sulfate, and brine, dried, filtered and concentrated in vacuo to afford the title compound as a brown oil (153.7 mg, 70%). MS APCI, m/z=426 (M+1). LC/MS: 2.74.

### j. (3,4-cis)-3-Azido-8-fluoro-4-phenyl-1,3,4,5-tetrahydro-2H-1-benzazepin-2-one (8j)

To a solution of (3,4-trans)-8-fluoro-2-oxo-4-phenyl-2,3,4,5-tetrahydro-1H-1-benzazepin-3-yl 4-methylbenzensulfonate (8i) (220.0 mg) in DMF (4 mL) was added sodium azide (135.2 mg, 4.0 equiv) at 25° C. The reaction mixture was heated to 90° C. for 24 h, cooled to 25° C., diluted with EtOAc (50 mL), and washed with water and brine, dried, filtered and concentrated in vacuo to provide a brown residue. Flash chromatography (4:1 hexane-EtOAc-) provided the title compound (87.0 mg, 58.7%). MS APCI, m/z=297 (M+1). LC/MS: 2.46 min.

### k. (3(4-cis)-3-Amino-8-fluoro-4-phenyl-1,3,4,5-tetrahydro-2H-1-benzazepin-2-one (8k)

To a solution of (3,4-cis)-3-azide-8-fluoro-4-phenyl-1,3,4,5-tetrahydro-2H-1-benzazepin-2-one (8j) (65.0 mg) in THF (5 mL) was added PS-triphenylphosphine (1.0 g, 1.2 mmol/g, 5.5 equiv) at 25° C. The reaction mixture was stirred at 25° C. for 24 h. The mixture was filtered, and the resin was extracted with THF (10 mL) and EtOAc (10 mL). The combined organic extracts were concentrated in vacuo to provide a brown residue. Flash chromatography (1:1 hexane-EtOAc) provided the title compound (45.7 mg, 70.0%). ¹H NMR (300 MHz, CDCl₃) δ 1.42-1.52 (m, 2H), 1.98 (s, 2H), 2.94 (m, 1H), 3.85 (d, 1H), 6.62-7.74 (m, 8H). ¹⁹F NMR (300 MHz, CDCl₃) δ-113.0. MS APCI, m/z=271 (M+1). LC/MS: 1.44 min.

### Example 9

### N²-[(3,5-Difluorophenyl)acetyl]-N¹-[(2R,3R)-4-oxo-2-phenyl-2,3,4,5-tetrahydro-1,5-benzothiazepin-3-yl]-L-alaninamide (9)

To a suspension of (2,3-cis)-3-amino-2-phenyl-2,3-dihydro-1,5-benzothiazepin-4(5H)-one hydrobromide (9d) (100 mg, 0.28 mmol) in DCM (20 ml) at 0° C. under nitrogen was added 1e (69 mg, 0.28 mmol) and NMM (31 μL, 0.28 mmol) until the solution cleared. To the solution was added HOBt-hydrate (96 mg, 0.63 mmol), EDAC-HCl (82 mg, 0.43 mmol) and NMM (50 μL, 0.43 mmol). The reaction mixture was stirred 2 h at 0° C., concentrated in vacuo and partitioned between H₂O (100 mL) and EtOAc (125 mL). The organic phase was collected and consecutively washed with HCl, H₂O, saturated NaHCO₃, and brine, dried and the solvent removed in vacuo to yield the title compound (9) as a 1:1 mixture with the 2S,3S diastereomer (130 mg, 92%). The crude product was purified by isocratic flash chromatography (50% EtOAc-hexanes) to yield 9 as an 84:16 mixture with the 2S,3S diastereomer (50 mg, 60%). Recrystallization from CHCl₃-hexanes gave pure title compound (9) (15 mg, >98% de) as a white solid.

¹H NMR (300 MHz, CDCl₃) δ 1.16 (d, 3H), 3.43 (s, 2H), 4.18 (m, 1H), 4.87 (t, 1H), 5.25 (d, 1H), 5.77 (d, 1H), 6.30 (d, 1H), 6.75 (m, 2H), 7.15 (d, 1H), 7.35 (m, 8H), 7.64 (bs, 1H), 7.71 (d, 1H). MS APCI, m/z=496 (M+1). LC/MS: 2.41 min.

The starting amine, (2,3-cis)-3-amino-2-phenyl-2,3-dihydro-1,5-benzothiazepin-4(5H)-one hydrobromide (9d), was prepared in the following manner:

### a. Methyl (2Z)-2-{[(benzyloxy)carbonyl]amino}-3-phenylacrylate (9a)

Using a procedure similar to that described in Example 1 part a, except using benzaldehyde (1.22 ml, 12.1 mmol) as the aldehyde component, the title compound (9a) was obtained as a 12:1 (Z:E) mixture of isomers as an oil (2.5 g, 66%). MS APCI, m/z=437 (M+1)

### b. Methyl β-[(2-aminophenyl)thio]-N-[(benzyloxy)carbonyl]-2,5-difluorophenylalaninate (9b)

To a deoxygenated solution of methyl (2Z)-2-{[(benzyloxy)carbonyl]amino}-3-phenylacrylate (9a) (2.5 g, 8.0 mmol) in anhydrous methanol (250 mL) under nitrogen was added the 2-aminothiophenol (10.6 g, 84.7 mmol) followed by addition of triethylamine (560 μL, 4.02 mmol). After 5 d an additional portion of triethylamine (560 uL, 4.02 mmol) was added and the reaction stirred at ambient temperature for 2 d. The reaction mixture was concentrated to −10 mL and partitioned between cold 1N hydrochloric acid (75 mL) and EtOAc (125 mL). The organic phase was separated and consecutively washed with 1N hydrochloric acid, dilute aqueous sodium bicarbonate and brine, dried, filtered and evaporated. The title compound (9b) was isolated as the hydrochloride salt (1.5 g, 40%, contaminated with 10% polymer). A 500 mg sample was recrystallized from hot EtOAc (10 mL) to afford analytically pure title compound (180 mg). MS APCI, m/z=437 (M+1). LC/MS: 2.68 min.

### c. Benzyl [(2,3-cis)-4-oxo-2-phenyl-2,3,4,5-tetrahydro-1,5-benzothiazepin-3-yl]carbamate (9c)

Using a procedure similar to that described in Example 1, part c, except using methyl β-[(2-aminophenyl)thio]-N-[(benzyloxy)carbonyl]phenylalaninate (9b) (850 mg, 1.9 mmol) as the anilino component, the title compound (9c) was obtained (730 mg, 92%) as a white solid. MS APCI, m/z=427 (M+Na), LC/MS: 2.67 min.

### d. (2,3-cis)-3-Amino-2-phenyl-2,3-dihydro-1,5-benzothiazepin-4(5H)-one hydrobromide (9d)

Using a procedure similar to that described in Example 1, part d Method D, except using benzyl [(2,3-cis)-4-oxo-2-phenyl-2,3,4,5-tetrahydro-1,5-benzothiazepin-3-yl]carbamate (9c), (550 mg, 1.36 mmol) as the protected amine component, the title compound (9d) was obtained (414 mg, 86%) as a white solid. MS APCI, m/z=293 (M+Na), LC/MS: 1.54 min.

### Example 10

### N¹-[(2R,3R)-2-(3,4-Dichlorophenyl)-4-oxo-2,3,4,5-tetrahydro-1,5-benzothiazepin-3-yl]-N²-[(3,5-Difluorophenyl)acetyl]-L-alaninamide (10)

To a suspension of (2,3-cis)-3-amino-2-(3,4-dichlorophenyl)-2,3-dihydro-1,5-benzothiazepin-4(5H)-one hydrobromide (10d) (236 mg, 0.56 mmol) in DCM (40 mL) at 0° C. under nitrogen was added the chiral acid (1e) (137 mg, 0.56 mmol) and NMM (62 μL, 0.56 mmol) until solution cleared. To the solution was added HOBt-hydrate (190 mg, 1.24 mmol), EDAC-HCl (162 mg, 0.85 mmol) and NMM (93 μL, 0.85 mmol). The reaction mixture was stirred 2 h at 0° C., then at 25° C. for 30 min, concentrated in vacuo and partitioned between H₂O (100 mL) and EtOAc (125 mL). The organic phase was collected and consecutively washed with HCl, H₂O, saturated NaHCO₃, and brine, dried and the solvent removed in vacuo to yield the title compound (10) as a 1:1 mixture with the 2S,3S diastereomer (130 mg, 92%). Crystallization from ether-hexanes gave 132 mg as a 3:1 mixture of predominantly the other 2S,3S diastereomer. The filtrate was evaporated and recrystallized from EtOAc/hexanes to give 65 mg of the title compound as a 6:1 mixture with the 2S,3S diastereomer as white solid.

¹H NMR (300 MHz, CDCl₃) δ 1.20 (d, 3H)[1.04 (d, 0.17H) 2S,3S], 3.45 (s, 2H), 4.23 (m, 1H), 4.82 (t, 1H), 5.25 (d, 1H), 5.76 (d, 1H), 6.53 (d, 1H), 6.6-6.8 (m, 3H), 7.15 (d, 1H), 7.31 (m, 1H), 7.4-7.5, (m, 3H), 7.50 (d, 1H), 7.70 (m, 2H). MS APCI, m/z=564 (M+1). LC/MS: 2.72 min.

The starting amine, (2,3-cis)-3-amino-2-(3,4-dichlorophenyl)-2,3-dihydro-1,5-benzothiazepin-4(5H)-one hydrobromide (10d), was prepared in the following manner:

### a. Methyl (2Z)-2-{[(benzyloxy)carbonyl]amino}-3-(3,4-dichlorophenyl)acrylate (10a)

Using a procedure similar to that described in Example 1 part a, except using 3,4-dichlorobenzaldehyde (1.05 g, 6.0 mmol) as the aldehyde component, the title compound (10a) was obtained as a 12:1 (Z:E) mixture of isomers as an oil (2.17 g, 92%)

### b. Methyl β-[(2-aminophenyl)thio]-N-[(benzyloxy)carbonyl]-3,4-dichlorophenylalaninate (10b)

To a deoxygenated solution of methyl (2Z)-2-{[(benzyloxy)carbonyl]amino}-3-(3,4-dichlorophenyl)acrylate (10a) (2.17 g, 5.7 mmol) in anhydrous methanol (40 mL) (warmed to dissolve) under nitrogen was added the 2-aminothiophenol (3.1 mL, 28.5 mmol) followed by addition of triethylamine (400 uL, 2.85 mmol). The mixture stirred 3 d at ambient temperature, concentrated to ca. 10 mL, partitioned between cold 1N hydrochloric acid (75 mL) and EtOAc (100 mL). The organic phase was separated and consecutively washed with 1N hydrochloric acid, dilute aqueous sodium bicarbonate and brine, dried, filtered and evaporated to give the title compound (10b) as an 4:1 mixture (erythro:threo) (2.8 g, 96%). A 100 mg sample was converted to the HCl salt to provide an analytically pure title compound (32 mg) as a (4:1) mixture. MS APCI, m/z=505 (M+1), 507 (M+3). LC/MS: 2.94 min.

### c. Benzyl [(2,3-cis)-2-(3,4-dichlorophenyl)-4-oxo-2,3,4,5-tetrahydro-1,5-benzothiazepin-3-yl]carbamate (10c)

A suspension of methyl β-[(2-aminophenyl)thio]-N-[(benzyloxy)carbonyl]-3,4-dichlorophenylalaninate (10b) (4:1, 2.5 g, 4.9 mmol) and pTSA (catalytic) in xylenes (30 mL) was heated to reflux for 1 h, using a Dean-Stark apparatus. The mixture was then cooled, resulting in precipitation of the trans product as a white solid (300 mg, pure trans). The filtrate (1.6 g, 85% pure cis +15% SM) was taken up in 20 mL xylenes, cat p-TSA, refluxed additional 1 h, cooled, evaporated, purified by flash column chromatography eluting with 50% ether-hexanes to give pure title compound (10c) (900 mg, 39%) as a white solid. MS APCI, m/z=495 (M+Na). LC/MS: 2.96 min.

### d. (2,3-cis)-3-Amino-2-phenyl-2,3-dihydro-1,5-benzothiazepin-4(5H)-one hydrobromide (10d)

Using a procedure similar to that described in Example 1, part d Method D, except using benzyl [(2,3-cis)-2-(3,4-dichlorophenyl)-4-oxo-2,3,4,5-tetrahydro-1,5-benzothiazepin-3-yl]carbamate (10c), (850 mg, 1.8 mmol) as the protected amine component, the title compound (10d) was obtained (750 mg, 99%) as a white solid. MS APCI, m/z=402 (M-NH₃), LC/MS: 1.88 min.

### Example 11

### N¹-[(2R,3R)-2-(4-chlorophenyl)-4-oxo-2,3,4,5-tetrahydro-1,5-benzothiazepin-3-yl]-N²-[(3,5-Difluorophenyl)acetyl]-L-alaninamide (11)

To a suspension of (2,3-cis)-3-amino-2-phenyl-2,3-dihydro-1,5-benzothiazepin-4(5H)-one hydrobromide (11d) (112 mg, 0.29 mmol) in DCM (25 mL) at 0° C. under nitrogen was added the chiral acid (1e) (71 mg, 0.29 mmol) and NMM (32 μL, 0.29 mmol) until solution cleared. To the solution was added HOBt-hydrate (98 mg, 0.64 mmol), EDAC.HCl (84 mg, 0.44 mmol) and NMM (51 μL, 0.44 mmol). The reaction mixture was stirred 2 h at 0° C., concentrated in vacuo and partitioned between H₂O (100 mL) and EtOAc (125 mL). The organic phase was collected and consecutively washed with HCl (2×), H₂O, saturated NaHCO₃, and brine, dried and the solvent removed in vacuo to yield the title compound (11) as a 1:1 mixture with the 2S,3S diastereomer (145 mg, 95%) as an off white solid.

¹H NMR (300 MHz, CDCl₃) δ0.99 (d, 1.5H), 1.18 (d, 1.5H), 3.43 (s, 1H), 3.45 (s, 1H), 4.28 (m, 0.5H), 4.63 (m, 0.5H), 4.83 (dt, 1H), 5.27 (dd, 1H), 5.87 (d, 0.5H), 6.18 (d, 0.5H), 6.55 (d, 0.5H), 6.74 (m, 3H), 6.85 (d, 0.5H), 7.05 (d, 0.5H), 7.16 (d, 0.5H), 7.34 (m, 6H), 7.63, (dd, 1H), 7.82 (s, 0.5H), 8.33 (s, 0.5H). MS APCI, m/z=530 (M+1), 552 (M+Na⁺). LC/MS: 2.58 min.

The starting amine, (2,3-cis)-3-amino-2-(4-chlorophenyl)-2,3-dihydro-1,5-benzothiazepin-4(5H)-one hydrobromide (11d), was prepared in the following manner:

### a. Methyl (2Z)-2-{[(benzyloxy)carbonyl]amino}-3-(4-chlorophenyl)acrylate (11a)

Using a procedure similar to that described in Example 1 part a, except using 4-chlorobenzaldehyde (844 mg, 6.0 mmol) as the aldehyde component, the title compound (11a) was obtained as white solid (2.0 g, 97%)

### b. Methyl β-[(2-aminophenyl)thio]-N-[(benzyloxy)carbonyl]-4-chlorophenylalaninate (11b)

Using a procedure similar to that described in Example 10, part b, except using methyl (2Z)-2-{[(benzyloxy)carbonyl]amino}-3-(4-chlorophenyl)acrylate (11a) (1.9 g, 5.5 mmol), the product was obtained as a crude oil. Recrystallization from ether-hexanes afforded the title compound (11b) (2.5 g, 97%) as white solid (95:5 mixture (erythro:threo)). MS APCI, m/z=471 (M+1). LC/MS: 2.82 min.

### c. Benzyl [(2,3-cis)-2-(4-chlorophenyl)-4-oxo-2,3,4,5-tetrahydro-1,5-benzothiazepin-3-yl]carbamate (11c)

A suspension of methyl β-[(2-aminophenyl)thio]-N-[(benzyloxy)carbonyl]-4-chlorophenylalaninate (11b) (19:1, 2.4 g, 4.9 mmol) and pTSA (catalytic) in xylenes (30 mL) was heated to reflux for 2 h, using a Dean-Stark apparatus. The mixture was then cooled, resulting in precipitation of the trans product as a white solid (350 mg, pure trans). The filtrate (2.0 g) purified by flash column chromatography eluting with 30% EtOAc-hexanes to give pure title compound (11c) (1.6 g, 72%) as a white solid. ¹H NMR (300 MHz, d6-DMSO) δ 4.54 (t, 1H, J=7.8 Hz), 4.95 (s, 2H), 5.19 (d, 1H, J=6.9 Hz), 6.41 (d, 1H), 7.23-7.34 (m, 6H), 7.42, (s, 5H), 7.51 (t, 1H, J=7.7 Hz), 7.67 (d, 1H, J=7.7 Hz), 10.50 (s, 1H). MS APCI, m/z=461 (M+Na). LC/MS: 2.83 min.

### d. (2,3-cis)-3-Amino-2-phenyl-2,3-dihydro-1,5-benzothiazepin-4(5H)-one hydrobromide (11d)

To benzyl [(2,3-cis)-2-(4-chlorophenyl)-4-oxo-2,3,4,5-tetrahydro-1,5-benzothiazepin-3-yl]carbamate (11c) (1.5 g, 3.4 mmol) in 5 mL acetic acid was added 30% HBr/acetic acid (7 mL). The stirred suspension was heated to 60 C for 40 min, cooled and then was diluted with ether to afford the hydrobromide salt of the title compound (11d) (1.1 g, 84%) as a white solid. ¹H NMR (300 MHz, d6-DMSO) δ 4.30 (d, 1H, J 6.6 Hz), 5.27 (d, 1H, J7.7 Hz), 6.41 (d, 1H), 7.29 (dd, 2H), 7.54 (m, 5H), 7.69 (d, 1H, J 7.9 Hz), 8.09 (bs, 3H), 10.88 (s, 1H). MS APCI, m/z=368 (M-NH₃). LC/MS: 1.73 min.

### Example 12

### N²-[(3,5-Difluorophenyl)acetyl]-N¹-[(2R,3R)-2-(4-methylphenyl)-4-oxo-2,3,4,5-tetrahydro-1,5-benzothiazepin-3-yl]-L-alaninamide (12)

Using a procedure similar to that described in Example 11, except using (2,3-cis)-3-amino-2-(4-methylphenyl)-2,3-dihydro-1,5-benzothiazepin-4(5H)-one hydrobromide (12d) (100 mg, 0.27 mmol) as the amine component, the title compound (12) was obtained in a 1:1 mixture with the 2S,3S diastereomer (130 mg, 93%) as an off white solid. ¹H NMR (300 MHz, d6-DMSO) δ0.93 (d, 1.5H), 1.01 (d, 1.5H), 2.29 (s, 1.5H), 2.30 (s, 1.5H), 3.38 (d, 2H), 4.17 (m, 1H), 4.68 (m, 1H), 5.08 (m, 1H), 6.90 (m, 2H), 7.26 (m, 7H), 7.49, (m, 2H), 7.67 (d, 1H), 8.17 (d, 0.5H), 8.28 (d, 0.5H), 10.47 (d, 1H). MS APCI, m/z=510 (M+1), 532 (M+Na). LC/MS: 2.53 min.

The starting amine, (2,3-cis)-3-amino-2-(4-methylphenyl)-2,3-dihydro-1,5-benzothiazepin-4(5H)-one hydrobromide (12d), was prepared in the following manner:

### a. Methyl (2Z)-2-{[(benzyloxy)carbonyl]amino}-3-(4-methylphenyl)acrylate (12a)

Using a procedure similar to that described in Example 1 part a, except using 4-methylbenzaldehyde (1.56 g, 13.0 mmol) as the aldehyde component, the title compound (12a) was obtained as white solid (4.2 g, 98%). MS APCI, m/z=326 (M+1)

### b. Methyl β-[2-aminophenyl)thio]-N-[(benzyloxy)carbonyl]-4-methylphenylalaninate (12b)

Using a procedure similar to that described in Example 10, part b, except using methyl (2Z)-2-{[(benzyloxy)carbonyl]amino}-3-(4-chlorophenyl)acrylate (12a) (2.1 g, 6.45 mmol) (stirred for 4 weeks, ˜85% complete) the title compound was obtained as a crude oil. Recrystallization from EtOAc-hexanes to afforded the title compound (12b) (1.2 g, 41%) as white solid (>98% erythro). MS APCI, m/z=451 (M+1). LC/MS: 2.79 min.

### c. Benzyl [(2,3-cis)-2-(4-methylphenyl)-4-oxo-2,3,4,5-tetrahydro-1,5-benzothiazepin-3-yl]carbamate (12c)

A suspension of methyl β-[(2-aminophenyl)thio]-N-[(benzyloxy)carbonyl]-4-chlorophenylalaninate (2) (1.1 g, 2.35 mmol) and pTSA (catalytic) in xylenes (30 mL) was heated to reflux for 2.5 h, using a Dean-Stark apparatus. The mixture was then cooled, evaporated to an oil, triturated with 10 mL methanol and filtered to yield the predominantly trans product as a white solid (50 mg, pure trans). The filtrate was evaporated and recrystallized from ether to afford pure title compound (12c) (750 mg, 76%) as a white solid. ¹H NMR (300 MHz, DMSO-d6) δ2.31 (s, 3H), 4.50 (m, 1H), 5.13 (d, 1H, J=7.0 Hz), 5.93 (br d, 1H, J=7.9 Hz), 7.12-7.37 (m, 11H), 7.50 (m, 1H), 7.67 (m, 1H), 10.49 (s, 1H). MS APCI, m/z=441 (M+Na). LC/MS: 2.78 min.

### d. (2,3-cis)-3-Amino-2-(4-methylphenyl)-2,3-dihydro-1,5-benzothiazepin-4(5H)-one hydrobromide (12d)

To benzyl [(2,3-cis)-2-(4-methylphenyl)-4-oxo-2,3,4,5-tetrahydro-1,5-benzothiazepin-3-yl]carbamate (12c) (500 mg, 1.2 mmol) in 4 mL acetic acid was added 30% HBr-acetic acid (3 mL). The stirred suspension was heated to 60° C. for 30 min, cooled and then was diluted with ether to afford the hydrobromide salt of the title compound (12d) (410 mg, 94%) as a white solid. ¹H NMR (300 MHz, d6-DMSO) δ2.34 (s, 3H), 4.24 (d, 1H, J=7.0 Hz), 5.20 (d, 1H, J=7.0 Hz), 7.24 (d, 3H), 7.30 (t, 1H, J=7.9 Hz), 7.42 (d, 2H, J=7.9 Hz), 7.54 (t, 1H, J=7.9 Hz), 7.68 (d, 1H, J=7.9 Hz), 8.00 (bs, 3H) 10.84 (s, 1H). MS APCI, m/z=268 (M+1−NH₃). LC/MS: 1.63 min.

### Example 13

### N¹-[(2R,3R)-7-Chloro-4-oxo-2-phenyl-2,3,4,5-tetrahydro-1,5-benzothiazepin-3-yl]-N²-[(3,5-Difluorophenyl)acetyl]-L-alaninamide (13)

Using a procedure similar to that described in Example 11, except using (2,3-cis)-3-amino-7-chloro-2-phenyl-2,3-dihydro-1,5-benzothiazepin-4(5H)-one hydrobromide (13c) (100 mg, 0.26 mmol) as the amine component, the title compound (13) was obtained in a 1:1 mixture with the 2S,3S diastereomer (130 mg, 94%) as an off white solid. ¹H NMR (300 MHz, CDCl₃) δ0.95 (d, 1.5H), 1.18 (d, 1.5H), 3.44 (s, 1H), 3.45 (s, 1H), 4.27 (m, 0.5H), 4.51 (m, 0.5H), 4.83 (dd, 1H), 5.24 (dd, 1H), 5.87 (d, 0.5H), 6.11 (d, 0.5H), 6.42 (d, 0.5H), 6.73 (m, 3.5H), 7.12 (d, 0.5H), 7.17 (d, 0.5H), 7.25 (m, 1H), 7.35 (m, 5H), 7.69 (dd, 1H), 7.89 (s, 0.5H), 8.57 (s, 0.5H). MS APCI, m/z=530 (M+1). LC/MS: 2.59 min.

The starting amine, (2,3-cis)-3-amino-7-chloro-2-phenyl-2,3-dihydro-1,5-benzothiazepin-4(5H)-one hydrobromide (13c), was prepared in the following manner:

### a. Methyl β-[(2-amino-4-chlorophenyl)thio]-N-[(benzyloxy)carbonyl]phenyl alaninate (13a)

Using a procedure similar to that described in Example 10, part b, except using methyl (2Z)-2-{[(benzyloxy)carbonyl]amino}-3-phenylacrylate (9a) (4.0 g, 12.9 mmol) as the olefin component, and 4-chloro-2-aminothiophenol (10 g, 62.7 mmol) as the thiol component (stirred for 6 d), the product was obtained as a crude oil. Flash column chromatography (5:1 hexanes-EtOAc) returned the title compound (1.7 g, 28%). Recrystallization from EtOAc-hexanes afforded the title compound (13a) (1.4 g) as a white solid (>98% erythro). MS APCI, m/z=471 (M+1). LC/MS: 2.90 min.

### b. Benzyl [(2,3-cis)-7-chloro-4-oxo-2-phenyl-2,3,4,5-tetrahydro-1,5-benzothiazepin-3-yl]carbamate (13b)

A suspension of methyl β-[(2-amino-4-chlorophenyl)thio]-N-[(benzyloxy)carbonyl]phenylalaninate (13a) (2.2 g, 4.67 mmol) and pTSA (catalytic) in xylenes (30 mL) was heated to reflux for 1.5 h using a Dean-Stark apparatus. The solids were filtered off after cooling. Washing with ether afforded pure title compound (13b) (1.7 g, 83%) as a white solid. ¹H NMR 300 MHz, d6-DMSO) δ4.56 (d, 1H, j=7.5 Hz), 4.96 (s, 2H), 5.19 (d, 1H, j=7.0 Hz), 6.12 (d, 1H), 7.2-7.4, (m, 12H), 7.69 (d, 1H, J=8.3 Hz), 10.59 (s, 1H). MS APCI, m/z=439 (M+1). LC/MS: 2.85 min.

### c. (2,3-cis)-3-Amino-7-chloro-2-phenyl-2,3-dihydro-1,5-benzothiazepin-4(5H)-one hydrobromide (13c)

To benzyl [(2,3-cis)-7-chloro-4-oxo-2-phenyl-2,3,4,5-tetrahydro-1,5-benzothiazepin-3-yl]carbamate (13b) (490 mg, 1.1 mmol) in 5 mL acetic acid was added 30% HBr-acetic acid (4 mL). The stirred suspension was heated to 60° C. for 60 min, cooled and then was diluted with ether to afford the title compound (13c) (280 mg, 65%) as a white solid. ¹H NMR (300 MHz, DMSO-d6) δ4.40 (d, 1H, J=7.0 Hz), 5.24 (d, 1H, J=7.0 Hz), 7.30 (m, 1H), 7.35-7.57 (m, 6H), 7.70 (d, 1H, J=8.3 Hz), 8.07 (br s, 3H), 10.94 (s, 1H). MS APCI, m/z=305 (M+1). LC/MS: 1.68 min.

### Example 14

### N¹-(2R,3R)-7-chloro-5-[2-(dimethylamino)ethyl]-4-oxo-2-phenyl-2,3,4,5-tetrahydro-1,5-benzothiazepin-3-yl-N²-[(3,5-Difluorophenyl)acetyl]-L-alaninamide (14)

Using a procedure similar to that described in Example 1, except using (2,3-cis)-3-amino-7-chloro-5-[2-(dimethylamino)ethyl]-2-phenyl-2,3-dihydro-1,5-benzothiazepin-4(5H)-one (14b) (93 mg, 0.25 mmol) as the amine component, the title compound (14) was obtained as a 1:1 mixture with the 2S,3S diastereomer (94 mg, 62%) as a white solid. ¹H NMR (300 MHz, CDCl₃) δ0.90 (d, 1.5H), 1.14 (d, 1.5H), 2.29 (s, 6H), 2.50 (m, 1H), 2.70 (m, 1H), 3.41 (s, 2H), 3.72 (m, 1H), 4.18 (m, 1H), 4.35 (m, 1H), 4.70 (td, 1H), 5.07 (d, 1H), 5.71 (d, 0.5H), 5.94 (d, 0.5H), 6.27 (t, 1H), 6.74 (m, 2.5H), 7.2-7.4 (m, 6.5H), 7.50 (m, 1H), 7.65 (d, 1H). MS APCI, m/z=601 (M+1). LC/MS: 2.18 min.

The starting amine, (2,3-cis)-3-amino-7-chloro-5-[2-(dimethylamino)ethyl]-2-phenyl-2,3-dihydro-1,5-benzothiazepin-4(5H)-one (4b), was prepared in the following manner:

### a. Benzyl {(2,3-cis)-7-chloro-5-[2-(dimethylamino)ethyl]-4-oxo-2-phenyl-2,3,4,5-tetrahydro-1,5-benzothiazepin-3-yl}carbamate (14a)

To a solution of benzyl [(2,3-cis)-7-chloro-4-oxo-2-phenyl-2,3,4,5-tetrahydro-1,5-benzothiazepin-3-yl]carbamate (13b) (750 mg, 1.71 mmol) in methyl isobutyl ketone (20 mL) was added 10N NaOH (0.86 mL, 8.55 mmol) followed by H₂O (3.3 mL) and N,N-dimethylaminoethylchloride hydrochloride (370 mg, 2.57 mmol). The reaction mixture was heated to 95° C. for 2.5 h, allowed to cool to 25° C. and diluted with EtOAc. The organic phase was collected and consecutively washed with H₂O, brine, dried, filtered and the solvent removed in vacuo to yield crude oil (contained 10% 13b). The crude oil was dissolved in EtOAc made acidic to pH 1 with 4N dioxane, added ether to precipitate pure title compound (14a) (650 mg, 69%). MS APCI, m/z=510 (M+1). LC/MS: 2.37 min.

### b. (2,3-cis)-3-Amino-7-chloro-5-[2-(dimethylamino)ethyl]-2-phenyl-2,3-dihydro-1,5-benzothiazepin-4(5H)-one dihydrobromide (14b)

To benzyl {(2,3-cis)-7-chloro-5-[2-(dimethylamino)ethyl]-4-oxo-2-phenyl-2,3,4,5-tetrahydro-1,5-benzothiazepin-3-yl}carbamate (14a) (270 mg, 0.49 mmol) in 1 mL acetic acid was added 30% HBr/acetic acid (2 mL). The stirred suspension was heated to 60° C. for 40 min, cooled and then was diluted with ether to afford the hydrobromide salt of the title compound (11d) (259 mg, 97%) as a grey solid.

### Example 15

### N¹-[(2R,3R)-2-(3-Chlorophenyl)-4-oxo-2,3,4,5-tetrahydro-1,5-benzothiazepin-3-yl]-N²-[(3,5-Difluorophenyl)acetyl]-L-alaninamide (15)

Using a procedure similar to that described in Example 10 except using (2,3-cis)-3-amino-2-(3-chlorophenyl)-2,3-dihydro-1,5-benzothiazepin-4(5H)-one hydrobromide (15d) (115 mg, 0.3 mmol) as the amine component, the title compound (15) was obtained as a 1:1 mixture with the 2S,3S diastereomer (143 mg, 90%) as a off white semi-solid. This was crystallized from EtOAc-hexanes to give 62 mg of a 92:8 mixture of the 2S,3S diastereomer. The mother liquor was evaporated, washed with ether, dried in vacuo to yield the title compound (15) as a 87:13 mixture of diastereomers (50 mg) as a light yellow solid. ¹H NMR (300 MHz, CDCl₃) δ0.99 (d, 1.5H), 1.18 (d, 1.5H), 3.43 (s, 1H), 3.45 (s, 1H), 4.28 (m, 0.5H), 4.63 (m, 0.5H), 4.83 (dt, 1H), 5.27 (dd, 1H), 5.87 (d, 0.5H), 6.18 (d, 0.5H), 6.55 (d, 0.5H), 6.74 (m, 3H), 6.85 (d, 0.5H), 7.05 (d, 0.5H), 7.16 (d, 0.5H), 7.34 (m, 6H), 7.70 (dd, 1H), 7.89 (s, 0.5H), 8.57 (s, 0.5H). MS APCI, m/z=530 (M+1). LC/MS: 2.60 min.

The starting amine, (2,3-cis)-3-amino-2-(3-chlorophenyl)-2,3-dihydro-1,5-benzothiazepin-4(5H)-one hydrobromide (15d), was prepared in the following manner:

### a. Methyl (2Z)-2-{[(benzyloxy)carbonyl]amino}-3-(3-chlorophenyl)acrylate (15a)

Using a procedure similar to that described in Example 1 part a, except using 3-chlorobenzaldehyde (844 mg, 5.50 mmol) as the aldehyde component, the title compound (15a) with 15% of the (2E) isomer was obtained as a semi-solid (1.9 g, 97%).

### b. Methyl β-[(2-aminophenyl)thio]-N-[(benzyloxy)carbonyl]-3-chlorophenylalaninate (15b)

Using a procedure similar to that described in Example 10, part b, except using methyl (2Z)-2-{[(benzyloxy)carbonyl]amino}-3-(3-chlorophenyl)acrylate (15a) (1.9 g, 5.5 mmol), to afford the title compound (15b) as a 4:1 mix (erythro:threo) (2.5 g, 97%) as an oil. ¹H NMR (300 MHz, CDCl₃) δ3.46 (s, 3H), 4.25 (br s, 2H), 4.59 (m, 1H), 4.79 (m, 1H), 5.10 (m, 2H), 5.85 (br, 1H), 6.59 (m, 1H), 6.68 (br d, 1H, J=7.9 Hz), 7.04-7.27 (m, 6H), 7.28-7.42 (m, 5H). MS APCI, m/z=471 (M+1). LC/MS: 2.82 min.

### c. Benzyl [(2,3-cis)-2-(3-chlorophenyl)-4-oxo-2,3,4,5-tetrahydro-1,5-benzothiazepin-3-yl]carbamate (15c)

A suspension of methyl β-[(2-aminophenyl)thio]-N-[(benzyloxy)carbonyl]-4-chlorophenylalaninate (11b) (70:30 erythro:threo, 1.7 g, 4.9 mmol) and pTSA (catalytic) in xylenes (30 mL) was heated to reflux for 2 h, using a Dean-Stark apparatus. The mixture was then cooled, the solid filtered off and washed with ether to afford the title compound (12c) (1.45 g) as a 2:1 mixture with 2,3-trans product. Purification by flash column chromatography (20% EtOAc-hexanes) gave pure title compound (15c) (0.95 g, 60%) as a white solid. ¹H NMR (300 MHz, d6-DMSO) δ 4.56 (t, 1H, J=7.4 Hz), 4.96 (s, 2H), 5.19 (d, 1H, J=7.0 Hz), 6.53 (d, 1H), 7.23-7.41 (m, 10H), 7.52, (m, 2H), 7.67 (d, 1H, J=7.5 Hz), 10.53 (s, 1H). MS APCI, m/z=439 (M+1), 461 (M+Na). LC/MS: 2.88 min.

### d. (2,3-cis)-3-Amino-2-(3-chlorophenyl-2,3-dihydro-1,5-benzothiazepin-4(5H)-one hydrobromide (15d)

To benzyl [(2,3-cis)-2-(3-chlorophenyl)-4-oxo-2,3,4,5-tetrahydro-1,5-benzothiazepin-3-yl]carbamate (15c) (230 mg, 0.52 mmol) in 5 mL acetic acid was added 30% HBr/acetic acid (1 mL). The stirred suspension was heated to 60° C. for 40 min, cooled and then was diluted with ether to afford the hydrobromide salt of the title compound (15d) (155 mg, 77%) as a white solid. ¹H NMR (300 MHz, d6-DMSO) δ4.32 (d, 1H, J=7.0 Hz), 5.26 (d, 1H, J=7.0 Hz), 7.29 m, 2H), 7.53 (m, 5H), 7.69 (d, 1H, J=7.5 Hz), 8.09 (bs, 3H), 10.91 (s, 1H). MS APCI, m/z=304 (M+1), 288 (M-NH₃). LC/MS: 1.74 min.

### Example 16

### N²-[(3,5-Difluorophenyl)acetyl]-N¹-[(2R,3R)-2-(3,5-difluorophenyl)-4-oxo-2,3,4,5-tetrahydro-1,5-benzothiazepin-3-yl]-L-alaninamide (16)

Using a procedure similar to that described in Example 11, except using (2,3-cis)-3-amino-2-(3,5-difluorophenyl)-2,3-dihydro-1,5-benzothiazepin-4(5H)-one (16d) (250 mg, 0.82 mmol) as the amine component, the title compound (16) was obtained in a 1:1 mixture with the 2S,3S diastereomer (430 mg, 98%) as a white solid. ¹H NMR (300 MHz, CDCl₃) δ1.05 (d, 1.5H), 1.20 (d, 1.5H), 3.45 (s, 1H), 3.47 (s, 1H), 4.34 (m, 0.5H), 4.70 (m, 0.5H), 4.78 (m, 1H), 5.30 (dd, 1H), 5.88 (d, 0.5H), 6.15 (d, 0.5H), 6.65-6.85 (m, 4H), 6.97 (m, 3H), 7.05 (d, 0.5H), 7.16 (d, 0.5H), 7.2-7.3 (m, 1H), 7.43 (q, 1H), 7.69 (m, 1H), 7.86 (bs, 0.5H), 8.64 (bs, 0.5H). MS APCI, m/z=532 (M+1). LC/MS: 2.57 min.

The starting amine, (2,3-cis)-3-amino-2-(3,5-difluorophenyl-2,3-dihydro-1,5-benzothiazepin-4(5H)-one hydrobromide (16d), was prepared in the following manner:

### a. Methyl (2Z)-2-{[(benzyloxy)carbonyl]amino}-3-(3,5-difluorophenyl)acrylate (16a)

Using a procedure similar to that described in Example 1 part a, except using 3,5-difluorobenzaldehyde (0.61 mL, 5.50 mmol) as the aldehyde component, the crude product was obtained as an oil (2.7 g) (9:1 Z:E). After recrystallization from EtOAc-ether-hexanes (cold) the pure title compound (16a) was obtained (1.3 g, 71%) as a white solid. MS APCI, m/z=334 (M+1).

### b. Methyl β-[(2-aminophenyl)thio]-N-[(benzyloxy)carbonyl]-3,5-difluorophenylalaninate (16b)

Using a procedure similar to that described in Example 9 part b, except using methyl (2Z)-2-{[(benzyloxy)carbonyl]amino}-3-(3,5-difluorophenyl)acrylate (16a) (1.2 g, 3.59 mmol) as the olefin component, the title compound (16b) (1.6 g, 97%) was obtained as a semi-solid. ¹H NMR (300 MHz, CDCl₃) δ3.50 (s, 3H), 4.27 (br s, 2H), 4.59 (m, 1H), 4.79 (m, 1H), 5.10 (m, 2H), 5.89 (br d, 1H, 9.2 Hz), 6.55-6.93 (m, 5H), 7.07-7.17 (m, 2H), 7.29-7.44 (m, 5H). MS APCI, m/z=473 (M+1).

### c. Benzyl [(2,3-cis)-4-oxo-2-(3,5-difluorophenyl-2,3,4,5-tetrahydro-1,5-benzothiazepin-3-yl]carbamate (16c)

Using a procedure similar to that described in Example 1, part c, except using methyl β-[(2-aminophenyl)thio]-N-[(benzyloxy)carbonyl](3,5-difluoro)phenylalaninate (9b) (850 mg, 1.9 mmol) as the anilino component, the title compound (2c) was obtained (730 mg, 92%) as a white solid. MS APCI, m/z=427 (M+Na), LC/MS: 2.67 min.

### d. (2,3-cis)-3-Amino-2-(3,5-difluorophenyl)-2,3-dihydro-1,5-benzothiazepin-4(5H)-one hydrobromide (16d)

Using a procedure similar to that described in Example 1, part d method D, except using benzyl [(2,3-cis)-4-oxo-2-(3,5-difluoro)phenyl-2,3,4,5-tetrahydro-1,5-benzothiazepin-3-yl]carbamate (16c), (550 mg, 1.29 mmol) as the protected amine component, the title compound (16d) was obtained (433 mg, 86%) as a white solid. ¹H NMR (300 MHz, DMSO-d6) δ4.38 (d, 1H, J=6.5 Hz), 5.30 (d, 1H, J=6.5 Hz), 7.17-7.44 (m, 5H), 7.57 (m, 1H), 7.71 (d, 1H), 8.18 (br, 3H), 10.97 (s, 1H). MS ES⁺, m/z=307 (M+1), HPLC: 2.30 min (Method C).

### Example 17

### N²-[(3,5-Difluorophenyl)acetyl]-N¹-[(2R,3R)-2-(3,5-difluorophenyl)-5-methyl-4-oxo-2,3,4,5-tetrahydro-1,5-benzothiazepin-3-yl]-L-alaninamide (17)

To a solution of N²-[(3,5-difluorophenyl)acetyl]-N¹-[(2R,3R)-2-(3,5-difluorophenyl)-4-oxo-2,3,4,5-tetrahydro-1,5-benzothiazepin-3-yl]-L-alaninamide (16) (85 mg, 0.16 mmol) in DMF (1 mL) was added a 60% suspension of NaH (6.6 mg, 0.165 mmol). The reaction was stirred at 25° C. for 5 min and methyl iodide (23 mg, 0.16 mmol) was added via syringe. The reaction was stirred at 25° C. for 3 h and was quenched with dropwise addition of 1N HCl, diluted with water and extracted with EtOAc. The organic phase was collected and washed with H₂O, dried, filtered and evaporated to yield a crude oil (85 mg). The crude product was purified by flash chromatography (50% EtOAc-hexanes) to afford the title compound (17) (70 mg, 80%) as a 1:1 mixture with the 2S,3S diastereomer as a white solid. ¹H NMR (300 MHz, CDCl₃) δ0.99 (d, 1.5H), 1.17 (d, 1.5H), 3.44 (s, 1H), 3.46 (s, 1H), 3.50 (s, 3H), 4.24 (m, 1H), 4.70 (m, 1H), 5.15 (m, 1H), 5.77 (d, 0.5H), 5.91 (d, 0.5H), 6.46 (dd, 1H), 6.65-6.92 (m, 6H), 7.31 (m, 2H), 7.51 (m, 1H), 7.71 (m, 1H). MS APCI, m/z=546 (M+1). LC/MS: 2.80 min.

### Example 18

### N²-[(3,5-Difluorophenyl)acetyl]-N¹-[(2R,3R)-2-(2-fluorophenyl)-5-methyl-4-oxo-2,3,4,5-tetrahydro-1,5-benzothiazepin-3-yl]-L-alaninamide (18)

Using a procedure similar to that described in Example 17, except using (2,3-cis)-3-amino-2-(2-fluorophenyl)-2,3-dihydro-1,5-benzothiazepin-4(5H)-one (90) (100 mg, 0.20 mmol) as the amine component, the title compound (18) was obtained in a 1:1 mixture with the 2S,3S diastereomer (70 mg, 68%) as a white solid. ¹H NMR (300 MHz, CDCl₃) δ 1.00 (d, 1.5H), 1.18 (d, 1.5H), 3.42 (s, 1H), 3.46 (s, 1H), 3.50 (s, 3H), 4.22 (m, 1H), 4.85 (m, 1H), 5.61 (m, 1H), 5.94 (m, 0.5H), 6.04 (m, 0.5H), 6.32 (m, 1H), 6.73 (m, 2H), 7.01 (m, 1H), 7.19-7.33 (m, 6H), 7.51 (m, 1H), 7.72 (m, 1H). MS APCI, m/z=550 (M+23). LC/MS: 2.78 min.

### Example 19

### N¹-(2R,3R)-2-(3-Chlorophenyl)-5-[2-(dimethylamino)ethyl]-4-oxo-2,3,4,5-tetrahydro-1,5-benzothiazepin-3-yl-N²-[(3,5-Difluorophenyl)acetyl]-L-alaninamide (19)

Using a procedure similar to that described in Example 1, part d, except using (2,3-cis)-3-amino-2-(3-chlorophenyl)-5-[2-(dimethylamino)ethyl]-2,3-dihydro-1,5-benzothiazepin-4(5H)-one (19b) (63 mg, 0.17 mmol) as the amine component, the title compound (19) was obtained as a 1:1 mixture with the 2S,3S diastereomer (45 mg, 45%) as a white solid (flash column chromatography conditions: 5-10% methanol-DCM). ¹H NMR (300 MHz, CDCl₃) δ0.95 (d, 1.5H), 1.16 (d, 1.5H), 2.29 (s, 6H), 2.43 (m, 1H), 2.65 (m, 1H), 3.43 (s, 1H), 3.45 (s, 1H), 3.64 (m, 1H), 4.19 (m, 1H), 4.48 (m, 1H), 4.66 (td, 1H), 5.15 (d, 1H), 5.73 (d, 0.5H), 5.95 (d, 0.5H), 6.34 (d, 0.5H), 6.42 (d, 0.5H), 6.74 (m, 3H), 7.2-7.4 (m, 3H), 7.45 (m, 4H), 7.72 (d, 1H). MS APCI, m/z=601 (M+1). LC/MS: 2.03 min.

The starting amine, (2,3-cis)-3-amino-2-(3-chlorophenyl)-5-[2-(dimethylamino)ethyl]-2,3-dihydro-1,5-benzothiazepin-4(5H)-one (19b), was prepared in the following manner:

### a. Benzyl {(2R,3R)-5-[2-(dimethylamino)ethyl]-4-oxo-2-phenyl-2,3,4,5-tetrahydro-1,5-benzothiazepin-3-yl}carbamate hydrochloride (19a)

Using a procedure similar to that described in Example 14, part a, except using benzyl [(2,3-cis)-4-oxo-2-(3-chlorophenyl-2,3,4,5-tetrahydro-1,5-benzothiazepin-3-yl]carbamate (15c) (311 mg, 0.71 mmol) as the amide component, the pure title compound (19a) (250 mg, 69%) was obtained as a white solid.

### b. (2,3-cis)-3-Amino-5-[2-(dimethylamino)ethyl]-2-(3-chlorophenyl)-2,3-dihydro-1,5-benzothiazepin-4(5H)-one (19b)

Using a procedure similar to that described in Example 14, part b, substituting benzyl {(2,3-cis)-5-[2-(dimethylamino)ethyl]-4-oxo-2-(3-chlorophenyl)-2,3,4,5-tetrahydro-1,5-benzothiazepin-3-yl}carbamate (19a) (95 mg, 0.19 mmol) as the protected amine component, the crude product was isolated as the hydrobromide salt. Partitioned between EtOAc and saturated sodium bicarbonate, the organic layer washed with brine, dried, filtered and evaporated to afford slightly crude title compound (19b) (63 mg, 90%) as a white semi-solid.

### Example 20

### N²-[(3,5-Difluorophenyl)acetyl]-N¹-[(2R,3R)-2-(2,5-difluorophenyl):4-oxo-2,3,4,5-tetrahydro-1,5-benzothiazepin-3-yl]-D-serinamide (20)

Using a procedure similar to that described in Example 4, except using N-[(3,5-difluorophenyl)acetyl]-D-serine as the acid component, the title compound (20) was obtained as a 1:1 mixture with the 2S,3S diastereomer (150 mg, 73%) as a white solid. ¹H NMR (300 MHz, DMSO) δ (d, 3H), 3.48 (s, 2H), 4.24 (m, 1H), 4.77 (m, 2H), 5.48 (dd, 1H), 6.93 (m, 2H), 7.05 (m, 1H), 7.18-7.35 (m, 5.5H), 7.45-7.55 (m, 2.5H), 7.64 (d, 0.5H), 7.72 (d, 1H), 7.79 (d, 0.5H), 8.13 (t, 1H), 10.64 (s, 1H). MS APCI, m/z=548 (M+1). LC/MS: 2.46 min.

### a. N-[(3,5-Difluorophenyl)acetyl]-D-serine

Was prepared as in Example 4 except using methyl D-serinate as the reactant

### Example 21

### N¹-[(2R,3R)-2-(3-Chlorophenyl)-5-methyl-4-oxo-2,3,4,5-tetrahydro-115 benzothiazepin-3-yl]-N²-[(3,5-difluorophenyl)acetyl]-L-alaninamide (21)

Using a procedure similar to that described in Example 11, except using (2,3-cis)-3-amino-2-(3-chlorophenyl)-5-methyl-2,3-dihydro-1,5-benzothiazepin-4(5H)-one (21b) (72 mg, 0.18 mmol) as the amine component, the title compound (21) was obtained in a 1:1 mixture with the 2S,3S diastereomer (90 mg, 92%) as a white solid. ¹H NMR (300 MHz, CDCl₃) δ0.95 (d, 1.5H), 1.16 (d, 1.5H), 3.43 (s, 1H), 3.45 (s, 1H), 3.50 (s, 3H), 4.21 (m, 1H), 4.72 (m, 1H), 5.13 (dd, 1H), 5.77 (m, 0.5H), 6.48 (m, 0.5H), 6.75 (m, 3H), 7.25-7.33 (m, 7H), 7.51 (t, 1H), 7.72 (d, 1H). MS APCI, m/z=544 (M+1). LC/MS: 2.84 min.

The starting amine, (2,3-cis)-3-amino-2-(3-chlorophenyl)-5-methyl-2,3-dihydro-1,5-benzothiazepin-4(5H)-one (21b) was prepared in the following manner:

### a. Benzyl [(2,3-cis)-4-oxo-2-(3-chlorophenyl)-2,3,4,5-tetrahydro-1,5-benzothiazepin-3-yl]carbamate (21a)

Using a procedure similar to that described in Example 5, part a, substituting benzyl [(2,3-cis)-2-(3-chlorophenyl)-4-oxo-2,3,4,5-tetrahydro-1,5-benzothiazepin-3-yl]carbamate (15c) (170 mg, 0.39 mmol) as the amide component, the title compound (2a) was obtained in a 1:1 mixture with the 2S,3S diastereomer (101 mg, 58%) as a white solid. (flash column chromatography: EtOAc). MS APCI, m/z=476 (M+Na). LC/MS: 3.25 min.

### b. (2,3-cis)-3-Amino-2-(3-chlorophenyl)-5-methyl-2,3-dihydro-1,5-benzothiazepin-4(5H)-one hydrobromide (21b)

Using a procedure similar to that described in Example 1, part d Method D, except using benzyl [(2,3-cis)-4-oxo-2-(3-chlorophenyl)-2,3,4,5-tetrahydro-1,5-benzothiazepin-3-yl]carbamate (21a), (98 mg, 0.22 mmol) as the protected amine component, the title compound (21b) was obtained (80 mg, 91%) as a white solid. ¹H NMR (300 MHz, DMSO-d6) δ3.48 (s, 3H), 4.27 (d, 1H, J=7.0 Hz), 5.14 (d, 1H, J=7.0 Hz), 7.37-7.56 (m, 5H), 7.66 (m, 2H), 7.75 (m, 1H), 8.07 (br, 3H). MS APCI, m/z=319 (M+1), LC/MS: 2.47 min.

### Example 22

### N²-[(3,5-Difluorophenyl)acetyl]-N¹-[(2R,3R)-5-methyl-4-oxo-2-phenyl-2,3,4,5-tetrahydro-1,5-benzothiazepin-3-yl]-L-alaninamide (22)

Using a procedure similar to that described in Example 11, except using (2,3-cis)-3-amino-5-methyl-2-phenyl-2,3-dihydro-1,5-benzothiazepin-4(5H)-one hydrobromide (22b) (99 mg, 0.27 mmol) as the amine component, the title compound (22) was obtained in a 1:1 mixture with the 2S,3S diastereomer (132 mg, 96%) as an off white solid. ¹H NMR (300 MHz, CDCl₃) δ0.89 (d, 1.5H), 1.14 (d, 1.5H), 3.41 (s, 1H), 3.43 (s, 1H), 3.51 (s, 3H), 4.17 (m, 1H), 4.75 (dt, 1H), 5.14 (d, 1H), 5.75 (m, 0.5H), 5.97 (m, 0.5H), 6.33 (t, 1H), 6.73 (m, 3H), 7.30-7.33 (m, 7H), 7.51 (m, 1H), 7.72 (d, 1H). MS APCI, m/z=510 (M+1). LC/MS: 2.41 min.

The starting amine, (2,3-cis)-3-amino-2-phenyl-5-methyl-2,3-dihydro-1,5-benzothiazepin-4(5H)-one (22b) was prepared in the following manner:

### a. Benzyl [(2R,3R)-5-methyl-4-oxo-2-phenyl-2,3,45-tetrahydro-1,5-benzothiazepin-3-yl]carbamate (22a)

Using a procedure similar to that described in Example 5, part a, substituting benzyl [(2,3-cis)-4-oxo-2-phenyl-2,3,4,5-tetrahydro-1,5-benzothiazepin-3-yl]carbamate (2c) (300 mg, 0.74 mmol) as the amide component, the title compound (22a) was obtained in a 1:1 mixture with the 2S,3S diastereomer (285 mg, 91%) as a white solid (recrystallized from EtOAc). MS APCI, m/z=441 (M+Na). LC/MS: 2.92 min.

### b. (2,3-cis)-3-Amino-5-methyl-2-phenyl-2,3-dihydro-1,5-benzothiazepin-4(5H)-one hydrobromide (22b)

Using a procedure similar to that described in Example 1, part d Method D, except using benzyl [(2,3-cis)-4-oxo-2-phenyl-2,3,4,5-tetrahydro-1,5-benzothiazepin-3-yl]carbamate (22a), (260 mg, 0.622 mmol) as the protected amine component, the title compound (22b) was obtained (170 mg, 75%) as a white solid. ¹H NMR (300 MHz, DMSO-d6) δ3.49 (s, 3H), 4.21 (d, 1H, J=7.0 Hz), 5.12 (d, 1H, J-7.0 Hz), 7.37-7.53 (m, 6H), 7.65 (m, 2H), 7.75 (d, 1H), 8.04 (br, 3H). MS APCI, m/z=353 (M+1). HPLC 2.25 min (Method C).

### Example 23

### N¹-[(2R,3R)-5-Cyclohexyl-4-oxo-2-phenyl-2,3,4,5-tetrahydro-1,5-benzothiazepin-3-yl]-N²-[(3,5-Difluorophenyl)acetyl]-L-alaninamide (23)

Using a procedure similar to that described in Example 11, except using (2,3-cis)-3-amino-5-cyclohexyl-2-phenyl-2,3-dihydro-1,5-benzothiazepin-4(5H)-one (23b) 130 mg, 0.37 mmol) as the amine component, the title compound (23) was obtained in a 1:1 mixture with the 2S,3S diastereomer (210 mg, 98%) as an off white solid. ¹H NMR (300 MHz, CDCl₃) δ0.84 (d, 1.5H), 1.10 (m, 2H), 1.13 (d, 1.5H), 1.2-1.9 (m, 7H), 2.19 (m, 1H), 3.41 (s, 2H), 4.15 (m, 1H), 4.55 (m, 2H), 5.03 (d, 1H), 5.69 (m, 0.5H), 5.99 (m, 0.5H), 6.35 (dd, 1H), 6.73 (d, 2.5H), 7.35 (m, 7H), 7.47 (m, 1.5H), 7.75 (d, 1H). MS APCI, m/z=578 (M+1). LC/MS: 3.35 min.

The starting amine, (2,3-cis)-3-amino-5-cyclohexyl-2-phenyl-2,3-dihydro-1,5-benzothiazepin-4(5H)-one (23b) was prepared in the following manner:

### a. Benzyl [(2,3-cis)-5-cyclohex-2-en-1-yl-4-oxo-2-phenyl-2,3,4,5-tetrahydro-1,5-benzothiazepin-3-yl]carbamate (23a)

Using a procedure similar to that described in Example 2, part a, substituting benzyl [(2,3-cis)-4-oxo-2-phenyl-2,3,4,5-tetrahydro-1,5-benzothiazepin-3-yl]carbamate (9c) (500 mg, 1.24 mmol) as the amide component, the title compound (23a) was obtained in a 1:1 mixture with the 2S,3S diastereomer (400 mg, 67%) as a white solid. (recrystallized from ether). MS APCI, m/z=485 (M+1). LC/MS: 3.31.

### b. (2,3-cis)-3-Amino-5-cyclohexyl-2-phenyl-2,3-dihydro-1,5-benzothiazepin-4(5H)-one (23b)

Using a procedure similar to that described in Example 1, part d Method C, except using benzyl [(2,3-cis)-5-cyclohex-2-en-1-yl-4-oxo-2-phenyl-2,3,4,5-tetrahydro-1,5-benzothiazepin-3-yl]carbamate (23a), (360 mg, 0.749 mmol) as the protected amine component, the title compound (23) was obtained (247 mg, 94%) as a white semi-solid. ¹H NMR (300 MHz, CDCl₃) δ1.31-1.89 (m, 12 h), 2.21 (m, 1H), 3.62 (d, 1H, J=7.0 Hz), 4.69 (d, 1H, J=7.0 Hz), 7.27-7.49 (m, 8H), 7.71 (m, 1H). MS APCI, m/z=353 (M+1). LC/MS: 2.45 min.

### Example 24

### N¹-[(2R,3R)-7-Chloro-5-cyclohexyl-4-oxo-2-phenyl-2,3,4,5-tetrahydro-1,5-benzothiazepin-3-yl]-N²-[(3,5-difluorophenyl)acetyl]-L-alaninamide (24)

Using a procedure similar to that described in Example 11, except using (2,3-cis)-3-amino-7-chloro-5-methyl-2-phenyl-2,3-dihydro-1,5-benzothiazepin-4(5H)-one hydrobromide (24b) (91 mg, 0.194 mmol) as the amine component, the title compound (24) was obtained in a 1:1 mixture with the 2S,3S diastereomer (114 mg, 96%) as an off white solid. The diastereomers were separated by flash column chromatography (Rf=0.25 vs 0.30 for (2S,3S), 35% EtOAc-hexanes) eluting with 25-50% EtOAc-hexanes to afford the pure title compound (35 mg) as a white solid. ¹H NMR (300 MHz, CDCl₃) δ1.14 (d, 3H), 1.2-2.1 (m, 9H), 2.19 (m, 1H), 3.41 (s, 2H), 4.14 (m, 1H), 4.52 (m, 2H), 5.00 (d, 1H), 5.70 (d, 1H), 6.34 (d, 1H), 6.40 (d, 1H), 6.76 (m, 2.5H), 7.3-7.4 (m, 6.5H), 7.67 (d, 1H). MS APCI, m/z=612 (M+1). LC/MS: 3.28 min.

The starting amine, (2,3-cis)-3-amino-7-chloro-5-cyclohexyl-2-phenyl-2,3-dihydro-1,5-benzothiazepin-4(5H)-one (24b) was prepared in the following manner:

### a. Benzyl [(2,3-cis)-7-chloro-5-cyclohex-2-en-1-yl-4-oxo-2-phenyl-2,3,4,5-tetrahydro-1,5-benzothiazepin-3-yl]carbamate (24a)

Using a procedure similar to that described in Example 2, part a, substituting benzyl [(2,3-cis)-7-chloro-4-oxo-2-phenyl-2,3,4,5-tetrahydro-1,5-benzothiazepin-3-yl]carbamate (13b) (290 mg, 0.66 mmol) as the amide component, the title compound (24a) was obtained in a 1:1 mixture with the 2S,3S diastereomer (220 mg, 64%) as a white solid. (recrystallized from ether). MS APCI, m/z=519 (M+1). LC/MS: 3.51 min.

### b. (2,3-cis)-3-Amino-7-chloro-5-cyclohexyl-2-phenyl-2,3-dihydro-1,5-benzothiazepin-4(5H)-one hydrobromide (24b)

A mixture of benzyl [(2,3-cis)-7-chloro-5-cyclohex-2-en-1-yl-4-oxo-2-phenyl-2,3,4,5-tetrahydro-1,5-benzothiazepin-3-yl]carbamate (24a), (200 mg, 0.622 mmol) and 10% Pd/C (200 mg, DeGussa type 50% wt H₂O) in glacial acetic acid (25 mL) was hydrogenated at 50 psi H₂ for ˜12 h. The reaction mixture was filtered through diatomaceous earth and concentrated in vacuo. The crude oil (still contained unreduced CBZ) taken up in acetic acid (2 mL) added 30% HBr/acetic acid (2 mL) heated to 50° C. for 1 h, evaporated, recrystallized from ether to afford the title compound (with 10% des Cl product) as a white solid (115 mg, 64%). ¹H NMR (300 MHz, d6-DMSO) δ1.03-2.14 (m, 11H), 4.13 (d, 1H, J=7.0 Hz), 4.96 (d, 1H, J=−7.0 Hz), 7.45 (s, 5H), 7.53-7.63 (m, 2H), 7.79 (m, 1H), 7.93 (br 3H). MS APCI, m/z=387 (M+1). LC/MS: 2.55 min.

### Example 25

### N²-[(3,5-Difluorophenyl)acetyl]-N¹-[(6R,7R)-7-(1-naphthyl)-5-oxo-1,4-thiazepan-6-yl]-L-alaninamide (25)

Using a procedure similar to that described in Example 11, except using (6,7-cis)-6-amino-7-(1-naphthyl)-1,4-thiazepan-5-one hydrobromide (25e) (110 mg, 0.30 mmol) as the amine component, the title compound (25) was obtained after recrystallization from ether in a 1:1 mixture with the 2S,3S diastereomer (82 mg, 55%) as a white solid. ¹H NMR (300 MHz, CDCl₃) δ0.57 (d, 1.5H), 1.12 (d, 1.5H), 2.88-3.10 (m, 2H), 3.24 (s, 1H), 3.41 (s, 1H), 3.75-3.95 (m, 2H), 4.25 (m, 1H), 5.39 (m, 1H), 5.66 (d, 0.5H), 6.09 (d, 0.5H), 6.35 (t, 1H), 6.6-6.8 (m, 2.5H), 6.76 (m, 2.5H), 6.86 (d, 0.5H), 7.03 (d, 0.5H), 7.3-7.5 (m, 3H), 7.61 (dd, 1H), 7.7-7.9 (m, 2H), 7.96 (m, 1H). MS APCI, m/z=498 (M+1). LC/MS: 2.1 min.

The starting amine, (6,7-cis)-6-amino-7-(1-naphthyl)-1,4-thiazepan-5-one hydrobromide (25e), was prepared in the following manner:

### a. Methyl (2Z)-2-{[(benzyloxy)carbonyl]amino}-3-(1-naphthyl)acrylate (25a)

Using a procedure similar to that described in Example 1 part a, except using 1-naphthylaldehyde (2.04 mL, 15.0 mmol) as the aldehyde component, the crude product was obtained as an oil (6.0 g). After recrystallization from EtOAc-ether-hexanes (cold) the pure title compound (25a) was obtained (3.9 g, 72%) as a white solid. MS APCI, m/z=362 (M+1), 2.77 min.

### b. Methyl N-[(benzyloxy)carbonyl]-S-{2-[(tert-butoxycarbonyl)amino]ethyl}-3-(1-naphthyl)cysteinate (25b)

Using a procedure similar to that described in Example 10 part b, substituting methyl (2Z)-2-{[(benzyloxy)carbonyl]amino}-3-(1-naphthyl)acrylate (25a) (2.8 g, 7.75 mmol) as the acrylate component and tert-butyl (2-mercaptoethyl)carbamate (5.3 g, 29.9 mmol) as the thio component (stirred 5 d), the title compound (25b) was obtained (3.3 g, 79%) as a semi-solid.

### c. Methyl S-(2-aminoethyl)-N-[(benzyloxy)carbonyl]-3-(1-naphthyl)cysteinate hydrochloride (25c)

To a stirred solution of methyl N-[(benzyloxy)carbonyl]-S-{2-[(tert-butoxycarbonyl)amino]ethyl}-3-(1-naphthyl)cysteinate (25b) (3.3 g, 6.13 mmol) in EtOAc (80 mL) at −30° C., was bubbled over HCl gas for 10 min, stirred at −30° C. for 15 min and then allowed to 25° C. for 3 h. Evaporation to near dryness and precipitation from ether afforded pure title compound (25c) (2.6 g, 89%) as a white solid. MS APCI, m/z=439 (M+1), 2.34 min.

### d. Benzyl [(6,7-cis)-7-(1-naphthyl)-5-oxo-1,4-thiazepan-6-yl]carbamate (25d)

To a stirred solution of methyl S-(2-aminoethyl)-N-[(benzyloxy)carbonyl]-3-(1-naphthyl)cysteinate hydrochloride (25c) (2.0 g, 4.2 mmol) in DCM (35 mL) under nitrogen at 0° C. was added via syringe a 2M solution of trimethyl aluminum (5 mL, 10.0 mmol), the reaction allowed to warm to 25° C. and stirred for ˜12 h. The reaction was cooled to 0° C. and 1 N HCl added carefully until bubbling stopped, then 0.5 N HCl was added (20 mL). The aqueous layer was extracted with DCM, the organic layer dried (MgSO₄), filtered and evaporated to yield 2 g of crude product as a 3:1 mixture with the 6,7-trans product. Purification using flash column chromatography (1% methanol-DCM) afforded the title compound (25d) (610 mg, 36%) as a white solid. MS APCI, m/z=407 (M+1), 2.37 min.

### e. (6,7-cis)-6-Amino-7-(1-naphthyl)-1,4-thiazepan-5-one hydrobromide (25e)

Using a procedure similar to that described in Example 1, part d Method D, except using benzyl [(6,7-cis)-7-(1-naphthyl)-5-oxo-1,4-thiazepan-6-yl]carbamate (25d), (590 mg, 1.45 mmol) as the protected amine component, the title compound (25e) was obtained (500 mg, 88%) as a light yellow solid. ¹H NMR (300 MHz, d6-DMSO) δ2.67-2.79 (m, 1H), 2.83-2.94 (m, 1H), 3.63-3.85 (m, 2H), 5.11 (m, 1H), 5.29 (d, 1H, J=4.0 Hz), 7.42-7.68 (m, 4H), 7.88-8.02 (m, 2H), 8.14 (d, 1H), 8.22 (br, 3H), 8.71 (br t, 1H). MS APCI, m/z=273 (M+Na), LC/MS: 1.23 min.

### Example 26

### (2S)-2-[(3,5-Difluorophenyl)acetyl]amino-N-[(6R,7R)-7-(1-naphthyl)-5-oxo-1,4-thiazepan-6-yl]-2-phenylacetamide (26)

To a solution of (2S)-2-amino-N-[(6,7-cis)-7-(1-naphthyl)-5-oxo-1,4-thiazepan-6-yl]-2-phenylacetamide hydrochloride (26b) (53 mg, 0.12 mmol) in DCM (15 mL) at 0° C. under nitrogen was added 1 eq NMM (13 μL, 0.12 mmol), followed by the 3,5-difluorophenylacetic acid (21 mg, 0.12 mmol), HOBt-hydrate (40 mg, 0.264 mmol), EDAC.HCl (35 mg, 0.18 mmol) and NMM (34 μL, 0.18 mmol). The reaction mixture was stirred 2 h at 0° C., concentrated in vacuo and partitioned between H₂O (100 mL) and EtOAc (125 mL). The organic phase was collected and consecutively washed with HCl, H₂O, saturated NaHCO₃, and brine, dried and the solvent removed in vacuo to yield a crude semi-solid. This material was purified by flash column chromatography (5% methanol-CHCl₃) to afford the title compound (26) as a 1:1 mixture with the 2S,3S diastereomer (46 mg, 69%) as a white solid. ¹H NMR (300 MHz, CDCl₃) δ2.77-2.96 (m, 2H), 3.47 (s, 2H), 3.57-3.78 (m, 2H), 5.25-5.40 (m, 2.5H), 5.49 (d, 0.5H), 6.31 (t, 0.5H), 6.54 (t, 0.5H), 6.66-6.86 (m, 6H), 6.95 (t, 1H), 7.06-7.25 (m, 4H), 7.32-7.49 (m, 3H), 7.54 (d, 0.5H), 7.62 (d, 0.5H), 7.79 (t, 1H), 6.86 (d, 0.5H), 7.95 (d, 0.5H). MS APCI, m/z=560 (M+1). LC/MS: 2.34 min.

The starting amine, (2S)-2-amino-N-[(6,7-cis)-7-(1-naphthyl)-5-oxo-1,4-thiazepan-6-yl]-2-phenylacetamide hydrochloride (26b), was prepared in the following manner:

### a. tert-Butyl ((1S)-2-{[(6,7-cis)-7-(1-naphthyl)-5-oxo-1,4-thiazepan-6-yl]amino}-2-oxo-1-phenylethyl)carbamate (26a)

To a solution of (6,7-cis)-6-amino-7-(1-naphthyl)-1,4-thiazepan-5-one (2e) (162 mg, 0.545 mmol) in DCM (15 mL) at 0° C. under nitrogen was added (2S)-[(tert-butoxycarbonyl)amino](phenyl)acetic acid (137 mg, 0.545 mmol) followed by HOBt-hydrate (190 mg, 1.24 mmol), EDAC.HCl (162 mg, 0.850 mmol) and NMM (93 μL, 0.850 mmol). The reaction mixture was stirred 1.5 h at 0° C. and for 30 min at 25° C., concentrated in vacuo and partitioned between H₂O (25 mL) and EtOAc (25 mL). The organic phase was collected and consecutively washed with 0.5 N HCl, H₂O, brine, dried and the solvent removed in vacuo to give the title compound (29a) (272 mg, 95%) as a light pink solid. MS APCI, m/z=528 (M+Na), LC/MS: 2.36 min.

### b. (2S)-2-Amino-N-[(6,7-cis)-7-(1-naphthyl)-5-oxo-1,4-thiazepan-6-yl]-2-phenylacetamide hydrochloride (26b)

Using a procedure similar to that described in Example 25, part c, except using tert-butyl ((1S)-2-{[(6,7-cis)-7-(1-naphthyl)-5-oxo-1,4-thiazepan-6-yl]amino}-2-oxo-1-phenylethyl)carbamate (26a) (130 mg, 0.257 mmol) as reactant, the title compound (26b) (114 mg, 99%) was obtained as a white solid. MS APCI, m/z=406 (M+Na), LC/MS: 1.55, 1.64 min.

### Example 27

### (2S)-2-Hydroxy-4-methyl-N-((1S)-2-[(6R,7R)-7-(1-naphthyl)-5-oxo-1,4-thiazepan-6-yl]amino-2-oxo-1-phenylethyl)pentanamide (27)

Using a procedure similar to that described in example 28, except using (2S)-2-amino-N-[(6,7-cis)-7-(1-naphthyl)-5-oxo-1,4-thiazepan-6-yl]-2-phenylacetamide hydrochloride (26b) (50 mg, 0.113 mmol) as the amine component, the title compound (27) was obtained in a 1:1 mixture with the 6S,7S diastereomer (30 mg, 51%) as a white solid. ¹H NMR (300 MHz, CDCl₃) δ0.82-0.88 (dd, 6H), 1.2-1.4 (m, 2H), 1.73 (m, 1H), 2.76-3.05 (m, 2H), 3.61 (m, 1.5H), 3.74-3.98 (m, 1.5H), 5.15-5.34 (m, 1.5H), 5.56 (q, 1H), 5.72 (d, 0.5H), 7.0-7.36 (m, 7H), 7.35-7.55 (t, 3H), 7.73 (dd, 1H), 7.8-8.0 (m, 3H), 8.13 (t, 1H), 8.32 (dd, 1H). MS APCI, m/z=520(M+1). LC/MS: 2.29 min.

### Example 28

### (2S)-2-Hydroxy-4-methyl-N-((1S)-2-oxo-2-[(2R,3S)-4-oxo-2-phenyl-2,3,4,5-tetrahydro-1,5-benzoxazepin-3-yl]amino-1-phenylethyl)pentanamide (28)

To a solution of (2S)-2-amino-N-[(2,3-cis)-4-oxo-2-phenyl-2,3,4,5-tetrahydro-1,5-benzoxazepin-3-yl]-2-phenylacetamide (58b) (110 mg, 0.284 mmol) in DCM (12 mL) at 0° C. under N₂ was added the (2S)-2-hydroxy-4-methylpentanoic acid (38 mg, 0.284 mmol) followed by addition of HOBt-hydrate (70 mg, 0.454 mmol), EDAC.HCl (65 mg, 0.341 mmol) and NMM (37 μL, 0.29 mmol). The reaction mixture was stirred 2 h at 0° C., concentrated in vacuo and partitioned between water (100 mL) and EtOAc (125mL). The organic phase was collected and consecutively washed with HCl, water, saturated NaHCO₃, and brine, dried and the solvent removed in vacuo to yield a crude semi-solid (140 mg). This material was purified by flash column chromatography eluting with 5% methanol-CHCl₃ to afford the title compound (28) as a 1:1 mixture with the 2S,3R diastereomer (110 mg, 77%) as a white solid. ¹H NMR (300 MHz, CDCl₃) δ0.77-0.88 (dd, 6H), 1.2-1.4 (m, 2H), 1.73 (m, 1H), 3.85 (m, 0.5H), 3.94 (m, 0.5H), 4.96 (t, 0.5H), 5.11 (t, 0.5H), 5.33 (t, 0.5H), 5.52-5.66 (m, 2.5H), 6.90 (d, 1H), 7.1-7.4 (m, 13H), 8.01 (dd, 1H), 8.19 (t, 1H), 10.23 (s, 0.5H), 10.29 (s, 0.5H). MS APCI, m/z=502(M+1). LC/MS: 2.36 and 2.44 min.

### Example 29

### N²-[(2S)-2-Hydroxy-4-methylpentanoyl]-N¹-[(2R,3S)-4-oxo-2-phenyl-2,3,4,5-tetrahydro-1,5-benzoxazepin-3-yl]-L-leucinamide (29)

Using a procedure similar to that described in example 28, except using N¹-[(2,3-cis)-4-oxo-2-phenyl-2,3,4,5-tetrahydro-1,5-benzoxazepin-3-yl]-L-leucinamide (29b) (80 mg, 0.217 mmol) as the amine component, the title compound (29) was obtained in a 1:1 mixture with the 2S,3S diastereomer (90 mg, 86%) as an off white solid. ¹H NMR (300 MHz, CDCl₃) δ 0.73-0.88 (m, 12H), 1.2-1.5 (m, 6H), 1.73 (m, 1H), 3.81 (m, 1H), 4.33 (m, 1H), 4.96 (t, 0.5H), 5.03 (t, 0.5H), 5.33 (dd, 1H), 5.60 (dd, 1H), 7.11-7.27 (m, 4H), 7.28-7.38 (m, 5H), 7.53 (d, 0.5H), 7.61 (t, 1H), 7.77 (d, 0.5H), 10.28 (s, 0.5H), 10.30 (s, 0.5H). MS APCI, m/z=482(M+1). LC/MS: 2.28 min.

The starting amine, N¹-[(2,3-cis)-4-oxo-2-phenyl-2,3,4,5-tetrahydro-1,5-benzoxazepin-3-yl]-L-leucinamide (29b, was prepared in the following manner:

### a. N²-[tert-Butoxycarbonyl]-N¹-[(2,3-cis)-4-oxo-2-phenyl-2,3,4,5-tetrahydro-1,5-benzoxazepin-3-yl]-L-leucinamide (29a)

To a suspension of (2,3-cis)-3-amino-2-phenyl-2,3-dihydro-1,5-benzoxazepin-4(5H)-one hydrochloride (6e) (85 mg, 0.292 mmol) in DCM (15 mL) at 0° C. under N₂ was added 1 eq NMM (32 uL, 0.292 mmol) and 4 drops DMF and the mixture stirred for 5 min. To the reaction mixture was added N-[(2,2-dimethylpropanoyl)oxy]-L-leucine (73 mg, 0.292 mmol), HOBt-hydrate (72 mg, 0.467 mmol), EDAC-HCl (67 mg, 0.351 mmol) and NMM (39 μL, 0.351 mmol). The reaction mixture was stirred 2 h at 0° C., concentrated in vacuo and partitioned between water (25 mL) and EtOAc (25 mL). The organic phase was collected and consecutively washed with water, brine, dried and the solvent removed in vacuo to give the title compound (29a) (135 mg, 98%) as a white solid.

### b. N¹-[(2,3-cis)-4-Oxo-2-phenyl-2,3,4,5-tetrahydro-1,5-benzoxazepin-3-yl]-L-leucinamide trifluoroacetate (29b)

To a solution of N²-[tert-butoxycarbonyl]-N¹-[(2,3-cis)-4-oxo-2-phenyl-2,3,4,5-tetrahydro-1,5-benzoxazepin-3-yl]-L-leucinamide (29a) in DCM (1 mL) at 0° C. under N₂ was added a solution of 30% TFA in DCM (2 mL). The reaction mixture stirred at 0° C. for 1 h and then at ambient temperature for 30 min., concentrated in vacuo to give the title compound (29b) as the TFA salt (105 mg, 88%) as a white solid. ¹H NMR (300 MHz, d6-DMSO) δ0.81 (m, 6H), 0.89 (m, 0.5H), 1.30 (m, 1H), 1.45 (m, 1H), 1.56 (m, 0.5H), 4.45 (bm, 3H), 5.11 (q, 1H), 5.61 (t, 1H), 7.22 (m, 3H), 7.39 (m, 4H), 8.08 (m, 2H), 8.48 (d, 0.5H), 8.53 (d, 0.5H), 10.30 (s, 0.5H), 10.40 (s, 0.5H).

### Example 30

### N²-[(3,5-Difluorophenyl)acetyl]-N¹-[(6S,7R)-4-methyl-5-oxo-7-phenyl-1,4-oxazepan-6-yl]-L-alaninamide (30)

Using a procedure similar to that described in example 11, except using (6,7-cis)-6-amino-4-methyl-7-phenyl-1,4-oxazepan-5-one hydrochloride (30d) (100 mg, 0.389 mmol) as the amine component, to afford 150 mg crude product. After filtering thru a small plug of silica (CHCl₃) the title compound (30) was obtained in a 1:1 mixture with the 6R,7S diastereomer (122 mg, 70%) as a white foam. ¹H NMR (300 MHz, CDCl₃) δ0.81 (d, 1.5H), 1.18 (d, 1.5H), 3.11 (s, 3H), 3.40 (m, 1H), 3.45 (s, 1H), 3.46 (s, 1H), 3.73 (m, 1H), 3.92 (m, 2H), 4.25 (m, 1H), 4.97 (dd, 1H), 5.19 (q, 1H), 5.77 (d, 0.5H), 6.08 (d, 0.5H), 6.7-6.9 (m, 3H), 7.2-7.3 (m, 6H). MS APCI, m/z=446(M+1). LC/MS: 1.85 min.

The starting amine, (6,7-cis)-6-amino-4-methyl-7-phenyl-1,4-oxazepan-5-one hydrochloride (30d), was prepared in the following manner:

### a. (2,3-trans)-N-(2-Hydroxyethyl)-N-methyl-3-phenyloxirane-2-carboxamide (30a)

To a solution of ethyl (2,3-trans)-3-phenyloxirane-2-carboxylate (10 g, 52.0 mmol) in methanol (20 mL) at −20° C. was added 2-(methylamino)ethanol (4.6 g, 62.0 mmol) followed by catalytic sodium methoxide (25%, 20 drops). The reaction was then set in a freezer (˜−20° C.) for 10 d, diluted with 10% HCl (10 drops), water (100 mL), extracted with DCM, and the organic layers combined, dried, filtered and evaporated to afford the title compound (30a) (9.7 g, 84%). MS APCI, m/z=222 (M+1). LC/MS: 1.03 min.

### b. (6,7-trans)-6-Hydroxy-4-methyl-7-phenyl-1,4-oxazepan-5-one (30b)

To a stirred solution of (2,3-trans)-N-(2-hydroxyethyl)-N-methyl-3-phenyloxirane-2-carboxamide (30a) (5.4 g, 24.4 mmol) in anhydrous THF (300 mL) was added 10 mol % of MgI₂ (670 mg, 2.44 mmol, 98% purity) and the mixture heated to reflux for ˜12 h, cooled, and evaporated to a crude orange oil. Isocratic flash column chromatography (1% methanol-DCM) returned the title compound (30b) (2.7 g, 50%). MS APCI, m/z=222 (M+1) LC: 1.78 min. (Method A)

### c. (6,7-cis)-6-Azido-4-methyl-7-phenyl-1,4-oxazepan-5-one (30c)

To an ice cooled solution of (6,7-trans)-6-hydroxy-4-methyl-7-phenyl-1,4-oxazepan-5-one (30b) (900 mg, 4.1 mmol) in DCM (20 mL) was added lutidine ((524 μL, 4.5 mmol), followed by dropwise addition of triflic anhydride (753 μL, 4.4 mmol) in DCM (3 mL) and the mixture stirred at 0° C. for 20 min, concentrated under vacuo to give crude triflate (containing 32% starting (30b)). The crude intermediate dissolved in DMF (5 mL), cooled to 0° C., sodium azide (1.33 g, 20.5 mmol) added all at once. The mixture stirred at 0° C. for 1 h., then at 25° C. for ˜12 h. The mixture diluted with water (50 mL) and extracted with EtOAc. The organic extract was collected, washed consecutively with saturated aqueous sodium bicarbonate, and brine, dried, filtered and evaporated (1.1 g dark oil). The crude product was purified by flash chromatography (3% methanol-DCM) to yield nearly pure title (30c) (385 mg, 38%). MS APCI, m/z=247 (M+1) 218 (M+1−N₂). LC/MS: 1.75 min.

### d. (6,7-cis)-6-Amino-4-methyl-7-phenyl-1,4-oxazepan-5-one hydrochloride (30d)

A mixture of (6,7-cis)-6-azido-4-methyl-7-phenyl-1,4-oxazepan-5-one (30a) (300 mg, 1.54 mmol) and 10% Pd/C (30 mg, DeGussa type 50% wt water) in absolute ethanol (45 mL) and 1N HCl (3 mL, 3.0 mmol) was hydrogenated at 20 psi for 3 d. The reaction mixture was filtered through diatomaceous earth and concentrated in vacuo to afford the title compound as a white solid (170 mg, 43%). ¹H NMR (300 MHz, d6-DMSO) δ3.02 (s, 3H), 3.43 (m, 2H), 3.91-4.17 (m, 2H), 4.85 (m, 1H), 5.10 (m, 1H), 7.27-7.45 (m, 5H), 8.32 (br, 3H). MS APCI, m/z=221 (M+1). LC/MS: 0.68 min.

### Example 31

### N²-[(3,5-Difluorophenyl)acetyl]-N¹-[(2S,6S,7R)-4-methyl-5-oxo-2,7-diphenyl-1,4-oxazepan-6-yl]-L-alaninamide (31)

Using a procedure similar to that described in example 11, except using (2SR,6SR,7RS)-6-amino-4-methyl-2,7-diphenyl-1,4-oxazepan-5-one hydrochloride (31d) (100 mg, 0.34 mmol) as the amine component, the title compound (31) was obtained in a 1:1 mixture with the 6R,7S diastereomer (160 mg, 90%) as a white solid. ¹H NMR (300 MHz, CDCl₃) δ0.73 (d, 1.5H), 1.13 (d, 1.5H), 2.88 (s, 1.5H), 2.92 (s, 1.5H), 3.45 (s, 2H), 3.53, (m, 1H), 4.1-4.3 (m, 2H), 4.86 (m, 1H), 5.13 (dd, 1H), 5.30 (m, 1H), 5.61 (d, 0.5H), 6.06 (d, 0.5H), 6.68-6.92 (m, 3H), 7.2-7.4 (m, 11H). MS ES⁺, m/z=522(M+1). HPLC: 2.93 min. (Method A).

The starting amine, (6,7-cis)-6-amino-4-methyl-7-phenyl-1,4-oxazepan-5-one hydrochloride (31d), was prepared in the following manner:

### a. (2,3-trans)-N-(2-Hydroxy-2-phenylethyl)-N-methyl-3-phenyloxirane-2-carboxamide (31a)

To a solution of ethyl (2,3-trans)-3-phenyloxirane-2-carboxylate (5.0 g, 23.9 mmol) in methanol (10 mL) at −20° C. was added 2-(methylamino)-1-phenylethanol (4.29 g, 28.4 mmol) followed by catalytic sodium methoxide (25%, 10 drops). The reaction was placed in a freezer (˜−20 C) for 2 d, filtered and washed with cold methanol to afford the title compound (31a) as a white solid (3.3 g, 46%).

### b. (2RS,6SR,7SR)-6-Hydroxy-4-methyl-2,7-diphenyl-1,4-oxazepan-5-one (31b)

To a stirred solution of (2,3-trans)-N-(2-hydroxy-2-phenylethyl)-N-methyl-3-phenyloxirane-2-carboxamide (31a) (2.1 g, 7.6 mmol) in anhydrous THF (300 mL) was added 10 mol % of MgI₂ (215 mg, 0.76 mmol, 98% purity) and the mixture heated to reflux over a weekend, cooled, and evaporated to an orange oil. Isocratic flash column chromatography (1% methanol-DCM) afforded the title compound (31b) (900 mg, 43%). MS APCI, m/z=298 (M+Na). LC: 2.84 min. (Method A).

### c. (2RS,6SR,7RS)-6-Azido-4-methyl-2,7-diphenyl-1,4-oxazepan-5-one (31c)

To an ice cooled solution of (2RS,6SR,7SR)-6-hydroxy-4-methyl-2,7-diphenyl-1,4-oxazepan-5-one (31b) (1.9 g, 6.39 mmol) in DCM (45 mL) was added lutidine (1.12 mL, 9.6 mmol), followed by dropwise addition of triflic anhydride (1.6 mL, 9.6 mmol) in DCM (5 mL), and the mixture stirred at 0° C. for 20 min, concentrated under vacuo (cold), then under high vacuum for 20 min to give crude triflate. The crude intermediate in DMF (5 mL) was cooled to 0° C. and sodium azide (2.1 g, 32.0 mmol) added in one portion. The mixture stirred at 0° C. for 1 h., then at 25° C. for ˜12 h. The reaction diluted with water (100 mL), extracted with EtOAc, the organic extract was collected and washed consecutively with saturated aqueous sodium bicarbonate, and brine, dried, filtered and evaporated to a dark oil. The crude product was purified by flash chromatography (3% methanol-DCM) to yield nearly pure title (31c) (1.0 g, 48%). MS APCI, m/z=272(M+1−N₂). LC/MS: 2.36 min.

### d. (2SR,6SR,7RS)-6-Amino-4-methyl-2,7-diphenyl-1,4-oxazepan-5-one hydrochloride (31d)

Using a procedure similar to that described in example 30, part d, except using (2RS,6SR,7RS)-6-azido-4-methyl-2,7-diphenyl-1,4-oxazepan-5-one (31c) (1.0 g, 3.1 mmol) as the azido component, the title compound (31d) was obtained as a white solid (940 mg, 94%). ¹H NMR (300 MHz, d6-DMSO) δ2.33 (s, 3H), 3.64 (m, 1H), 4.22 (m, 1H), 4.86 (m, 1H), 5.08 (m, 1H), 5.26 (d, 1H, J=6.6 Hz), 7.27-7.50 (m, 10H), 8.18 (br, 3H). MS APCI, m/z=297(M+1).

### Example 32

### N²-[(3,5-Difluorophenyl)acetyl]-N¹-[(6R,7R)-4-methyl-5-oxo-7-phenyl-1,4-thiazepan-6-yl]-L-alaninamide (32)

Using a procedure similar to that described in example 11, except using (6,7-cis)-6-amino-4-methyl-7-phenyl-1,4-thiazepan-5-one (32c) (50 mg, 0.211 mmol) as the amine component, the title compound (32) was obtained in a 1:1 mixture with the 6S,7S diastereomer (72 mg, 74%) as a white foam. ¹H NMR (300 MHz, CDCl₃) δ 0.87 (d, 1.5H), 1.18 (d, 1.5H), 2.87-3.08 (m, 2H), 3.13 (s, 3H), 3.46 (s, 1H), 3.48 (s, 1H), 3.6-3.7 (m, 1H), 3.9-4.1 (m, 1H), 4.25 (m, 1H), 4.37 (dd, 1H), 5.35 (q, 1H), 5.83 (d, 0.5H), 6.07 (d, 0.5H), 6.68-6.96 (m, 4H), 7.18 (m, 2H), 7.26 (m, 3H). MS APCI, m/z=462(M+1). LC/MS: 1.88 min.

The starting amine, (6,7-cis)-6-amino-4-methyl-7-phenyl-1,4-thiazepan-5-one (32c)), was prepared in the following manner:

### a. tert-Butyl [(6,7-cis)-5-oxo-7-phenyl-1,4-thiazepan-6-yl]carbamate (32a)

To an ice cooled solution of (6,7-cis)-6-amino-7-phenyl-1,4-thiazepan-5-one hydrobromide (7d) (165 mg, 0.52 mmol) in DCM (5 mL) under N₂ was added TEA (153 μL, 1.09 mmol), followed by di-tert-butyl carbonate (124 mg, 0.565 mmol). The mixture was stirred at 0° C. for 30 min. and at 25° C. for 2 h, the volume reduced, and the residue partitioned between water and EtOAc. The organic extract was washed with brine, dried, filtered and evaporated to yield the title compound (32a) as a clear oil.

### b. tert-Butyl [(6,7-cis)-4-methyl-5-oxo-7-phenyl-1,4-thiazepan-6-yl]carbamate (32b)

Using a procedure similar to that described in example 2 part, except using tert-butyl [(6,7-cis)-5-oxo-7-phenyl-1,4-thiazepan-6-yl]carbamate (32a) (74 mg, 0.22 mmol) as the amide component, and iodomethane (14 μL, 0.22 mmol) as the electrophile afforded crude title compound as an oil. Isocratic flash column chromatography (25% EtOAc-hexanes) afforded pure title compound (32b) (63 mg, 82%) as a clear oil.

### c. (6,7-cis)-6-Amino-4-methyl-7-phenyl-1,4-thiazepan-5-one trifluoracetate (32c)

To an ice cooled solution of tert-butyl [(6,7-cis)-4-methyl-5-oxo-7-phenyl-1,4-thiazepan-6-yl]carbamate (32b) (133 mg, 0.396 mmol) was added 20% TFA in DCM (3 mL), the mixture stirred at 0° C. for 1.5 h and at 25° C. for 1 h, evaporated and placed under high vacuum for ˜12 h. Trituration with ether returned the title compound (32c) as a white solid (115 mg, 82%). ¹H NMR (300 MHz, d6-DMSO) δ2.84-2.94 (m, 1H), 3.07 (s, 3H), 3.18 (m, 1H), 3.80-3.91 (m, 1H), 4.21 (m, 2H), 5.17 (d, 1H, J=3.5 Hz), 7.22 (m, 2H), 7.33 (m, 3H), 8.17 (br, 3H). MS APCI, m/z 237(M+1). LC/MS: 0.79 min.

### Example 33

### N²-[(3,5-Difluorophenyl)acetyl]-N¹-[(3R,6S,7R)-4-methyl-5-oxo-3,7-diphenyl-1,4-oxazepan-6-yl]-L-alaninamide (33)

Using a procedure similar to that described in example 11, except using (3R,-6,7-cis)-6-amino-4-methyl-3,7-diphenyl-1,4-oxazepan-5-one hydrochloride (33d) (60 mg, 0.180 mmol) as the amine component, afforded crude product (100 mg). After filtering thru a small plug of silica (CHCl₃) the title compound (30) was obtained in a 1:1 mixture with the 6R,7S diastereomer (55 mg, 59%) as a white foam.

¹H NMR (300 MHz, CDCl₃) δ1.04 (d, 3H), 2.78 (s, 3H), 3.42 (s, 2H), 4.00, (m, 2H), 4.41 (t, 1H), 5.00 (d, 1H), 5.23 (m, 1H), 5.50 (m, 2H), 6.76 (m, 3.5H), 7.25-7.35 (m, 7.5H), 7.44 (m, 3H). MS APCI, m/z=522(M+1). LC/MS: 2.30 min.

The starting amine, (3R-6,7-cis)-6-amino-4-methyl-3,7-diphenyl-1,4-oxazepan-5-one hydrochloride (33d), was prepared in the following manner:

### a. (2RS,3RS)—N-[(1R)-2-Hydroxy-1-phenylethyl]-N-methyl-3-phenyloxirane-2-carboxamide (33a)

To a solution of ethyl (2,3-trans)-3-phenyloxirane-2-carboxylate (13.4 g, 70.2 mmol) in methanol (35 mL) at −20° C. was added the (2R)-2-(methylamino)-2-phenylethanol (12.6 g, 83.4 mmol) [Karim, A., et al., J. Organometallic Chem. (1986), 317(1), 93-104.], followed by catalytic sodium methoxide (25%, 30 drops). The reaction mixture was placed in a freezer (˜−20° C.) for 3 d, diluted with 10% HCl (10 drops), water (100 mL), and extracted with DCM. The combined organic layers were dried, filtered and evaporated to afford the title compound (33a) (12.2 g, 63%). MS APCI, m/z=276 (M+1), 298 (M+Na). HPLC: 5.68, 5.74 min (Method E).

### b. (3R,6R,7R)-6-Hydroxy-4-methyl-3,7-diphenyl-1,4-oxazepan-5-one (33b)

To a stirred solution of (2RS,3RS)—N-[(1S)-2-hydroxy-1-phenylethyl]-N-methyl-3-phenyloxirane-2-carboxamide (30a) (1.7 g, 5.72 mmol) in anhydrous toluene (120 mL) was added MgI₂ (1.6 g, 5.72 mmol, 98% purity) and the mixture vigorously stiffed at 25° C. for 32 h. The mixture was treated with saturated ammonium chloride (50 mL) and water (50 mL) and stirred for 30 min. The separated organic layer was washed consecutively with saturated ammonium chloride and brine, dried, filtered and evaporated to an orange oil (1.6 g). Isocratic flash column chromatography (2:1 hexanes-EtOAc) separated the title compound (33b) (400 mg, 24%) as a white solid from the (3S,6S,7R) diastereomer (1.0 g, 59%). There was a significant NOE observed between proton 3 and proton 7. HPLC: 1.78 min (Method A).

### c. (3R,6S,7R)-6-Azido-4-methyl-3,7-diphenyl-1,4-oxazepan-5-one (33c)

Using a procedure similar to Example 31 part c, except using (3R,6R,7R)-6-hydroxy-4-methyl-3,7-diphenyl-1,4-oxazepan-5-one (33b) (305 mg, 1.02 mmol) as reactant, the pure title compound (33c) (385 mg, 38%) was obtained as an off white solid.

### d. (3R,6S,7R)-6-amino-3,7-diphenyl-1,4-oxazepan-5-one hydrochloride (33d)

Using a procedure similar to that described in example 30, part d, except using (3R,6S,7R)-6-azido-4-methyl-3,7-diphenyl-1,4-oxazepan-5-one (33c) (1.0 g, 3.1 mmol) as the azido component, gave the title compound (31d) (170 mg, 96%) as a white solid. ¹H NMR (300 MHz, d6-DMSO) δ2.61 (s, 3H), 4.03 (m, 1H), 4.29 (m, 1H), 5.10 (d, 1H, J=7.9 Hz), 5.38 (m, 1H), 5.52 (m, 1H), 7.31-7.52 (m, 10H), 7.94 (br, 3H). MS APCI, m/z=297(M+1).

### Example 34

### (2S)-2-Hydroxy-4-methyl-N-((1S)-2-[(6S,7R)-4-methyl-5-oxo-7-phenyl-1,4-oxazepan-6-yl]amino-2-oxo-1-phenylethyl)pentanamide (34)

Using a procedure similar to that described in example 28, except using (2S)-2-amino-N-[(6,7-cis)-4-methyl-5-oxo-7-phenyl-1,4-oxazepan-6-yl]-2-phenylacetamide (34b) (73 mg, 0.207 mmol) as the amine component, afforded crude product as an oil. After filtering thru a small plug of silica (CHCl₃), the title compound (34) was obtained in a 1:1 mixture with the 6R,7S diastereomer (80 mg, 83%) as an off white solid. ¹H NMR (300 MHz, CDCl₃) δ 0.92 (dd, 6H), 1.52 (m, 3H), 1.82 (m, 1H), 2.58 (d, 1H), 3.03 (s, 1.5H), 3.09 (s, 1.5H), 3.17 (m, 0.5H), 3.41 (m, 0.5H), 3.70 (m, 1.5H), 3.90 (m, 1.5H), 4.92 (d, 0.5H), 5.03 (d, 0.5H), 5.11 (t, 0.5H), 5.17 (d, 0.5H), 5.23 (t, 0.5H), 5.33 (d, 0.5H), 6.63 (d, 0.5H), 6.79 (d, 0.5H), 6.99 (m, 2H), 7.09 (t, 1H), 7.1-7.4 (m, 7H). MS APCI, m/z=468(M+1). LC/MS: 2.06 min.

The starting amine, (2S)-2-amino-N-[(6,7-cis)-4-methyl-5-oxo-7-phenyl-1,4-oxazepan-6-yl]-2-phenylacetamide (34b), was prepared in the following manner:

### a. tert-Butyl ((1S)-2-{[(6,7-cis)-4-methyl-5-oxo-7-phenyl-1,4-oxazepan-6-yl]amino}-2-oxo-1-phenylethyl)carbamate (34a)

To a suspension of (6,7-cis)-6-amino-4-methyl-7-phenyl-1,4-oxazepan-5-one hydrochloride (30d) (300 mg, 1.17 mmol) in DCM (30 ml) at 0° C. under N₂ was added (2S)-[(tert-butoxycarbonyl)amino](phenyl)acetic acid (293 mg, 1.17 mmol) followed by NMM (128 μL, 1.17 mmol) and the mixture stirred 5 min. until solution cleared. To the reaction was added HOBt-hydrate (389 mg, 2.60 mmol), EDAC.HCl (336 mg, 1.76 mmol) and NMM (193 μL, 1.76 mmol). The reaction mixture was stirred 2 h at 0° C., concentrated in vacuo and partitioned between water and EtOAc. The organic phase was collected and consecutively washed with 0.25 N HCl, water, brine, dried and the solvent removed in vacuo to give the title compound (34a) (380 mg, 72%) as a off white solid. MS APCI, m/z=454 (M+1) 476 (M+Na), LC/MS: 2.15 min.

### b. (2S)-2-Amino-N-[(6,7-cis)-4-methyl-5-oxo-7-phenyl-1,4-oxazepan-6-yl]-2-phenylacetamide trifluoroacetate (34b)

Using a procedure similar to that described in example 29, part b, except using tert-butyl ((1S)-2-{[(6,7-cis)-4-methyl-5-oxo-7-phenyl-1,4-oxazepan-6-yl]amino}-2-oxo-1-phenylethyl)carbamate (26a) (320 mg, 0.706 mmol), as the protected amine component, the title compound (26b) (220 mg, 67%) was obtained as a white solid. ¹H NMR (300 MHz, d6-DMSO) δ2.77 (s, 1.5H), 2.95 (s, 1.5H), 3.22 (m, 1H), 3.72 (m, 2H), 3.98 (m, 1H), 4.75 (m, 0.5H), 4.90 (m, 1H), 4.96 (m, 0.5H), 5.17 (m, 1H), 6.89-7.05 (m, 2H), 7.12 (m, 0.5H), 7.21-7.52 (m, 8.5H), 8.55 (br, 3H). MS APCI, m/z=354(M+1).

### Example 35

### (2S)-2-[(3,5-Difluorophenyl)acetyl]amino-N-[(6S,7R)-4-methyl-5-oxo-7-phenyl-1,4-oxazepan-6-yl]-2-phenylacetamide (35)

Using a procedure similar to that described in example 28, except using (2S)-2-amino-N-[(6,7-cis)-4-methyl-5-oxo-7-phenyl-1,4-oxazepan-6-yl]-2-phenylacetamide (26b) (73.0 mg, 0.207 mmol) as the amine component and (3,5-difluorophenyl)acetic acid (36.0 mg, 0.207 mmol) as the acid component, afforded crude product as an oil. After filtering thru a small plug of silica (CHCl₃), the title compound (35) was obtained in a 1:1 mixture with the 6R,7S diastereomer (75 mg, 71%) as a white foam. ¹H NMR (300 MHz, CDCl₃) 3.00 (s, 1.5H), 3.08 (s, 1.5H), 3.13 (m, 1H), 3.39 (m, 1H), 3.49 (s, 1H), 3.50 (s, 1H), 3.69 (m, 1.5H), 3.88 (m, 1.5H), 4.89 (d, 0.5H), 5.0-5.1 (m, 1.5H), 5.18 (t, 0.5H), 5.28 (d, 0.5H), 6.56 (d, 0.5H), 6.6-6.8 (m, 4H), 6.94 (t, 2H), 7.05 (t, 1H), 7.1-7.3 (m, 6.5H). MS APCI, m/z=508(M+1). LC/MS: 2.15 min.

### Example 36

### (2S)-2-Cyclohexyl-2-[(3,5-difluorophenyl)acetyl]amino-N-[(3R,6S,7R)-4-methyl-5-oxo-3,7-diphenyl-1,4-oxazepan-6-yl]acetamide (36)

Using a procedure similar to that described in example 11, except using (2S)-2-amino-2-cyclohexyl-N-[(3R,6S,7R)-4-methyl-5-oxo-3,7-diphenyl-1,4-oxazepan-6-yl]acetamide (36b) (42 mg, 0.097 mmol) as the amine component and (3,5-difluorophenyl)acetic acid (17 mg, 0.10 mmol) as the acid component, afforded crude product. Recrystallized from ether returned the title compound (36) (33 mg, 58%) as a white solid. ¹H NMR (300 MHz, CDCl₃) δ0.66 (m, 1H), 0.82 (m, 1H), 1.11 (m, 4H), 1.59, (m, 4H), 2.77 (s, 3H), 3.45 (s, 2H), 3.87 (dd, 1H), 3.99 (dd, 1H), 4.37 (t, 1H), 4.98 (d, 1H), 5.23 (m, 1H), 5.51 (t, 1H), 5.64 (d, 1H), 6.7-6.85 (m, 3H), 7.26 (m, 7H), 7.36 (d, 2H), 7.44 (m, 13H). MS APCI, m/z=590(M+1). LC/MS: 2.59 min.

The starting amine, (2S)-2-amino-2-cyclohexyl-N-[(3R,6S,7R)-4-methyl-5-oxo-3,7-diphenyl-1,4-oxazepan-6-yl]acetamide (36b), was prepared in the following manner:

### a. tert-Butyl ((1S)-1-cyclohexyl-2-{[(6S,7R)-4-methyl-5-oxo-7-phenyl-1,4-oxazepan-6-yl]amino}-2-oxoethyl)carbamate (36a)

Using a procedure similar to that described in example 11, except using (3R,6S,7R)-6-amino-4-methyl-3,7-diphenyl-1,4-oxazepan-5-one hydrochloride (33d) (53.0 mg, 0.159 mmol) as the amine component and (2S)-[(tert-butoxycarbonyl)amino](cyclohexyl)acetic acid (41.0 mg, 0.159 mmol) as the acid component, yielded the title compound (36a (55 mg, 64%) as a white solid.

### b. (2S)-2-Amino-2-cyclohex-1-N-[(3R,6S,7R)-4-methyl-5-oxo-3,7-diphenyl-1,4-oxazepan-6-yl]acetamide (36b)

Using a procedure similar to that described in example 29, part b, except using tert-butyl ((1S)-1-cyclohexyl-2-{[(6S,7R)-4-methyl-5-oxo-7-phenyl-1,4-oxazepan-6-yl]amino}-2-oxoethyl)carbamate (36a) (52 mg, 0.10 mmol), as the protected amine component, the title compound was obtained as the TFA salt. The salt was partitioned between saturated NaHCO₃-EtOAc, the organic extract washed with brine, dried, filtered and evaporated to afford pure title compound (36b) (42 mg, 99%) as a white solid. ¹H NMR (300 MHz, CDCl₃) δ0.77-1.75 (m, 13H), 2.80 (s, 3H), 2.84 (m, 1H), 4.01 (m, 1H), 4.43 (m, 1H), 4.99 (d, 1H, J=7.4 Hz), 5.23 (m, 1H), 5.56 (m, 1H), 7.26-7.49 (m, 10H), 7.78 (br, 1H). MS APCI, m/z=436(M+1), LC/MS: 1.73

### Example 37

### (2S)-2-[(3,5-Difluorophenyl)acetyl]amino-N-[(3R,6S,7R)-4-methyl-5-oxo-3,7-diphenyl-1,4-oxazepan-6-yl]-2-phenylacetamide (37)

To a solution of (2S)-2-amino-N-[(3R,6S,7R)-4-methyl-5-oxo-3,7-diphenyl-1,4-oxazepan-6-yl]-2-phenylacetamide (37b) (42 mg, 0.098 mmol) in DCM (5 mL) at 0° C. under N₂ was added the (3,5-difluorophenyl)acetic acid (17 mg, 0.098 mmol), HOBt-hydrate (33 mg, 0.215 mmol), EDAC.HCl (28 mg, 0.147 mmol) and NMM (18 μL, 0.147 mmol). The reaction mixture was stirred 1 h at 0° C., concentrated in vacuo and partitioned between 0.25N HCl (10 ml) and EtOAc (10 mL). The organic phase was collected and consecutively washed with 0.25N HCl, water, saturated NaHCO₃, and brine, dried and the solvent removed in vacuo to yield an oil. After filtering thru a small plug of silica (CHCl₃) the title compound (37) was obtained (35 mg, 61%) as a white solid. ¹H NMR (300 MHz, CDCl₃) δ2.67 (s, 3H), 3.49 (s, 2H), 3.97 (dd, 1H), 4.34 (t, 1H), 4.80 (d, 1H), 5.01 (d, 1H), 5.21 (dd, 1H), 5.46 (t, 1H), 6.48 (d, 1H), 6.7-6.8 (m, 3H), 7.02 (m, 2H), 7.2-7.3 (m, 10.5H), 7.40 (m, 3.5H). MS APCI, m/z=584(M+1). LC/MS: 2.51 min.

The starting amine, racemic (2S)-2-amino-N-[(3R,6S,7R)-4-methyl-5-oxo-3,7-diphenyl-1,4-oxazepan-6-yl]-2-phenylacetamide (37b), was prepared in the following manner:

### a. tert-Butyl (2-{[(3S,6R,7S)-4-methyl-5-oxo-3,7-diphenyl-1,4-oxazepan-6-yl]amino}-2-oxo-1-phenylethyl)carbamate (37a)

Using a procedure similar to that described in example 11, except using (3R,6S,7R)-6-amino-4-methyl-3,7-diphenyl-1,4-oxazepan-5-one hydrochloride (33d) (53 mg, 0.159 mmol) as the amine component and (2S)-[(tert-butoxycarbonyl)amino]phenylacetic acid (40 mg, 0.159 mmol) as the acid component, the title compound (37a) (55 mg, 64%) was obtained as a white solid. MS APCI, m/z=552(M+Na). LC/MS:2.61 min.

### b. (2S)-2-Amino-N-[(3R,6R,7S)-4-methyl-5-oxo-3,7-diphenyl-1,4-oxazepan-6-yl]-2-phenylacetamide (37b)

Using a procedure similar to that described in example 25, part c, except using tert-butyl (2-{[(3S,6R,7S)-4-methyl-5-oxo-3,7-diphenyl-1,4-oxazepan-6-yl]amino}-2-oxo-1-phenylethyl)carbamate (37a) (52 mg, 0.10 mmol), the title compound (27b) (42 mg, 99%) was obtained as a white solid. ¹H NMR (300 MHz, CDCl₃) δ2.77 (s, 3H), 3.99 (m, 1H), 4.16 (m, 1H), 4.40 (m, 1H), 4.97 (d, 1H, J=7.4 Hz), 5.25 (m, 1H), 5.57 (m, 1H), 6.84 (m, 2H), 7.13-7.52 (m, 15H), 7.88 (m, 1H). MS APCI, m/z=430 (M+1). LC/MS: 1.66 min.

### Example 38

### N²-[(3,5-Difluorophenyl)acetyl]-N¹-[(6S,7R)-4-(4-methoxybenzyl)-5-oxo-7-phenyl-1,4-oxazepan-6-yl]-L-alaninamide (38)

Using a procedure similar to that described in example 11, except using (6,7-cis)-6-amino-4-(4-methoxybenzyl)-7-phenyl-1,4-oxazepan-5-one hydrochloride (38d) (100 mg, 0.275 mmol) as the amine component, the title compound (38) was obtained in a 1:1 mixture with the 6R,7S diastereomer (139 mg, 91%) as a white foam. ¹H NMR (300 MHz, CDCl₃) δ0.87 (d, 1.5H), 1.21 (d, 1.5H), 3.34 (m, 1H), 3.45 (s, 1H), 3.46 (s, 1H), 3.59 (m, 2H), 3.72 (m, 1H), 3.39 (s, 3H), 4.30 (m, 1H), 4.49 (dd, 1H), 4.79 (dd, 1H), 5.00 (dd, 1H), 5.22 (q, 1H), 5.79 (d, 0.5H), 6.08 (d, 0.5H), 6.7-6.9 (m, 5.5H), 6.97 (d, 0.5H), 7.16 (t, 2H), 7.29 (m, 5H). MS APCI, m/z=552 (M+1). LC/MS: 2.29 min.

The starting amine, (6,7-cis)-6-amino-4-methyl-7-phenyl-1,4-oxazepan-5-one hydrochloride (38d), was prepared in the following manner:

### a. (2,3-trans)-N-(2-Hydroxyethyl)-N-(4-methoxybenzyl)-3-phenyloxirane-2-carboxamide (38a)

Using a procedure similar to that described in example 31 part a, except using 2-[(4-methoxybenzyl)amino]ethanol (5.85 g, 32.3 mmol) as the amine component, the title compound (38a) (8.3 g, 93%) was obtained as a white solid.

### b. (6,7-trans)-6-Hydroxy-4-(4-methoxybenzyl)-7-phenyl-1,4-oxazepan-5-one (38b)

To a stirred solution of (2,3-trans)-N-(2-hydroxyethyl)-N-(4-methoxybenzyl)-3-phenyloxirane-2-carboxamide (38a) (3.9 g, 11.9 mmol) in anhydrous THF (350 mL) was added 20 mol % of MgI₂ (667 mg, 2.4 mmol, 98% purity) and the mixture heated to reflux for 1 h, cooled, evaporated, and partitioned between EtOAc and saturated ammonium chloride. The organic layer was washed with saturated ammonium chloride, dried, filtered, and evaporated to an orange oil. Isocratic flash column chromatography (40% EtOAc-hexanes) returned the title compound (38b) (3.4 g, 89%) as a white solid.

### c. (6,7-cis)-6-Azido-4-(4-methoxybenzyl)-7-phenyl-1,4-oxazepan-5-one (38c)

Using a procedure similar to that described in example 31 part c, except using (6,7-trans)-6-hydroxy-4-(4-methoxybenzyl)-7-phenyl-1,4-oxazepan-5-one (38b) (2.3 g, 7.03 mmol) as the alcohol component, the title compound (38c) (1.4 g, 58%) was obtained as a light yellow oil. MS APCI, m/z=353(M+1). LC/MS: 2.63 min (Method D).

### d. (6,7-cis)-6-Amino-4-(4-methoxybenzyl)-7-phenyl-1,4-oxazepan-5-one hydrochloride (38d)

Using a procedure similar to that described in example 30, part d, except using (6,7-cis)-6-azido-4-(4-methoxybenzyl)-7-phenyl-1,4-oxazepan-5-one (38c) (1.35 g, 3.84 mmol) as the azido component, the title compound (38d) (1.3 g, 93%) was obtained as a white solid. ¹H NMR (300 MHz, CDCl₃) δ1.62 (bs, 2H), 3.28 (dd, 1H J=6.6, 10.0 Hz), 3.50 (m, 1H), 3.79 (s, 3H), 3.82-3.92 (m, 2H), 4.08 (s, 1H), 4.59 (dd, 2H, J=14, 19 Hz), 4.83 (s, 1H), 6.83 (d, 2H, J=8.8 Hz), 7.17 (d, 2H, J=8.8 Hz), 7.27-7.44 (m, 5H). MS APCI, m/z=327 (M+1). LC/MS: 1.46 min.

### Example 39

### N²-[(3,5-Difluorophenyl)acetyl]-N¹-[(2R,3S,5aS,9aS)-5-methyl-4-oxo-2-phenyldecahydro-1,5-benzoxazepin-3-yl]-L-alaninamide (39)

Using a procedure similar to that described in example 11, except using (2R,3S,5aS,9aS)-3-amino-5-methyl-2-phenyloctahydro-1,5-benzoxazepin-4(5H)-one (39d) (110 mg, 0.401 mmol) as the amine component, afforded a crude oil (200 mg). Flash column chromatography (60-100% EtOAc-hexanes) returned the title compound (19) (95 mg, 48%) as a white solid. ¹H NMR (300 MHz, CDCl₃) δ1.28 (d, 3H), 1.45 (m, 4H), 1.87 (m, 2H), 2.17 (m, 2H), 3.05 (s, 3H), 3.15 (dt, 1H), 3.52 (s, 2H), 4.23 (m, 1H), 4.29 (t, 1H), 5.25 (d, 1H), 5.43 (t, 1H), 6.02 (d, 1H), 6.46 (d, 1H), 6.73 (dt, 1H), 6.82 (m, 1.5H), 7.10 (m, 2H), 7.35 (m, 3.5H). MS APCI, m/z=500(M+1). LC/MS: 2.42 min.

The starting amine, (2R,3S,5aS,9aS)-3-amino-5-methyl-2-phenyloctahydro-1,5-benzoxazepin-4(5H)-one (39d), was prepared in the following manner:

### a. (2,3-trans)-N-[(1S,2S)-2-Hydroxycyclohexyl]-N-methyl-3-phenyloxirane-2-carboxamide (39a)

To a solution of ethyl (2,3-trans)-3-phenyloxirane-2-carboxylate (4.45 g, 23.3 mmol) in methanol (12 mL) at −20° C. was added (1S,2S)-2-(methylamino)cyclohexanol (3.0 g, 23.3 mmol) [Lu, X. et al, Org. Proc. R&D, 2001, 5, 184-185.] followed addition of catalytic sodium methoxide (25%, 12 drops). The reaction was placed in a freezer (˜−20 C) for 6 d, the solvent evaporated and the residue purified by flash column chromatography eluting (2% methanol-CHCl₃) to afford the title compound (39a) (3.5 g, 55%). MS APCI, m/z=276(M+1). LC/MS: 1.77 min.

### b. (2R,3S,5aR,9aS)-3-Hydroxy-5-methyl-2-phenyloctahydro-1,5-benzoxazepin-4(5H)-one (39b)

To a stirred solution of (2,3-trans)-N-[(1S,2S)-2-hydroxycyclohexyl]-N-methyl-3-phenyloxirane-2-carboxamide (39a) (3.6 g, 13.1 mmol) in anhydrous THF (500 mL) was added MgI₂ (730 mg, 2.62 mmol, 98% purity) and the mixture stirred at reflux 16 h, cooled, the volume reduced to ˜150 mL under reduced pressure. The residue was partitioned between EtOAc (300 mL) and saturated ammonium chloride (200 mL), stirred 15 min and the organic layer washed with saturated ammonium chloride, brine, dried, filtered, and evaporated to a crude orange oil (3.8 g). After recrystallization from EtOAc-hexanes, the title compound (39b) (1.35 g, 75%, 37.5% based on total 39a) was obtained as a white solid. MS APCI, m/z=276(M+1). LC/MS: 2.13 min.

### c. (2R,3R,5aR,9aS)-3-Azido-5-methyl-2-phenyloctahydro-1,5-benzoxazepin-4(5H)-one (30c)

Using a procedure similar to that described in example 31 part c, except using (2R,3S,5aR,9aS)-3-hydroxy-5-methyl-2-phenyloctahydro-1,5-benzoxazepin-4(5H)-one (39b) (1.3 g, 4.72 mmol) as the alcohol component, pure title compound (30c) (500 mg, 35%) was obtained. MS APCI, m/z=301 (M+1) 272(M+1-N₂). LC/MS: 6.84 min (Method E).

### d. (2S,3R,5aR,9aS)-3-Amino-5-methyl-2-phenyloctahydro-1,5-benzoxazepin-4(5H)-one hydrochloride (39d)

Using a procedure similar to that described in example 31 part c, except using (2R,3R,5aR,9aS)-3-azido-5-methyl-2-phenyloctahydro-1,5-benzoxazepin-4(5H)-one (39b), (350 mg, 1.17 mmol) as the azido reactant, afforded a crude semi-solid. Free based was obtained (EtOAc-saturated NaHCO3) and the material purified by flash column chromatography (5% methanol-DCM) to afford the title compound (39d) (110 mg, 34%) as an off white solid. ¹H NMR (300 MHz, CDCl₃) δ1.24-1.41 (m, 4H), 1.48 (br 2H), 1.85 (m, 2H), 2.16 (m, 2H), 3.04 (s, 3H), 3.14 (m, 1H), 4.05 (m, 1H), 4.36 (d, 1H, J=6.0 Hz), 5.02 (d, 1H, J=6.0 Hz), 7.21-7.34 (m, 5H). MS APCI, m/z=275 (M+1). LC/MS: 1.40 min.

### Example 40

### (2S)-2-[(3,5-Difluorophenyl)acetyl]amino-N-[(6S,7R)-4-(4-methoxybenzyl)-5-oxo-7-phenyl-1,4-oxazepan-6-yl]-2-phenylacetamide (40)

Using a procedure similar to that described in example 28, except using (2S)-2-amino-N-[(6,7-cis)-4-(4-methoxybenzyl)-5-oxo-7-phenyl-1,4-oxazepan-6-yl]-2-phenylacetamide hydrochloride (10) (120 mg, 0.242 mmol) as the amine component and (3,5-difluorophenyl)acetic acid (42 mg, 0.242 mmol) as the acid component, afforded crude product as an oil. Flash column chromatography (20% EtOAc-DCM) returned the title compound (40) (pure diastereomer) (42 mg, 57%) as a white solid. ¹H NMR (300 MHz, CDCl₃) δ 3.33 (m, 1H), 3.51 (s, 2H), 3.52-3.75 (m, 4H), 3.77 (t, 3H), 4.45 (d, 1H), 4.72 (d, 1H), 4.93 (d, 1H), 5.22 (t, 1H), 5.31 (t, 1H), 6.64-6.82 (m, 6H), 6.93-7.31 (m, 12H). MS APCI, m/z=614 (M+1). HPLC: 4.48 min (Method B).

The starting amine, (2S)-2-amino-N-[(6,7-cis)-4-(4-methoxybenzyl)-5-oxo-7-phenyl-1,4-oxazepan-6-yl]-2-phenylacetamide hydrochloride (40b), was prepared in the following manner:

### a. tert-Butyl ((1S)-2-{[(6,7-cis)-4-(4-methoxybenzyl)-5-oxo-7-phenyl-1,4-oxazepan-6-yl]amino}-2-oxo-1-phenylethyl)carbamate (40a)

Using a procedure similar to that described in example 26 part a, except using (2S-6,7-cis)-6-amino-4-methyl-2,7-diphenyl-1,4-oxazepan-5-one (38d) (150 mg, 0.46 mmol) as the amine component, the title compound (40a) (220 mg, 86%) was obtained as a white solid. MS APCI, m/z=560(M+1). LC/MS: 2.78 min.

### b. (2S)-2-Amino-N-[(6,7-cis)-4-(4-methoxybenzyl)-5-oxo-7-phenyl-1,4-oxazepan-6-yl]-2-phenylacetamide hydrochloride (40b)

Using a procedure similar to that described in example 29 part b, except using tert-butyl ((1S)-2-{[(6,7-cis)-4-(4-methoxybenzyl)-5-oxo-7-phenyl-1,4-oxazepan-6-yl]amino}-2-oxo-1-phenylethyl)carbamate (40a) (195 mg, 0.35 mmol) as the protected amine component, the title compound was obtained as the TFA salt. The salt was freed (EtOAc-saturated sodium bicarbonate) and an EtOAc solution of the free base treated with HCl-ether until pH 1, evaporated and treated with ether to afford the title compound (40b) (147 mg, 85%) as a white solid. ¹H NMR (300 MHz, D6 DMSO) δ3.56 (m, 2H), 3.72 (s, 1.5H), 3.74 (s, 1.5H), 3.83 (m, 1H), 4.11 (d, 0.5H), 4.42 (d, 0.5H), 4.56 (s, 0.5H), 4.61, (s, 0.5H), 4.77, (s, 0.5H), 5.02, (m, 1.5H), 5.22 (m, 1H), 6.74 (d, 1H), 6.87-6.97 (m, 4H), 7.01 (t, 1H), 7.12-7.23 (m, 2H), 7.24-7.50 (m, 7H), 8.52 (d, 0.5H), 8.62 (m, 3H), 8.84 (d, 0.5H). MS APCI, m/z=460(M+1). LC/MS 1.88/1.96 min.

### Example 41

### N²-[(3,5-Difluorophenyl)acetyl]-N¹-[(2R,3R)-2-(4-methoxyphenyl)-4-oxo-2,3,4,5-tetrahydro-1,5-benzothiazepin-3-yl]-L-alaninamide (41)

To a stirred solution of (2,3-cis)-3-amino-2-(4-methoxyphenyl)-2,3-dihydro-1,5-benzothiazepin-4(5H)-one hydrobromide (41d) (70 mg, 0.184 mmol) in DCM (1 mL) under nitrogen was added N-[(3,5-difluorophenyl)acetyl]-L-alanine (1e) (45 mg, 0.185 mmol), HOBt (31 mg, 0.229 mmol), NMM (46 mg, 0.459 mmol) and EDAC-HCl (43 mg, 0.224 mmol). The mixture was stirred for ˜12 h at 25° C. then evaporated. The residue was dissolved in EtOAc and extracted in succession with saturated sodium bicarbonate, 1N aqueous HCl, and brine. The organic solution was dried, filtered and evaporated. The residue was purified by flash chromatography on silica gel eluting with 1:1 (v/v) DCM:EtOAc to afford the title compound in a 1:1 mixture with the 2S,3S diastereomer (88 mg, 90%) as a white solid. ¹H NMR (300 MHz, CDCl₃) δ0.99 (d, 1.5H, J=7.0 Hz), 1.18 (d, 1.5H, J=7.0 Hz), 3.42 (s, 1H), 3.44 (s, 1H), 3.78 (s, 1.5H), 3.81 (s, 1.5H), 4.24 (m, 0.5H), 4.43 (m, 0.5H), 4.84 (q, 1H, J=7.0 Hz), 5.22 (m, 1H), 5.90 (d, 0.5H), 6.10 (d, 0.5H), 6.38 (d, 0.5H), 6.55 (d, 0.5H), 6.64-6.92 (m, 4H), 7.11 (m, 1H), 7.19-7.46 (m, 5H), 7.71 (m, 1.5H), 8.09 (s, 0.5H). MS APCI, m/z=548 (M+Na). LC/MS: 4.43 min.

The precursor (2,3-cis)-3-amino-2-(4-methoxyphenyl)-2,3-dihydro-1,5-benzothiazepin-4(5H)-one hydrobromide (41d) was prepared as follows:

### a. Methyl (2Z)-2-{[(benzyloxy)carbonyl]amino}-3-(4-methoxyphenyl)acrylate (41a)

Using a procedure similar to that described in example 1 part a, except using 4-methoxybenzaldehyde (730 μl, 6.0 mmol) as the aldehyde component, the title compound was obtained as an oil (1.6 g, 78%). ¹H NMR (300 MHz, CDCl₃) δ3.79 (s, 3H), 3.82 (s, 3H), 5.15 (s, 2H), 6.20 (bs, 1H), 6.85 (d, 2H), 7.34 (m, 5H), 7.49 (d, 2H). MS APCI, m/z=342 (M+1).

### b. Methyl β-[(2-aminophenyl)thio]-N-[(benzyloxy)carbonyl]-4-methoxyphenylalaninate (41b)

Using a procedure similar to that described in example 10 (stirred 12 d), part b, except using methyl (2Z)-2-{[(benzyloxy)carbonyl]amino}-3-(4-methoxyphenyl)acrylate (41a) (1.5 g, 4.4 mmol), the product was obtained as a crude solid (2.0 g). Recrystallized from EtOAc-hexanes to afford the title compound (1.1 g, 54%) as white solid (91:9 mixture (erythro:threo)). ¹H NMR (300 MHz, CDCl₃) δ 3.43(s, 3H), 3.78 (s, 3H) 4.25 (s, 2H), 4.56 (d, 2H, J=4.8 Hz), 4.77 (m, 1H), 5.10, (dd, 2H,), 5.83 (d, 1H), 6.58 (t, 1H, J=6.0 Hz), 6.67, (d, 1H, J=8.3 Hz), 6.77 (d, 2H, J=8.7 Hz), 7.09 (t, 1H, J=7.9 Hz), 7.20 (m, 3H), 7.36 (m, 4H). MS APCI, m/z=467 (M+1). LC/MS: 2.65 min.

### c. Benzyl [(2,3-cis)-2-(4-methoxyphenyl)-4-oxo-2,3,4,5-tetrahydro-1,5-benzothiazepin-3-yl]carbamate (41c)

A suspension of methyl β-[(2-aminophenyl)thio]-N-[(benzyloxy)carbonyl]-4-methoxyphenylalaninate (41b) (9:1, 1.1 g, 2.36 mmol) and pTSA (catalytic) in xylenes (20 mL) was heated to reflux for 2 h, using a Dean-Stark apparatus. The mixture was then cooled, resulting in precipitation of the trans product as a white solid (80 mg, 95:5 trails). The filtrate (950 mg) was evaporated and crystallized from ether to afford a still impure product (740 mg). This solid was heated in EtOAc and filtered to give pure title compound (600 mg, 64%) as a white solid. ¹H NMR (300 MHz, DMSO-d6) δ 3.76 (s, 3H), 4.49 (t, 1H, J=7.4 Hz)), 4.95 (s, 3H), 5.13 (d, 1H, J=7.0 Hz), 5.96 (d, 1H), 6.90 (d, 2H, J=8.3 Hz), 7.31 (m, 9H), 7.49 (t, 1H), 7.66 (t, 1H, 7.5 Hz), 10.48 (s, 1H). MS APCI, m/z=457(M+Na). LC/MS: 2.66 min.

### d. (2,3-cis)-3-Amino-2-(4-methoxyphenyl)-2,3-dihydro-1,5-benzothiazepin-4(5H)-one hydrobromide (41d)

To benzyl [(2,3-cis)-2-(4-methoxyphenyl)-4-oxo-2,3,4,5-tetrahydro-1,5-benzothiazepin-3-yl]carbamate (41c) (380 mg, 0.87 mmol) in 6 ml HOAc was added 30% HBr/HOAc (2 mL). The stirred suspension became a homogeneous solution over 20 min. The reaction stirred at 25° C. for 2 h. The volume was reduced to ˜2-3 ml, diluted with ether to afford the hydrobromide salt of the title compound (0.75 g, 95%) as a white solid. ¹H NMR (300 MHz, DMSO-d6) δ 3.79 (s, 3H), 4.23 (d, 1H, J=7.4 Hz), 5.19 (d, 1H, J=7.4 Hz), 6.99 (d, 2H,), 7.25 (d, 1H), 7.30 (t, 1H), 7.45 (d, 2H), 7.51 (t, 1H), 7.68 (d, 1H, 7.5 Hz), 7.98 (bs, 3H), 10.83 (s, 1H). MS APCI, m/z=323(M+Na). LC/MS: 1.63 min.

### Example 42

### N¹-[(2R,3R)-7-chloro-2-(2,5-difluorophenyl)-4-oxo-2,3,4,5-tetrahydro-1,5-benzothiazepin-3-yl]-N²-[(2S)-2-(3,5-difluorophenyl)-2-hydroxyacetyl]-L-alaninamide (42)

To a stirred solution of (2,3-cis)-3-amino-7-chloro-2-(2,5-difluorophenyl)-2,3-dihydro-1,5-benzothiazepin-4(5H)-one (92d) (131 mg, 0.384 mmol) in DCM (3 mL) and DMF (0.5 mL) under nitrogen was added N-[(2S)-2-(3,5-difluorophenyl)-2-hydroxyacetyl]-L-alanine (78b) (100 mg, 0.386 mmol), HOBt (66 mg, 0.488 mmol), NMM (49 mg, 0.484 mmol) and EDAC-HCl (100 mg, 0.521 mmol). The mixture was stirred for ˜12 h at 25° C. The reaction was diluted with water and extracted with EtOAc. The organic solution was dried, filtered and evaporated. The residue was purified by flash chromatography on silica gel eluting with 2:1 (v/v) hexanes:EtOAc to afford the title compound in a 1:1 mixture with the 2S,3S diastereomer (119 mg, 90%) as a white solid. ¹H NMR (300 MHz, CDCl₃) δ1.14 (d, 1.5H, J=7.0 Hz), 1.26 (d, 1.5H, J=7.0 Hz), 3.92 (d, 0.5H, J=4.4 Hz), 4.08 (d, 0.5H, J=4.4 Hz), 4.41 (m, 0.5H), 4.56 (m, 0.5H), 4.82-5.03 (m, 2H), 5.64 (d, 0.5H, J=5.6 Hz), 5.68 (d, 0.5H, J=5.6 Hz), 6.72 (m, 1H), 6.85-7.32 (m, 8H), 7.50 (m, 1H), 7.63 (m, 1H), 8.26 (s, 0.5H), 8.73 (s, 0.5H). MS APCI, m/z=582 (M+1). LC/MS: 2.61 min.

### Example 43

### N²-[(2S)-2-hydroxy-4-methyl-1-oxopentyl]-N¹-[(2R,3S)-4-oxo-2-phenyl-2,3,4,5-tetrahydro-1,5-benzoxazepin-3-yl]-L-alaninamide (43)

To a stirred solution of N¹-[(2,3-cis)-4-oxo-2-phenyl-2,3,4,5-tetrahydro-1,5-benzoxazepin-3-yl]-L-alaninamide (43a) (160 mg, 0.492 mmol) in 1:1 (v/v) DCM:DMF (4 mL) under nitrogen was added (S)-2-hydroxyisocaproic acid (71 mg, 0.537 mmol), HOBt (81 mg, 0.600 mmol), NMM (61 mg, 0.603 mmol) and EDAC-HCl (115 mg, 0.599 mmol). The mixture was stirred for ˜12 h at 25° C. then diluted with aqueous sodium bicarbonate and extracted with EtOAc. The organic extract was washed with 15% aqueous citric acid and brine then dried and evaporated. The residue was purified by flash chromatography on silica gel eluting first with 1:1 (v/v) hexanes:EtOAc then with EtOAc to afford the title compound in a 1:1 mixture with the 2S,3R diastereomer (125 mg, 58%) as a yellow solid. ¹H NMR (300 MHz, Acetone-d6) δ0.91 (m, 7H), 1.21 (m, 3H), 1.49 (m, 1H), 1.86 (m, 1H), 2.80 (br, 1H), 4.01 (m, 1H), 4.46 (m, 1H), 5.14 (m, 1H), 5.76 (m, 1H), 6.92 (m, 1H), 7.19-7.54 (m, 10H), 9.33 (br, 1H). MS APCI, m/z=462 (M+Na). LC/MS: 2.06 min.

The precursor N¹-[(2,3-cis)-4-oxo-2-phenyl-2,3,4,5-tetrahydro-1,5-benzoxazepin-3-yl]-L-alaninamide (43a was prepared as follows:

### a. N¹-[(2,3-cis)-4-Oxo-2-phenyl-2,3,4,5-tetrahydro-1,5-benzoxazepin-3-yl]-L-alaninamide (43a)

Trifluoroacetic acid (5 mL) was added to a solution of N²-[tert-butoxycarbonyl]-N¹-[(2,3-cis)-4-oxo-2-phenyl-2,3,4,5-tetrahydro-1,5-benzoxazepin-3-yl]-L-alaninamide (6f) (250 mg, 0.587 mmol) in DCM (10 mL) at 0° C. under nitrogen. The mixture was stirred for 2 h then evaporated. The residue was dissolved in EtOAc and extracted with saturated aqueous sodium bicarbonate. The organic solution was dried, filtered and evaporated to afford the title compound as a yellow oil (165 mg, 86%). MS APCI, m/z=326 (M+1). LC/MS: 1.41 and 1.49 min. (diastereomers evident).

### Example 44

### N²-[(3,5-Difluorophenyl)acetyl]-N¹-[(2R,3S)-5-methyl-4-oxo-2-phenyl-2,3,4,5-tetrahydro-1,5-benzoxazepin-3-yl]-L-alaninamide (44)

Sodium hydride (7 mg, 0.183 mmol) was added to a solution of N²-[(3,5-difluorophenyl)acetyl]-N¹-[(2,3-cis)-4-oxo-2-phenyl-2,3,4,5-tetrahydro-1,5-benzoxazepin-3-yl]-L-alaninamide (44a) (77 mg, 0.161 mmol) in DMF (1 mL) under nitrogen. After stirring for 5 min. at ambient temperature, iodomethane (25 mg, 0.176 mmol) was added. After 4 h the reaction was quenched with 1N aqueous HCl, diluted with water and extracted with EtOAc. The residue obtained after evaporation of the organic extract was purified by flash chromatography on silica gel eluting with 1:1 (v/v) hexanes-EtOAc then with EtOAc to afford the title compound in a 1:1 mixture with the 2S,3R diastereomer (70 mg, 78%) as a white powder. ¹H NMR (300 MHz, CDCl₃) δ1.08 (d, 1.5H, J=7.0 Hz), 1.21 (d, 1.5H, J=7.0 Hz), 3.42-3.52 (m, 5H), 4.17-4.34 (m, 1H), 5.08 (m, 1H), 5.69 (m, 1H), 5.88-6.03 (m, 1H), 6.25 (m, 1H), 6.65-6.83 (m, 3H), 7.25-7.40 (m, 9H). MS APCI, m/z=516 (M+Na). LC/MS: 2.29 min.

The required N²-[(3,5-difluorophenyl)acetyl]-N¹-[(2,3-cis)-4-oxo-2-phenyl-2,3,4,5-tetrahydro-1,5-benzoxazepin-3-yl]-L-alaninamide (44a) was prepared as follows:

### a. N²-[(3,5-Difluorophenyl)acetyl]-N-[(2,3-cis)-4-oxo-2-phenyl-2,3,4,5-tetrahydro-1,5-benzoxazepin-3-yl]-L-alaninamide (44a)

To a stirred solution of (2,3-cis)-3-amino-2-phenyl-2,3-dihydro-1,5-benzoxazepin-4(5H)-one hydrochloride (6e) (103 mg, 0.356 mmol) in DMF (4 mL) under nitrogen at ambient temperature was added N-[(3,5-difluorophenyl)acetyl]-L-alanine (1e) (95 mg, 0.391 mmol), HOBt (58 mg, 0.427 mmol), NMM (71 mg, 0.700 mmol) and EDAC-HCl (82 mg, 0.427 mmol). After 6 h the reaction was diluted with water and extracted three times with EtOAc. The combined organic extracts were dried, filtered and evaporated. The residue was purified by flash chromatography on silica gel eluting first with 1:1 (v/v) hexanes:EtOAc and then with EtOAc to afford the title compound (75 mg, 43%) as a white solid. MS APCI, m/z=480 (M+1). LC/MS: 2.31 min. ¹H NMR (300 MHz, DMSO-d6) δ1.05 (d, 1.5H, J=7 Hz), 1.11 (d, 1.5H, J=7 Hz), 3.44 (m, 2H), 4.27 (m, 1H), 4.97 (m, 1H), 5.60 (t, 1H, J=6 Hz), 6.86-7.50 (m, 13H), 8.24 (d, 0.5H, 1=7 Hz), 8.33 (d, 0.5H, J=7 Hz), 10.30 (d, 1H, J=7 Hz).

### Example 45

### N¹-[(2R,3R)-7-Chloro-2-(2,5-difluorophenyl)-5-methyl-4-oxo-2,3,4,5-tetrahydro-1,5-benzothiazepin-3-yl]-N²-[(3,5-difluorophenyl)acetyl]-L-alaninamide (45)

To a stirred solution of N¹-[(2R,3R)-7-chloro-2-(2,5-difluorophenyl)-4-oxo-2,3,4,5-tetrahydro-1,5-benzothiazepin-3-yl]-N²-[(3,5-difluorophenyl)acetyl]-L-alaninamide (92) (100 mg, 0.177 mmol) in DMF (800 uL) under nitrogen at ambient temperature was added sodium hydride powder (60% in mineral oil) (7 mg, 0.182 mmol). After 5 minutes, iodomethane (25 mg, 0.176 mmol) was added via micro-syringe. After stirring for 3.5 hours, the reaction was carefully quenched with 1N aqueous HCl then diluted with water and extracted with EtOAc. The organic extracts were combined and evaporated. The residue was purified by flash chromatography on silica gel eluting first with 1:1 (v/v) hexanes-EtOAc and finally with EtOAc to afford the title compound (85 mg, 82%) as a white solid. ¹H NMR (300 MHz, Acetone-d6) δ1.07 (d, 1.5H, J=7.0 Hz), 1.13 (d, 1.5H, J=7.0 Hz), 3.46-3.58 (m, 5H), 4.28 (m, 0.5H), 4.40 (m, 0.5H), 4.87 (m, 1H), 5.52 (m, 1H), 6.80-6.98 (m, 3H), 7.11-7.26 (m, 3H), 7.33 (m, 0.5H), 7.40-7.51 (m, 2.5H), 7.70 (d, 1H, J=2.2 Hz), 7.81 (d, 1H, J=8.3 Hz). MS APCI, m/z=580 (M+1). LC/MS: 2.89 min.

### Example 46

### N²-[(3,5-Difluorophenyl)acetyl]-N¹-(2R,3S)-5-[2-(dimethylamino)ethyl]-4-oxo-2-phenyl-2,3,4,5-tetrahydro-1,5-benzoxazepin-3-yl-L-alaninamide hydrochloride (46)

A gentle stream of hydrogen chloride gas was bubbled through a solution of tert-butyl {(2,3-cis)-5-[2-(dimethylamino)ethyl]-4-oxo-2-phenyl-2,3,4,5-tetrahydro-1,5-benzoxazepin-3-yl}carbamate (46b) (72 mg, 0.169 mmol) in EtOAc (10 mL) at 0° C. for 5 min. The solvent was evaporated and the residue dissolved in DMF (3 mL). N-[(3,5-difluorophenyl)acetyl]-L-alanine (1e) (73 mg, 0.300 mmol), HOBt (54 mg, 0.400 mmol), NMM (61 mg, 0.600 mmol) and EDAC-HCl were added in succession to the stirred solution and the mixture kept at 25° C. for ˜12 h. The reaction was diluted with aqueous sodium carbonate and extracted with EtOAc. The residue obtained after evaporation of the organic phase was purified by flash chromatography on silica gel eluting first with 40:1 (v/v) then with 20:1 (v/v) CHCl₃-methanol. Evaporation of the product containing fractions afforded an oil which was dissolved in DCM and treated with a slight excess of ethereal hydrogen chloride. Evaporation of the solution afforded the title compound in a 1:1 mixture with the 2S,3R diastereomer (30 mg, 30%) as a white solid. ¹H NMR (300 MHz, DMSO-d6) δ1.03 (d, 1.5H, J=7.0 Hz), 1.08 (d, 1.5H, J=7.0 Hz), 2.85 (m, 6H), 3.40 (m, 4H), 4.25 (m, 3H), 5.00 (m, 1H), 5.54 (m, 1H), 6.85-7.48 (m, 12H), 7.61 (m, 1H), 8.23 (d, 0.5H, J=7.0), 8.34 (d, 0.5H, J=7.0). MS APCI, m/z=551 (M+1). LC/MS: 2.16 min.

The precursor tert-butyl {(2,3-cis)-5-[2-(dimethylamino)ethyl]-4-oxo-2-phenyl-2,3,4,5-tetrahydro-1,5-benzoxazepin-3-yl}carbamate (46b) was prepared as follows:

### a. tert-Butyl [(2,3-cis)-4-oxo-2-phenyl-2,3,4,5-tetrahydro-1,5-benzoxazepin-3-yl]carbamate (46a)

To a stirred solution of (2,3-cis)-3-amino-2-phenyl-2,3-dihydro-1,5-benzoxazepin-4(5H)-one (6e) (700 mg, 3.207 mmol) in DCM (30 mL) under nitrogen at 0° C. was added triethylamine (324 mg, 3.21 mmol) and di-tert-butyl dicarbonate (700 mg, 3.208 mmol). The mixture was allowed to warm to ambient temperature and stirred for 24 h. The solvent was evaporated and the residue purified by flash chromatography on silica gel eluting first with DCM and finally with 20:1 (v/v) DCM-EtOAc to afford the title compound (943 mg, 83%) as a white solid. TLC 2:1 (v/v) hexane-EtOAc R_{f}=0.50. ¹H NMR (300 MHz, CDCl₃) δ1.39 (s, 9H), 4.90-5.05 (m, 2H), 5.76 (d, 1H, J=6.8 Hz), 7.05 (m, 1H), 7.14-7.29 (m, 3H), 7.36-7.53 (m, 6H). MS ES⁺, m/z=377 (M+Na). LC/MS: 1.93 min.

### b. tert-Butyl {(2,3-cis)-5-[2-(dimethylamino)ethyl]-4-oxo-2-phenyl-2,3,4,5-tetrahydro-1,5-benzoxazepin-3-yl}carbamate (46b)

To a stirred solution of tert-butyl [(2,3-cis)-4-oxo-2-phenyl-2,3,4,5-tetrahydro-1,5-benzoxazepin-3-yl]carbamate (46a) (70 mg, 0.197 mmol) and 2-(dimethylamino)ethylchloride hydrochloride (43 mg, 0.296 mmol) in methyl isobutyl ketone (5 mL) was added 1M aqueous sodium hydroxide (985 μL) and a catalytic amount of tetrabutylammonium iodide. The biphasic mixture was stirred and heated at reflux for 8 h. The reaction was diluted with aqueous sodium carbonate and extracted with EtOAc. The residue from the organic extract was purified by flash chromatography on silca gel eluting with 20:1 (v/v) CHCl₃-methanol to afford the title compound (72 mg, 86%) white solid. MS ES⁺, m/z=448 (M+Na). HPLC (Method A): 2.52 min.

### Example 47

### N¹-[(2R,3R)-7-Chloro-2-(2,5-difluorophenyl)-4-oxo-5-phenyl-2,3,4,5-tetrahydro-1,5-benzothiazepin-3-yl]-N²-[(3,5-difluorophenyl)acetyl]-L-alaninamide (47)

To a stirred solution of (2,3-cis)-3-amino-7-chloro-2-(2,5-difluorophenyl)-5-phenyl-2,3-dihydro-1,5-benzothiazepin-4(5H)-one (47b) (157 mg, 0.376 mmol) in DMF (1 mL) was added N-[(3,5-difluorophenyl)acetyl]-L-alanine (1e) (131 mg, 0.539 mmol), HOBt (92 mg, 0.681 mmol), NMM (55 mg, 0.544 mmol) and EDAC-HCl (130 mg, 0.678 mmol). The mixture was stirred at ambient temperature under nitrogen for ˜12 h. The reaction mixture was diluted with water and extracted with EtOAc three times. The combined organic extracts were washed in succession with saturated aqueous sodium bicarbonate and 1N aqueous HCl. The organic solution was dried, filtered and evaporated. The residue was purified by flash chromatography on silica gel eluting first with 5:1 (v/v) hexanes-EtOAc, then with 3:1 (v/v) hexanes-EtOAc and finally with 1:1 (v/v) hexanes-EtOAc to afford the title compound (45 mg, 19%) as a white solid. The (2S,3S) diastereomer eluted first followed by the title (2R,3R) compound. ¹H NMR (300 MHz, CDCl₃) δ1.23 (d, 3H, J=6.6 Hz), 3.47 (s, 2H), 4.25 (m, 1H), 5.00 (t, 1H, J=7.4 Hz), 5.60 (d, 1H), 5.92 (d, 1H), 6.38 (d, 1H), 6.69-6.84 (m, 3H), 6.88-7.03 (m, 2H), 7.07 (d, 1H), 7.27 (m, 3H), 7.42 (m, 4H), 7.74 (d, 8.3 Hz). ES⁺, m/z=664 (M+Na).

The required (2,3-cis)-3-amino-7-chloro-2-(2,5-difluorophenyl)-5-phenyl-2,3-dihydro-1,5-benzothiazepin-4(5H)-one (47b) was prepared as follows:

### a. Benzyl [(2,3-cis)-7-chloro-2-(2,5-difluorophenyl)-4-oxo-5-phenyl-2,3,4,5-tetrahydro-1,5-benzothiazepin-3-yl]carbamate (47a)

A stirred mixture of benzyl [(2R,3R)-7-chloro-2-(2,5-difluorophenyl)-4-oxo-2,3,4,5-tetrahydro-1,5-benzothiazepin-3-yl]carbamate (92b) (237 mg, 0.500 mmol), bromobenzene (2 mL), potassium acetate (75 mg, 0.760 mmol) and copper powder (125 mg, 1.960 mmol) was heated at 150° C. for 24 h. The reaction mixture was cooled to ambient temperature and applied to a silica gel column (10 g) pre-equilibrated with 2:1 (v/v) hexanes-DCM. The column was eluted with DCM and the product containing fractions were pooled and evaporated. The residue was triturated with a small volume of diethyl ether to afford the title compound (215 mg, 78%) as a white solid. TLC R_{f}=0.40 (DCM). ¹H NMR (300 MHz, DMSO-d6) δ4.80 (m, 1H), 4.99 (s, 2H), 5.45 (d, 1H, J=7.4 Hz), 7.09-7.64 (m, 16H), 7.88 (d, 1H, J=8.3 Hz). MS APCI, m/z=551 (M+1). HPLC (Method A): 4.09 min.

### b. (2,3-cis)-3-Amino-7-chloro-2-(2,5-difluorophenyl)-5-phenyl-2,3-dihydro-1,5-benzothiazepin-4(5H)-one (47b)

Benzyl [(2,3-cis)-7-chloro-2-(2,5-difluorophenyl)-4-oxo-5-phenyl-2,3,4,5-tetrahydro-1,5-benzothiazepin-3-yl]carbamate (47a) (208 mg, 0.377 mmol) was suspended in 30% HBr-acetic acid (2 mL) and stirred for 90 min. at ambient temperature as the solid gradually dissolved forming a yellow solution. Diethyl ether (15 mL) was added to the reaction and the mixture carefully poured into saturated aqueous sodium carbonate. The organic phase was separated. The aqueous phase was extracted twice with EtOAc. The combined organic extracts were evaporated to afford the title compound (157 mg, 99%). MS APCI, m/z=417 (M+1). LC/MS: 2.35 min.

### Example 48

### N-[(3,5-Difluorophenyl)acetyl]-N-[(6R,7R)-5-oxo-7-phenyl-1,4-thiazepan-6-yl]-L-phenylalaninamide (48)

To a stirred solution of N-[(6,7-cis)-5-oxo-7-phenyl-1,4-thiazepan-6-yl]-L-phenylalaninamide (48c) (88 mg, 0.239 mmol) in DCM (4 mL) was added 3,5-difluorophenylacetic acid (52 mg, 0.302 mmol), HOBt (41 mg, 0.304 mmol), NMM (40 mg, 0.396 mmol) and EDAC-HCl (58 mg, 0.303 mmol). The mixture was stirred at ambient temperature under nitrogen for ˜12 h. The solvent was evaporated and the residue was purified by flash chromatography on silica gel eluting with a gradient from 50% EtOAc: 50% hexanes to 100% EtOAc to afford the title compound in a 1:1 mixture with the 6S,7S diastereomer (99 mg, 79%) as a white solid. ¹H NMR (300 MHz, CDCl₃) δ2.70-3.09 (m, 4H), 3.35-3.44 (m, 2H), 3.62-3.88 (m, 2H), 4.27 (t, 1H, J=4.4 Hz), 4.73 (m, 0.5H), 4.83 (m, 0.5H), 5.26 (m, 1H), 5.85 (br d, 0.5H), 6.05 (br d, 0.5H), 6.29 (br t, 0.5H), 6.56 (br, t, 0.5H), 6.58-6.80 (m, 3H), 6.98 (m, 2H), 7.11-7.28 (m, 9H). MS APCI, m/z=524 (M+1). LC/MS: 2.63 min.

The required N-[(6,7-cis)-5-oxo-7-phenyl-1,4-thiazepan-6-yl]-L-phenylalaninamide (48c) was prepared as follows:

### a. (6,7-cis)-6-Amino-7-phenyl-1,4-thiazepan-5-one hydrobromide (48a)

Benzyl (6,7 cis)-5-oxo-7-phenyl-1,4-thiazepan-6-ylcarbamate (7c) (890 mg, 2.497 mmol) was suspended in 30% HBr/HOAc (3 mL). After stirring for 1 h at ambient temperature the suspension became a yellow solution. The reaction was poured into diethyl ether (60 mL) and the resulting precipitate was collected and dried in-vacuo to afford the title compound (680 mg, 90%) as a white solid. ¹H NMR (300 MHz, DMSO-d6) δ2.78-3.06 (m, 2H), 3.61-3.87 (m, 2H), 4.14 (d, 1H, J=3.0 Hz), 5.00 (d, 1H, 3.0 Hz), 7.35 (s, 5H), 8.19 (br s, 3H), 8.61 (br t, 1H, J=6.0 Hz). MS APCI, m/z=223 (M+1).

### b. N-(tert-Butoxycarbonyl)-N-[(6,7-cis)-5-oxo-7-phenyl-1,4-thiazepan-6-yl]-L-phenylalaninamide (48b)

To a stirred solution of (6,7-cis)-6-amino-7-phenyl-1,4-thiazepan-5-one hydrobromide (48a) (150 mg, 0.497 mmol) in DMF (2 mL) under nitrogen was added, N-(tert-butoxycarbonyl)-L-phenylalanine (144 mg, 0.544 mmol), HOBt (100 mg, 0.742 mmol), NMM (75 mg, 0.742 mmol) and EDAC-HCl (142 mg, 0.742 mmol). The mixture was stirred at ambient temperature for ˜12 h. The reaction mixture was diluted with saturated aqueous sodium bicarbonate and extracted with EtOAc. The organic solution was separated, extracted in succession with 1N aqueous HCl and brine then dried, filtered and evaporated to afford the title compound (227 mg, 97%) as a white solid. ¹H NMR (300 MHz, CDCl₃) δ1.33 (s, 4.5H), 1.37 (s, 4.5H), 2.70-3.10 (m, 5H), 3.60-3.90 (m, 2H), 4.26 (d, 0.5H, J=3.9 Hz), 4.34 (d, 0.5H, J=3.9 Hz), 4.41 (br, 1H), 4.78 (br, 0.5H), 4.92 (br, 0.5H), 5.30 (m, 1H), 6.16 (m, 0.5H), 6.32 (br, 0.5H), 7.08-7.32 (m, 10H). MS APCI, m/z=470 (M+1). LC/MS: 2.68 min.

### c. N-[(6,7-cis)-5-Oxo-7-phenyl-1,4-thiazepan-6-yl]-L-phenylalaninamide (48c)

N-(tert-butoxycarbonyl)-N-[(6,7-cis)-5-oxo-7-phenyl-1,4-thiazepan-6-yl]-L-phenylalaninamide (48b) (227 mg, 0.483 mmol) was dissolved in 3:1 (v/v) DCM:trifluoroacetic acid (10 mL) and kept at ambient temperature for 1 h. The solution was evaporated and the residue partitioned between EtOAc and saturated aqueous sodium bicarbonate. The organic solution was separated, dried, filtered and evaporated to afford the title compound (177 mg, 99%) as a yellow oil. MS APCI, m/z=370 (M+1). LC/MS: 1.22 min. MS APCI, m/z=370 (M+1). LC/MS: 1.49 min. (diastereomers evident)

### Example 49

### N²-[(2S)-2-Hydroxy-4-methylpentanoyl]-N¹-[(6R,7R)-5-oxo-7-phenyl-1,4-thiazepan-6-yl]-L-phenylalaninamide (49)

To a stirred solution of N-[(6,7-cis)-5-oxo-7-phenyl-1,4-thiazepan-6-yl]-L-phenylalaninamide (48c) (88 mg, 0.239 mmol) in DCM (4 mL) was added (S)-2-hydroxyisocaproic acid (40 mg, 0.294 mmol), HOBt (41 mg, 0.304 mmol), NMM (40 mg, 0.396 mmol) and EDAC-HCl (58 mg, 0.303 mmol). The mixture was stirred at ambient temperature under nitrogen for ˜12 h. The solvent was evaporated and the residue was purified by flash chromatography on silica gel eluting with a gradient from 50% EtOAc: 50% hexanes to 100% EtOAc to afford the title compound in a 1:1 mixture with the 6S,7S diastereomer (101 mg, 87%) as a white solid. ¹H NMR (300 MHz, CDCl₃) δ0.88 (m, 6H), 0.95 (m, 1H), 1.22-1.50 (m, 2H), 1.72 (m, 1H), 2.69-3.15 (m, 5H), 3.64-3.87 (m, 2H), 4.02 (m, 1H), 4.22 (d, 0.5H, J=3.9 Hz), 4.28 (d, 0.5H, J=3.9 Hz), 4.70-4.89 (m, 1H), 5.30 (m, 1H) 6.32 (br t, 0.5H), 6.55 (br t, 0.5H), 6.86 (d, 0.5H, J=8.5 Hz), 7.00 (d, 0.5H, J=8.5 Hz), 7.07-7.30 (m, 10H). MS APCI, m/z=484 (M+1). LC/MS: 2.47 min.

### Example 50

### (2S)-2-[(3,5-Difluorophenyl)acetyl]amino-N-[(6R,7R)-5-oxo-7-phenyl-1,4-thiazepan-6-yl]-2-phenylacetamide (50)

To a stirred solution of (2S)-2-amino-N-[(6,7-cis)-5-oxo-7-phenyl-1,4-thiazepan-6-yl]-2-phenylacetamide (50b) (82 mg, 0.230 mmol) in DCM (4 mL) was added 3,5-difluorophenylacetic acid (52 mg, 0.302 mmol), HOBt (41 mg, 0.304 mmol), NMM (40 mg, 0.396 mmol) and EDAC-HCl (58 mg, 0.303 mmol). The mixture was stirred at ambient temperature under nitrogen for ˜12 h. The solvent was evaporated and the residue was purified by flash chromatography on silica gel eluting with a gradient from 50% EtOAc: 50% hexanes to 100% EtOAc to afford the title compound in a 1:1 mixture with the 6S,7S diastereomer (86 mg, 73%) as a white solid. ¹H NMR (300 MHz, CDCl₃) δ2.72 (m, 1H), 2.98 (m, 0.5H), 3.14 (m, 0.5H), 3.53 (s, 1H), 3.54 (s, 1H), 3.55-3.90 (m, 2H), 3.97 (d, 0.5H, J=3.5 Hz), 4.33 (d, 0.5H, J=3.5 Hz), 2.26 (m, 1H), 5.48 (d, 0.5H, J=7.0 Hz), 5.70 (d, 0.5H, J=7.0 Hz), 6.28 (br t, 0.5H), 6.64-6.87 (m, 4.5H), 6.90-7.05 (m, 1.5H), 7.11 (m, 1H), 7.18-7.36 (m, 8.5H). MS APCI, m/z=510 (M+1). LC/MS: 2.57 min.

The precursor (2S)-2-amino-N-[(6,7-cis)-5-oxo-7-phenyl-1,4-thiazepan-6-yl]-2-phenylacetamide (50b) was prepared as follows:

### a. tert-Butyl ((1S)-2-oxo-2-{[(6,7-cis)-5-oxo-7-phenyl-1,4-thiazepan-6-yl]amino}-1-phenylethyl)carbamate (50a)

To a stirred solution of (6,7-cis)-6-amino-7-phenyl-1,4-thiazepan-5-one hydrobromide (48a) (150 mg, 0.497 mmol) in DMF (2 mL) under nitrogen was added, (25)-[(tert-butoxycarbonyl)amino](phenyl)acetic acid (137 mg, 0.544 mmol), HOBt (100 mg, 0.742 mmol), NMM (75 mg, 0.742 mmol) and EDAC-HCl (142 mg, 0.742 mmol). The mixture was stirred at 25° C. for ˜12 h. The reaction mixture was diluted with saturated aqueous sodium bicarbonate and extracted with EtOAc. The organic solution was separated, extracted in succession with 1N aqueous HCl and brine then dried, filtered and evaporated to afford the title compound (221 mg, 97%) as a white solid. ¹H NMR (300 MHz, CDCl₃) δ1.40 (br s, 9H), 2.74 (m, 1H), 2.98-3.20 (m, 1H), 3.56-3.95 (m, 2H), 4.03 (br d, 0.5H), 4.37 (d, 0.5H, J=3.9 Hz), 5.12-5.44 (m, 2H), 5.61 (br d, 0.5H), 5.84 (br d, 0.5H), 6.21 (br 0.5H), 6.58 (br, 0.5H), 6.79 (br d, 1H), 6.96-7.19 (m, 2H), 7.22-7.38 (m, 8H). MS APCI, m/z=456 (M+1). LC/MS: 2.63 min.

### b. (2S)-2-Amino-N-[(6,7-cis)-5-oxo-7-phenyl)-1,4-thiazepan-6-yl]-2-phenylacetamide (50b)

tert-Butyl ((1S)-2-oxo-2-{[(6,7-cis)-5-oxo-7-phenyl-1,4-thiazepan-6-yl]amino}-1-phenylethyl)carbamate (50) (221 mg, 0.485 mmol) was dissolved in 3:1 (v/v) DCM:trifluoroacetic acid (10 mL) and kept at ambient temperature for 1 h. The solution was evaporated and the residue partitioned between EtOAc and saturated aqueous sodium bicarbonate. The organic solution was separated, dried, filtered and evaporated to afford the title compound (165 mg, 95%) as a yellow oil. MS APCI, m/z=356 (M+1). LC/MS: 1.10 min. MS APCI, m/z=356 (M+1). LC/MS: 1.33 min. (diastereomers evident).

### Example 51

### (2S)-2-Hydroxy-4-methyl-N-((1S)-2-oxo-2-[(6R,7R)-5-oxo-7-phenyl-1,4-thiazepan-6-yl]amino-1-phenylethyl)pentanamide (51)

To a stirred solution of (2S)-2-amino-N-[(6,7-cis)-5-oxo-7-phenyl-1,4-thiazepan-6-yl]-2-phenylacetamide (50b) (82 mg, 0.230 mmol) in DCM (4 mL) was added (S)-2-hydroxyisocaproic acid (40 mg, 0.294 mmol), HOBt (41 mg, 0.304 mmol), NMM (40 mg, 0.396 mmol) and EDAC-HCl (58 mg, 0.303 mmol). The mixture was stirred at ambient temperature under nitrogen for ˜12 h. The solvent was evaporated and the residue was purified by flash chromatography on silica gel eluting with a gradient from 50% EtOAc: 50% hexanes to 100% EtOAc to afford the title compound in a 1:1 mixture with the 6S,7S diastereomer (81 mg, 75%) as a white solid. ¹H NMR (300 MHz, CDCl₃) δ0.93 (m, 7H), 1.82 (m, 2H), 2.74 (m, m, 1H), 2.97-3.22 (m, 1H), 3.56-3.90 (m, 2H), 4.03 (d, 0.5H, J=3.5 Hz), 4.17 (m, 1H), 4.36 (d, 0.5H, J=3.5 Hz), 5.25-5.38 (m, 1H), 5.48 (d, 0.5H, J=7.4 Hz), 5.71 (d, 0.5H, J=7.4 Hz), 6.30 (m, 0.5H), 6.72 (m, 0.5H), 6.80 (d, 1H, J=7.4 Hz), 7.01 (t, 1H, J=7.4 Hz), 7.11-7.36 (m, 10H), 7.50 (br d, 0.5H), 7.69 (br d, 0.5H). MS APCI, m/z=470 (M+1). LC/MS: 2.39 min.

### Example 52

### N²-[(3,5-Difluorophenyl)acetyl]-N¹-[(6R,7R)-5-oxo-7-phenyl-1,4-thiazepan-6-yl]-L-leucinamide (52)

To a stirred solution of N⁴-[(6,7-cis)-5-oxo-7-phenyl-1,4-thiazepan-6-yl]-L-leucinamide (52b) (67 mg, 0.200 mmol) in DCM (4 mL) was added 3,5-difluorophenylacetic acid (52 mg, 0.302 mmol), HOBt (41 mg, 0.304 mmol), NMM (40 mg, 0.396 mmol) and EDAC-HCl (58 mg, 0.303 mmol). The mixture was stirred at ambient temperature under nitrogen for ˜12 h. The solvent was evaporated and the residue was purified by flash chromatography on silica gel eluting with a gradient from 50% EtOAc: 50% hexanes to. 100% EtOAc to afford the title compound in a 1:1 mixture with the 6S,7S diastereomer (76 mg, 0.155 mmol, 77%) as a white solid. ¹H NMR (300 MHz, CDCl₃) δ0.74-0.89 (m, 6H), 1.32 (m, 3H), 2.75 (m, 1H), 3.00 (m, 1H), 3.49 (s, 1H), 3.51 (s, 1H), 3.63-3.89 (m, 2H), 4.24 (d, 0.5H, 4.1 Hz), 4.31 (d, 0.5H, J=4.1 Hz), 4.59 (m, 0.5H), 4.70 (m, 0.5H), 5.13 (m, 0.5H), 5.32 (m, 0.5H), 6.05 (br d, 0.5H), 6.17 (br d, 0.5H), 6.54 (m, 0.5H), 6.66-6.88 (m, 3H), 6.95 (m, 0.5H), 7.12-7.30 (m, 5.5H), 7.36 (br d, 0.5H). MS APCI, m/z=490 (M+1). LC/MS: 2.56 min.

The precursor N¹-[(6,7-cis)-5-oxo-7-phenyl-1,4-thiazepan-6-yl]-L-leucinamide (52b) was prepared as follows:

### a. N²-(tert-Butoxycarbonyl)-N¹-[(6,7-cis)-5-oxo-7-phenyl-1,4-thiazepan-6-yl]-L-leucinamide (52a)

To a stirred solution of (6,7-cis)-6-amino-7-phenyl-1,4-thiazepan-5-one hydrobromide (48a) (150 mg, 0.497 mmol) in DMF (2 mL) under nitrogen was added, N-(tert-butoxycarbonyl)-L-leucine (126 mg, 0.544 mmol), HOBt (100 mg, 0.742 mmol), NMM (75 mg, 0.742 mmol) and EDAC-HCl (142 mg, 0.742 mmol). The mixture was stirred at ambient temperature for ˜12 h. The reaction mixture was diluted with saturated aqueous sodium bicarbonate and extracted with EtOAc. The organic solution was separated, extracted in succession with 1N aqueous HCl and brine then dried, filtered and evaporated to afford the title compound (211 mg, 97%) as a white solid. ¹H NMR (300 MHz, CDCl₃) δ0.80-0.95 (m, 6H), 1.41 (m, 9H), 1.56 (m, 3H), 2.79 (m, 1H), 3.04 (m, 1H), 3.63-3.93 (m, 2H), 4.19 (m, 1H), 4.36 (t, 1H, 4.0 Hz), 4.75 (br, 0.5H), 4.87 (br, 0.5H), 5.27 (br, 0.5H), 6.49 (br, 0.5H), 7.27 (m, 6H). MS APCI, m/z=436 (M+1). LC/MS: 2.64 min.

### b. N¹-[(6,7-cis)-5-Oxo-7-phenyl-1,4-thiazepan-6-yl]-L-leucinamide (52b)

N²-(tert-Butoxycarbonyl)-N²-[(6,7-cis)-5-oxo-7-phenyl-1,4-thiazepan-6-yl]-L-leucinamide (52a) (210 mg, 0.482 mmol) was dissolved in 3:1 (v/v) DCM-trifluoroacetic acid (10 mL) and kept at ambient temperature for 1 h. The solution was evaporated and the residue partitioned between EtOAc and saturated aqueous sodium bicarbonate. The organic solution was separated, dried, filtered and evaporated to afford the title compound (135 mg, 83%) as a yellow oil, MS APCI, m/z=336 (M+1). LC/MS: 1.06 min. MS APCI, m/z=336 (M+1). LC/MS: 1.36 min. (diastereomers evident).

### Example 53

### N²-[(2S)-2-Hydroxy-4-methylpentanoyl]-N¹-[(6R,7R)-5-oxo-7-phenyl-1,4-thiazepan-6-yl]-L-leucinamide (53)

To a stirred solution of N¹-[(6,7-cis)-5-oxo-7-phenyl-1,4-thiazepan-6-yl]-L-leucinamide (52b) (67 mg, 0.200 mmol) in DCM (4 mL) was added (S)-2-hydroxyisocaproic acid (40 mg, 0.294 mmol), HOBt (41 mg, 0.304 mmol), NMM (40 mg, 0.396 mmol) and EDAC-HCl (58 mg, 0.303 mmol). The mixture was stirred at ambient temperature under nitrogen for ˜12 h. The solvent was evaporated and the residue was purified by flash chromatography on silica gel eluting with a gradient from 50% EtOAc: 50% hexanes to 100% EtOAc to afford the title compound in a 1:1 mixture with the 6S,7S diastereomer (73 mg, 81%) as a white solid. ¹H NMR (300 MHz, CDCl₃) δ0.80-1.00 (m, 14H), 1.33-1.93 (m, 5H), 2.74-2.86 (m, 1H), 2.97-3.14 (m, 1H), 3.68-3.90 (m, 2H), 4.10 (m, 1H), 4.32 (d, 1H, 4 Hz), 4.51 (m, 0.5H), 4.61 (m, 0.5H), 5.23 (m, 0.5H), 5.32 (m, 0.5H), 6.48 (br, 0.5H), 6.78 (m, 1H), 6.93 (br d, 0.5H), 7.27 (m, 6H). MS APCI, m/z=450 (M+1). LC/MS: 2.40 min.

### Example 54

### N²-[(3,5-Difluorophenyl)acetyl]-N¹-[(6R,7R)-5-oxo-7-phenyl-1,4-thiazepan-6-yl]-L-valinamide (54)

To a stirred solution of N¹-[(6,7-cis)-5-oxo-7-phenyl-1,4-thiazepan-6-yl]-L-valinamide (54b) (65 mg, 0.202 mmol) in DCM (4 mL) was added 3,5-difluorophenylacetic acid (52 mg, 0.302 mmol), HOBt (41 mg, 0.304 mmol), NMM (40 mg, 0.396 mmol) and EDAC-HCl (58 mg, 0.303 mmol). The mixture was stirred at ambient temperature under nitrogen for ˜12 h. The solvent was evaporated and the residue was purified by flash chromatography on silica gel eluting with a gradient from 50% EtOAc: 50% hexanes to 100% EtOAc to afford the title compound in a 1:1 mixture with the 6S,7S diastereomer (62 mg, 64%) as a white solid. ¹H NMR (300 MHz, CDCl₃) δ0.68-0.89 (m, 6H), 1.78-2.05 (m, 1H), 2.69-2.80 (m, 1H), 3.02 (m, 1H), 3.51 (s, 1H), 3.54 (s, 1H), 3.67-3.91 (m, 2H), 4.24 (d, 0.5H, J=4.0 Hz), 4.29 (d, 0.5H, J=4.0 Hz), 4.38 (m, 0.5H), 4.53 (m, 0.5H), 5.22 (m, 0.5H), 5.35 (m, 0.5H), 6.32 (m, 1H), 6.63-6.94 (m, 5H), 7.13-7.31 (m, 5H). MS APCI, m/z=476 (M+1). LC/MS: 2.46 min.

The precursor N¹-[(6,7-cis)-5-oxo-7-phenyl-1,4-thiazepan-6-yl]-L-valinamide (54b) was prepared as follows:

### a. N²-(tert-Butoxycarbonyl)-N¹-[(6,7-cis)-5-oxo-7-phenyl-1,4-thiazepan-6-yl]-L-valinamide (54a)

To a stirred solution of (6,7-cis)-6-amino-7-phenyl-1,4-thiazepan-5-one hydrobromide (48a) (150 mg, 0.497 mmol) in DMF (2 mL) under nitrogen was added, N-(tert-butoxycarbonyl)-L-valine (118 mg, 0.544 mmol), HOBt (100 mg, 0.742 mmol), NMM (75 mg, 0.742 mmol) and EDAC-HCl (142 mg, 0.742 mmol). The mixture was stirred at ambient temperature for ˜12 h. The reaction mixture was diluted with saturated aqueous sodium bicarbonate and extracted with EtOAc. The organic solution was separated, extracted in succession with 1N aqueous HCl and brine then dried, filtered and evaporated to afford the title compound (189 mg, 90%) as a white solid. ¹H NMR (300 MHz, CDCl₃) δ0.84 (m, 6H), 1.42 (m, 9H), 1.89-2.14 (m, 1H), 2.73-2.84 (m, 1H), 2.98-3.14 (m, 1H), 3.80 (m, 2H), 4.32 (d, 1H, J=4.0 Hz), 5.03 (m, 1H), 5.36 (m, 1H), 6.37-6.60 (br m, 1H), 7.09-7.31 (m, 3H), 7.44 (m, 2H), 7.80 (m, 2H). MS APCI, m/z=422 (M+1). LC/MS: 2.50 min.

### b. N¹-[(6,7-cis)-5-oxo-7-phenyl-1,4-thiazepan-6-yl]-L-valinamide (54b)

N²-(tert-Butoxycarbonyl)-N¹-[(6,7-cis)-5-oxo-7-phenyl-1,4-thiazepan-6-yl]-L-valinamide (54a) (189 mg, 0.449 mmol) was dissolved in 3:1 (v/v) DCM:trifluoroacetic acid (10 mL) and kept at ambient temperature for 1 h. The solution was evaporated and the residue partitioned between EtOAc and saturated aqueous sodium bicarbonate. The organic solution was separated, dried, filtered and evaporated to afford the title compound (130 mg, 90%) as a yellow oil. MS APCI, m/z=322 (M+1). LC/MS: 0.70 min. MS APCI, m/z=322 (M+1). LC/MS: 1.20 min. (diastereomers evident).

### Example 55

### N²-[(2S)-2-Hydroxy-4-methylpentanoyl]-N¹-[(6R,7R)-5-oxo-7-phenyl-1,4-thiazepan-6-yl]-L-valinamide (55)

To a stirred solution of N¹-[(6,7-cis)-5-oxo-7-phenyl-1,4-thiazepan-6-yl]-L-valinamide (54b) (65 mg, 0.202 mmol) in DCM (4 mL) was added (S)-2-hydroxyisocaproic acid (40 mg, 0.294 mmol), HOBt (41 mg, 0.304 mmol), NMM (40 mg, 0.396 mmol) and EDAC-HCl (58 mg, 0.303 mmol). The mixture was stirred at ambient temperature under nitrogen for ˜12 h. The solvent was evaporated and the residue was purified by flash chromatography on silica gel eluting with a gradient from 50% EtOAc: 50% hexanes to 100% EtOAc to afford the title compound in a 1:1 mixture with the 6S,7S diastereomer (63 mg, 72%) as a white solid. ¹H NMR (300 MHz, CDCl₃) δ0.70-1.04 (m, 13H), 1.60 (m, 2H), 1.80 (br 1H), 1.90-2.19 (m, 1H), 2.78 (m, 1H), 3.06 (m, 1H), 3.68-3.94 (m, 2H), 4.13 (m, 1H), 4.24-4.48 (m, 2H), 5.13 (m, 0.5H), 5.31 (m, 0.5H), 5.38 (m, 0.5H), 6.53 (m, 0.5H), 6.63 (m, 0.5H), 7.01 (m, 1H), 7.16 (m, 0.5H), 7.23-7.32 (m, 5H). MS APCI, m/z=436 (M+1). LC/MS: 2.31 min

### Example 56

### N¹-[(2R,3S)-7-Chloro-4-oxo-2-phenyl-2,3,4,5-tetrahydro-1,5-benzoxazepin-3-yl]-N²-[(3,5-difluorophenyl)acetyl]-L-alaninamide (56)

To a stirred solution of (2,3-cis)-3-amino-7-chloro-2-phenyl-2,3-dihydro-1,5-benzoxazepin-4(5H)-one (56e) (100 mg, 0.346 mmol) in DCM (3 mL) was added N-[(3,5-difluorophenyl)acetyl]-L-alanine (1e) (93 mg, 0.382 mmol), HOBt (57 mg, 0.422 mmol), NMM (43 mg, 0.425 mmol) and EDAC-HCl (80 mg, 0.417 mmol). The mixture was stirred at ambient temperature under nitrogen for ˜12 h. The reaction mixture was diluted with water and extracted with EtOAc three times. The combined organic extracts were washed in succession with saturated aqueous sodium bicarbonate and 1N aqueous HCl. The organic solution was dried, filtered and evaporated. The residue was purified by flash chromatography on silica gel eluting with 1:1 (v/v) hexane-EtOAc to afford the title compound in a 1:1 mixture with the 2S,3R diastereomer (120 mg, 67%) as a white solid. ¹H NMR (300 MHz, CDCl₃) δ1.08 (d, 1.5H, J=7.0 Hz), 1.24 (d, 1.5H, J=7.0 Hz), 3.44 (s, 1H), 3.50 (s, 1H), 4.48 (m, 0.5H), 4.62 (m, 0.5H), 4.98 (t, 0.5H), 5.11 (t, 0.5H), 5.76 (m, 1H), 6.11 (d, 0.5H), 6.24 (d, 0.5H), 6.53-6.87 (m, 3H), 7.04 (m, 1H), 7.18-7.42 (m, 8H), 7.84 (s, 0.5H),8.41 (s, 0.5H). MS APCI, m/z=514 (M+1). LC/MS: 2.47 min.

The precursor (2,3-cis)-3-amino-7-chloro-2-phenyl-2,3-dihydro-1,5-benzoxazepin-4(5H)-one (56e) was prepared as follows:

### a. erythro Ethyl 2-hydroxy-3-(4-chloro-2-nitrophenoxy)-3-phenylpropanoate (56a)

To a stirred mixture of 4-chloro-2-nitrophenol (11.68 g, 67.32 mmol), ethyl 3-phenyloxirane-2-carboxylate (9.61 g, 50.00 mmol) in ethanol (200 mL) was added portionwise 60% sodium hydride (738 mg, 20.00 mmol) and the red mixture stirred at reflux for 3 d. The solvent was removed in vacuo. The residue was dissolved in CHCl₃ and extracted three times with 10% aqueous potassium carbonate. The organic layer was washed with water and brine, dried, filtered and evaporated to afford a brown residue. The residue was dissolved in a minimal volume of CHCl₃ and precipitated by adding diethyl ether to afford the title compound (5.970 g, 33%) as an off-white solid. ¹H NMR (300 MHz, DMSO-d6) δ1.16 (t, 3H, J=7.0 Hz), 4.09 (q, 2H, J=7.0 Hz), 4.29 (t, 1H, J=7.0 Hz), 5.63 (d, 1H, J=7.0 Hz), 6.09 (d, 1H, J=7.0 Hz), 7.19-7.60 (m, 7H), 7.97 (d, 1H, J=2.6 Hz). MS APCI, m/z=388 (M+Na). LC/MS: 2.78 min.

### b. erythro Ethyl 2-hydroxy-3-(4-chloro-2-nitrophenoxy)-3-phenylpropanoate (56b)

To a solution of erythro ethyl 2-hydroxy-3-(4-chloro-2-nitrophenoxy)-3-phenylpropanoate (56a) (5.870 g, 16.048 mmol) in ethanol (200 mL) was added 5% palladium on carbon (200 mg) and the mixture was hydrogenated at 35 psi on a Parr shaker for 45 min. The reaction mixture was filtered through diatomaceous earth and the resulting solution concentrated in-vacuo. The residue was purified by flash chromatography on silica gel eluting first with CHCl₃ and then 60:1 (v/v) CHCl₃-ethanol to afford the title compound (3.150 g, 58%) as a red oil. TLC R_{f}=0.21 (60:1 CHCl₃-ethanol). MS APCI, m/z=336 (M+1). LC/MS: 2.00 min.

### c. (2,3-trans)-7-Chloro-3-hydroxy-2-phenyl-2,3-dihydro-1,5-benzoxazepin-4(5H)-one (56c)

To a stirred solution of erythro ethyl 2-hydroxy-3-(4-chloro-2-nitrophenoxy)-3-phenylpropanoate (56b) (3.070 g, 9.143 mmol) in THF (40 mL) cooled to 0° C. was added a solution of lithium hydroxide monohydrate (422 mg, 10.057 mmol) in water (20 mL) and methanol (2 mL). After 1 h the cooling bath was removed and the mixture stirred an additional 2 h at ambient temperature. The reaction was re-cooled to 0° C. and 1N aqueous hydrochloric acid (14.3 mL) was added. Solvent was then removed in-vacuo. The residue was dissolved in DMF (50 mL), HOBt (2.00 g, 14.804 mmol), NMM (1.510 g, 14.950 mmol), and EDAC (2.880 g, 15.023 mmol) were added and the mixture stirred for ˜12 h under nitrogen at ambient temperature. Solvent was removed in-vacuo at 40° C. and the residue partitioned between water and EtOAc. The organic solution was separated, washed in succession with saturated aqueous sodium bicarbonate, 1N aqueous HCl and brine, then dried, filtered and evaporated. The residue was purified by flash chromatography (3:1 hexane-EtOAc) to afford the title compound (1.120 g, 42%) as a white solid. TLC R_{f}=0.19 (3:1 hexane:EtOAc). ¹H NMR (300 MHz, CDCl₃) δ3.65 (d, 1H, J=4.8 Hz), 4.62 and 4.65 (dd, 1H, J=4.8 Hz), 5.29 (d, 1H, J=9.6 Hz), 6.80 (m, 1H), 7.02-7.09 (m, 2H), 7.41 (s, 5H), 7.89 (s, 1H). MS APCI, m/z=290 (M+1). LC/MS: 2.35 min.

### d. (2,3-cis)-3-Azido-7-chloro-2-phenyl-2,3-dihydro-1,5-benzoxazepin-4(5H)-one (56d)

Trifluoromethanesulfonyl chloride (926 mg, 5.495 mmol) was added via syringe to a solution of (2,3-trans)-7-chloro-3-hydroxy-2-phenyl-2,3-dihydro-1,5-benzoxazepin-4(5H)-one (56c) (1.064 g, 3.672 mmol) and triethylamine (634 mg, 6.277 mmol) in DCM (25 mL) under nitrogen at −20° C. The mixture was kept at −20° C. for 18 h. Additional triethylamine (1.268 g, 12.554 mmol) and trifluoromethanesulfonyl chloride (1.852 g, 10.990 mmol) were added and the mixture kept at −20° C. for an additional 24 h. Again, additional trifluoromethanesulfonyl chloride (926 mg, 5.495 mmol) and triethylamine (634 mg, 6.277 mmol) were added and the mixture kept at −20° C. for an additional 5 h. The reaction was concentrated in-vacuo without heating, and the resulting residue immediately dissolved in DMF (10 mL) at 0° C. under nitrogen. Sodium azide (1.190 g, 18.305 mmol) was added to the solution and the mixture allowed to warm to ambient temperature over 30 min. After an additional 2 h the reaction was diluted with water and extracted with EtOAc three times. The combined organic extracts were washed in succession with saturated aqueous sodium bicarbonate, 1N aqueous HCl and brine. The organic solution was dried, filtered and evaporated and the residue purified by flash chromatography on silica gel eluting with DCM and then with 100:2 (v/v) DCM:methanol to afford the title compound (410 mg, 35%) as a foamy white solid. TLC R_{f}=0.20 (DCM) ¹H NMR (300 MHz, CDCl₃) δ4.46 (d, 1H, J=5.7 Hz), 5.54 (d, 1H, J=5.7 Hz), 7.05 (s, 1H), 7.16 (s, 2H), 7.39-7.47 (m, 3H), 7.49-7.57 (m, 2H), 7.86 (s, 1H). MS APCI, m/z=287 (M+1−N₂). LC/MS: 2.54 min.

### e. (2,3-cis)-3-Amino-7-chloro-2-Phenyl-2,3-dihydro-1,5-benzoxazepin-4(5H)-one (56e)

To a stirred solution of (2,3-cis)-3-azido-7-chloro-2-phenyl-2,3-dihydro-1,5-benzoxazepin-4(5H)-one (56d) (385 mg, 1.223 mmol) in THF (10 mL) was added water (33 μL) and triphenylphosphine (337 mg, 1.285 mmol). After 18 h at ambient temperature water (1 mL) was added. After an additional 1 h the solvent was evaporated and the residue was purified by flash chromatography on silica gel eluting with 100:3 (v/v) DCM:methanol to afford the title compound (216 mg, 61%) as a white solid. TLC R_{f}=0.25 (100:3 (v/v) DCM:methanol). ¹H NMR (300 MHz, CDCl₃) δ1.46 (br, 2H), 4.12 (d, 1H, J=6.6 Hz), 5.55 (d, 1H, J=6.6 Hz), 7.05 (s, 1H), 7.18 (m, 2H), 7.36-7.73 (m, 6H). MS APCI, m/z=289 (M+1). LC/MS: 1.54 min.

### Example 57

### (2S)—N-((1S)-2-[(2R,3S)-7-Chloro-4-oxo-2-phenyl-2,3,4,5-tetrahydro-1,5-benzoxazepin-3-yl]amino-2-oxo-1-phenylethyl)-2-hydroxy-4-methylpentanamide (57)

To a stirred solution of (2S)-2-amino-N-[(2,3-cis)-7-chloro-4-oxo-2-phenyl-2,3,4,5-tetrahydro-1,5-benzoxazepin-3-yl]-2-phenylacetamide (57b) (80 mg, 0.199 mmol) in DCM (4 mL) were added (S)-2-hydroxyisocaproic acid (30 mg, 0.192 mmol), HOBt (34 mg, 0.251 mmol), NMM (26 mg, 0.257 mmol) and EDAC-HCl (48 mg, 0.250 mol) in succession and the mixture stirred for ˜12 h under nitrogen at ambient temperature. The reaction mixture was diluted with water and extracted with EtOAc. The combined organic extracts were washed in succession with saturated aqueous sodium bicarbonate and 1N aqueous HCl. The organic solution was dried, filtered and evaporated. The residue was purified by flash chromatography on silica gel eluting with 50:1 (v/v) CHCl₃:methanol to afford the title compound as a 1:1 mixture with the 2S,3R diastereomer (96 mg, 93%) as a white solid. ¹H NMR (300 MHz, CDCl₃) δ0.93 (m, 7H), 1.81 (m, 2H), 2.60 (br, 1H), 4.09 (m, 0.5H), 4.21 (m, 0.5H), 5.14 (m, 1H), 5.45 (m, 0.5H), 5.53-5.68 (m, 1H), 5.82 (m, 0.5H), 6.57 (m, 1H), 6.90-7.11 (m, 4H), 7.15-7.60 (m, 10H), 7.78 (s, 0.5H), 8.34 (s, 0.5H). MS APCI, m/z=536 (M+1). LC/MS: 2.43 min.

The precursor (2S)-2-amino-N-[(2,3-cis)-7-chloro-4-oxo-2-phenyl-2,3,4,5-tetrahydro-1,5-benzoxazepin-3-yl]-2-phenylacetamide (57b) was prepared as follows:

### a. tert-Butyl ((1S)-2-{[(2,3-cis)-7-chloro-4-oxo-2-phenyl-2,3,4,5-tetrahydro-1,5-benzoxazepin-3-yl]amino}-2-oxo-1-phenylethyl)carbamate (57a)

To a stirred solution of (2,3-cis)-3-amino-7-chloro-2-phenyl-2,3-dihydro-1,5-benzoxazepin-4(5H)-one (56e) (90 mg, 0.312 mmol) in DCM (3 mL) was added (2S)-[(tert-butoxycarbonyl)amino](phenyl)acetic acid (86 mg, 0.342 mmol), HOBt (47 mg, 0.347 mmol), NMM (50 mg, 0.495 mmol) and EDAC-HCl (66 mg, 0.344 mmol). The mixture was stirred at ambient temperature under nitrogen for ˜12 h. The reaction mixture was diluted with water and extracted with EtOAc. The combined organic extracts were washed in succession with saturated aqueous sodium bicarbonate and 1N aqueous HCl. The organic solution was dried, filtered and evaporated. The residue was purified by flash chromatography on silica gel eluting with 3:1 (v/v) hexane:EtOAc to afford the title compound (111 mg, 68%) as a white solid. ¹H NMR (300 MHz, CDCl₃) δ1.35 (s, 4.5H), 1.45 (s, 4.5H), 5.03-5.29 (m, 1.5H), 5.41-5.63 (m, 1.5H), 5.74-5.86 (m, 1H), 6.54-6.80 (br, 1H), 6.92 (m, 1.5H), 6.99-7.13 (m, 2.5H), 7.15-7.41 (m, 9H), 7.75 (br, 0.5H), 8.44 (br, 0.5H). MS APCI, m/z=544 (M+Na). LC/MS: 2.75 min.

### b. (2S)-2-Amino-N-[(2,3-cis)-7-chloro-4-oxo-2-phenyl-2,3,4,5-tetrahydro-1,5-benzoxazepin 3-yl]-2-phenylacetamide (57b)

Trifluoroacetic acid (1 mL) was added to a solution of tert-butyl ((1S)-2-{[(2,3-cis)-7-chloro-4-oxo-2-phenyl-2,3,4,5-tetrahydro-1,5-benzoxazepin-3-yl]amino}-2-oxo-1-phenylethyl)carbamate (57a) (100 mg, 0.192 mmol) in dichloromethane (3 mL) at 0° C. The solution was allowed to warm to ambient temperature and stirred 1 h and then the solvent was evaporated. The residue was dissolved in EtOAc and extracted with aqueous sodium bicarbonate. The organic solution was dried and evaporated to afford the title compound (80 mg, 99%) which was used in the next step without additional purification. ¹H NMR (300 MHz, CDCl₃) δ1.72 (br s, 2H), 4.55 (m, 1H), 5.22 (m, 1H), 5.67 (m, 1H), 7.04 (m, 2H), 7.13-7.44 (m, 11H), 7.55 (m, 2H). MS APCI, m/z=422 (M+1). LC/MS: 2.00 min.

### Example 58

### (2S-2-[(3,5-Difluorophenyl)acetyl]amino-N-[(2R,3S)-4-oxo-2-phenyl-2,3,4,5-tetrahydro-1,5-benzoxazepin-3-yl]-2-phenylacetamide (58)

To a stirred solution of (2S)-2-amino-N-[(2,3-cis)-4-oxo-2-phenyl-2,3,4,5-tetrahydro-1,5-benzoxazepin-3-yl]-2-phenylacetamide (58b) (108 mg, 0.279 mmol) in DCM (3 mL) under nitrogen was added 3,5-difluorophenylacetic acid (58 mg, 0.336 mmol), HOBt (50 mg, 0.370 mmol), NMM (42 mg, 0.416 mmol) and EDAC-HCl (71 mg, 0.370 mmol). The mixture was stirred for ˜12 h at ambient temperature then evaporated. The residue was dissolved in EtOAc and extracted in succession with saturated sodium bicarbonate, 1N aqueous HCl, and brine. The organic solution was dried, filtered and evaporated. The residue was purified by flash chromatography on silica gel eluting with 100:1 (v/v) CHCl₃:methanol to afford the title compound as a 1:1 mixture with (2S)-2-[(3,5-difluorophenyl)acetyl]amino-N-[(2S,3R)-4-oxo-2-phenyl-2,3,4,5-tetrahydro-1,5-benzoxazepin-3-yl]-2-phenylacetamide (110 mg, 73%) as a white solid. ¹H NMR (300 MHz, CDCl₃) δ3.47 (s, 1H), 3.56 (s, 1H), 5.07 (m, 1H), 5.45 (d, 0.5H, 7.0 Hz), 5.56 (d, 0.5H, 7.0 Hz), 5.71 (d, 0.5H, 7.0 Hz), 5.82 (d, 0.5H, 7.0 Hz), 6.52 (m, 1H), 6.72 (m, 2H), 6.80-7.09 (m, 4H), 7.11-7.40 (m, 12H), 7.65 (s, 0.5H), 8.29 (s, 0.5H). MS APCI, m/z=542 (M+1). LC/MS: 2.58 min.

The precursor (2S)-2-amino-N-[(2,3-cis)-4-oxo-2-phenyl-2,3,4,5-tetrahydro-1,5-benzoxazepin-3-yl]-2-phenylacetamide (58b) was prepared as follows:

### a. tert-Butyl ((1S)-2-oxo-2-{[(2,3-cis)-4-oxo-2-phenyl-2,3,4,5-tetrahydro-1,5-benzoxazepin-3-yl]amino}-1-phenylethyl)carbamate (58a)

To a stirred solution of (2,3-cis)-3-amino-2-phenyl-2,3-dihydro-1,5-benzoxazepin-4(5H)-one (6e) (98 mg, 0.337 mmol) in DCM (3 mL) was added (2S)-[(tert-butoxycarbonyl)amino](phenyl)acetic acid (93 mg, 0.370 mmol), HOBt (55 mg, 0.407 mmol), NMM (68 mg, 0.673 mmol) and EDAC-HCl (78 mg, 0.407 mmol). The mixture was stirred at ambient temperature under nitrogen for ˜12 h. The reaction mixture was diluted with water and extracted with EtOAc three times. The combined organic extracts were washed in succession with saturated aqueous sodium bicarbonate and 1N aqueous HCl. The organic solution was dried, filtered and evaporated. The residue was purified by flash chromatography on silica gel eluting with 2:1 (v/v) hexane:EtOAc to afford the title compound (146 mg, 89%) as a yellow solid. ¹H NMR (300 MHz, CDCl₃) δ1.34 (s, 4.5H), 1.44 (s, 4.5H), 5.17 (m, 1H), 5.55 (m, 1H), 5.73 (d, 0.5H, J=7.5 Hz), 5.85 (d, 0.5H, J=7.0 Hz), 6.44-6.73 (br, 1H), 6.91-7.12 (m, 3H), 7.14-7.41 (m, 12H), 7.63 (br s, 0.5H), 8.15 (br, 0.5H). MS APCI, m/z=510 (M+Na). LC/MS: 2.60 min.

### b. (2S)-2-Amino-N-[(2,3-cis)-4-oxo-2-phenyl-2,3,4,5-tetrahydro-1,5-benzoxazepin-3-yl]-2-phenylacetamide (58b)

Trifluoroacetic acid (1 mL) was added to a solution of tert-butyl ((1S)-2-oxo-2-{[(2,3-cis)-4-oxo-2-phenyl-2,3,4,5-tetrahydro-1,5-benzoxazepin-3-yl]amino}-1-phenylethyl)carbamate (58a) (136 mg, 0.279 mmol) in dichloromethane (3 mL) at 0° C. The solution was allowed to warm to ambient temperature and stirred 1 h. The solvent was evaporated and the residue was dissolved in EtOAc and extracted with aqueous sodium bicarbonate. The organic solution was dried and evaporated to afford the title compound (108 mg, 99%) which was used in the next step without additional purification. ¹H NMR (300 MHz, CDCl₃) δ1.71 (br s, 2H), 4.44 (m, 1H), 5.23 (m, 1H), 5.70 (m, 1H), 6.98-7.60 (m, 16H). MS APCI, m/z=388 (M+1). LC/MS: 1.72 min. MS APCI, m/z=388 (M+1). LC/MS: 1.82 min. (diastereomers evident).

### Example 59

### N²-[(3,5-Difluorophenyl)acetyl]-N¹-[(2R,3S)-4-oxo-2-phenyl-2,3,4,5-tetrahydro-1,5-benzoxazepin-3-yl]-L-serinamide (59)

Using a procedure similar to that described in Example 1, except using (2,3-cis)-3-amino-2-phenyl-2,3-dihydro-1,5-benzoxazepin-4(5H)-one hydrochloride (6e) (61.1 mg) as the amine component, and N-[(3,5-difluorophenyl)acetyl]-L-serine (4b) (66.5 mg) as the acid component, the title compound (59) was obtained as a white solid. Purification by flash chromatography (2-10% methanol gradient in DCM) provided the white solid title compound (97.5 mg) as a 1:1 mixture with the 2S,3R diastereomer. ¹H NMR (300 MHz, CDCl₃) δ3.00-3.179 (m, 1H), 3.37-3.54 (m, 4H), 3.70-3.83 (m, 1H), 4.37-4.50 (m, 1H), 5.09-5.18 (m, 1H), 5.74 (d, 1H, J=7 Hz), 6.55-7.51 (m, 13H), 8.24-8.39 (m, 1H). MS APCI, m/z=496 (M+1). LC/MS: 2.14 min.

### Example 60

### (2S)-2-Cyclohexyl-2-[(3,5-difluorophenyl)acetyl]amino-N-[(2R,3S)-4-oxo-2-phenyl-2,3,4,5-tetrahydro-1,5-benzoxazepin-3-yl]acetamide (60)

To a stirred solution of (25)-2-amino-N-[(2,3-cis)-4-oxo-2-phenyl-2,3,4,5-tetrahydro-1,5-benzoxazepin-3-yl]-2-cyclohexylacetamide (60b) (137 mg, 0.350 mmol), 3,5-difluorophenylacetic acid (67 mg, 0.389 mmol), HOBt (59 mg, 0.437 mmol) and NMM (45 mg, 0.455 mmol) in DCM (5 mL) under nitrogen at 0° C. was added EDAC-HCl (83 mg, 0.432 mmol). The mixture was kept at 0° C. for 30 min. then stirred for ˜12 h at ambient temperature. The reaction was diluted with EtOAc and extracted in succession with saturated sodium bicarbonate, 1N aqueous HCl, and brine. The organic solution was dried, filtered and evaporated. The residue was purified by flash chromatography on silica gel eluting with 1:1 (v/v) hexane-EtOAc to afford the title compound as a 1:1 mixture with the 2S,3R diastereomer (110 mg, 57%) as a white solid. ¹H NMR (300 MHz, DMSO-d6) δ0.72-1.22 (m, 5H), 1.29-1.70 (m, 5H), 3.27 (s, 2H), 3.52 (m, 1H), 4.13 (m, 0.5H), 4.28 (m, 0.5H), 4.98 (m, 0.5H), 5.08 (m, 0.5H), 5.58 (m, 1H), 6.87-7.56 (m, 13H), 8.03 (br d, 0.5H), 8.17 (br d, 0.5H), 10.26 (s, 0.5H), 10.30 (s, 0.5H). MS APCI, m/z=548 (M+1). LC/MS: 2.49 min.

The precursor (2S)-2-amino-N-[(2,3-cis)-4-oxo-2-phenyl-2,3,4,5-tetrahydro-1,5-benzoxazepin-3-yl]-2-cyclohexylacetamide (60b) was prepared as follows:

### a. tert-Butyl ((1S)-1-cyclohexyl-2-oxo-2-{[(2,3-cis)-4-oxo-2-phenyl-2,3,4,5-tetrahydro-1,5-benzoxazepin-3-yl]amino}ethyl)carbamate (60a)

To a stirred solution of (2,3-cis)-3-amino-2-phenyl-2,3-dihydro-1,5-benzoxazepin-4(5H)-one (6e) (254 mg, 1.000 mmol) in DCM (10 mL) under argon was added BOC-L-cyclohexylglycine (257 mg, 1.000 mmol) and HOBt (176 mg, 1.300 mmol). The solution was cooled to 0° C., NMM (135 mg, 1.366 mmol) and EDAC-HCl (249 mg, 1.299 mmol) were added. The reaction was allowed to warm to ambient temperature and stirred under argon for ˜12 h. The reaction mixture was diluted with EtOAc and extracted in succession with saturated aqueous sodium bicarbonate, 1N aqueous HCl, and brine. The organic solution was dried, filtered and evaporated. The residue was purified by flash chromatography on silica gel eluting with 3:1 (v/v) hexane-EtOAc to afford the title compound (408 mg, 83%) as a yellow solid. TLC R_{f}=0.23 (3:1 (v/v) hexane:EtOAc). ¹H NMR (300 MHz, CDCl₃) δ0.77-1.78 (m, 20H), 3.90 (m, 0.5H), 4.17 (m, 0.5H), 4.97 (m, 1H), 5.17 (m, 1H), 5.80 (m, 1H), 6.42-6.62 (m, 1H), 7.08 (m, 1H), 7.14-7.47 (m, 8H), 7.59 (br s, 0.5H), 8.05 (br s, 0.5H). MS APCI, m/z=494 (M+1). LC/MS: 2.61 min.

### b. (2S)-2-Amino-N-[(2,3-cis)-4-oxo-2-phenyl-2,3,4,5-tetrahydro-1,5-benzoxazepin-3-yl]-2-cyclohexylacetamide (60b)

Trifluoroacetic acid (2 mL) was added to a stirred solution of tert-butyl ((1S)-1-cyclohexyl-2-oxo-2-{[(2,3-cis)-4-oxo-2-phenyl-2,3,4,5-tetrahydro-1,5-benzoxazepin-3-yl]amino}ethyl)carbamate (60a) (345 mg, 0.699 mmol) at ambient temperature. The solution was stirred for 90 min. then evaporated. The residue was dissolved in EtOAc and extracted in succession with aqueous sodium bicarbonate and brine. The organic solution was dried and evaporated to afford the title compound (275 mg, 99%) as a yellow solid which was used in the next step without additional purification. MS APCI, m/z=394 (M+1). LC/MS: 1.54 min. MS APCI, m/z=394 (M+1). LC/MS: 1.71 min. (diastereomers evident).

### Example 61

### (2S)—N-((1S)-1-Cyclohexyl-2-oxo-2-[(2R,3S-4-oxo-2-phenyl-2,3,4,5-tetrahydro-1,5-benzoxazepin-3-yl]aminoethyl)-2-hydroxy-4-methylpentanamide (61)

To a stirred solution of (2S)-2-amino-N-[(2,3-cis)-4-oxo-2-phenyl-2,3,4,5-tetrahydro-1,5-benzoxazepin-3-yl]-2-cyclohexylacetamide (60b) (137 mg, 0.350 mmol), (S)-2-hydroxyisocaproic acid (52 mg, 0.394 mmol), HOBt (59 mg, 0.437 mmol) and NMM (45 mg, 0.455 mmol) in DCM (5 mL) under nitrogen at 0° C. was added EDAC-HCl (83 mg, 0.432 mmol). The mixture was kept at 0° C. for 30 min. then stirred for ˜12 h at ambient temperature. The reaction was diluted with EtOAc and extracted in succession with saturated sodium bicarbonate, 1N aqueous HCl, and brine. The organic solution was dried, filtered and evaporated. The residue was purified by flash chromatography on silica gel eluting with 1:1 (v/v) hexane-EtOAc to afford the title compound in a 1:1 mixture with the 2S,3R diastereomer (144 mg, 81%) as a white solid. ¹H NMR (300 MHz, DMSO-d6) δ0.75-1.86 (m, 20H), 3.83 (m, 1H), 4.22 (m, 0.5H), 4.37 (m, 0.5H), 5.06 (m, 1H), 5.46 (m, 1H), 5.58 (m, 1H), 7.10-7.27 (m, 4H), 7.31-7.44 (m, 5.5H), 7.55 (br d, 0.5H), 7.71 (m, 0.5H), 7.88 (m, 0.5H), 10.25 (s, 0.5H), 10.28 (s, 0.5H). MS APCI, m/z=508 (M+1). LC/MS: 2.41 min.

### Example 62

### 3-Cyclohexyl-N²-[(3,5-difluorophenyl)acetyl]-N¹-[(6R,7R)-5-oxo-7-phenyl-1,4-thiazepan-6-yl]-L-alaninamide (62)

To a stirred solution of 3-cyclohexyl-N¹-[(6,7-cis)-5-oxo-7-phenyl-1,4-thiazepan-6-yl]-L-alaninamide (62b) (115 mg, 0.306 mmol) in DCM (6 mL) was added 3,5-difluorophenylacetic acid (56 mg, 0.325 mmol), HOBt (50 mg, 0.370 mmol), NMM (37 mg, 0.366 mmol) and EDAC-HCl (70 mg, 0.365 mmol). The mixture was stirred at ambient temperature under nitrogen for ˜12 h. The solvent was evaporated, the residue dissolved in EtOAc and extracted in succession with saturated sodium bicarbonate, 1N aqueous HCl, and brine. The organic solution was dried, filtered and evaporated. The residue was purified by flash chromatography on silica gel eluting with 1:1 (v/v) DCM-EtOAc to afford the title compound in a 1:1 mixture with the 6S,7S diastereomer (45 mg, 28%) as a white solid. TLC R_{f}=0.27 (1:1 DCM:EtOAc). ¹H NMR (300 MHz, CDCl₃) δ0.84 (m, 3H), 1.00-1.44 (m, 6H), 1.48-1.70 (m, 4H), 2.80 (m, 1H), 3.07 (m, 1H), 3.50 (m, 2H), 3.80 (m, 2H), 4.33 (d, 1H, J=4.4 Hz), 4.41 (m, 1H), 5.32 (m, 1H), 5.81 (d, 1H, J=7.9 Hz), 6.23 (br t, 1H), 6.68-6.87 (m, 3H), 7.04, d, J=6.1 Hz), 7.27 (s, 5H). MS APCI, m/z=530 (M+1). LC/MS: 2.55 min.

The precursor 3-cyclohexyl-N¹-[(6,7-cis)-5-oxo-7-phenyl-1,4-thiazepan-6-yl]-L-alaninamide (62b) was prepared as follows:

### a. N²-(tert-butoxycarbonyl)-3-cyclohexyl-N¹-[(6,7-cis)-5-oxo-7-phenyl-1,4-thiazepan-6-yl]-L-alaninamide (62a)

To a stirred solution of (6,7-cis)-6-amino-7-phenyl-1,4-thiazepan-5-one hydrobromide (48a) (208 mg, 0.686 mmol) in DCM (10 mL) under nitrogen was added, N-(tert-butoxycarbonyl)-3-cyclohexyl-L-alanine (205 mg, 0.755 mmol), HOBt (112 mg, 0.829 mmol), NMM (166 mg, 1.643 mmol) and EDAC-HCl (158 mg, 0.824 mmol). The mixture was stirred at ambient temperature for ˜12 h. The reaction mixture was diluted with saturated aqueous sodium bicarbonate and extracted with EtOAc. The organic solution was separated, extracted in succession with 1N aqueous HCl and brine then dried, filtered and evaporated to afford the title compound (326 mg, 99%) as a white solid. ¹H NMR (300 MHz, CDCl₃) δ0.87 (m, 2H), 1.03-1.86 (m, 20H), 2.79 (m, 1H), 3.03 (m, 1H), 3.78 (m, 2H), 4.19 (m, 1H), 4.36 (t, 1H, J=4.0 Hz), 4.65-4.90 (br m, 1H), 5.31 (m, 1H), 6.18-6.47 (br m, 1H), 7.15 (br, 1H), 7.29 (s, 5H). MS APCI, m/z=498 (M+Na). LC/MS: 2.50 min.

### b. 3-Cyclohexyl-N¹-[(6,7-cis)-5-oxo-7-phenyl-1,4-thiazepan-6-yl]-L-alaninamide (62b)

N²-(tert-Butoxycarbonyl)-3-cyclohexyl-N¹-[(6,7-cis)-5-oxo-7-phenyl-1,4-thiazepan-6-yl]-L-alaninamide (62a) (325 mg, 0.683 mmol) was dissolved in 3:1 (v/v) DCM:trifluoroacetic acid (8 mL) and kept at ambient temperature for 2 h. The solution was evaporated and the residue partitioned between EtOAc and saturated aqueous sodium bicarbonate. The organic solution was separated, dried, filtered and evaporated to afford the title compound (252 mg, 98%) as a yellow oil. ¹H NMR (300 MHz, CDCl₃) δ0.91 (m, 2H), 1.02-1.79 (m, 13H), 2.85 (m, 1H), 3.03 (m, 1H), 3.32 (m, 1H), 3.66-3.94 (m, 2H), 4.40 (m, 1H), 5.35 (m, 1H), 6.16 (m, 1H), 7.29 (m, 5H), 7.86 (br d, 0.5H), 8.06 (br d, 0.5H). MS APCI, m/z=376 (M+1). LC/MS: 1.48 min. MS APCI, m/z=376 (M+1). LC/MS: 1.68 min. (diastereomers evident).

### Example 63

### N²-[(3,5-Difluorophenyl)acetyl]-N¹-[(2S,3R)-5-(2-morpholin-4-ylethyl)-4-oxo-2-phenyl-2,3,4,5-tetrahydro-1,5-benzoxazepin-3-yl]-L-alaninamide hydrochloride (63)

A solution of 63d (260 mg) in methanol (10 mL) was treated with morpholine (87 μL) and acetic acid (172 μL) followed by sodium cyanoborohydride (80 mg). The reaction mixture was stirred for 16 h, acidified with hydrochloric acid (5 drops) and stirred for additional 1 h. At the end of this period upon concentration under reduced pressure the reaction mixture was diluted with DCM (100 mL) and washed with aqueous sodium carbonate solution. The organic layer was dried over potassium carbonate and concentrated under reduced pressure and the crude product was purified by column chromatography over silica gel. Product obtained after elution with DCM:methanol (20:1) was dissolved in methanol (1 mL) and treated with HCl (1 mL of 2M solution in ether) and diluted with ether (70 mL). The solid thus obtained was filtered to afford the title compound in 1:1 mixture with the (2R,3R) diastereomer (150 mg) as a white solid: ¹H NMR (300 MHz, d6-DMSO) δ 1.07 (m, 3H), 3.79 (m, 2H), 3.95 (m, 2H), 4.22 (m, 2H), 4.40 (m, 1H), 4.938 (m, 1H), 5.54 (t, 1H, J=6 Hz), 7.899-7.39 (m, 13H), 7.63 (m, 1H), 8.27 (d, 0.5H, J=7 Hz), 8.355 (d, 0.5H, J=7 Hz). MS APCI, m/z=593 (M+1). LC/MS: 2.02 min.

The starting aldehyde N²-[(3,5-difluorophenyl)acetyl]-N¹-[(2S,3R)-4-oxo-5-(2-oxoethyl)-2-phenyl-2,3,4,5-tetrahydro-1,5-benzoxazepin-3-yl]-L-alaninamide (63d) was prepared in the following manner:

### a. tert-Butyl [(2,3-cis)-5-allyl-4-oxo-2-phenyl-2,3,4,5-tetrahydro-1,5-benzoxazepin-3-yl]carbamate (63a)

A solution of tert-butyl [(2,3-cis)-4-oxo-2-phenyl-2,3,4,5-tetrahydro-1,5-benzoxazepin-3-yl]carbamate (46a) (750 mg) in THF (10 mL) was treated with powdered KOH (123 mg), tetrabutylammonium bromide (64 mg) and allylbromide (484 mg). Upon stirring for 16 h the reaction mixture was filtered, and the precipitate was washed with EtOAc. The solvent were removed under reduced pressure. The product was purified by column chromatography over silica gel. Elution with 9:1 DCM-EtOAc afforded the title product (792 mg). ¹H NMR (300 MHz, CDCl₃) δ 1.37 (s, 9H), 4.46 (dd, 1H, J=5 Hz, J=16 Hz), 4.97 (d, 1H, J=4 Hz), 5.23 (m, 2H), 5.66 (t, 1H, J=3 Hz), 5.93 (m, 1H), 7.30 (m, 9H). MS APCI, m/z=295 (M+1). LC/MS: 2.72 min.

### b. (2,3-cis)-5-Allyl-3-amino-2-phenyl-2,3-dihydro-1,5-benzoxazepin-4(5H)-one (63b)

A solution of tert-butyl [(2S,3R)-5-allyl-4-oxo-2-phenyl-2,3,4,5-tetrahydro-1,5-benzoxazepin-3-yl]carbamate (63a) (792 mg) in DCM (10 mL) was treated with phenol (470 mg) and trifluoroacetic acid (10 mL). Upon stirring for 30 min the reaction mixture was concentrated under reduced pressure, treated with 5% HCl and extracted with ether (100 mL). The aqueous layer was basified with potassium carbonate and extracted with DCM. The organic layer was dried over anhydrous potassium carbonate and concentrated under reduced pressure to afford the title product (430 mg). ¹H NMR (300 MHz, CDCl₃) δ 4.12 (d, 1H, J=6 Hz), 4.50 (d, d, 1H, J=6 Hz, J=15 Hz), 4.62 (d, d, 1H, J=6 Hz, J=15 Hz), 5.25 (m, 2H), 5.46 (d, 1H, J=7 Hz), 5.98 (m, 1H), 7.30 (m, 9H).

### c. N¹-[(2R,3S)-5-Allyl-4-oxo-2-phenyl-2,3,4,5-tetrahydro-1,5-benzoxazepin-3-yl]-N²-[(3,5-difluorophenyl)acetyl]-L-alaninamide (63c)

A method similar to that described for 97e was used except that (2,3-cis)-5-allyl-3-amino-2-phenyl-2,3-dihydro-1,5-benzoxazepin-4(5H)-one (63b) (430 mg) was the amine component and N-[(3,5-difluorophenyl)acetyl]-L-alanine (1e) was the acid component to afford the title compound as a 1:1 mixture with the (2S,3R) diastereomer (582 mg). ¹H NMR (300 MHz, C₆D₆) δ 0.93 (d, 1.5H, J=7 Hz), 1.05 (d, 1.5H, J=7 Hz), 3.47 (two peaks, 2H), 4.22-4.67 (m, 3H), 5.06 (m, 1H), 5.26 (m, 2H), 5.68 (m, 1H), 5.90 (m, 1H), 6.25 (d, 1H, J=7 Hz), 6.72 (m, 2H), 6.25-7.38 (m, 10H). MS APCI, m/z=520 (M+1). LC/MS: 2.46 min.

### d. N²-[(3,5-Difluorophenyl)acetyl]-N¹-[(2R,3S)-4-oxo-5-(2-oxoethyl)-2-phenyl-2,3,4,5-tetrahydro-1,5-benzoxazepin-3-yl]-L-alaninamide (63d)

A solution of 63c (582 mg) in THF (10 mL) was treated with a solution sodium periodate (530 mg) in water (10 mL) followed by a 4% solution of OsO₄(1 mL). The reaction mixture was stirred for 16 h, diluted with sodium bisulfite solution (20 mL) and extracted with EtOAc. The organic layers were washed with NaCl solution, dried over magnesium sulfate and concentrated under reduced pressure. The product thus obtained was dissolved in THF (50 mL) and treated with a solution sodium periodate (530 mg) in water (50 mL). After stirring for 16 h the reaction mixture was concentrated under reduced pressure and extracted with DCM (2×100 mL). The organics were concentrated under reduced pressure and the product was purified by chromatography to obtain the title compound in 1:1 mixture with the (2S,3R) diastereomer (417 mg). ¹H NMR (300 MHz, CDCl₃) δ 1.05 (m, 3H), 3.47 (two peaks, 2H), 6.74 (m, 2H), 7.25-7.60 (m, 12H).

### Example 64

### N²-[(3,5-Difluorophenyl)acetyl]-N¹-[(2R,3S)-4-oxo-2-phenyl-2,3,4,5-tetrahydro-1,5-benzoxazepin-3-yl]-L-leucinamide (64)

To a stirred solution of N¹-[(2R,3S)-4-oxo-2-phenyl-2,3,4,5-tetrahydro-1,5-benzoxazepin-3-yl]-L-leucinamide (64g) (65 mg, 0.177 mmol) in DCM (2 mL) was added 3,5-difluorophenylacetic acid (35 mg, 0.230 mmol), HOBt (31 mg, 0.230 mmol), NMM (25 mg, 0.248 mmol) and EDAC-HCl (44 mg, 0.230 mmol). The mixture was stirred at ambient temperature under nitrogen for ˜12 h. The reaction mixture was diluted with water and extracted with EtOAc. The combined organic extracts were washed in succession with saturated aqueous sodium bicarbonate and 1N aqueous HCl. The organic solution was dried, filtered and evaporated. The residue was purified by flash chromatography on silica gel eluting with 6:1 (v/v) DCM-EtOAc to afford the title compound in a 1:1 mixture with the 2S,3R diastereomer (65 mg, 70%) as a white solid. TLC R_{f}=0.31 (6:1 DCM:EtOAc). ¹H NMR (300 MHz, CDCl₃) δ0.82 (m, 6H), 1.21-1.55 (m, 3H), 3.48 (s, 2H), 4.28 (m, 1H), 5.15 (t, 1H, J=7.2 Hz), 5.76 (m, 2H), 6.27 (d, 1H, J=7.0 Hz), 6.78 (m, 3H), 7.06 (m, 1H), 7.15-7.42 (m, 8H), 7.55 (br s, 1H). MS APCI, m/z=510 (M+1). LC/MS: 2.40 min.

The precursor N¹-[(2R,3S)-4-oxo-2-phenyl-2,3,4,5-tetrahydro-1,5-benzoxazepin-3-yl]-L-leucinamide (64g) was prepared as follows:

### a. Potassium (2R,3S)-3-phenyloxirane-2-carboxylate (64a)

A stirred suspension of (1R)-1-phenylethanaminium (2R,3S)-3-phenyloxirane-2-carboxylate [K. Horada, J. Org. Chem., 31, 1407, 1966] (28.54 g, 0.10 mole) in ethanol (60 mL) and water (15 mL) was treated rapidly with a solution of KOH (9.5 g, 0.17 mole) in ethanol (60 mL). The mixture was stirred at ambient temperature for 20 min, ethyl ether (150 mL) added, the mixture stirred an additional 10 min and the white solid filtered off and washed with ethyl ether. A second crop was obtained by dilution of the filtrate with ethyl ether to 500 mL. Both samples were dried in vacuo. The pH of a small sample of crop 1 in water was 8 to 9 while a sample of crop 2 had pH 14. Crop 2 was thus stirred with 15 mL of absolute ethanol for 15 min, filtered, washed with acetone and dried in vacuo. Crop 1 (16.23 g, 80%) [α]D 25=−146° (c=1.0, H₂O) and crop 2 (3.03 g 15%) [α]D 25=−143° (c=1.2, H₂O). ¹H NMR (300 MHz, DMSO-d6) δ2.97 (d, 1H, J=1.8 Hz), 3.66 (d, 1H, J=1.8 Hz), 7.22-7.35 (m, 5H). HPLC (Method B): 2.74 min.

### b. (2R,3S)—N-(2-Hydroxyphenyl)-3-phenyloxirane-2-carboxamide (64b)

To a stirred suspension of potassium (2R,3S)-3-phenyloxirane-2-carboxylate (64a) (4.040 g, 20.00 mmol) in dry THF (100 mL) under nitrogen cooled in an ice-water bath was added isobutyl chloroformate (2.730 g, 20.00 mmol) slowly via syringe. NMM (0.460 mg, 4.55 mmol) was added and the mixture stirred while gradually warming to 10° C. over 75 min. The mixture was cooled to 0° C. and 2-aminophenol was added, then the cooling bath was removed and the reaction stirred at ambient temperature for 24 h. The reaction was diluted with diethyl ether (100 mL) then filtered through diatomaceous earth to remove suspended solids. Rotary evaporation of the solution afforded a yellow solid that was triturated with diethyl ether and collected by filtration. The solid was rinsed on the filter with additional diethyl ether to afford pure title compound (3.920 g, 77%) as an off-white solid. ¹H NMR (300 MHz, DMSO-d6) δ4.00 (d, 1H, J=1.8 Hz), 4.22 (d, 1H, J=1.8 Hz), 6.80 (m, 1H), 6.87-7.00 (m, 2H), 7.40 (s, 5H), 7.95 (d, 1H, J=7.9 Hz), 9.27 (s, 1H), 9.97 (s, 1H). ¹³C-DEPT NMR (75 MHz, DMSO-d6) δ57.7 (CH), 58.4 (CH), 115.5 (CH), 119.4 (CH), 121.6 (CH), 125.1 (CH), 125.8 (C), 126.5 (CH), 128.9 (CH), 129.1 (CH), 135.9 (C), 147.7 (C), 165.4 (C). MS APCI, m/z=256 (M+1). LC/MS: 2.12 min.

### c. (2R,3R)-3-Hydroxy-2-phenyl-2,3-dihydro-1,5-benzoxazepin-4(5H)-one (64c)

Scandium triflate (730 mg, 1.48 mmol) was added to a stirred suspension of (2R,3S)—N-(2-hydroxyphenyl)-3-phenyloxirane-2-carboxamide (64b) in dry acetonitrile (150 mL) under nitrogen. The mixture was stirred at ambient temperature for 24 h then heated at reflux for an additional 1 h. The solvent was evaporated, the residue dissolved in EtOAc, and filtered through diatomaceous earth. Evaporation afforded an off-white solid which was purified by flash chromatography on silica gel eluting with 10:1 (v/v) DCM-EtOAc to afford the title compound (2.290 g, 60%) as a white solid. TLC R_{f}=0.20 (10:1 DCM:EtOAc). ¹H NMR (300 MHz, CDCl₃) δ3.70 (d, 1H, J=5 Hz), 4.63 (m, 1H), 5.28 (d, 1H, J=10 Hz), 6.89 (m, 1H), 7.02-7.16 (m, 3H), 7.35-7.47 (m, 5H), 7.78 (br, 1H). MS APCI, m/z=256 (M+1). LC/MS: 1.84 min. [α]D 25=+288° (c=5.0, CHCl₃).

### d. (2R,3S)-3-Azido-2-phenyl-2,3-dihydro-1,5-benzoxazepin-4(5H)-one (64d)

Triethylamine (1.667 g, 16.472 mmol) was added via syringe to a stirred solution of (2R,3R)-3-hydroxy-2-phenyl-2,3-dihydro-1,5-benzoxazepin-4(5H)-one (64c) (2.532 g, 9.922 mmol) in DCM (90 mL) under nitrogen at 40° C. Next trifluoromethanesulfonyl chloride (2.541 g, 15.080 mmol) was added slowly via syringe and the mixture kept at −25° C. for 2 h. Additional triethylamine (1.667 g, 16.472 mmol) was added followed by trifluoromethanesulfonyl chloride (2.541 g, 15.080 mmol) and the reaction was kept for 8 h at −25° C. Again, additional triethylamine (1.667 g, 16.472 mmol) was added via syringe followed by trifluoromethanesulfonyl chloride (2.541 g, 15.080 mmol) and the reaction was kept for 24 h at −25 C. The solvent was evaporated without heating under reduced pressure and the residue dissolved in DMF (25 mL). Sodium azide (3.000 g, 46.15 mmol) was added to the stirred DMF solution at −10° C. and the reaction was allowed to warm to ambient temperature. After 2 h the reaction was diluted with water (300 mL) and extracted three times with EtOAc. The combined organic extracts were washed with saturated aqueous sodium bicarbonate and then with brine. The organic solution was dried, filtered and evaporated. The residue was purified by flash chromatography an silica gel eluting with 30:1 (v/v) DCM-EtOAc to afford the title compound as an off-white solid. TLC R_{f}=0.35 (20:1 (v/v) DCM-EtOAc). ¹H NMR (300 MHz, CDCl₃) δ4.45 (d, 1H, J=6 Hz), 5.56 (d, 1H, J=6 Hz), 7.00-7.07 (m, 1H), 7.10-7.26 (m, 3H), 7.40-7.46 (m, 3H), 7.51-7.61 (m, 3H). MS APCI, m/z=253 (M+1−N₂). LC/MS: 2.25 min. [α]D 25=−179° (c=5.0, CHCl₃)

### e. (2R,3S)-3-Amino-2-phenyl-2,3-dihydro-1,5-benzoxazepin-4(5H)-one (64e)

To a solution of (2R,3S)-3-azido-2-phenyl-2,3-dihydro-1,5-benzoxazepin-4(5H)-one (64d) (1.128 g, 4.024 mmol)) in ethanol (80 mL) was added 1N hydrochloric acid (4.43 mL) and 5% palladium on carbon (60 mg). The reaction was purged with hydrogen and stirred for 2 h under a balloon of hydrogen. The reaction was purged with nitrogen then filtered through diatomaceous earth. The solvent was evaporated to afford a white solid that was then partitioned between EtOAc and saturated aqueous sodium bicarbonate while stirring. The organic phase was separated and the aqueous phase extracted an additional two times with EtOAc. The combined organic extracts were dried, filtered and evaporated to afford the title compound (973 mg, 95%) as an off-white solid. ¹H NMR (300 MHz, DMSO-d6) δ1.36 (br, 2H), 3.93 (d, 1H, J=6.2 Hz), 5.45 (d, 1H, J=6.2 Hz), 7.06-7.22 (m, 4H), 7.32-7.46 (m, 5H), 9.96 (br, 1H). MS APCI, m/z=255 (M+1). LC/MS: 1.29 min.

### f. N²-[tert-Butoxycarbonyl]-N¹-[(2R,3S)-4-oxo-2-phenyl-2,3,4,5-tetrahydro-1,5-benzoxazepin-3-yl]-L-leucinamide (64f)

To a stirred solution of (2R,3S)-3-amino-2-phenyl-2,3-dihydro-1,5-benzoxazepin-4(5H)-one (64e) (50 mg, 0.197 mmol), and HOBt (33 mg, 0.2144 mmol), in DCM (2 mL) was added NMM (50 mg, 0.495 mmol), and EDAC-HCl (46 mg, 0.240 mmol). The mixture was stirred at ambient temperature for ˜12 h under nitrogen. The reaction was diluted with EtOAc and extracted with 10% aqueous citric acid. The organic solution was dried and evaporated. The residue was purified by flash chromatography on silica gel eluting with 10:1 (v/v) DCM-EtOAc to afford the title compound (85 mg, 92%) as a white solid. ¹H NMR (300 MHz, CDCl₃) δ0.88 (t, 6H, J=5.9 Hz), 1.19-1.65 (m, 12H), 3.96 (m, 1H), 4.65 (m, 1H), 5.17 (t, 1H, J=7.0 Hz), 5.84 (d, 1H), J=7.0 Hz), 6.45 (br d, 1H, J=7.0 Hz), 7.06 (m, 1H), 7.23 (m, 3H), 7.38 (m, 5H), 7.60 (br s, 1H). MS APCI, m/z=490 (M+Na). LC/MS: 2.42 min.

### g. N¹-[(2R,3S)-4-Oxo-2-phenyl-2,3,4,5-tetrahydro-1,5-benzoxazepin-3-yl]-L-leucinamide (64g)

N²-[tert-Butoxycarbonyl]-N¹-[(2R,3S)-4-oxo-2-phenyl-2,3,4,5-tetrahydro-1,5-benzoxazepin-3-yl]-L-leucinamide (64f) (83 mg, 0.177 mmol) was dissolved in 5:1 (v/v) DCM:trifluoroacetic acid and kept at ambient temperature for 1 h. The solution was evaporated and the residue dissolved in EtOAc. The solution was extracted in succession with saturated aqueous sodium bicarbonate and brine then the organic solution was dried, filtered and evaporated to afford the title compound (65 mg, 99%) as a yellow oil. MS APCI, m/z=368 (M+1). LC/MS: 1.63 min.

### Example 65

### (2S)-2-[(3,5-Difluorophenyl)acetyl]amino-2-(4-fluorophenyl)-N-[(2R,3S)-4-oxo-2-phenyl-2,3,4,5-tetrahydro-1,5-benzoxazepin-3-yl]acetamide (65)

To a stirred solution of (2S)-2-amino-2-(4-fluorophenyl)-N-[(2R,3S)-4-oxo-2-phenyl-2,3,4,5-tetrahydro-1,5-benzoxazepin-3-yl]acetamide (65b) (81 mg, 0.199 mmol) in DCM (2 mL) under nitrogen was added 3,5-difluorophenylacetic acid (40 mg, 0.232 mmol), HOBt (35 mg, 0.259 mmol), NMM (30 mg, 0.297 mmol) and EDAC-HCl (50 mg, 0.261 mmol). The mixture was stirred 5 h at ambient temperature then evaporated. The residue was dissolved in EtOAc and extracted in succession with saturated sodium bicarbonate, 1N aqueous HCl, and brine. The organic solution was dried, filtered and evaporated. The residue was purified by flash chromatography on silica gel eluting with 6:1 (v/v) DCM-EtOAc (TLC R_{f}=0.42) to afford the title compound (85 mg, 76%) as a white solid. ¹H NMR (300 MHz, CDCl₃) δ3.54 (s, 2H), 5.08 (t, 1H, J=7.0 Hz), 5.19 (d, 1H, J=6.5 Hz), 5.79 (d, 1H, J=7.0 Hz), 6.16 (br d, 1H, J=6.5 Hz), 6.62 (br d, 1H, J=7.0 Hz), 6.66-6.86 (m, 3H), 6.87-7.04 (m, 3H), 7.08-7.39 (m, 10H), 7.50 (br s, 1H). MS APCI, m/z=560 (M+1). LC/MS: 2.61 min.

The precursor (2S)-2-amino-2-(4-fluorophenyl)-N-[(2R,3S)-4-oxo-2-phenyl-2,3,4,5-tetrahydro-1,5-benzoxazepin-3-yl]acetamide (65b) was prepared as follows:

### a. tert-Butyl ((1S)-1-(4-fluorophenyl)-2-oxo-2-{[(2R,3S)-4-oxo-2-phenyl-2,3,4,5-tetrahydro-1,5-benzoxazepin-3-yl]amino}ethyl)carbamate (65a)

To a stirred solution of (2S)-[(tert-butoxycarbonyl)amino](4-fluorophenyl)acetic acid (269 mg, 1.00 mmol) and (2,3-cis)-3-amino-2-phenyl-2,3-dihydro-1,5-benzoxazepin-4(5H)-one (6e) (254 mg, 1.00 mmol) in DCM (10 ml) under nitrogen was added NMM (111 mg, 1.100 mmol), HOBt (162 mg, 1.20 mmol) and EDAC-HCl (211 mg, 1.10 mmol). The mixture was stirred for ˜12 h at ambient temperature then the solvent was evaporated and the residue partitioned between EtOAc and 1N aqueous HCl. The organic phase was separated then washed with saturated aqueous sodium bicarbonate, dried and evaporated. The residue was purified by flash chromatography on silica gel eluting with 10:1 (v/v) DCM-EtOAc. The desired 2R,3S diastereomer eluted first (R_{f}=0.27) and the 2S,3R diastereomer eluted later (R_{f}=0.20). The fractions containing the early eluting diastereomer were combined and evaporated to afford the title compound (202 mg, 40%) as a white solid. ¹H NMR (300 MHz, CDCl₃) δ1.41 (s, 9H), 4.94 (br, 1H), 5.09 (m, 1H), 5.48 (d, 1H, J=6.6 Hz), 5.83 (d, 1H, J=7.0 Hz), 6.33 (br, 1H), 6.91-7.04 (m, 3H), 7.12-7.28 (m, 5H), 7.38 (s, 5H), 7.48 (br, 1H). MS APCI, m/z=528 (M+Na). LC/MS: 2.06 min.

### b. (2S)-2-Amino-2-(4-fluorophenyl)-N-[(2R,3S)-4-oxo-2-phenyl-2,3,4,5-tetrahydro-1,5-benzoxazepin-3-yl]acetamide (65b)

tert-Butyl ((1S)-1-(4-fluorophenyl)-2-oxo-2-{[(2R,3S)-4-oxo-2-phenyl-2,3,4,5-tetrahydro-1,5-benzoxazepin-3-yl]amino}ethyl)carbamate (65a) (202 mg, 0.400 mmol) was dissolved in 5:1 (v/v) DCM-trifluoroacetic acid (4 mL) and kept at ambient temperature for 1 h. The solution was evaporated and the residue was dissolved in EtOAc and extracted with saturated aqueous sodium bicarbonate. The organic solution was dried and evaporated to afford the title compound (160 mg, 99%) as a yellow oil. MS APCI, m/z=406 (M+1). LC/MS: 1.60 min.

### Example 66

### (2S)-2-[(Cyclohexylacetyl)amino]-2-(4-fluorophenyl)-N-[(2R,3S)-4-oxo-2-phenyl-2,3,4,5-tetrahydro-1,5-benzoxazepin-3-yl]acetamide (66)

To a stirred solution of (2S)-2-amino-2-(4-fluorophenyl)-N-[(2R,3S)-4-oxo-2-phenyl-2,3,4,5-tetrahydro-1,5-benzoxazepin-3-yl]acetamide (65b) (81 mg, 0.199 mmol) in DCM (2 mL) under nitrogen was added cyclohexylacetic acid (35 mg, 0.246 mmol), HOBt (35 mg, 0.259 mmol), NMM (30 mg, 0.297 mmol) and EDAC-HCl (50 mg, 0.261 mmol). The mixture was stirred 5 h at ambient temperature then evaporated. The residue was dissolved in EtOAc and extracted in succession with saturated sodium bicarbonate, 1N aqueous HCl, and brine. The organic solution was dried, filtered and evaporated. The residue was purified by flash chromatography on silica gel eluting with 6:1 (v/v) DCM-EtOAc to afford the title compound (89 mg, 84%) as a white solid. TLC R_{f} 0.42 (6:1 DCM-EtOAc). ¹H NMR (300 MHz, CDCl₃) δ0.81-1.37 (m, 5H), 1.59-1.87 (m, 6H), 2.09 (d, 2H, J=6.5 Hz), 5.09 (t, 1H, J=7.0 Hz), 5.26 (d, 1H, J=7.0 Hz), 5.83 (d, 1H, J=7.0 Hz), 6.24 (br d, 1H, J=6.5 Hz), 6.48 (br d, 1H, J=6.5 Hz), 6.88-7.05 (m, 3H), 7.12-7.30 (m, 5H), 7.36 (s, 5H), 7.56 (br s, 1H). MS APCI, m/z=530 (M+1). LC/MS: 2.69 min.

### Example 67

### N²-[(3,5-Difluorophenyl)acetyl]-N¹-[(2R,3S)-4-oxo-2-phenyl-5-prop-2-yn-1-yl-2,3,4,5-tetrahydro-1,5-benzoxazepin-3-yl]-L-alaninamide (67)

To a stirred solution of (2R,3S)-3-amino-2-phenyl-5-prop-2-yn-1-yl-2,3-dihydro-1,5-benzoxazepin-4(5H)-one (67c) (65 mg, 0.222 mmol) in DCM (3 mL) was added N-[(3,5-difluorophenyl)acetyl]-L-alanine (1e) (65 mg, 0.267 mmol), HOBt (40 mg, 0.296 mmol), NMM (31 mg, 0.306 mmol) and EDAC-HCl (55 mg, 0.286 mmol). After stirring at ambient temperature Linder nitrogen for ˜12 h the solvent was evaporated and the residue partitioned between EtOAc and saturated aqueous sodium bicarbonate. The organic solution was separated, washed with 1N aqueous HCl then dried, filtered and evaporated. The residue was purified by recrystallization from 1:1 (v/v) EtOAc-hexanes to afford the title compound (86 mg, 74%) as a white solid. TLC R_{f}=0.25 (diethyl ether). ¹H NMR (300 MHz, CDCl₃) δ1.22 (d, 3H, J=7.0 Hz), 2.31 (m, 1H), 3.48 (s, 2H), 4.23 (m, 1H), 4.69 (d, 2H, J=2.2 Hz), 5.11 (t, 1H, J=7.0 Hz), 5.69 (d, 1H, J=7.4 Hz), 5.87 (d, 1H), 6.18 (d, 1H), 6.69-6.86 (m, 3H), 7.26-7.53 (m, 9H). MS APCI, m/z=518 (M+1). LC/MS: 2.63 min.

The precursor (2R,3S)-3-amino-2-phenyl-5-prop-2-yn-1-yl-2,3-dihydro-1,5-benzoxazepin-4(5H)-one (67c) was prepared as follows:

### a. tert-Butyl [(2R,3S)-4-oxo-2-phenyl-2,3,4,5-tetrahydro-1,5-benzoxazepin-3-yl]carbamate (67a)

To a stirred solution of (2R,3S)-3-amino-2-phenyl-2,3-dihydro-1,5-benzoxazepin-4(5H)-one (64e) (700 mg, 3.207 mmol) in DCM (30 mL) under nitrogen at 0° C. was added triethylamine (324 mg, 3.21 mmol) and di-tert-butyl dicarbonate (700 mg, 3.208 mmol). The mixture was allowed to warm to ambient temperature and stirred for 24 h. The solvent was evaporated and the residue purified by flash chromatography on silica gel eluting first with DCM and finally with 20:1 (v/v) DCM-EtOAc to afford the title compound (943 mg, 83%) as a white solid. TLC R_{f}=0.50 (2:1 hexane-EtOAc). ¹H NMR (300 MHz, CDCl₃) δ1.39 (s, 9H), 4.90-5.05 (m, 2H), 5.76 (d, 1H, J=6.8 Hz), 7.05 (m, 1H), 7.14-7.29 (m, 3H), 7.36-7.53 (m, 6H). MS ES⁺, m/z=377 (M+Na). LC/MS: 1.93 min.

### b. tert-Butyl [(2R,3S)-4-oxo-2-phenyl-5-prop-2-yn-1-yl-2,3,4,5-tetrahydro-1,5-benzoxazepin-3-yl]carbamate (67b)

To a stirred solution of tert-butyl [(2R,3S)-4-oxo-2-phenyl-2,3,4,5-tetrahydro-1,5-benzoxazepin-3-yl]carbamate (67a) (160 mg, 0.451 mmol) in DMF (2 mL) was added propargyl bromide (80 mg, 0.672 mmol) and powdered cesium carbonate (219 mg, 0.672 mmol). The mixture was stirred under nitrogen at ambient temperature for 18 h. The reaction was diluted with water and extracted twice with EtOAc. The residue obtained from the combined organic extracts was purified by flash chromatography on silica gel eluting with 5:1 (v/v) hexane-EtOAc to afford the title compound (143 mg, 80%) as a white solid. TLC R_{f}=0.19 (6:1 hexane-EtOAc). ¹H NMR (300 MHz, CDCl₃) δ1.38 (s, 9H), 2.30 (m, 1H), 4.69 (m, 2H), 4.96 (m, 2H), 5.66 (m, 1H), 7.28 (m, 3H), 7.39 (s, 5H), 7.48 (m, 1H). MS APCI, m/z=293 (M+1). LC/MS: 2.86 min.

### c. (2R,3S)-3-Amino-2-phenyl-5-prop-2-yn-1-yl-2,3-dihydro-1,5-benzoxazepin-4(5H)-one (67c)

tert-Butyl [(2R,3S)-4-oxo-2-phenyl-5-prop-2-yn-1-yl-2,3,4,5-tetrahydro-1,5-benzoxazepin-3-yl]carbamate (67b) (125 mg, 0.318 mmol) was dissolved in 5:1 (v/v) DCM-trifluoroacetic acid (4 mL) and kept at ambient temperature for 90 min. The solution was evaporated and the residue was dissolved in EtOAc and extracted with saturated aqueous sodium bicarbonate. The organic solution was dried and evaporated to afford the title compound (92 mg, 99%) as a white solid. ¹H NMR (300 MHz, CDCl₃) δ1.36 (br s, 2H), 2.29 (t, 1H, J=2.3 Hz), 4.10 (m, 1H), 4.56 and 4.61 (dd, 1H, J=2.4 Hz), 4.76 and 4.81 (dd, 1H, J=2.4 Hz), 5.46 (d, 1H, J=7.4 Hz), 7.27 (m, 3H), 7.38 (m, 3H), 7.47 (m, 3H). MS APCI, m/z=293 (M+1). LC/MS: 1.63 min.

### Example 68

### N²-[(3,5-Difluorophenyl)acetyl]-N¹-[(2R,3S)-7-methoxy-4-oxo-2-phenyl-2,3,4,5-tetrahydro-1,5-benzoxazepin-3-yl]-L-alaninamide (68)

To a stirred solution of (2,3-cis)-3-amino-7-methoxy-2-phenyl-2,3-dihydro-1,5-benzoxazepin-4(5H)-one (68e) (230 mg, 0.810 mmol) in DCM (8 mL) was added N-[(3,5-difluorophenyl)acetyl]-L-alanine (1e) (243 mg, 1.003 mmol), HOBt (135 mg, 1.000 mmol), NMM (101 mg, 1.000 mmol) and EDAC-HCl (192 mg, 1.001 mmol). The mixture was stirred at ambient temperature under nitrogen for ˜12 h. The reaction mixture was diluted with water and extracted with EtOAc. The combined organic extracts were washed in succession with saturated aqueous sodium bicarbonate and 1N aqueous HCl. The organic solution was dried, filtered and evaporated. The residue was purified by flash chromatography on silica gel eluting with 3:1 (v/v) DCM-EtOAc to afford the title compound in a 1:1 mixture with the 2S,3R diastereomer (310 mg 75%) as a white solid. TLC R_{f}=0.57 (EtOAc). ¹H NMR (300 MHz, CDCl₃) δ1.07 (d, 1.5H, J=7.0 Hz), 1.22 (d, 1.5H, J=7.0 Hz), 3.43 (s, 1H), 3.48 (s, 1H), 3.81 (s, 3H), 4.43 (m, 1H), 5.09 (m, 1H), 5.71 (m, 1H), 6.05 (d, 0.5H, J=7.4 Hz), 6.19 (d, 0.5H, J=7.4 Hz), 6.41 (d, 1H, J=6.6 Hz), 6.58 (m, 1H), 6.63-6.87 (m, 4H), 7.18 (d, 1H, J=8.8 Hz), 7.30, (s, 2.5H), 7.36 (s, 2.5H), 7.63 (br, 0.5H), 8.00 (br, 0.5H). MS APCI, m/z=510 (M+1). LC/MS: 2.40 min.

The precursor (2,3-cis)-3-amino-7-methoxy-2-phenyl-2,3-dihydro-1,5-benzoxazepin-4(5H)-one (68e) was prepared as follows:

### a. erythro Ethyl 2-hydroxy-3-(4-methoxy-2-nitrophenoxy)-3-phenylpropanoate (68a)

To a stirred mixture of 4-methoxy-2-nitrophenol (11.87 g, 70 mmol), ethyl 3-phenyloxirane-2-carboxylate (10.39 g, 54 mmol) and ethanol (175 mL) was added portionwise 60% sodium hydride (0.65 g, 16 mmol) and the red mixture stirred at reflux for 9 d. The solvent was removed in vacuo, the residue dissolved in EtOAc and extracted with 10% aqueous potassium carbonate. The organic layer was washed with water and brine, dried (MgSO₄), filtered and the solvent stripped in-vacuo to yield an oil. Column chromatography (DCM then 5% methanol-DCM) afforded (9.5 g, 49%) of slightly impure title compound that was used in the next step without further purification. ¹H NMR (300 MHz, CDCl₃) δ1.21 (t, 3H) 3.25 (d, 1H, J=7.5 Hz) 3.77 (s, 3H), 4.20 (q, 2H) 4.67 (q, 1H, J=3.5, 7.5 Hz), 5.51 (d, 1H, J=3.5 Hz), 6.82 (d, 1H, J=9.2 Hz), 6.92 (dd, 1H, J=9.2, 3.0 Hz), 7.3-7.4 (m, 6H). HPLC (Method A): 3.24 min.

### b. erythro Ethyl 2-hydroxy-3-(4-methoxy-2-nitrophenoxy)-3-phenylpropanoate (68b)

A mixture of erythro ethyl 2-hydroxy-3-(4-methoxy-2-nitrophenoxy)-3-phenylpropanoate (68a), 5% Pd/C, (50% H₂O Degaussa catalyst, 0.5 g) and ethanol (225 mL) was hydrogenated in a Parr apparatus at 42 psi hydrogen for 2.5 h. The catalyst was filtered off through a pad of diatomaceous earth and washed with ethanol. Removal of the solvent returned an orange oil that was purified by column chromatography (DCM, then 10% ethanol-DCM) to yield impure title compound as an viscous orange oil (6.5 g, 74%) which was used in the next step without further purification. ¹H NMR (300 MHz, CDCl₃) δ1.17 (t, 3H), 3.69 (s, 3H), 4.1-4.2 (m, 4H), 4.58 (d, 1H, J=3.5 Hz), 5.31 (d, 1H, J=3.1 Hz), 6.10 (dd, 1H, J=3, 8.8 Hz), 6.32 (d, 1H, J=3 Hz), 6.58 (dd, 1H, J=8.8 Hz), 7.3-7.4 (m, 7H). MS APCI, m/z=332 (M+1). LC/MS: 1.61 min.

### c. (2,3-trans)-3-Hydroxy-7-methoxy-2-phenyl-2,3-dihydro-1,5-benzoxazepin-4(5H)-one (68c)

A stirred warmed solution of the above impure erythro ethyl 2-hydroxy-3-(4-methoxy-2-nitrophenoxy)-3-phenylpropanoate (68b) (6.48 g, 19.6 mmol) in xylene (200 mL) was treated with a catalytic amount of pTSA (0.4 g) and the dark solution was refluxed under Dean-Stark conditions for 18 h. The xylene was stripped iii-vacuo and the residue in acetone was preabsorbed on silica gel. Column chromatography (DCM then 4:1 DCM-EtOAc) gave a pale yellow solid that was dissolved in a small volume of DCM and treated with hexane. The title compound was obtained by filtration as a pale yellow solid (2.59 g, 46%). ¹H NMR (300 MHz, CDCl₃) δ3.66 (d, 1H, J=5.3 Hz), 3.78 (s, 3H), 4.64 (q, J=5.3 Hz, J=9.7 Hz), 5.25 (d, 1H, J=9.7 Hz), 6.58 (d, 1H, J=3.1 Hz), 6.62 (dd, 1H, J=2.6 Hz, J=8.8 Hz), 6.76 (d, 1H, J=8.8 Hz), 7.4-7.5 (m, 5H), 7.65 (bs, 1H, NH). MS APCI, m/z=286 (M+1). LC/MS: 1.73 min.

### d. (2,3-cis)-3-Azido-7-methoxy-2-phenyl-2,3-dihydro-1,5-benzoxazepin-4(5H)-one (68d)

Triethylamine (1.353 g, 13.370 mmol) was added via syringe to a stirred solution of (2,3-trans)-3-hydroxy-7-methoxy-2-phenyl-2,3-dihydro-1,5-benzoxazepin-4(5H)-one (68c) (2.542 g, 8.916 mmol) in DCM (100 mL) under nitrogen at −10° C. Next trifluoromethanesulfonyl chloride (2.253 g, 13.369 mmol) was added slowly via syringe and the mixture kept at −10° C. for ˜12 h. Additional triethylamine (1.353 g, 13.370 mmol) was added via syringe followed by trifluoromethanesulfonyl chloride (2.253 g, 13.369 mmol) and the reaction was kept for 6 h at −25° C. The reaction was warmed to 0° C. and quenched by addition of 1N aqueous hydrochloric acid (50 mL). The organic phase was separated, dried, filtered and evaporated. Ethyl acetate was added to the residue and the resulting precipitate collected (recovered alcohol starting material, 1.712 g, 67%). The liquor was evaporated and the residue dissolved in DMF (5 mL). Sodium azide (2.000 g, 30.769 mmol) was added and the mixture stirred for 2 h at ambient temperature. The reaction was diluted with water and extracted with EtOAc. The residue from the organic phase was purified by flash chromatography on silica gel eluting with EtOAc to afford the title compound (260 mg, 29% accounting for recovered starting material) as a white solid. ¹H NMR (300 MHz, CDCl₃) δ3.79. (s, 3H), 4.43 (d, 1H, J=6.3 Hz), 5.52 (d, 1H, J=6.3 Hz), 6.57 (d, 1H, J=3.1 Hz), 6.73 (dd, 1H, J=8.8, 3.0), 7.15 (d, 1H, J=9.2 Hz), 7.43 (m, 3H), 7.56 (m, 2H), 7.77 (br, 1H). MS APCI, m/z=283 (M+1−N₂). LC/MS: 2.35 min

### e. (2,3-cis)-3-Amino-7-methoxy-2-phenyl-2,3-dihydro-1,5-benzoxazepin-4(5H)-one (68e)

To a solution of (2,3-cis)-3-azido-7-methoxy-2-phenyl-2,3-dihydro-1,5-benzoxazepin-4(5H)-one (68d) (254 mg, 0.818 mmol) in ethanol (10 mL) was added 5% palladium on carbon (30 mg) and 1N hydrochloric acid (1.0 mL). The mixture was stirred for 2 h under a balloon of hydrogen. The mixture was filtered through diatomaceous earth and the solution was evaporated. The solid residue was suspended between EtOAc and saturated aqueous sodium bicarbonate and stirred until dissolved. The organic phase was separated, dried, filtered and evaporated. The residue was purified by passing through a plug of silica gel (5 g) eluting with EtOAc to afford the title compound (230 mg, 99%) as an off-white solid. TLC R_{f}=0.27 (EtOAc). ¹H NMR (300 MHz, CDCl₃) δ1.43 (br, 2H), 3.80 (s, 3H), 4.12 (d, 1H, J=7.0 Hz), 5.50 (d, 1H, J=7.0 Hz), 6.56 (d, 1H, J=3.1 Hz), 6.73 (dd, 1H, J=8.7, 3.1), 7.17 (d, 1H, 8.8 Hz), 7.38 (m, 4H), 7.50 (m, 2H). MS APCI, m/z=285 (M+1). LC/MS: 1.47 min.

### Example 69

### N²-[(3,5-Difluorophenyl)acetyl]-N¹-[(2R,3S)-5-isopropyl-4-oxo-2-phenyl-2,3,4,5-tetrahydro-1,5-benzoxazepin-3-yl]-L-alaninamide (69)

To a stirred solution of (2R,3S)-3-amino-5-isopropyl-2-phenyl-2,3-dihydro-1,5-benzoxazepin-4(5H)-one (69b) (59 mg, 0.199 mmol) in DCM (2 mL) was added N-[(3,5-difluorophenyl)acetyl]-L-alanine (1e) (59 mg, 0.242 mmol), HOBt (33 mg, 0.245 mmol), NMM (30 mg, 0.297 mmol) and EDAC-HCl (47 mg, 0.245 mmol). After stirring at ambient temperature under nitrogen for 90 min. the solvent was evaporated and the residue partitioned between EtOAc and saturated aqueous sodium bicarbonate. The organic solution was separated, washed with 1N aqueous HCl then dried, filtered and evaporated. The residue was purified by flash chromatography on silica gel eluting with 1:1 (v/v) hexane-EtOAc to afford the title compound (64 mg, 62%) as a white solid. TLC R_{f}=0.17 (1:1 hexane-EtOAc). ¹H NMR (300 MHz, CDCl₃) δ1.21 (d, 3H, J=7.0 Hz), 1.29 (d, 3H, J=7.0 Hz), 1.54 (d, 3H, J=7.0 Hz), 3.47 (s, 2H), 4.22 (m, 1H), 4.77 (m, 1H), 4.93 (t, 1H, J=7.2 Hz), 5.60 (d, 1H, J=7.4 Hz), 5.87 (br d, 1H, J=7.4 Hz), 6.25 (br d, 1H, J=7.0 Hz), 6.67-6.86 (m, 3H), 7.21-7.43 (m, 9H). MS APCI, m/z=522 (M+1). LC/MS: 2.75 min.

The precursor (2R,3S)-3-amino-5-isopropyl-2-phenyl-2,3-dihydro-1,5-benzoxazepin-4(5H)-one (69b) was prepared as follows:

a. tert-Butyl [(2R,3S)-5-isopropyl-4-oxo-2-phenyl-2,3,4,5-tetrahydro-1,5-benzoxazepin-3-yl]carbamate (69a)

To a stirred solution of tert-butyl [(2R,3S)-4-oxo-2-phenyl-2,3,4,5-tetrahydro-1,5-benzoxazepin-3-yl]carbamate (67a) (125 mg, 0.352 mmol) in dry DMF (3 mL) under nitrogen was added 2-iodopropane (90 mg, 0.529 mmol) and powdered cesium carbonate (172 mg, 0.529 mmol). The mixture was stirred for ˜12 h at ambient temperature then diluted with water and extracted with EtOAc. The residue from the organic extract was purified by flash chromatography on silica gel eluting with 5:1 (v/v) hexane-EtOAc to afford the title compound (82 mg, 58%) as a white solid. TLC R_{f}=0.35 (5:1 hexane-EtOAc). ¹H NMR (300 MHz, CDCl₃) δ1.28 (d, 6H, J=7.0 Hz), 1.37 (s, 9H), 4.81 (m, 2H), 4.99 (m, 1H), 5.58 (d, 1H, J=7.0 Hz), 7.19-7.48 (m, 9H). MS APCI, m/z=397 (M+1). LC/MS: 2.75 min.

### b. (2R,3S)-3-Amino-5-isopropyl-2-phenyl-2,3-dihydro-1,5-benzoxazepin-4(5H)-one (69b)

tert-Butyl [(2R,3S)-5-isopropyl-4-oxo-2-phenyl-2,3,4,5-tetrahydro-1,5-benzoxazepin-3-yl]carbamate (69a) (80 mg, 0.202 mmol) was dissolved in 5:1 (v/v) DCM-trifluoroacetic acid (3 mL) and kept at ambient temperature for 30 min. The solution was evaporated and the residue was dissolved in EtOAc and extracted with saturated aqueous sodium bicarbonate. The organic solution was dried and evaporated to afford the title compound (59 mg, 99%) as a white solid. MS APCI, m/z=297 (M+1). LC/MS: 1.79 min

### Example 70

### Methyl [(2R,3S)-3-(N-[(3,5-difluorophenyl)acetyl]-L-alanylamino)-4-oxo-2-phenyl-3,4-dihydro-1,5-benzoxazepin-5(2H)-yl]acetate (70)

To a stirred solution of methyl [(2R,3S)-3-amino-4-oxo-2-phenyl-3,4-dihydro-1,5-benzoxazepin-5(2H)-yl]acetate (70b) (130 mg, 0.398 mmol) in DCM (4 mL) was added N-[(3,5-difluorophenyl)acetyl]-L-alanine (1e) (125 mg, 0.514 mmol), HOBt (71 mg, 0.525 mmol), NMM (53 mg, 0.525 mmol) and EDAC-HCl (100 mg, 0.522 mmol). After stirring at ambient temperature under nitrogen for 48 h the solvent was evaporated and the residue partitioned between EtOAc and saturated aqueous sodium bicarbonate. The organic solution was separated, washed with 1N aqueous HCl then dried, filtered and evaporated. The residue was purified by flash chromatography on silica gel eluting with 1:1 (v/v) hexane-EtOAc to afford the title compound (185 mg, 82%) as a white solid. TLC R_{f}=0.13 (1:1 hexane-EtOAc). ¹H NMR (300 MHz, CDCl₃) δ1.21 (d, 3H, J=7.0 Hz), 3.46 (s, 2H), 3.80 (s, 3H), 4.23 (m, 1H), 4.55 and 4.68 (AB quartet, 2H, J=17.1 Hz), 5.16 (t, 1H, J=7.2 Hz), 5.72 (d, 1H, J=7.4 Hz), 5.87 (br d, 1H, J=7.0 Hz), 6.17 (br d, 1H, J=6.6 Hz), 6.66-6.87 (m, 3H), 7.19-7.33 (m, 4H), 7.37 (s, 5H). MS APCI, m/z=552 (M+1). LC/MS: 2.52 min.

The precursor methyl [(2R,3S)-3-amino-4-oxo-2-phenyl-3,4-dihydro-1,5-benzoxazepin-5(2H)-yl]acetate (70b) was prepared as follows:

### a. Methyl [(2R,3S)-3-[(tert-butoxycarbonyl)amino]-4-oxo-2-phenyl-3,4-dihydro-1,5-benzoxazepin-5(2H)-yl]acetate (70a)

To a stirred solution of tert-butyl [(2R,3S)-4-oxo-2-phenyl-2,3,4,5-tetrahydro-1,5-benzoxazepin-3-yl]carbamate (67a) (150 mg, 0.423 mmol) in dry DMF (4 mL) under nitrogen was added methyl bromoacetate (97 mg, 0.634 mmol) and powdered cesium carbonate (207 mg, 0.635 mmol). The mixture was stirred for ˜12 h at ambient temperature then diluted with water and extracted with EtOAc. The residue from the organic extract was purified by flash chromatography on silica gel eluting with 4:1 (v/v) hexane-EtOAc to afford the title compound (175 mg, 97%) as a white solid. TLC R_{f}=0.20 (4:1 hexane:EtOAc). ¹H NMR (300 MHz, CDCl₃) δ1.38 (s, 9H), 3.80 (s, 3H), 4.54 and 4.69 (AB quartet, 2H, J=17.1 Hz), 4.93 (m, 1H), 5.02 (m, 1H), 5.70 (d, 1H, J=7.0 Hz), 7.19-7.29 (m, 4H), 7.35-7.48 (m, 5H). MS APCI, m/z=327 (M-BOC). LC/MS: 2.76 min.

### b. Methyl [(2R,3S)-3-amino-4-oxo-2-phenyl-3,4-dihydro-1,5-benzoxazepin-5(2H)-yl]acetate (70b)

Methyl [(2R,3S)-3-[(tert-butoxycarbonyl)amino]-4-oxo-2-phenyl-3,4-dihydro-1,5-benzoxazepin-5(2H)-yl]acetate (70a) (175 mg, 0.410 mmol) was dissolved in 5:1 (v/v) DCM-trifluoroacetic acid (4 mL) and kept at ambient temperature for 30 min. The solution was evaporated and the residue was dissolved in EtOAc and extracted with saturated aqueous sodium bicarbonate. The organic solution was dried and evaporated and the residue used immediately in the next step.

### Example 71

### [(2R,3S)-3-(N-[(3,5-Difluorophenyl)acetyl]-L-alanylamino)-4-oxo-2-Phenyl-3,4-dihydro-1,5-benzoxazepin-5(2H)-yl]acetic acid (71)

To a stirred solution of methyl [(2R,3S)-3-({N-[(3,5-difluorophenyl)acetyl]-L-alanyl}amino)-4-oxo-2-phenyl-3,4-dihydro-1,5-benzoxazepin-5(2H)-yl]acetate (70) (133 mg, 0.241 mmol) in THF (4 mL) was added a solution of lithium hydroxide (11 mg, 0.262 mmol) in water (1 mL). Several small drops of methanol were then added until a clear homogeneous solution was achieved. The mixture was stirred for 1 h at ambient temperature then acidified with 1N aqueous HCl and extracted with EtOAc. The organic solution was dried, filtered and evaporated. The glass-like solid residue was dissolved in DCM (2 mL) then precipitated by addition of hexanes to afford the title compound (124 mg, 96%) as a white solid. ¹H NMR (300 MHz, DMSO-d6) δ1.08 (d, 3H, J=7.0 Hz), 3.41 and 3.48 (AB quartet, 2H, J=14.5 Hz), 4.21 (m, 1H), 4.58 (s, 2H), 5.02 (t, 1H, J=7.0 Hz), 5.55 (d, 1H, J=7.0 Hz), 6.94 (m, 2H), 7.08 (m, 1H), 7.24-7.49 (m, 10H), 8.33 (d, 1H, J=7.0 Hz), 12.94 (br, 1H). MS APCI, m/z=538 (M+1). LC/MS: 2.27 min.

### Example 72

### N¹-[(2R,3S)-5-(Cyclopropylmethyl)-4-oxo-2-phenyl-2,3,4,5-tetrahydro-1,5-benzoxazepin-3-yl]-N²-[(3,5-difluorophenyl)acetyl]-L-alaninamide (72)

To a stirred solution of (2R,3S)-3-amino-5-(cyclopropylmethyl)-2-phenyl-2,3-dihydro-1,5-benzoxazepin-4(5H)-one (72b) (150 mg, 0.486 mmol) in DCM (4 mL) was added N-[(3,5-difluorophenyl)acetyl]-L-alanine (1e) (130 mg, 0.534 mmol), HOBt (75 mg, 0.555 mmol), NMM (60 mg, 0.594 mmol) and EDAC-HCl (103 mg, 0.537 mmol). After stirring at ambient temperature under nitrogen for ˜12 h the solvent was evaporated and the residue partitioned between EtOAc and saturated aqueous sodium bicarbonate. The organic solution was separated, washed with 1N aqueous HCl then dried, filtered and evaporated. The residue was purified by flash chromatography on silica gel eluting with 5:1 (v/v) DCM-EtOAc to afford the title compound (230 mg, 88%) as a white solid. TLC R_{f}=0.22 (5:1 DCM-EtOAc). ¹H NMR (300 MHz, CDCl₃) δ0.20 (m, 1H), 0.36-0.64 (m, 3 h), 1.14 (m, 1H), 1.21 (d, 3H, J=7.0 Hz), 3.47 (s, 2H), 3.52 (m, 1H), 4.10-4.29 (m, 2H), 5.05 (t, 1H, J=7.0 Hz), 5.68 (d, 1H, J=7.4 Hz), 5.90 (d, 1H), 6.24 (d, 1H), 6.68-6.85 (m, 3H), 7.28 (s, 4H), 7.36, s, 5H). MS APCI, m/z=534 (M+1). LC/MS: 2.78 min.

The precursor (2R,3S)-3-amino-5-(cyclopropylmethyl)-2-phenyl-2,3-dihydro-1,5-benzoxazepin-4(5H)-one (72b) was prepared as follows:

### a. tert-Butyl [(2R,3S)-5-(cyclopropylmethyl)-4-oxo-2-Phenyl-2,3,4,5-tetrahydro-1,5-benzoxazepin-3-yl]carbamate (72a)

To a stirred solution of tert-butyl [(2R,3S)-4-oxo-2-phenyl-2,3,4,5-tetrahydro-1,5-benzoxazepin-3-yl]carbamate (67a) (190 mg, 0.536 mmol) in dry DMF (2 mL) under nitrogen was added (bromomethyl)cyclopropane (108 mg, 0.800 mmol) and powdered cesium carbonate (264 mg, 0.810 mmol). The mixture was stirred for ˜12 h at ambient temperature then diluted with water and extracted with EtOAc. The residue from the combined organic extracts was purified by flash chromatography on silica gel eluting with 6:1 (v/v) hexane-EtOAc to afford the title compound (201 mg, 92%) as a white solid. TLC R_{f}=0.31 (6:1 hexane-EtOAc). ¹H NMR (300 MHz, CDCl₃) δ0.19 (m, 1H), 0.35-0.62 (m, 3H), 1.15 (m, 1H), 1.37 (s, 9H), 3.50 and 3.55 (dd, 1H, J=7.0 Hz), 4.12 and 4.17 (dd, 1H, J=7.0 Hz), 4.93 (m, 2H), 5.65 (d, 1H, J=7.0 Hz), 7.29 (m, 4H), 7.41 (m, 5H). MS APCI, m/z=309 (M-BOC). LC/MS: 3.08 min.

### b. (2R,3S)-3-Amino-5-(cyclopropylmethyl)-2-phenyl-2,3-dihydro-1,5-benzoxazepin-4(5H)-one (72b)

tert-Butyl [(2R,3S)-5-(cyclopropylmethyl)-4-oxo-2-phenyl-2,3,4,5-tetrahydro-1,5-benzoxazepin-3-yl]carbamate (72a) (201 mg, 0.492 mmol) was dissolved in 5:1 (v/v) DCM-trifluoroacetic acid (5 mL) and kept at ambient temperature for 1 h. The solution was evaporated and the residue was dissolved in EtOAc and extracted with saturated aqueous sodium bicarbonate. The organic solution was dried and evaporated and the residue used immediately in the next step. MS APCI, m/z=309 (M+1). LC/MS: 1.82 min.

### Example 73

### N¹-[(2R,3S)-5-(Cyclopropylmethyl)-7-methoxy-4-oxo-2-phenyl-2,3,4,5-tetrahydro-1,5-benzoxazepin-3-yl]-N²-[(3,5-difluorophenyl)acetyl]-L-alaninamide (73)

To a stirred solution of N²-[(3,5-difluorophenyl)acetyl]-N¹-[(2,3-cis)-7-methoxy-4-oxo-2-phenyl-2,3,4,5-tetrahydro-1,5-benzoxazepin-3-yl]-L-alaninamide (68) (95 mg, 0.186 mmol) in DMF (1 mL) under nitrogen was added (bromomethyl)cyclopropane (70 mg, 0.518 mmol) and powdered cesium carbonate (170 mg, 0.521 mmol). The mixture was stirred for ˜12 h at ambient temperature then diluted with water and extracted with EtOAc. The residue obtained from the organic extract was purified by flash chromatography on silica gel eluting with diethyl ether to afford the title compound in a 1:1 mixture with the 2S,3R diastereomer (65 mg, 62%) as a white solid. TLC R_{f}=0.26 (diethyl ether). ¹H NMR (300 MHz, CDCl₃) δ0.23 (m, 1H), 0.37-0.65 (m, 3H), 1.07 (d 1.5H, J=7.0 Hz), 1.21 (d, 1.5H, J=7.0 Hz), 3.43 (s, 1H), 3.47 (s, 1H), 3.83 (s, 3H), 4.09 (m, 1H), 4.25 (m, 1H), 5.05 (m, 1H), 5.61 (m, 1H), 5.89 (br d, 1H), 5.99 (br d, 1H), 6.20 (br t, 1H), 6.66-6.85 (m, 5H) 7.19 (d, 1H, 8.3 Hz), 7.30-7.42 (m, 6H).MS APCI, m/z=564 (M+1). LC/MS: 2.77 min.

### Example 74

### N¹-[(2R,3S)-5-(2-Azetidin-1-yl-2-oxoethyl)-4-oxo-2-phenyl-2,3,4,5-tetrahydro-1,5-benzoxazepin-3-yl]-N²-[(3,5-difluorophenyl)acetyl]-L-alaninamide (74)

To a stirred solution of [(2R,3S)-3-(N-[(3,5-difluorophenyl)acetyl]-L-alanylamino)-4-oxo-2-phenyl-3,4-dihydro-1,5-benzoxazepin-5(2H)-yl]acetic acid (71) (56 mg, 0.098 mmol) in DCM (2 mL) under nitrogen was added HOBt (20 mg, 0.148 mmol), NMM (21 mg, 0.207 mmol), azetidine (20 mg, 0.351 mmol) and EDAC-HCl (29 mg, 0.151 mmol). The mixture was stirred at ambient temperature for 48 h. The reaction was diluted with EtOAc and extracted in succession with saturated aqueous sodium bicarbonate and 1N aqueous HCl. The residue obtained from the organic solution was purified by recrystallization from EtOAc-hexanes to afford the title compound (46 mg, 81%) as a white solid. TLC R_{f}=0.09 (EtOAc). ¹H NMR (300 MHz, CDCl₃) δ1.21 (d, 3H, J=7.0 Hz), 2.36 (m, 2H), 3.46 (s, 2H), 4.02-4.45 (m, 5H), 4.38 (m, 1H), 4.71 (d, 1H, J=16.2 Hz), 5.14 (t, 1H, J=7.2 Hz), 5.74 (d, 1H, J=7.0 Hz), 5.96 (br d, 1H, J=7.0 Hz), 6.21 (br d, 1H, J=7.0 Hz), 6.67-6.85 (m, 3H), 7.22-7.51 (m, 9H). MS APCI, m/z=577 (M+1). LC/MS: 2.30 min.

### Example 75

### N²-[(3,5-Difluorophenyl)acetyl]-N¹-[(2R,3S)-7-fluoro-4-oxo-2-phenyl-2,3,4,5-tetrahydro-1,5-benzoxazepin-3-yl]-L-alaninamide (75)

To a solution of (2,3-cis)-3-amino-7-fluoro-2-phenyl-2,3-dihydro-1,5-benzoxazepin-4(5H)-one (75h) (75 mg), N-[(3,5-difluorophenyl)acetyl]-L-alanine, HOBt (56 mg), and NMM (28 mg) in DCM (8 ml) was added EDAC-HCl (79 mg). The mixture was stirred at 25° C. under nitrogen for 2 h. The reaction was diluted with 1 N hydrochloric acid and extracted with DCM. The organic extracts were combined and washed with 1 N potassium carbonate. The organic layer was dried, filtered and evaporated. The crude product was purified by flash chromatography (2% methanol-DCM) to afford the off-white solid title compound (110 mg) as a 1:1 mixture with the 2S,3R diastereoruer. ¹H NMR (300 MHz, d6-DMSO) δ 1.03-1.12 (m, 3H), 3.41-3.45 (m, 2H), 4.21-4.35 (m, 1H), 5.58-5.62 (m, 1H), 6.91-7.11 (m, 3H), 7.28-7.50 (m, 9H), 8.22-8.33 (m, 1H), 10.38-10.42 (m, 1H). MS APCI, m/z=498 (M+1). LC/MS: 2.38 min.

The starting (2,3-cis)-3-amino-7-fluoro-2-phenyl-2,3-dihydro-1,5-benzoxazepin-4(5H)-one (75h) was prepared in the following manner:

### a. Ethyl (2RS,3RS)-3-(4-fluoro-2-nitrophenoxy)-2-hydroxy-3-phenylpropanoate (75a)

The title compound was prepared according to the published procedure of Carlo Banzatti, Franco Heidempergher, and Piero Melloni; J. Heterocyclic Chem. 20, 259 (1983).

### b. Ethyl (2SR,3RS)-2-azido-3-(4-fluoro-2-nitrophenoxy)-3-phenylpropanoate (75b)

To a solution of ethyl (2RS,3RS)-3-(4-fluoro-2-nitrophenoxy)-2-hydroxy-3-phenylpropanoate (75a) (3.00 g) and trifluoromethane sulfonic anhydride (3.63 g) in DCM (125 mL) was added via syringe 2,6-lutidine under nitrogen at 0° C. The mixture was kept at 0° C. for 1 h. Additional trifluoromethane sulfonic anhydride (987 mg) and 2,6-lutidine (375 mg) was added and the mixture kept at 0° C. for 30 min. The reaction was concentrated in vacuo without heating, and the resulting residue immediately dissolved in DMF (90 ml). Sodium azide (838 mg) was added to the solution and the mixture was heated to 45° C. for ˜12 h. The DMF was removed in vacuo with heating. The residue was purified by flash chromatography (10:1 hexane:Ethyl acetate) to afford the title compound (2.82 g). ¹H NMR (300 MHz, d6-DMSO) δ 1.09 (t, 3H, J=7 Hz), 4.16 (m, 2H), 4.73 (d, 1H, J=4 Hz), 6.16 (d, 1H, 4 Hz), 7.20-7.48 (m, 7H), 7.87 (dd, 1H, J=3 Hz, J=8 Hz). MS APCI, m/z=347 (M+1−N²) LC/MS: 2.81 min.

### c. Ethyl (2SR,3RS)-2-amino-3-(4-fluoro-2-nitrophenoxy)-3-phenylpropanoate (75c)

To a solution of ethyl (2SR,3RS)-2-azido-3-(4-fluoro-2-nitrophenoxy)-3-phenylpropanoate (75b) (2.60 g) and triphenylphosphine (2.01 g) in THF (100 ml) was added water (2 mL) and the mixture was refluxed for 1 h then stiffed at 25° C. for ˜12 h. The reaction was concentrated in vacuo and the resulting residue was purified by flash chromatography (5:2 hexane:EtOAc) to afford the title compound (1.55 g) as a yellow oil. ¹H NMR (300 MHz, d6-DMSO) δ 1.01 (t, 3H, J=7 Hz), 1.85 (s, 2H), 3.76 (d, 1H, J=5 Hz), 3.99 (q, 2H, J=7 Hz), 5.75 (d, 1H, J=5 Hz), 6.89-7.44 (m, 7H), 7.85 (dd, 1H, J=3 Hz, J=9 Hz). MS APCI, m/z=349 (M+1). LC/MS: 1.82 min.

### d. Ethyl (2SR,3RS)—N-(tert-butoxycarbonyl)-3-(4-fluoro-2-nitrophenoxy)-3-phenylpropanoate (75d)

To a solution of ethyl (2SR,3RS)-2-amino-3-(4-fluoro-2-nitrophenoxy)-3-phenylpropanoate (75c) (1.72 g) in THF (55 ml) was added di-tert-butyl dicarbonate (1.19 g) in THF (12 ml) and the mixture was stirred at 25° C. for 70 h. The reaction was concentrated in vacuo and the resulting residue was purified by flash chromatography (5:1 hexane-EtOAc) to afford the title compound (2.21 g). ¹H NMR (300 MHz, d6-DMSO) δ 0.95 (t, 3H, J=7 Hz), 1.32 (s, 9H), 3.95 (q, 2H, J=7 Hz), 4.53 (m, 1H), 5.86 (d, 1H, J=5.85 Hz), 7.09-7.20, (m, 2H), 7.31-7.43 (m, 6H), 7.83 (dd, 1H, J=3 Hz, J=8 Hz). MS APCI, m/z=349 (M+1-Boc). LC/MS: 2.96 min.

### e. Ethyl (2SR,3RS)-3-(2-amino-4-fluorophenoxy)-N-(tert-butoxycarbonyl)-3-phenylpropanoate (75e)

To a solution of ethyl (2SR,3RS)—N-(tert-butoxycarbonyl)-3-(4-fluoro-2-nitrophenoxy)-3-phenylpropanoate (75d) (2.16 g) in ethanol (80 ml) was added 5% palladium on carbon (500 mg) and the mixture hydrogenated on a Parr apparatus at 35 psi. The mixture was filtered through diatomaceous earth and the filtrate was evaporated and purified by flash chromatography (10:3 hexane:EtOAc) to afford the title compound (1.55 g) as a tan solid. ¹H NMR (300 MHz, d6-DMSO) δ 1.07-1.13 (m, 3H), 1.27 (s, 9H), 4.06-4.16 (m, 2H), 4.54-4.61 (m, 1H), 5.75-6.04 (m, 1H), 6.24-6.32 (m, 0.5H), 6.46-6.54 (m, 1H), 6.63-6.67 (m, 0.5H), 7.22-7.49 (m, 6H), 7.82 (d, 1H, J=10 Hz), 8.53 (s, 0.5H), 8.81 (s, 0.5H). MS APCI m/z=418 (M+1). LC/MS: 2.74 min.

### f. (2SR,3RS)-3-(2-Amino-4-fluorophenoxy)-N-(tert-butoxycarbonyl)-3-phenylpropanoic acid (75f)

To a solution of ethyl (2SR,3RS)-3-(2-amino-4-fluorophenoxy)-N-(tert-butoxycarbonyl)-3-phenylpropanoate (75e) (1.52 g) in THF (50 mL) was added a solution of lithium hydroxide (168 mg) in water (25 ml) under nitrogen at 0° C. The mixture was stirred at 0° C. for 1.5H, then at 25° C. for 1H. The reaction was diluted with water and extracted with EtOAc. To the aqueous layer was added 1 N HCl until the pH=1 and this mixture was extracted with Ethyl acetate. The EtOAc layers were combined, washed with brine, dried, filtered and evacuated to give the title compound (1.68 g) as a crude tacky solid. No further analysis or purification was performed.

### a. tert-Butyl [(2,3-cis)-7-fluoro-4-oxo-2-phenyl-2,3,4,5-tetrahydro-1,5-benzoxazepin-3-yl]carbamate (75g)

To a solution of crude (2SR,3RS)-3-(2-amino-4-fluorophenoxy)-N-(tert-butoxycarbonyl)-3-phenylpropanoic acid (75f) (580 mg), HOBt (251 mg), and NMM (150 mg) in DCM (60 ml) was added EDAC.HCl (353 mg). The mixture was stirred at 25° C. for 2 h. The reaction was then evaporated and purified with flash chromatography (5:1 hexane-EtOAc) to give the title compound (242 mg) as an off white solid. ¹H NMR (300 MHz, d6-DMSO) δ 1.32 (s, 9H), 4.74 (t, 1H, J=7 Hz), 5.33 (d, 1H, J=8 HzO, 5.64 (d, 1H, J=6 Hz), 6.94-7.06 (m, 2H), 7.26-7.45 (m, 5H), 10.39 (s, 1H). MS APCI m/z=373 (M+1) LC/MS: 2.63 min.

### h. (2,3-cis)-3-Amino-7-fluoro-2-phenyl-2,3-dihydro-1,5-benzoxazepin-4(5H)-one (75h)

To a solution of tert-butyl [(2,3-cis)-7-fluoro-4-oxo-2-phenyl-2,3,4,5-tetrahydro-1,5-benzoxazepin-3-yl]carbamate (75g) in DCM was added trifluoroacetic acid (5 ml). The mixture was stirred at 25° C. for 1 h. The solvent was removed in vacuo and the residue dissolved in DCM. The solution was washed with 1 N potassium chloride, dried, filtered, and evaporated to afford the title compound (362 mg) as a yellow solid. ¹H NMR (300 MHz, d6-DMSO) δ 1.46 (s, 2H), 3.81 (d, 1H, J=6 Hz), 5.45 (d, 1H, J=7 Hz), 6.88-7.01 (m, 2H), 7.19-7.25 (m, 1H), 7.35-7.42 (m, 5H), 10.07 (s, 1H). MS APCI m/z=273 (M+1) LC/MS:1.50 min.

### Example 76

### (2S)—N-((1S)-2-[(2R,3S)-7-Fluoro-4-oxo-2-phenyl-2,3,4,5-tetrahydro-1,5-benzoxazepin-3-yl]amino-2-oxo-1-phenylethyl)-2-hydroxy-4-methylpentanamide (76)

To a solution of (2,3-cis)-3-amino-7-fluoro-2-phenyl-2,3-dihydro-1,5-benzoxazepin-4(5H)-one (75h) (75 mg), (2S)-{[(2S)-2-hydroxy-4-methylpentanoyl]amino}(phenyl)acetic acid (76b) (73 mg), HOBt (56 mg), and NMM (28 mg) in DCM (8 ml) was added EDAC-HCl (79 mg). The mixture was stirred at 25° C. under nitrogen for 2 h. The reaction was diluted with 1 N hydrochloric acid and extracted with DCM. The organic extracts were combined and washed with 1 N potassium carbonate. This organic layer was dried, filtered and evaporated. The crude product was purified by flash chromatography (2% methanol-DCM) to afford the off-white solid title compound (110 mg) as a 1:1 mixture with the 2S,3R diastereomer. ¹H NMR (300 MHz, d6-DMSO) δ 0.81-0.94 (m, 8H), 1.27-1.46 (m, 1H), 1.56-1.80 (m, 1H), 3.84-3.95 (m, 1H), 4.97-5.15 (m, 1H), 5.33-5.35 (m, 1H), 5.47-5.75 (m, 3H), 6.89-7.06 (m, 4H), 7.12-7.43 (m, 8H), 7.97-8.22 (m, 2H), 10.32-10.39 (m, 1H). MS APCI, m/z=520 (M+1). LC/MS: 2.51 min.

The (2S)-{[(2S)-2-hydroxy-4-methylpentanoyl]amino}(phenyl)acetic acid (76b) was prepared in the following manner:

### a. Methyl (2S)-{[(2S)-2-hydroxy-4-methylpentanoyl]amino}(phenyl)acetate (76a)

To a cooled (0° C.) solution of methyl (2S)-amino(phenyl)acetate (3.06 g), (2S)-2-hydroxy-4-methylpentanoic acid hydrochloride, NMM (1.85 mL), and HOBt (6.15 g) in DCM (50 ml) was added EDAC-HCl (5.81 g). Additional NMM (2.31 ml) was then added. The mixture was stirred for 2 h at 0° C., then for ˜12 h at 25° C. The reaction was concentrated and the residue was taken up in Ethyl acetate washed with 0.1N hydrochloric acid, saturated sodium bicarbonate, brine, dried, filtered and evaporated to afford the title compound (4.20 g). ¹H NMR (300 MHz, CDCl₃) δ 0.92-0.95 (m, 6H), 1.47-1.67 (m, 2H), 1.76-2.04 (m, 1H), 2.63 (bs, 1H), 3.74 (s, 3H), 4.21 (d, 1H, J=9 Hz), 5.58 (d, 1H, J=8 Hz), 7.26-7.42 (m, 5H). MS APCI, m/z=280 (M+1). LC/MS: 1.95 min.

### b. (2S)-{[(2S)-2-Hydroxy-4-methylpentanoyl]amino}(phenyl)acetic acid (76b)

To a solution of methyl (2S)-{[(2S)-2-hydroxy-4-methylpentanoyl]amino}(phenyl)acetate (76a) (4.185 g) in THF (30 mL) and water (15 ml) cooled to 0° C. was added lithium hydroxide (1.45 g) portion wise. The mixture was stirred at 25° C. for ˜12 h. The reaction was acidified with 1N hydrochloric acid until pH=1. The mixture was concentrated to remove THF and extracted with EtOAc. The organic layers were washed with water, brine, dried, filtered and evaporated to afford the title compound as a tacky white solid. Trituration with DCM-diethyl ether yielded a free flowing solid (3.85 g). ¹H NMR (300 MHz, CDCl₃) δ 0.89-0.94 (m, 6H), 1.45-1.63 (m, 2H), 1.76-1.83 (m, 1H), 3.25 (bs, 2H), 4.20-4.25 (m, 1H), 5.56 (d, 1H, J=7 Hz), 7.35-7.39 (m, 5H), 7.53 (d, 1H, J=7 Hz). MS APCI, m/z=266 (M+1). LC/MS: 1.68 min.

### Example 77

### N²-[(2R)-2-(3,5-Difluorophenyl)-2-hydroxyacetyl]-N¹-[(2R,3S)-4-oxo-2-phenyl-2,3,4,5-tetrahydro-1,5-benzoxazepin-3-yl]-L-alaninamide (77)

To a solution of (2,3-cis)-3-amino-2-phenyl-2,3-dihydro-1,5-benzoxazepin-4(5H)-one hydrochloride (6e) (54 mg, 0.185 mmol) in DCM (10 mL) under nitrogen was added N-[((2R)-2-(3,5-difluorophenyl)-2-hydroxylacetyl]-L-alaninate acid (77d) (48 mg, 0.185 mmol), HOBt (32 mg, 0.237 mmol), triethylamine (75 μL, 0.538 mmol) and EDAC-HCl (50 mg, 0.261 mmol). The mixture was stirred for ˜12 h at ambient temperature, diluted with DCM (40 mL), then extracted with 1N aqueous HCl, 20% K₂CO₃, and brine. The organic solution was dried (MgSO₄), filtered and evaporated. The residue was purified by flash chromatography on silica gel eluting with DCM, then 100:1 and 50:1 (v/v) DCM-methanol to afford the title compound as a 1:1 mixture with the 2S,3R diastereomer (40 mg, 43%) as a white solid. ¹H NMR (300 MHz, DMSO-d₆) δ 1.00-1.20 (m, 3H), 4.25-4.42 (m, 1H), 4.90-5.07 (m, 2H), 5.59 (s, 0.5H), 5.61 (s, 0.5H), 6.44 (d, J=5 Hz, 0.5H), 6.47 (d, J=5 Hz, 0.5H), 7.00-7.45 (m, 12H), 7.61-7.78 (m, 1H), 7.92-8.15 (m, 1H), 10.27 (s, 0.5H), 10.31 (s, 0.5H). MS APCI, m/z=496 (M+1). LC/MS: 2.12 min.

The requisite acids were prepared as follows:

### a. Methyl N-[((2S)-2-(3,5-difluorophenyl)-2-hydroxylacetyl]-L-alaninate (77a)

### b. Methyl N-[((2R)-2-(3,5-difluorophenyl)-2-hydroxylacetyl]-L-alaninate (77b)

To a solution of 3,5-difluoromandelic acid (2.50 g, 13.3 mmol) in DCM (60 mL) under nitrogen was added L-alanine methyl ester hydrochloride (1.86 g, 13.3 mmol), HOBt (3.6 g, 26.6 mmol), EDAC-HCl (4.2 g, 21.9 mmol) and NMM (3.84 mL, 34.9 mmol). The mixture was stirred for ˜12 h at ambient temperature then evaporated. The residue was dissolved in EtOAc and extracted in succession with saturated sodium bicarbonate, 1N aqueous HCl, and brine. The organic solvent was dried, filtered and evaporated. The residue was purified by flash chromatography on silica gel eluting with 100:1 (v/v) CHCl₃-methanol to afford the title compound as a 1:1 mixture of diastereomers (2.0 g, 56%) as a white solid. The diastereomers was separated using the chiral HPLC at the following conditions: elute (hexane-isopropanol)=90:10, Column Chiralpak AD

Methyl N-[((2S)-2-(3,5-difluorophenyl)-2-hydroxylacetyl]-L-alaninate (77a) as a white solid. ¹H NMR (300 MHz, CD₃OD) δ 1.23 (d, 3H), 3.64 (s, 3H), 4.43 (q, 1H), 5.33 (d, 1H), 6.85 (m, 3H). MS m/z=274 (M+1). LC/MS: 1.48 min.

Methyl N-[((2R)-2-(3,5-difluorophenyl)-2-hydroxylacetyl]-L-alaninate (77b) as a white solid. ¹H NMR (300 MHz, CD₃OD) δ 1.23 (d, 3H), 3.64 (s, 3H), 4.43 (q, 1H), 5.33 (d, 1H), 6.85 (m, 3H). MS m/z=274 (M+1). LC/MS: 1.40 min

### c. N-[((2S)-2-(3,5-Difluorophenyl)-2-hydroxylacetyl]-L-alaninate (77c)

To a solution of methyl N-[((2S)-2-(3,5-difluorophenyl)-2-hydroxylacetyl]-L-alaninate (77a) (500 mg, 1.93 mmol) in DCM (5 mL) was added dropwise a solution of LiOH (100 mg, 4.18 mmol) in water (4 mL). The mixture was stirred for ˜12 h under nitrogen and the solution was acidified with 1N HCl until pH ˜1. The mixture was extracted with EtOAc. The organic phase was washed with brine, dried, filtered and evaporated. The title compound was obtained as a white solid (470 mg, 99%). ¹H NMR (300 MHz, CD₃OD) δ 1.37 (d, 3H), 4.63 (q, 1H), 5.33 (d, 1H), 6.85 (m, 3H). MS m/z=260 (M+1). LC/MS: 1.20 min.

### d. N-[(2R)-2-(3,5-Difluorophenyl)-2-hydroxylacetyl]-L-alaninate (77d)

To a solution of methyl N-[((2R)-2-(3,5-difluorophenyl)-2-hydroxylacetyl]-L-alaninate (77b) (500 mg, 1.93 mmol) in DCM (5 mL) was added dropwise a solution of LiOH (100 mg, 4.18 mmol) in water (4 mL). The mixture was stirred for ˜12 h under nitrogen and the solution was acidified with 1N HCl until pH ˜1. The mixture was extracted with EtOAc. The organic phase was washed with brine and dried, filtered and evaporated. The title compound was obtained as a white solid (470 mg, 99%). ¹H NMR (300 MHz, CD₃OD) δ 1.37 (d, 3H), 4.63 (q, 1H), 5.33 (d, 1H), 6.85 (m, 3H). MS m/z=260 (M+1). LC/MS: 1.21 min.

### Example 78

### N²-[(2S)-2-(3,5-Difluorophenyl)-2-hydroxyacetyl]-N¹-[(2R,3S)-4-oxo-2-phenyl-2,3,4,5-tetrahydro-1,5-benzoxazepin-3-yl]-L-alaninamide (78)

To a solution of (2,3-cis)-3-amino-2-phenyl-2,3-dihydro-1,5-benzoxazepin-4(5H)-one hydrochloride (6e) (76 mg, 0.263 mmol) in DCM (10 mL) under nitrogen was added N-[((2S)-2-(3,5-difluorophenyl)-2-hydroxylacetyl]-L-alaninate acid (77c) (68 mg, 0.263 mmol), HOBt (45 mg, 0.333 mmol), triethylamine (100 μL, 0.717 mmol) and EDAC-HCl (71 mg, 0.370 mmol). The mixture was stirred for ˜12 h at ambient temperature, diluted with DCM (40 mL), then extracted with 1N aqueous HCl, 20% K₂CO₃, and brine. The organic solution was dried (MgSO₄), filtered and evaporated. The residue was purified by flash chromatography on silica gel eluting with DCM, then 100:1 to 50:1 (v/v) DCM-methanol to afford the title compound in a 1:1 mixture with the 2S,3R diastereomer (60 mg, 46%) as a white solid. ¹H NMR (300 MHz, DMSO-d₆) δ 1.00-1.20 (m, 3H), 4.25-4.42 (m, 1H), 4.90-5.07 (m, 2H), 5.59 (s, 0.5H), 5.61 (s, 0.5H), 6.44 (d, J=5 Hz, 0.5H), 6.48 (d, J=5 Hz, 0.5H), 7.00-7.45 (m, 12H), 7.61-7.78 (m, 1H), 7.92-8.15 (m, 1H), 10.27 (s, 0.5H), 10.31 (s, 0.5H). MS APCI, m/z=496 (M+1). LC/MS: 2.12 min.

### Example 79

### N²-[(3,5-Difluorophenyl)acetyl]-N¹-[(3S,4R)-2-oxo-4-phenyl-2,3,4,5-tetrahydro-1H-1-benzazepin-3-yl]-L-alaninamide (79)

To a solution of (3,4-cis)-3-amino-4-phenyl-1,3,4,5-tetrahydro-2H-1-benzazepin-2-one hydrochloride (79g) (50 mg, 0.172 mmol) in DCM (5 mL) under nitrogen was added N-[(3,5-difluorophenyl)acetyl]-L-alanine (1e) (50 mg, 0.205 mmol), HOBt (35 mg, 0.260 mmol), triethylamine (95 μL, 0.682 mmol) and EDAC-HCl (50 mg, 0.261 mmol). The mixture was stirred for ˜12 h at ambient temperature, diluted with DCM (40 mL), then extracted with 1N aqueous HCl, 20% K₂CO₃, and brine. The organic solution was dried (MgSO₄), filtered and evaporated. The residue was purified by flash chromatography on silica gel eluting with DCM, then 100:1 to 50:1 (v/v) DCM-methanol to afford the title compound as a 1:1 mixture with the 3R,4S diastereomer (50 mg, 61%) as a white solid. ¹H NMR (300 MHz, DMSO-d₆) δ 0.78 (d, J=7 Hz, 1.5H), 0.94 (d, J=7 Hz, 1.5H), 2.90-3.03 (m, 2H), 3.22-3.47 (m, 2H), 3.82-3.94 (m, 1H), 4.02 (quin, J=7 Hz, 0.5H), 4.14 (quin, J=7 Hz, 0.5H), 4.40 (t, J=8 Hz, 0.5H), 4.48 (t, J=8 Hz, 0.5H), 6.82-7.36 (m, 12H), 7.41 (d, J=7 Hz, 1H), 8.12 (d, J=8 Hz, 0.5H), 8.21 (d, J=7 Hz, 0.5H), 10.24 (s, 0.5H), 10.25 (s, 0.5H). MS APCI, m/z=478 (M+1). LC/MS: 2.40 min.

The required (3,4-cis)-3-amino-4-phenyl-1,3,4,5-tetrahydro-2H-1-benzazepin-2-one hydrochloride (79g) was prepared as follows:

### a. 1-(3,3-Diethoxy-2-phenylpropyl)-2-nitrobenzene (79a)

A suspension of sodium hydride (6.0 g, 0.15 mol, 60% oil dispersion) in DMF (375 mL) was treated with diethyl benzalmalonate (24.8 g, 0.099 mol) followed by a solution of 2-nitrotoluene (15.1 g, 0.110 mol) in DMF (25 mL) and the resulting reaction mixture was stirred for 16 h. At the end of this period the reaction mixture was treated with a solution of acetic acid (20 mL) in methanol (50 mL), followed by water (1 L) and extracted with EtOAc. The organic layer was washed with 1N HCl, sodium bicarbonate solution, brine, dried over anhydrous magnesium sulfate and concentrated under reduced pressure. The material thus obtained was purified by column chromatography over silica gel. Elution with 9:1 hexane-EtOAc afforded the title compound (11.74 g). ¹H NMR (300 MHz, CDCl₃) δ 0.91 (t, 3H, J=7 Hz), 1.30 (t, 3H, J=7 Hz), 3.27 (dd, 1H, J=10 Hz, J=14 Hz), 4.48 (dd, 1H, J=4 Hz, J=10 Hz), 3.87 (m, 3H), 4.23 (q, 1H, J=7 Hz), 6.99-7.60 (m, 8H), 7.74 (d, 1H, J=1 Hz).

### b. [2-(3,3-Diethoxy-2-phenylpropyl)phenyl]amine-acetate (79b)

A solution of 1-(3,3-diethoxy-2-phenylpropyl)-2-nitrobenzene (11.74 g, 30.5 mmol) (79a) in acetic acid (30 mL) was hydrogenated under hydrogen atmosphere (40 psi) in the presence of Pd/C (100 mg) for 16 h. At the end of this period the reaction mixture was filtered through a pad of diatomaceous earth and the pad was washed with acetic acid. Upon concentration under reduced pressure the title compound was obtained (11.36 g). ¹H NMR (300 MHz, CDCl₃) δ 0.86 (t, 3H, J=10 Hz), 1.30 (t, 3H, J=7 Hz), 2.27 (dd, 1H, J=11 Hz, J=14 Hz), 3.08 (dd, 1H, J=3 Hz, J=14 Hz), 3.62 (dt, 1H, J=11, 15 Hz), 3.84 (m, 3H), 4.27 (q, 1H, J=7 Hz), 6.36 (m, 2H), 6.61 (d, 1H, J=8 Hz), 6.87-7.26 (m, 5H). MS APCI, m/z=356 (M+1). LC/MS: 2.11 min.

### c. Ethyl 2-oxo-4-phenyl-2,3,4,5-tetrahydro-1H-1-benzazepine-3-carboxylate (79c)

A solution of [2-(3,3-diethoxy-2-phenylpropyl)phenyl]amine-acetate (11.36 g, 31.9 mmol) (79b) was dissolved in DCM (100 mL), washed with solution of sodium carbonate (2×50 mL) and concentrated under reduced pressure upon drying over anhydrous potassium carbonate. The resulting product was dissolved in o-xylene (150 mL), treated with pTSA monohydrate (190 mg, 1.02 mmol) and heated to reflux for 3 h. At the end of this period the reaction mixture was allowed to cool and then cooled in ice. The solid precipitate was filtered to afford the title compound (6.9 g): ¹H NMR (300 MHz, CDCl₃) δ 1.04 (t, 3H, J=7 Hz), 2.81 (dd, 1H, J=4 Hz, J=14 Hz), 3.58 (dd, 1H, J=7 Hz, 14 Hz), 3.76 (d, 1H, J=9 Hz), 4.00 (m, 3H), 4.21 (m, 1H), 6.91-7.33 (m, 9H), 7.58 (s, 1H). MS APCI, m/z=310 (M+1). LC/MS: 2.21 min.

### d. 4(RS)-Phenyl-1,3,4,5-tetrahydro-2H-1-benzazepin-2-one (79d)

A stirred slurry of ethyl 2-oxo-4-phenyl-2,3,4,5-tetrahydro-1H-1-benzazepine-3-carboxylate (79c) (5.14 g, 16.6 mmol), 4-aminothiophenol (4.49 g, 35.9 mmol) and LiBr (8.66 g, 99.7 mmol) in 50 mL N,N′-dimethylformamide was heated to 160° C. for 24 h. The cooled mixture was diluted with 200 mL 1N HCl and extracted with EtOAc (3×100 mL). The combined extracts were washed with 1N HCl, 20% K₂CO₃, and brine. The organic solution was dried (MgSO₄), filtered, and evaporated. The crude product was triturated (EtOH/EtOAc) to give a white powder (2.96 g, 75%). ¹H NMR (300 MHz, CDCl₃) δ 2.55-2.72 (m, 2H), 2.90 (dd, J=7 Hz, J=14 Hz, 1H), 3.19 (dd, J=7 Hz, J=14 Hz, 1H), 3.70 (quin, J=7 Hz, 1H), 7.03 (d, J=7 Hz, 1H), 7.10-7.40 (m, 8H), 7.71 (br s, 1H). MS APCI, m/z=238 (M+1). LC/MS: 2.24 min.

### e. (3,4-trans)-3-Iodo-4-phenyl-1,3,45-tetrahydro-2H-1-benzazepin-2-one (79e)

To a stirred solution of 4(RS)-phenyl-1,3,4,5-tetrahydro-2H-1-benzazepin-2-one (7d) (3.54 g, 14.9 mmol) and triethylamine (10.5 mL, 75.0 mmol) in 80 mL DCM cooled to −20° C. was slowly added iodotrimethylsilane (6.0 g, 30 mmol). When addition was complete, the mixture was stirred for 10 min, and then iodine was added (7.61 g, 30.0 mmol) all in one portion. The mixture was stirred for 1.5 h at −20° C., diluted with 800 mL DCM, and extracted with 10% aqueous sodium bisulfite, water, and brine. The organic solution was dried (MgSO₄), filtered, and evaporated to give a light yellow solid (4.07 g, 75%). ¹H NMR (300 MHz, DMSO-d₆) δ 2.75 (dd, J=5 Hz, J=14 Hz, 1H), 3.20 (dd, J=7 Hz, J=14 Hz, 1H), 3.70 (dd, J=7 Hz, J=14 Hz, 1H), 4.57 (d, J=8 Hz, 1H), 7.00-7.20 (m, 5H), 7.21-7.42 (m, 4H), 10.07 (br s, 1H). MS APCI, m/z=364 (M+1). LC/MS: 2.49 min.

### f. (3,4-cis)-3-Azido-4-phenyl-1,3,4,5-tetrahydro-2H-1-benzazepin-2-one (79f)

To a stirred solution of (3,4-trans)-3-iodo-4-phenyl-1,3,4,5-tetrahydro-2H-1-benzazepin-2-one (79e) (4.07 g, 11.2 mmol) in DMF (200 mL) was added sodium azide (5.6 g, 86 mmol). The mixture was stirred for 18 h, the DMF was removed under reduced pressure, and residue was dissolved in 500 mL CHCl₃. The solution was extracted with water, saturated aqueous sodium bicarbonate, and brine. The organic solution was dried (MgSO₄), filtered, and evaporated to give 3.24 g (quantitative) of an off-white solid. ¹H NMR (300 MHz, CDCl₃) δ 2.99 (dd, J=7 Hz, J=14 Hz, 1H), 3.22 (t, J=13 Hz, 1H), 3.81 (dd, J=7 Hz, J=13 Hz, 1H), 4.18 (d, J=8 Hz, 1H), 7.04-7.09 (m, 1H), 7.15-7.43 (m, 8H), 7.93 (br s, 1H). MS APCI, m/z=251 (M+1−N₂). LC/MS: 2.40 min.

### g. (3,4-cis)-3-Amino-4-phenyl-1,3,4,5-tetrahydro-2H-1-benzazepin-2-one hydrochloride (79g)

To a Parr hydrogen flask was added 10% Pd/C (120 mg) followed by 150 mL absolute ethanol. To this was then added (3,4-cis)-3-azido-4-phenyl-1,3,4,5-tetrahydro-2H-1-benzazepin-2-one (79f) (1.70 g, 6.06 mmol). The mixture was gently warmed with a heat gun to dissolve the azide. Aqueous 1N HCl was added (20 mL), the flask was placed on the Parr® shaker, and evacuated/backfilled with hydrogen (4 cycles). The mixture was shaken under 50 psi hydrogen for 24 h, filtered through diatomaceous earth, and the ethanol was evaporated. The residue was stirred with 200 mL Et₂O for 30 min and the precipitated product was filtered off, washed with ether, and dried under vacuum for 18 h. to yield the title compound as a white powder (1.61 g, 92%) which contained residual ether. ¹H NMR (300 MHz, DMSO-d₆) δ 2.94 (t, J=13 Hz, 1H), 3.04 (dd, J=7 Hz, J=14 Hz, 1H), 3.89 (d, J=8 Hz, 1H), 3.97 (dd, J=7 Hz, J=12 Hz, 1H), 7.13 (d, J=7 Hz, 1H), 7.22 (d, J=8 Hz, 1H), 7.32-7.46 (m, 7H), 8.04 (br s, 3H), 10.60 (s, 1H). MS APCI, m/z=253 (M+1-HCl). LC/MS: 1.48 min.

### Example 80

### N²-[(3,5-Difluorophenyl)acetyl]-N¹-[(3S,4R)-8-fluoro-1-methyl-2-oxo-4-phenyl-2,3,4,5-tetrahydro-1H-1-benzazepin-3-yl]-L-alaninamide (80)

Using a procedure similar to that described in Example 1, except using (3,4-cis)-3-amino-8-fluoro-1-methyl-4-phenyl-1,3,4,5-tetrahydro-2H-1-benzazepin-2-one (80c) (51.1 mg) as the amine component, the title compound (80) was obtained in a 1:1 mixture with the 3R,4S diastereomer (37 mg, 65%) as a white solid. ¹H NMR (300 MHz, CD₃OD) δ 0.89 (d 1.5H), 0.97 (d, 1.5H), 2.90 (m, 1H), 3.47 (s, 3H), 3.52 (m, 2H), 3.80 (s, 2H), 4.67 (m, 1H), 4.79 (q, 1H), 6.76-6.72 (m, 11H). MS APCI, m/z=510 (M+1), 532 (M+Na). LC/MS: 2.54 min.

The starting amine, (3,4-cis)-3-amino-8-fluoro-1-methyl-4-phenyl-1,3,4,5-tetrahydro-2H-1-benzazepin-2-one (80c) was prepared in the following manner:

### a. tert-Butyl-[(3,4-cis)-8-fluoro-2-oxo-4-phenyl-2,3,4,5-tetrahydro-1H-1-benzazepin-3-yl]-carbamate (80a)

To a solution of (3,4-cis)-3-amino-8-fluoro-4-phenyl-1,3,4,5-tetrahydro-2H-1-benzazepin-2-one (8k) (50 mg) in THF (10 mL) at 0° C. under nitrogen was added (Boc)₂O (61 mg). After the solid was completely dissolved, triethylamine (0.044 mL) was added and the mixture was stirred at 0° C. for 1 h, then at 25° C. for 1 h. The mixture was concentrated in vacuo and then partitioned between H₂O (10 mL) and EtOAc (20 mL). The organic phase was separated and consecutively washed with 0.1 N HCl, saturated NaHCO₃, and brine, dried, filtered and evaporated to yield the title compound (68 mg, 99%) as a viscous oil. ¹H NMR (300 MHz, CD₃OD) δ 1.42 (s 9H), 3.04 (m, 1H), 3.11 (m, 2H), 3.46 (m, 2H), 6.54-7.70 (m, 8H). MS APCI, m/z=371 (M+1), 532 (M+Na). LC/MS: 2.88 min.

### b. tert-Butyl-[(3,4-cis)-8-fluoro-1-methyl-2-oxo-4-phenyl-2,3,4,5-tetrahydro-1H-1-benzazepin-3-yl]-carbamate (80b)

To a mixture of tert-butyl-[(3,4-cis)-8-fluoro-2-oxo-4-phenyl-2,3,4,5-tetrahydro-1H-1-benzazepin-3-yl]-carbamate (80a) (68.5 mg) and KOH (13.4 mg) in THF (10 mL) at 25° C. under nitrogen was added Bu₄NBr (6.0 mg). MeI (11.0 μL) was then added dropwise. The mixture was stirred at 25° C. for 18 h, concentrated in vacuo and then partitioned between H₂O (10 mL) and EtOAc (20 mL). The organic phase was washed with brine, dried, filtered and evaporated to yield the title compound (65 mg, 92%) as a tan solid. ¹H NMR (300 MHz, CD₃OD) δ 1.42 (s 9H), 3.04 (m, 1H), 3.11 (m, 2H), 3.18 (s, 3H), 3.46 (m, 2H), 6.54-7.70 (m, 8H). MS APCI, m/z=285 (M+1-Boc). LC/MS: 2.98 min.

### c. (3,4-cis)-3-Amino-8-fluoro-1-methyl-4-phenyl-1,3,4,5-tetrahydro-2H-1-benzazepin-2-one (80c)

To a solution of tert-butyl [(3,4-cis)-8-fluoro-1-methyl-2-oxo-4-phenyl-2,3,4,5-tetrahydro-1H-1-benzazepin-3-yl]carbamate (80b) (65.0 mg) in DCM (3.0 mL) at 25° C. under nitrogen was added TFA (0.2 mL). The mixture was stirred at 25° C. for 1 h. The mixture was concentrated in vacuo, redissolved in DCM (10 mL), washed with saturated NaHCO₃ (2×) and brine, dried, filtered and evaporated to yield the title compound (42.0 mg, 87.5%) as yellow gum. ¹H NMR (300 MHz, CD₃OD) δ 2.42 (d, 2H), 2.77 (m, 2H), 2.97 (m, 1H), 3.17 (m, 1H), 3.88 (m, 1H), 6.54-7.47 (m, 8H). MS APCI, m/z=285 (M+1). LC/MS: 1.60 min.

### Example 81

### (2S)—N-((1S)-2-{[(3S,4R)-8-Fluoro-2-oxo-4-phenyl-2,3,4,5-tetrahydro-1H-1-benzazepin-3-yl]amino}-2-oxo-1-phenylethyl)-2-hydro-4-methylpentanamide (81)

Using a procedure similar to that described in Example 1, except using (3,4-cis)-3-amino-8-fluoro-4-phenyl-1,3,4,5-tetrahydro-2H-1-benzazepin-2-one (8k) (27 mg) as the amine component, and (2S)-{[(2S)-2-hydroxy-4-methylpentanoyl]amino}(phenyl)acetic acid (27 mg) as the acid component, the title compound (81) was obtained as a 1:1 mixture with the 3R,4S diastereomer (45 mg, 87%) as a white solid. ¹H NMR (300 MHz, CD₃OD) δ 0.76 (m, 1H), 0.87 (m, 6H), 1.55 (m, 1H), 1.96 (m, 1H), 2.15 (m, 2H), 3.05 (m, 1H), 3.92 (m, 1H), 4.47 (m, 1H), 5.30 (s, 1H), 6.62-7.36 (m, 13H). MS APCI, m/z=518 (M+1), 540 (M+Na). LC/MS: 2.46 min.

### Example 82

### (2S)-2-Hydroxy-4-methyl-N-((1S)-2oxo-2-{[(3S,4R)-2-oxo-4-phenyl-2,3,4,5-tetrahydro-1H-1-benzazepin-3-yl]amino}-1-phenylethyl)pentanamide (82)

Using a procedure similar to that described in Example 1, except using (3,4-cis)-3-amino-4-phenyl-1,3,4,5-tetrahydro-2H-1-benzazepin-2-one (50 mg) as the amine component, and (2S)-{[(2S)-2-hydroxy-4-methylpentanoyl]amino}(phenyl)acetic acid (46 mg) as the acid component, the title compound (82) was obtained in a 1:1 mixture with the 3R,4S diastereomer (80 mg, 93%) as a white solid. ¹H NMR (300 MHz, CD₃OD) δ 0.76 (m, 1H), 0.87 (m, 6H), 1.55 (m, 1H), 1.96 (m, 1H), 2.15 (m, 2H), 3.04 (m, 1H), 3.95 (m, 1H), 4.48 (m, 1H), 5.30 (s, 1H), 6.61-7.49 (m, 14H). MS APCI, m/z=500 (M+1). LC/MS: 2.43 min.

### Example 83

### N²-[(3,5-Difluorophenyl)acetyl]-N¹-[(3S,4R)-2-oxo-4-phenyl-1-prop-2-yn-1-yl-2,3,4,5-tetrahydro-1H-1-benzazepin-3-yl]-L-alaninamide (83)

Using a procedure similar to that described in Example 1, except using (3,4-cis)-3-amino-4-phenyl-1-prop-2-yn-1-yl-1,3,4,5-tetrahydro-2H-1-benzazepin-2-one (83c) (77 mg) as the amine component, the white solid title compound (83) was obtained as a 1:1 mixture with the 3R,4S diastereomer (110 mg, 79%). ¹H NMR (400 MHz, CD₃OD) δ 0.80 (d 1.5H), 1.12 (d, 1.5H), 2.28 (d, 1H), 2.90 (m, 1H), 3.46 (m, 2H), 3.60 (s, 2H), 4.37 (m, 2H), 4.73 (q, 1H), 6.58-6.78 (m, 3H), 6.99 (m, 4H), 7.17-7.39 (m, 5H). MS APCI, m/z=516 (M+1), 538 (M+Na). LC/MS: 2.56 min.

The starting amine, (3,4-cis)-3-amino-4-phenyl-1-prop-2-yn-1-yl-1,3,4,5-tetrahydro-2H-1-benzazepin-2-one (83c), was prepared in the following manner:

### a. tert-Butyl-[(3,4-cis)-2-oxo-4-phenyl-2,3,4,5-tetrahydro-1H-1-benzazepin-3-yl]-carbamate (83a)

To a solution of (3,4-cis)-3-amino-4-phenyl-1,3,4,5-tetrahydro-2H-1-benzazepin-2-one (300 mg) in dioxane (20 mL) and H₂O (5 mL) at 0° C. under nitrogen was added (Boc)₂O (229 mg). After the solid was completely dissolved, TEA (0.42 mL) was added and the mixture was stirred at 0° C. for 1 h. The mixture was concentrated in vacuo and then partitioned between H₂O (10 mL) and Ethyl acetate (20 mL). The organic phase was separated and consecutively washed with 0.1 N HCl, saturated NaHCO₃, and brine, dried, filtered and evaporated to yield the title compound (360 mg, 98%) as a tan solid. ¹H NMR (300 MHz, CD₃OD) δ 1.42 (s 9H), 2.15 (m, 2H), 2.95 (m, 1H), 3.16 (m, 1H), 6.60-7.80 (m, 9H). MS APCI, m/z=253 (M+1-Boc). LC/MS: 2.55 min.

### b. tert-Butyl-[(3,4-cis)-2-oxo-4-phenyl-1-prop-2-yl-1-yl-2,3,4,5-tetrahydro-1H-1-benzazepin-3-yl]-carbamate (83b)

To a mixture of tert-butyl-[(3,4-cis)-2-oxo-4-phenyl-2,3,4,5-tetrahydro-1H-1-benzazepin-3-yl]-carbamate (83a) (100 mg) and Cs₂CO₃ (184 mg) in DMF (5 mL) at 25° C. under nitrogen was added propargyl bromide (35 μL, 80% wt in toluene). The mixture was stirred at 25° C. for 18 h, concentrated in vacuo and then partitioned between 0.5 N HCl (10 mL) and Ethyl acetate (20 mL). The organic phase was washed with saturated NaHCO₃, and brine, dried, filtered and evaporated to yield the title compound (105 mg, 96%) as a yellow gum. ¹H NMR (300 MHz, CD₃OD) δ 1.42 (s 9H), 2.28 (s, 1H), 3.09 (m, 1H), 3.16 (m, 1H), 3.46 (m, 2H), 4.47 (s, 2H), 7.72-7.71 (m, 9H). MS APCI, m/z=291 (M+1-Boc). LC/MS: 2.82 min.

### c. (3,4-cis)-3-Amino-4-phenyl-1-prop-2-yn-1-yl-1,3,4,5-tetrahydro-2H-1-benzazepin-2-one (83c)

To a solution of tert-butyl [(3,4-cis)-2-oxo-4-phenyl-1-prop-2-yn-1-yl-2,3,4,5-tetrahydro-1H-1-benzazepin-3-yl]carbamate (83b) (110 mg) in DCM (5 mL) at 25° C. under nitrogen was added TFA (0.3 mL). The mixture was stirring at 25° C. for 30 min. The mixture was concentrated in vacuo, redissolved in DCM (10 mL), washed with saturated NaHCO₃ (2×) and brine, dried, filtered and evaporated to yield the title compound (77 mg, 95%) as a yellow gum. ¹H NMR (300 MHz, CD₃OD) δ 2.27 (s, 1H), 2.77 (m, 2H), 2.99 (m, 1H), 3.91 (d, 1H), 4.40 (s, 2H), 7.25-7.39 (m, 9H). MS APCI, m/z=291 (M+1). LC/MS: 1.68 min.

### Example 84

### N¹-[(3S,4R)-1-(Cyclopropylmethyl)-2-oxo-4-phenyl-2,3,4,5-tetrahydro-1H-1-benzazepin-3-yl]-N²-[(3,5-difluorophenyl)acetyl]-L-alaninamide (84)

Using a procedure similar to that described in Example 1, except using (3,4-cis)-3-amino-1-(cyclopropylmethyl)-4-phenyl-1,3,4,5-tetrahydro-2H-1-benzazepin-2-one (84b) (50 mg) as the amine component, the title compound (84) was obtained in a 1:1 mixture with the 3R,4S diastereomer (72 mg, 85%) as a white solid. ¹H NMR (300 MHz, CD₃OD) δ 0.50 (m, 2H), 0.67 (m, 2H), 1.21 (m, 1H), 1.29 (d, 3H), 3.05 (m, 1H), 3.46 (d, 2H), 3.53 (m, 2H), 3.80 (s, 2H), 4.70 (q, 1H), 4.78 (d, 1H), 6.53-7.52 (m, 12H). MS APCI, m/z=532 (M+1), 554 (M+Na). LC/MS: 2.74 min.

The starting amine, (3,4-cis)-3-amino-1-(cyclopropylmethyl)-4-phenyl-1,3,4,5-tetrahydro-2H-1-benzazepin-2-one (4b), was prepared in the following manner:

### a. tert-Butyl-[(3,4-cis)-1-(cyclopropylmethyl)-2-oxo-4-phenyl-2,3,4,5-tetrahydro-1H-1-benzazepin-3-yl]-carbamate (84a)

Using a procedure similar to that described in Example 83b, except using cyclopropylmethyl bromide, the title compound (84a) was obtained as a yellow gum (81%). ¹H NMR (300 MHz, CD₃OD) δ 0.50 (m, 2H), 0.69 (m, 2H), 1.19 (m, 1H), 1.42 (s 9H), 3.09 (m, 1H), 3.16 (m, 1H), 3.46 (m, 2H), 4.47 (s, 2H), 7.72-7.71 (m, 9H). MS APCI, in/Z=307 (M+1-Boc). LC/MS: 3.01 min.

### b. (3,4-cis)-3-Amino-1-(cyclopropylmethyl)-4-phenyl-1,3,4,5-tetrahydro-2H-1-benzazepin-2-one (84b)

Using a procedure similar to that described in Example 83c, the title compound (84b) was obtained as a yellow gum (78%). ¹H NMR (300 MHz, CD₃OD) δ 0.50 (m, 2H), 0.69 (m, 2H), 1.19 (m, 1H), 3.09 (m, 1H), 3.16 (m, 1H), 3.46 (m, 2H), 4.47 (s, 2H), 7.72-7.71 (m, 9H). MS APCI, m/z=307 (M+1). LC/MS: 1.90 min.

### Example 85

### N²-[(3,5-Difluorophenyl)acetyl]-N¹-[(3S,4R)-1-isopropyl-2-oxo-4-phenyl-2,3,4,5-tetrahydro-1H-1-benzazepin-3-yl]-L-alaninamide (85)

Using a procedure similar to that described in Example 1, except using (3,4-cis)-3-amino-1-isopropyl-4-phenyl-1,3,4,5-tetrahydro-2H-1-benzazepin-2-one (85b) (47 mg) as the amine component, the white solid title compound (85) was obtained as a 1:1 mixture with the 3R,4S diastereomer (65 mg, 78%). ¹H NMR (300 MHz, CD₃OD) δ 1.17 (d, 6H), 1.29 (d, 3H), 3.16 (m, 1H), 3.50 (m, 2H), 3.80 (s, 2H), 4.70 (q, 1H), 4.88 (m, 1H), 4.95 (m, 1H), 6.58-7.56 (m, 12H). MS APCI, m/z=520 (M+1), 542 (M+Na). LC/MS: 2.71 min.

The starting amine, (3,4-cis)-3-amino-1-isopropyl-4-phenyl-1,3,4,5-tetrahydro-2H-1-benzazepin-2-one (85b), was prepared in the following manner:

### a. tert-Butyl-[(3,4-cis)-1-isopropyl-2-oxo-4-phenyl-2,3,4,5-tetrahydro-1H-1-benzazepin-3-yl]-carbamate (85a)

Using a procedure similar to that described in Example 83b, except using 2-iodopropane, the title compound (85a) was obtained as yellow gum (96%). ¹H NMR (300 MHz, CD₃OD) δ 1.17 (d, 6H), 1.42 (s 9H), 3.09 (m, 1H), 3.16 (m, 1H), 3.46 (m, 2H), 4.95 (m, 1H), 7.72-7.71 (m, 9H). MS APCI, m/z=295 (M+1-Boc). LC/MS: 2.99 min.

### b. (3,4-cis)-3-Amino-1-isopropyl-4-phenyl-1,3,4,5-tetrahydro-2H-1-benzazepin-2-one (85b)

Using a procedure similar to that described in Example 83c, the title compound (85b) was obtained as yellow gum (61%). ¹H NMR (300 MHz, CD₃OD) δ 1.17 (d, 6H), 2.83 (m, 2H), 3.12 (m, 1H), 4.08 (m, 1H), 5.02 (m, 1H), 6.58-7.65 (m, 9H). MS APCI, m/z=295 (M+1). LC/MS: 1.81 min.

### Example 86

### N²-[(2S)-2-Hydroxy-4-methyl-1-oxopentyl]-N¹-[(2R,3R)-2-(4-methoxyphenyl)-4-oxo-2,3,4,5-tetrahydro-1,5-benzothiazepin-3-yl]-L-alaninamide (86)

Using a procedure similar to that described in Example 1, except with (2,3-cis)-3-amino-2-(4-methoxyphenyl)-2,3-dihydro-1,5-benzothiazepin-4(5H)-one (41d) (70 mg, 0.184 mmol) as the amine component, N-(2S)-2-hydroxy-4-methyl-1-oxopentyl-L-alanine (37.5 mg, 0.185 mmol) as the acid component and DCM/DMF as solvent gave after aqueous workup and column chromatography (1:1 hexane-EtOAc) the solid title compound as a 1:1 mixture with the (2S,3S) diastereomer (52 mg, 58%). ¹H NMR (300 MHz, CDCl₃) δ0.93 (m, 6H), 1.10 (d, 1.5H, J=7.0 Hz), 1.22 (d, 1.5H, J=7.0 Hz), 1.4-1.65 (m, 4H), 3.81 (s, 3H), 3.97-4.03 (m, 1H), 4.24-4.36 (m, 1H), 4.84-4.91 (m, 1H), 5.20-5.26 (m, 1H), 6.43 (d, 0.5H), 6.64 (d, 1H), 6.80 (d, 0.5H), 6.88 (d, 2H, J=8.3 Hz), 7.14 (d, 1H, J=7.5 Hz), 7.24-7.28 (m, 1H), 7.36 (d, 2H, J=8.8 Hz), 7.37-7.44 (m, 1H), 7.70 (d, 1H, J=7.9 Hz), 7.74 (s, 0.5H), 7.83 (s, 0.5H). MS APCI, m/z=508(M+Na). LC/MS: 2.23 min.

### Example 87

### N¹-[(2R,3R)-2-(2-Chlorophenyl)-4-oxo-2,3,4,5-tetrahydro-1,5-benzothiazepin-3-yl]-N²-[(2S)-2-hydroxy-4-methyl-1-oxopentyl]-L-alaninamide (87)

Using a procedure similar to that described in Example 1, except with (2,3-cis)-3-amino-2-(2-chlorophenyl)-2,3-dihydro-1,5-benzothiazepin-4(5H)-one (87d) (70 mg, 0.181 mmol) as the amine component, N-(2S)-2-hydroxy-4-methyl-1-oxopentyl-L-alanine (36.7 mg, 0.181 mmol) as the acid component and DCM-DMF as solvent gave after aqueous workup and column chromatography (hexane/EtOAc gradient) the solid title compound as a 1:1 mixture with the (2S,3S) diastereomer (42 mg, 47%). ¹H NMR (300 MHz, CDCl₃) δ0.92 (m, 6H), 1.09 (d, 1.5H, J=7.0 Hz), 1.24 (d, 1.5H, J=7.0 Hz), 1.4-1.6 (m, 2H), 1.75-1.86 (m, 1H), 2.64 (t, 1H), 3.99-4.05 (m, 1H), 4.32-4.45 (m, 1H), 4.96-5.06 (m, 1H), 5.93-5.99 (m, 1H), 6.52 (d, 0.5H), 6.68 (d, 0.5H), 6.77 (d, 0.5H), 6.85 (d, 0.5H), 7.16 (d, 1H), 720-7.47 (m, 5H), 7.74 (t, 1H), 7.86-7.93 (m, 1.5H), 7.99 (s, 0.5H). MS APCI, m/z=512(M+Na). LC/MS: 2.33 min.

The starting amine, (2,3-cis)-3-amino-2-(2-chlorophenyl)-2,3-dihydro-1,5-benzothiazepin-4(5H)-one hydrobromide (87d), was prepared in the following manner:

### a. Methyl (2Z)-2-{[(benzyloxy)carbonyl]amino}-3-(2-chlorophenyl)acrylate (87a)

Using a procedure similar to that described in example 1 part a, except using 2-chlorobenzaldehyde (1.82 g, 13.0 mmol) as the aldehyde component, the title compound (87a) was obtained as an oil (3.0 g, 67%) contaminated with 30% (2E)-isomer. MS APCI, m/z=346 (M+1).

### b. Methyl β-[(2-aminophenyl)thio]-N-[(benzyloxy)carbonyl]-2-chlorophenylalaninate (87b)

Using a procedure similar to that described in example 10, part b, (stirred 40 h) except using methyl (2Z)-2-{[(benzyloxy)carbonyl]amino}-3-(2-chlorophenyl)acrylate (87a) (2.0 g, 5.8 mmol), gave the title compound (87b) (2.5 g, 91%) as a clear oil (70:30 mixture (erythro:threo)). MS APCI, m/z=471(M+1).

### c. Benzyl [(2,3-cis)-2-(2-chlorophenyl)-4-oxo-2,3,4,5-tetrahydro-1,5-benzothiazepin-3-yl]carbamate (87c)

Using a procedure similar to that described in example 41, part b, except using methyl β-[(2-aminophenyl)thio]-N-[(benzyloxy)carbonyl]-2-chlorophenylalaninate (87b) (2.0 g, 4.2 mmol) as the aniline component, afforded the title compound (1.65 g) containing 10% of the 2,3 trans product. Recrystallization from hot EtOAc (75 mL) gave pure title compound (87c) (1.3 g, 65%) as a white solid. MS APCI, m/z=439(M+1). LC/MS:2.77 Min

### d. (2,3-cis)-3-Amino-2-(2-chlorophenyl)-5-methyl-2,3-dihydro-1,5-benzothiazepin-4(5H)-one hydrobromide (87d)

Using a procedure similar to that described in example 41, part c, except using benzyl [(2,3-cis)-2-(2-chlorophenyl)-4-oxo-2,3,4,5-tetrahydro-1,5-benzothiazepin-3-yl]carbamate (87c) (1.2 g, 2.7 mmol) as the protected amine component, the title compound (87d) (930 mg, 88%) as a white solid. ¹H NMR (300 MHz, d6-DMSO) δ4.41 (d, 1H, J=7.4 Hz), 5.78 (d, 1H, J=7.4 Hz), 7.31 (m, 2H), 7.4-7.6 (m, 6H), 7.72 (d, 1H, J=7.4 Hz), 7.87 (d, 2H, J=7.4 Hz), 8.15 (bs, 3H), 10.95 (s, 1H). MS APCI, m/z=305(M+1). LC/MS: 1.62 min.

### Example 88

### N¹-[(2R,3R)-2-(2-Chlorophenyl)-4-oxo-2,3,4,5-tetrahydro-1,5-benzothiazepin-3-yl]-N²-[(3,5-difluorophenyl)acetyl]-L-alaninamide (88)

Using a procedure similar to that described in Example 1, except with (2,3-cis)-3-amino-2-(2-chlorophenyl)-2,3-dihydro-1,5-benzothiazepin-4(5H)-one (87d) (70 mg, 0.181 mmol) as the amine component and DCM/DMF as solvent gave after aqueous workup and column chromatography (hexane-EtOAc gradient) the solid title compound as a 1:1 mixture with the (2S,3S) diastereomer (64 mg, 67%). ¹H NMR (300 MHz, CDCl₃) δ0.99 (d, 1.5H, J=7.0 Hz), 1.21 (d, 1.5H, J=6.6 Hz), 3.45 (d, 2H, J=7.5 Hz), 4.28-4.39 (m, 0.5H), 4.52-4.60 (m, 0.5H), 4.91 (t, 0.5H, J=7.5 Hz), 4.99 (t, 0.5H, J=7.0 Hz), 5.93 (d, 0.5H, J=7.0 Hz), 5.99 (d, 0.5H, J=7.5 Hz), 6.05 (d, 0.5H, J=7.5 Hz), 6.22 (d, 0.5H, J=7.5 Hz), 6.50 (d, 0.5H, J=7.9 Hz), 6.66-6.79 (m, 2.5H), 7.07 (d, 0.5H, J=7.9 Hz), 7.15 (d, 0.5H, J=7.5 Hz), 7.23-7.46 (m, 6H), 7.73 (d, 1H, J=7.9 Hz), 7.84 (s, 0.5H), 7.84-7.89 (m, 1H), 8.42 (s, 0.5H). MS APCI, m/z=552(M+Na). LC/MS: 2.53 min.

### Example 89

### N¹-[(2R,3R)-7-Chloro-5-methyl-4-oxo-2-phenyl-2,3,4,5-tetrahydro-1,5-benzothiazepin-3-yl]-N²-[(3,5-difluorophenyl)acetyl]-L-alaninamide (89)

Using a procedure similar to that described in Example 1, except with (2,3-cis)-3-amino-7-chloro-5-methyl-2-phenyl-2,3-dihydro-1,5-benzothiazepin-4(5H)-one hydrobromide (89b) (70 mg, 0.175 mmol) as the amine component and DCM-DMF as solvent gave after aqueous workup and column chromatography (30:1 methanol-CHCl₃) the title compound as a 1:1 mixture with the (2S,3S) diastereomer; solid (54 mg, 57%). ¹H NMR (300 MHz, CDCl₃) δ0.92 (d, 1.5H, J=7.0 Hz), 1.15 (d, 1.5H, J=7.0 Hz), 3.42 (s, 1H), 3.43 (s, 1H), 3.50 (s, 3H), 4.11-4.22 (m, 1H), 4.72-4.78 (m, 1H), 5.11 (d, 1H, J=7.5 Hz), 5.75 (d, 0.5H, J=7.9 Hz), 5.94 (d, 0.5H, J=7.9 Hz), 6.30 (t, 1H), 6.67-6.78 (m, 2H), 7.27-7.36 (8H), 7.66 (d, 1H, J=8.3 Hz). MS APCI, m/z=566(M+Na). LC/MS: 2.78 min.

The starting amine, (2,3-cis)-3-amino-7-chloro-5-methyl-2-phenyl-2,3-dihydro-1,5-benzothiazepin-4(5H)-one hydrobromide (89b), was prepared in the following manner:

### a. Benzyl [(2,3-cis)-7-chloro-5-methyl-4-oxo-2-phenyl-2,3,4,5-tetrahydro-1,5-benzothiazepin-3-yl]carbamate (89a)

Using a procedure similar to that described in example 5 part a, substituting benzyl [(2,3-cis)-7-chloro-4-oxo-2-phenyl-2,3,4,5-tetrahydro-1,5-benzothiazepin-3-yl]carbamate (13b) (1.1 g, 2.51 mmol) as the amide component, the title compound (89a) (970 mg, 85%) was obtained as a white solid. MS APCI, m/z=453(M+Na). LC/MS: 3.04 min.

### b. (2,3-cis)-3-Amino-7-chloro-5-methyl-2-phenyl-2,3-dihydro-1,5-benzothiazepin-4(5D)-one hydrobromide (89b)

Using a procedure similar to that described in example 41, part c, substituting benzyl [(2,3-cis)-7-chloro-5-methyl-4-oxo-2-phenyl-2,3,4,5-tetrahydro-1,5-benzothiazepin-3-yl]carbamate (89a) (700 mg, 1.59 mmol) as the protected amine component, the title compound (89b) (500 mg, 78%) as a white solid. ¹H NMR (300 MHz, d6-DMSO) δ3.48 (s, 3H), 4.36 (d, 1H, J=7.0 Hz), 5.09 (d, 1H, J=7.0 Hz), 7.48 (m, 6H), 7.75 (d, 1H), 7.86 (s, 1H), 8.04 (bs, 3H). MS APCI, m/z=319(M+H). LC/MS: 1.82 min

### Example 90

### N²-[(3,5-Difluorophenyl)acetyl]-N¹-[(2R,3R)-2-(2-fluorophenyl)-4-oxo-2,3,45-tetrahydro-1,5-benzothiazepin-3-yl]-L-alaninamide (90)

Using a procedure similar to that described in Example 1, except with (2,3-cis)-3-amino-2-(2-fluorophenyl)-2,3-dihydro-1,5-benzothiazepin-4(5H)-one hydrobromide (90d) (70 mg, 0.189 mmol) as the amine component and DCM-DMF as solvent gave after aqueous workup the title compound as a 1:1 mixture with the (2S,3S) diastereomer; solid (96 mg, 99%). ¹H NMR (300 MHz, CDCl₃) δ1.03 (d, 1.5H, J=6.6 Hz), 1.21 (d, 1.5H, J=7.0 Hz), 3.43 (s, 1H), 3.47 (s, 1H), 4.31-4.44 (m, 0.5H), 4.50-4.59 (m, 0.5H), 4.86-4.97 (m, 1H), 5.75 (q, 1H), 6.01 (d, 0.5H, J=7.5H), 6.20 (d, 0.5H, J=7.9 Hz), 6.54 (d, 0.5H, J=7.9 Hz), 6.65-6.80 (m, 3.5H), 6.92-7.46 (m, 6H), 7.72 (d, 1H, J=7.5 Hz), 7.80 (s, 0.5H), 7.82 (d, 1H, J=8.3 Hz), 8.31 (s, 05H). MS APCI, m/z=514(M+1). LC/MS: 2.43 min.

The starting amine, (2,3-cis)-3-amino-2-(2-fluorophenyl)-2,3-dihydro-1,5-benzothiazepin-4(5H)-one hydrobromide (90d), was prepared in the following manner:

### a. Methyl (2Z)-2-{[(benzyloxy)carbonyl]amino}-3-(2-fluorophenyl)acrylate (90a)

Using a procedure similar to that described in example 1 part a, except using 2-fluorobenzaldehyde (596 μL, 5.50 mmol) as the aldehyde component, the title compound (90a) was obtained as an oil (1.8 g, 98%) contaminated with 15% (2E)-isomer. MS APCI, m/z=330(M+1).

### b. Methyl β-[(2-aminophenyl)thio]-N-[(benzyloxy)carbonyl]-2-fluorophenylalaninate (90b)

Using a procedure similar to that described in example 10, part b, (stirred 40 h) except using methyl (2Z)-2-{[(benzyloxy)carbonyl]amino}-3-(2-fluorophenyl)acrylate (90a) (1.8 g, 5.5 mmol), to afford crude title compound. After recrystallization from methanol-ether the title compound (90b) was obtained (1.2 g, 49%) as a white solid (95:5 mixture (erythro:threo)). MS APCI, m/z=455(M+1). LC/MS: 2.69 min.

### c. Benzyl [(2,3-cis)-2-(2-fluorophenyl)-4-oxo-2,3,4,5-tetrahydro-1,5-benzothiazepin-3-yl]carbamate (90c)

Using a procedure similar to that described in example 41, part b, substituting methyl β-[(2-aminophenyl)thio]-N-[(benzyloxy)carbonyl]-2-fluorophenylalaninate (90b) (1.2 g, 2.6 mmol) as the aniline component, the title compound (90c) (650 mg, 58%) was obtained as a white solid. MS APCI, m/z=445(M+Na). LC/MS: 2.77 min.

### d. (2R,3R)-3-Amino-2-(2-fluorophenyl)-5-methyl-2,3-dihydro-1,5-benzothiazepin-4(5H)-one hydrobromide (90d)

Using a procedure similar to that described in example 41, part c, substituting benzyl [(2,3-cis)-2-(2-fluorophenyl)-4-oxo-2,3,4,5-tetrahydro-1,5-benzothiazepin-3-yl]carbamate (90c) (580 mg, 1.37 mmol) as the protected amine component, the title compound (90d) (480 mg, 94%) was obtained as a white solid. ¹H NMR (300 MHz, d6-DMSO) δ4.37 (d, 1H, J=7.0 Hz), 5.61 (d, 1H, J=7.0 Hz), 7.26-7.36 (m, 4H), 7.46-7.59 (m, 2H), 7.75 (m, 2H), 8.19 (bs, 3H), 10.95 (s, 1H). MS APCI, m/z=289(M+1). LC/MS: 1.51 min.

### Example 91

### N²-[(3,5-Difluorophenyl)acetyl]-N-[(2R,3R)-2-(4-fluorophenyl)-4-oxo-2,3,4,5-tetrahydro-1,5-benzothiazepin-3-yl]-L-alaninamide (91)

Using a procedure similar to that described in Example 1, except with (2,3-cis)-3-amino-2-(4-fluorophenyl)-2,3-dihydro-1,5-benzothiazepin-4(5H)-one hydrobromide (91d) (70 mg, 0.189 mmol) as the amine component and DCM-DMF as solvent gave after aqueous workup the title compound as a 1:1 mixture with the (2S,3S) diastereomer; solid (64 mg, 66%). ¹H NMR (300 MHz, CDCl₃) δ0.98 (d, 1.5H, J=6.6 Hz), 1.18 (d, 1.5H, J=7.0 Hz), 3.43 (s, 1H), 3.45 (s, 1H), 4.17-4.27 (m, 0.5H), 4.45-4.54 (m, 0.5H), 4.83 (q, 1H), 5.28 (q, 1H), 5.82 (d, 0.5H, J=6.6 Hz), 6.06 (d, 0.5H, J=7.0 Hz), 6.41 (d, 0.5H, J=7.0 Hz), 6.65 (d, 0.5H, J=7.0 Hz), 6.72-6.79 (m, 3H), 6.98-7.16 (m, 3H), 7.20-7.31 (m, 1H), 7.37-7.44 (m, 3H), 7.70 (d, 1.5H, J=7.5 Hz), 8.17 (s, 0.5H). MS APCI, m/z=536(M+Na). LC/MS: 2.51 min.

The starting amine, (2,3-cis)-3-amino-2-(4-fluorophenyl)-2,3-dihydro-1,5-benzothiazepin-4(5H)-one hydrobromide (91d), was prepared in the following manner:

### a. Methyl (2Z)-2-{[(benzyloxy)carbonyl]amino}-3-(4-fluorophenyl)acrylate (91a)

Using a procedure similar to that described in example 1 part a, except using 4-fluorobenzaldehyde (600 μL, 5.50 mmol) as the aldehyde component, the title compound (91a) was obtained as an oil (1.8 g, 98%) contaminated with 15% (2E) isomer. MS APCI, m/z=330(M+1).

### b. Methyl β-[(2-aminophenyl)thio]-N-[(benzyloxy)carbonyl]-4-fluorophenylalaninate (91b)

Using a procedure similar to that described in example 10, part b, (stirred 40 h) except using methyl (2Z)-2-{[(benzyloxy)carbonyl]amino}-3-(4-fluorophenyl)acrylate (91a) (1.8 g, 5.5 mmol) as reactant, to afford crude title compound. After recrystallization from EtOAc-hexanes the title compound (91b) (1.3 g, 52%) was obtained as a white solid (97:3 mixture (erythro:threo)). MS APCI, m/z=455(M+1). LC/MS: 2.69 min.

### c. Benzyl [(2,3-cis)-2-(4-fluorophenyl)-4-oxo-2,3,4,5-tetrahydro-1,5-benzothiazepin-3-yl]carbamate (91c)

Using a procedure similar to that described in example 41, part b, substituting methyl β-[(2-aminophenyl)thio]-N-[(benzyloxy)carbonyl]-4-fluorophenylalaninate (91b) (1.3 g, 2.86 mmol) as the aniline component, the title compound (91c) (900 mg, 73%) was obtained as a white solid. MS APCI, m/z=445(M+Na). LC/MS: 2.77 min.

### d. (2,3-cis)-3-Amino-2-(4-fluorophenyl)-5-methyl-2,3-dihydro-1,5-benzothiazepin-4(5H)-one hydrobromide (91d)

Using a procedure similar to that described in example 41, part c, substituting benzyl [(2,3-cis)-2-(4-fluorophenyl)-4-oxo-2,3,4,5-tetrahydro-1,5-benzothiazepin-3-yl]carbamate (91c) (850 mg, 2.01 mmol) as the protected amine component, the title compound (91d) (700 mg, 94%) as a white solid. MS APCI, m/z=313(M+Na). LC/MS: 1.57 min. ¹H NMR (300 MHz, d6-DMSO) δ4.29 (d, 1H, J=7.0 Hz), 5.28 (d, 1H, J=7.0 Hz), 7.26-7.36 (m, 4H), 7.55 (m, 3H), 7.69 (d, 1H, J=7.9 Hz), 8.06 (bs, 3H), 10.89 (s, 1H).

### Example 92

### N¹-[(2R,3R)-7-chloro-2-(2,5-difluorophenyl)-4-oxo-2,3,4,5-tetrahydro-1,5-benzothiazepin-3-yl]-N²-[(3,5-difluorophenyl)acetyl]-L-alaninamide (92)

A stirred slurry of (2,3-cis)-3-amino-7-chloro-2-(2,5-difluorophenyl)-2,3-dihydro-1,5-benzothiazepin-4(5H)-one hydrobromide (92c) (0.127 g, 0.30 mmol) in DCM (12 mL), under N², was treated with NMM (0.05 mL, 0.45 mmol) and the clear solution cooled in an ice-bath. N-[(3,5-difluorophenyl)acetyl]-L-alanine (1e) (0.080 g, 0.33 mmol), EDAC HCl (0.0863 g, 0.45 mmol) and NMM (0.05 mL, 0.45 mmol) were added successively and the mixture stirred for 2 h. The solvent was removed in vacuo and the residue portioned between EtOAc (50 mL) and water (50 mL). The material from the washed (sat. NaHCO₃ and brine) EtOAc solution was a mixture of two diastereomers and was partially purified by column chromatography (eluent: 3:1, 2:1 and 1:1 hexane-EtOAc) to yield the white title compound as a 9:1 mixture with the 2R,3R diastereomer (0.041 g, 24%). ¹H NMR (300 MHz, CDCl₃) δ1.08 (d, 0.3H, J=7.0 Hz, 2R,3R diastereomer), 1.21 (d, 3H, J=7.0 Hz), 3.44 (s, 0.2H, 2R,3R diastereomer), 3.48 (s, 2H), 4.32 (m, 1H), 4.91 (t, 1H), 5.66 (d, 1H, J=8.3 Hz), 5.99 (d, 1H, J=7.0 Hz), 6.54 (d, 1H, J=7.5 Hz), 6.79 (d, 1H, J=7.9 Hz), 6.9-7.0 (m, 2H), 7.16 (s, 1H), 7.50-7.56 (m, 1H), 7.65 (d, 1H, J=7.9 Hz), 7.85 (m, 5H). MS APCI, m/z=566(M+1). HPLC Method A: 3.34 min.

The required 3-amino-7-chloro-2-(2,5-difluorophenyl)-2,3-dihydro-1,5-benzothiazepin-4(5H)-one•hydrobromide was prepared in the following manner:

### a. Methyl β-[(2-amino-4-chlorophenyl)thio]-N-[(benzyloxy)carbonyl]-2,5-difluorophenylalaninate (92a)

The title compound was obtained from 2-amino-4-chlorobenzenethiol (5.0 g, 31.3 mmol) and (1a) (2.17 g, 6.26 mmol) as a yellow foam (2.68 g, 85%) following Method B. The Z:E ratio of the material was 85:15 as determined by ¹H-NMR (methyl integration). ¹H NMR (300 MHz, CDCl₃) δ3.55 (s, 3H, Z isomer), 3.72 (s, 0.5H, E isomer), 4.37 (br. s, 2H), 4.85-4.9 (m, 2H), 5.0-5.15 (m, 2H), 5.73 (d, 1H, J=9.7 Hz), 6.54 (dd, 1H, J=8.3, 2.0 Hz), 6.67 (d, 1H, J=2.0 Hz), 6.9-7.0 (m, 2H), 7.07 (d, 1H, J=8.3 Hz), 7.13-7.17 (m, 1H), 7.3-7.4 (m, 5H). MS APCI, m/z=507(M+1). HPLC Method A: 3.62 min.

### b. Benzyl [(2,3-cis)-7-chloro-2-(2,5-difluorophenyl)-4-oxo-2,3,4,5-tetrahydro-1,5-benzothiazepin-3-yl]carbamate (92b)

A solution of (91a) (2.68 g, 5.29 mmol) and pTSA (0.1 g) in xylenes (75 mL) was stirred in an oil bath at 150° C. for 3 h, 170° C. for 2 h, allowed to stand for ˜12 h and the precipitated white solid (2.0 g) collected. The material was dissolved in hot methyl ethyl ketone (150 mL), treated with ethyl ether (150 mL), refrigerated and the pure title compound obtained as a white solid (1.52 g, 76%), mp 233-235° C. ¹H NMR (300 MHz, d6-DMSO) δ 4.64 (t, 1H), 4.96 (s, 2H), 5.49 (d, 1H, J=7.0 Hz), 7.10 (d, 1H, J=7.9 Hz), 7.24-7.38 (m, 9H) 7.72 (d, 1H, J=7.9 Hz), 10.65 (s, 1H). MS APCI, m/z=507(M+1). HPLC Method A: 3.62 min.

### c. (2,3-cis)-3-Amino-7-chloro-2-(2,5-difluorophenyl)-2,3-dihydro-1,5-benzothiazepin-4(5H)-one hydrobromide (92c)

A mixture of (92b) (1.0 g, 2.1 mmol), acetic acid (5 mL) and 30% HBr/acetic acid (10 mL) was stirred in a 60° oil bath for 1 h, cooled, treated with ethyl ether (75 mL) and the white solid collected (0.86 g, 97%), mp 254-256° C. dec. The material contained 1 mole of acetic acid after drying at 50° C. at 0.1 torr for ˜12 h. ¹H NMR (300 MHz, d6-DMSO) δ1.91 (s, 3H), 4.49 (d, 1H, J=7.0 Hz), 5.57 (d, 1H, J=7.0 Hz), 7.3-7.5 (m, 5H), 7.75 (d, 1H, J=8.3 Hz), 8.27 (br. S, 3H), 11.06 (s, 1H). MS APCI, m/z=341(M+1). HPLC Method A: 2.22 min.

### Example 93

### N²-[(2S)-2-Hydroxy-4-methyl-1-oxopentyl]-N¹-[(6R,7R)-5-oxo-7-phenyl-1,4-thiazepan-6-yl]-L-alaninamide (93)

Using a procedure similar to that described in Example 1, except with (6,7-cis)-6-amino-7-phenyl-1,4-thiazepan-5-one (7d) (84.3 mg, 0.38 mmol) as the amine component, N-(2S)-2-hydroxy-4-methyl-1-oxopentyl-L-alanine (85.4 mg, 0.42 mmol) as the acid component and DMF (3 mL) as solvent gave after aqueous workup and column chromatography (2% methanol-CHCl₃) the title compound as a 1:1 mixture with the (6S,7S) diastereomer; white solid (0.12 g, 78%). ¹H NMR (300 MHz, CDCl₃) δ0.94 (d, 6H, J=6.1 Hz), 1.20 (d, 1.5H, J=7.5 Hz), 1.31 (d, 1.5H, J=7.0 Hz), 1.4-1.65 (m, 2H), 1.75-1.9 (m, 1H), 2.77-2.94 (m, 2H), 3.0-3.1 (m, 1H), 3.7-3.9 (m, 2H), 4.04-4.11 (m, 1H), 4.34 (d, 1H, J=3.9 Hz), 4.4-4.5 (m, 1H), 5.29-5.36 (m, 1H), 6.32-6.43 (m, 1H), 6.90 (d, 0.5H), 6.99 (d, 0.5H), 7.08 (d, 0.5H), 7.22 (d, 0.5H), 7.28 (s, 5H). MS APCI, m/z=408(M+1). HPLC Method A: twined peak at 2.26 and 2.31 min.

### Example 94

### N²-[(3,5-Difluorophenyl)acetyl]-N¹-[(2S,3R)-2-(3-methyl-2-thienyl)-4-oxo-2,3,4,5-tetrahydro-1,5-benzothiazepin-3-yl]-L-alaninamide (94)

To a solution of (2,3-cis)-3-amino-2-(3-methyl-2-thienyl)-2,3-dihydro-1,5-benzothiazepin-4(5H)-one hydrobromide (94d) (0.501 g, 1.35 mmol), HOBt (0.237 g, 1.76 mmol), N-[(3,5-difluorophenyl)acetyl]-L-alanine (1e) (0.352 g, 1.45 mmol), and N,N-diisopropylethylamine (0.401 g, 3.10 mmol) in dry DCM (7 mL) was added EDAC-HCl (0.336 g, 1.75 mmol). The mixture was stirred at ambient temperature under nitrogen for 4 h then loaded directly onto a silica gel flash column, eluting with 100 mL each of 9:1, 8:2, and 7:3 (v/v) DCM-EtOAc to afford the title compound as an oil, which crystallized from 10:1 petroleum ether-ether as a 1:1 mixture with the 2R,3S diastereomer as a white solid (285 mg, 41%). ¹H NMR (300 MHz, d₆-DMSO) δ0.97 (d, 1.5H, J=7 Hz), 1.04 (d, 1.5H, J=7 Hz), 1.98 (s, 1.5H), 2.05 (s, 1.5H), 3.40 (m, 2H), 4.29 (m, 1H), 4.71 (m, 1H), 5.55 (d, 0.5H, J=6.6 Hz), 5.59 (d, 0.5H, J=7.0 Hz), 6.81-7.29 (m, 6H), 7.46-7.67 (m, 4H), 8.25 (t, 1H, J=7.9 Hz), 10.50 (d, 1H, J=5.7 Hz). MS APCI, m/z=516 (M+1). LC/MS: 2.27 min.

The starting amine, (2,3-cis)-3-amino-2-(3-methyl-2-thienyl)-2,3-dihydro-1,5-benzothiazepin-4(5H)-one hydrobromide (24d), was prepared in the following manner:

### a. Methyl (2Z)-2-{[(benzyloxy)carbonyl]amino}-3-(3-methyl-2-thienyl)acrylate (94a)

A stirred solution of N-(benzyloxycarbonyl)-α-phosphonoglycine trimethyl ester (5.01 g, 15.1 mmol) and 3-methylthiophene-2-carbaldehyde (7.5:1 mixture with 4-methylthiophene-2-carbaldehyde, 2.48 g, 19.6 mmol) in dry DCM (100 mL), was cooled to 0° C. and treated dropwise with DBU (2.76 g, 18.1 mmol). The mixture was warmed to ambient temperature and stirred for ˜12 h. The solution was concentrated, taken up in EtOAc (150 mL) and washed with 5% hydrochloric acid. The organic extract was washed with saturated aqueous sodium bicarbonate, water (75 mL), dried (magnesium sulfate), filtered and evaporated. The residue was purified by flash chromatography (20-40% EtOAc-hexanes gradient) to afford the title compound (4.07 g, 81%) as an orange oil. The product is a mixture of 3- and 4-methylthiophene isomers (6.8:1). ¹H NMR major isomer: (300 MHz, d₆-DMSO) δ2.33 (s, 3H), 3.72, (s, 3H), 5.13 (bs, 2H), 7.01 (d, 1H, J=5.3 Hz), 7.18-7.52 (m, 5H), 7.60-7.85 (m, 2H), 8.83. (s, 1H). MS APCI, m/z=354(M+Na). LC/MS: 2.43 min.

### b. Methyl β-[(2-aminophenyl)thio]-N-[(benzyloxy)carbonyl]-3-(3-methyl-2-thienyl)alaninate (94b)

To a degassed solution of methyl (2Z)-2-{[(benzyloxy)carbonyl]amino}-3-(3-methyl-2-thienyl)acrylate (94a) (4.07 g, 12.3 mmol) and triethylamine (0.871 g, 8.61 mmol) in anhydrous methanol (50 mL) was added 2-aminothiophenol (6.14 g, 49.1 mmol) under N². The reaction mixture was heated at reflux for ˜12 h then cooled to ambient temperature and evaporated under reduced pressure. The mixture was partitioned between 20% aqueous potassium carbonate (100 mL) and EtOAc (100 mL). The organic phase was separated and washed with 20% aqueous potassium carbonate, dried, filtered and evaporated to yield 5.62 g of orange oil. The crude product was passed through a flash column eluting with a 20-40% EtOAc-hexanes solvent gradient affording the title compound as a mixture with the 4-methylthiophene isomer (4.31 g, 77%). The mixture was used directly in the next step without further purification. MS APCI, m/z=457 (M+1). LC/MS: 2.74 min.

### c. Benzyl [(2,3-cis)-2-(3-methyl-2-thienyl)-4-oxo-2,3,4,5-tetrahydro-1,5-benzothiazepin-3-yl]carbamate (94c)

To a suspension of methyl β-[(2-aminophenyl)thio]-N-[(benzyloxy)carbonyl]-3-(3-methyl-2-thienyl)alaninate (94b) (4.31 g, 9.44 mmol) in o-xylene (95 mL) was added a catalytic amount of p-toluenesulfonic acid hydrate (0.107 g, 0.565 mmol). The mixture was heated at 170° C. for 2 h then cooled to ambient temperature. The mixture was filtered, the filtrate was concentrated under reduced pressure and partitioned between EtOAc (150 mL) and 5% hydrochloric acid (100 mL). The organic phase was separated, washed with aqueous saturated sodium bicarbonate, brine, dried (magnesium sulfate), filtered and concentrated leaving an orange oil. Purification by flash chromatography on silica gel eluting with a 20-80% EtOAc-hexane solvent gradient afforded the title compound in a 9:1 mixture with the 4-methylthiophene isomer (1.42 g, 35%) as a pale yellow solid. ¹H NMR major isomer (300 MHz, d₆-DMSO) δ2.01 (s, 3H), 4.56 (t, 1H, J=7.5 Hz), 4.98 (s, 2H), 5.58 (d, 1H, J=7.0 Hz), 6.22 (d, 1H, J=7.9 Hz), 6.86 (d, 1H, J=5.3 Hz), 7.19-7.52 (m, 9H), 7.66 (d, 1H, J=7.5 Hz), 10.49 (s, 1H). MS APCI, m/z=425 (M+1). LC/MS: 2.52 min.

### d. (2,3-cis)-3-Amino-2-(3-methyl-2-thienyl)-2,3-dihydro-1,5-benzothiazepin-4(5H)-one hydrobromide (94d)

To benzyl [(2,3-cis)-2-(3-methyl-2-thienyl)-4-oxo-2,3,4,5-tetrahydro-1,5-benzothiazepin-3-yl]carbamate (94c) (1.37 g, 3.22 mmol) was added 4.1M HBr in acetic acid (10 mL). The stirred suspension became a homogeneous solution over 20 min. The reaction stirred at ambient temperature for an additional 2 h and was diluted with ether to afford the hydrobromide salt of the title compound in a 9.3:1 mixture with the 4-methylthiophene isomer (0.919 g, 77%) as a white solid. ¹H NMR major product (300 MHz, d₆-DMSO) δ2.23 (s, 3H), 4.24 (d, 1H, J=6.6 Hz), 5.69 (d, 1H, J=6.6 Hz), 6.91 (d, 1H, J=4.8 Hz), 7.23-7.33 (m, 2H), 7.54 (t, 1H), 7.61 (d, 1H, J=4.8 Hz), 7.67 (d, 1H, J=7.5 Hz), 8.09 (bs, 2H), 10.87 (s, 1H). MS APCI, m/z=291 (M+1). LC/MS: 1.55 min.

### Example 95

### N²-[(3,5-Difluorophenyl)acetyl]-N¹-[(2S,3R)-2-(4-methyl-2-thienyl)-4-oxo-2,3,4,5-tetrahydro-1,5-benzothiazepin-3-yl]-L-alaninamide (95)

The title compound was synthesized according to the method of Example 94, employing (2,3-cis)-3-amino-2-(4-methyl-2-thienyl)-2,3-dihydro-1,5-benzothiazepin-4(5H)-one hydrobromide (95e) (97.0 mg, 0.261 mmol), HOBt (51.0 mg, 0.377 mmol), N-[(3,5-difluorophenyl)acetyl]-L-alanine 1e (69.0 mg, 0.284 mmol), diisopropylethylamine (77.8 mg, 0.602 mmol), and EDAC-HCl (64.5 mg, 0.336 mmol) in dry DCM (2 mL). Purification by flash chromatography on silica gel eluting with a 10-50% EtOAc-DCM solvent gradient afforded the title compound as a 1:1 mixture with the 2R,3S diastereomer (87 mg, 64%) as a pale yellow solid. ¹H NMR (300 MHz, d₆-DMSO) δ1.05 (d, 1.5H, J=7 Hz), 1.09 (d, 1.5H, J=7 Hz), 2.15 (s, 1.5H), 2.17 (s, 1.5H), 3.42 (m, 2H), 4.29 (m, 1H), 4.64 (m, 1H), 5.34 (m, 1H), 6.89-7.29 (m, 7H), 7.47-7.75 (m, 3H), 8.27 (d, 0.5H, J=7.5 Hz), 8.37 (d, 0.5H, J=7.5 Hz), 10.50 (s, 0.5H), 10.54 (s, 0.5H). MS APCI, m/z=516 (M+1). LC/MS: 2.35 min.

The starting amine, (2,3-cis)-3-amino-2-(4-methyl-2-thienyl)-2,3-dihydro-1,5-benzothiazepin-4(5H)-one hydrobromide (95e), was prepared in the following manner:

### a. 4-Methylthiophene-2-carbaldehyde (95a)

The title compound was prepared as a 4:1 mixture of the desired isomer and 3-methylthiophene-2-carbaldehyde, according to the published procedure of Jean Sicé; J. Org. Chem. 19, 70 (1954).

### b. Methyl (2Z)-2-{[(benzyloxy)carbonyl]amino}-3-(4-methyl-2-thienyl)acrylate (95b)

A stirred solution of N-(benzyloxycarbonyl)-α-phosphonoglycine trimethyl ester (5.01 g, 15.1 mmol) and 4-methylthiophene-2-carbaldehyde (95a) (2.50 g, 19.8 mmol) in dry DCM (100 mL) was cooled to 0° C. and treated dropwise with DBU (2.76 g, 18.1 mmol). The mixture was warmed to ambient temperature and stirred for ˜12 h. The solution was concentrated, taken up in EtOAc (150 mL) and washed with 5% hydrochloric acid. The organic extract was washed with saturated aqueous sodium bicarbonate, water (200 mL), dried (magnesium sulfate), filtered and evaporated. The residue was purified by crystallization from 4:1 (v/v) EtOAc-hexanes to afford the title compound (3.38 g, 67%) as the sole product. ¹H NMR (300 MHz, d₆-DMSO) δ2.20 (s, 3H), 3.70, (s, 3H), 5.12 (s, 2H), 7.20-7.52 (m, 7H), 7.69 (s, 1H), 8.82 (s, 1H). MS APCI, m/z=354 (M+Na). LC/MS: 2.43 min.

### c. Methyl β-[(2-aminophenyl)thio]-N-[(benzyloxy)carbonyl]-3-(4-methyl-2-thienyl)alaninate (95c)

The title compound was prepared according to the method of Example 94b, employing methyl (2Z)-2-{[(benzyloxy)carbonyl]amino}-3-(4-methyl-2-thienyl)acrylate (95b) (3.38 g, 10.2 mmol), triethylamine (0.722 g, 7.14 mmol), and 2-aminothiophenol (6.43 g, 51.4 mmol) in anhydrous methanol (41 mL). The crude product was crystallized from 4:1 (v/v) petroleum ether/ether affording the title compound (2.74 g, 59%) as a pale yellow solid. ¹H NMR (300 MHz, d₆-DMSO) δ2.04 (s, 3H), 3.38 (s, 3H), 4.56 (t, 1H, J=8.3 Hz), 4.85 (d, 1H, J=7.9 Hz), 5.09 (m, 2H), 5.42 (bs, 2H), 6.36 (t, 1H, J=7.5 Hz), 6.56 (s, 1H), 6.65 (d, 1H, J=7.9 Hz), 6.94-7.02 (m, 3H), 7.30-7.42 (m, 5H), 8.21 (d, 1H, J=9.3 Hz). MS APCI, m/z=457 (M+1). LC/MS: 2.64 min.

### d. Benzyl [(2,3-cis)-2-(4-methyl-2-thienyl)-4-oxo-2,3,4,5-tetrahydro-1,5-benzothiazepin-3-yl]carbamate (95d)

The title compound was prepared according to the method of Example 94c, employing methyl β-[(2-aminophenyl)thio]-N-[(benzyloxy)carbonyl]-3-(4-methyl-2-thienyl)alaninate (95c) (2.71 g, 5.93 mmol), p-toluenesulfonic acid hydrate (0.0882 g, 0.464 mmol), and o-xylene (60 mL). The reaction was heated at reflux for ˜12 h. Upon treatment of the crude product with ethanol-ether-petroleum ether, a solid impurity precipitated and was filtered off. The filtrate was concentrated and crystallized from EtOAc-hexane affording a yellow solid (189 mg, 7%). ¹H NMR (300 MHz, d₆-DMSO) δ2.17 (s, 3H), 4.49 (dd, 1H, J=7.5, J=6.6 Mz), 4.99 (m, 2H), 5.42 (d, 1H, J=6.6 Hz), 6.23 (m, 1H), 6.92 (s, 1H), 7.12 (s, 1H), 7.20-7.52 (m, 8H), 7.65 (d, 1H, J=7.5 Hz), 10.48 (s, 1H). MS APCI, m/z=447 (M+Na). LC/MS: 2.66 min.

### e. (2,3-cis)-3-Amino-2-(4-methyl-2-thienyl)-2,3-dihydro-1,5-benzothiazepin-4(5H)-one hydrobromide (95e)

The title compound was prepared according to the method of Example 94d, employing benzyl [(2,3-cis)-2-(4-methyl-2-thienyl)-4-oxo-2,3,4,5-tetrahydro-1,5-benzothiazepin-3-yl]carbamate (95d) (189 mg, 0.444 mmol) and 4.1M HBr in acetic acid (4.0 mL). The title compound was isolated as the hydrobromide salt (97 mg, 59%). ¹H NMR (300 MHz, d₆-DMSO) δ2.22 (s, 3H), 4.25 (d, 1H), 5.50 (d, 1H), 7.09 (s, 1H), 7.20-7.70 (m, 5H), 8.15 (bs, 2H), 10.85 (s, 1H). MS APCI, m/z=291 (M+1). LC/MS: 1.43 min.

### Example 96

### Methyl 5-[(2S,3R)-3-(N-[(3,5-difluorophenyl)acetyl]-L-alanylamino)-4-oxo-2,3,4,5-tetrahydro-1,5-benzothiazepin-2-yl]thiophene-3-carboxylate (96)

The title compound was synthesized according to the method of Example 94, employing methyl 5-[(2,3-cis)-3-amino-4-oxo-2,3,4,5-tetrahydro-1,5-benzothiazepin-2-yl]thiophene-3-carboxylate hydrobromide (96e) (200 mg, 0.482 mmol), HOBt (93.4 mg, 0.691 mmol), N-[(3,5-difluorophenyl)acetyl]-L-alanine 1e (128 mg, 0.526 mmol), diisopropylethylamine (143 mg, 1.11 mmol), and EDAC-HCl (127 mg, 0.665 mmol) in dry DCM (5 mL). Purification by flash chromatography on silica gel (20-80% EtOAc-hexane solvent gradient) afforded the title compound in a 1:1 mixture with the 2R,3S diastereomer (151 mg, 56%) as a white solid. ¹H NMR (300 MHz, d₆-DMSO) δ1.05 (d, 1.5H, J=7 Hz), 1.09 (d, 1.5H, J=7.1 Hz), 3.35-3.51 (m, 2H), 3.78 (s, 1.5H), 3.79 (s, 1.5H), 4.20-4.36 (m, 1H), 4.66-4.74 (m, 1H), 5.43 (m, 1H), 6.90-7.31 (m, 5H), 7.43-7.69 (m, 4H), 7.84 (d, 0.5H, J=7 Hz), 8.22 (d, 0.5H, J=7 Hz), 8.32 (s, 1H), 10.56 (s, 0.5H), 10.57 (s, 0.5H). MS APCI, m/z=516 (M+1). LC/MS: 2.35 min.

The starting amine, methyl 5-[(2S,3R)-3-amino-4-oxo-2,3,4,5-tetrahydro-1,5-benzothiazepin-2-yl]thiophene-3-carboxylate hydrobromide (96e), was prepared in the following manner:

### a. Methyl (2Z)-2-{[((benzyloxy)carbonyl]amino}-3-(4-bromo-2-thienyl)acrylate (96a)

The title compound was prepared according to the method of Example 94a, employing N-(benzyloxycarbonyl)-α-phosphonoglycine trimethyl ester (10.0 g, 30.2 mmol), 4-bromothiophene-2-carbaldehyde (7.52 g, 39.4 mmol), and DBU (5.34 g, 35.1 mmol) in dry DCM (200 mL). Recrystallization from 4:1 (v/v) hexane-EtOAc afforded the title compound (8.13 g, 20.5 mmol, 68%) as a white solid. ¹H NMR (300 MHz, d₆-DMSO) δ3.71 (s, 3H), 5.12 (bs, 2H), 7.10-7.51 (bm, 5H), 7.59 (s, 1H), 7.71 (s, 1H), 7.87 (s, 1H), 8.98 (bs, 1H). MS APCI, m/z=354, 352. LC/MS: 2.53 min.

### b. Methyl β-[(2-aminophenyl)thio]-N-[(benzyloxy)carbonyl]-3-(4-bromo-2-thienyl)alaninate (96b)

The title compound was prepared according to the method of Example 94b, employing methyl (2Z)-2-{[(benzyloxy)carbonyl]amino}-3-(4-bromo-2-thienyl)acrylate (96a) (8.13 g, 20.5 mmol), triethylamine (1.43 g, 14.1 mmol), and 2-aminothiophenol (14.0 g, 112 mmol) in anhydrous methanol (80 mL). Purification by flash chromatography on silica gel eluting with 4:1 (v/v) hexane-EtOAc yielded a solid, which was recrystallized from 6:1 (v/v) hexane-EtOAc to afford the title compound (4.90 g, 46%) as a white solid. ¹H NMR (300 MHz, d₆-DMSO) δ3.44 (s, 3H), 4.63 (t, 1H, J=8.1 Hz), 4.92 (d, 1H, J=7.4 Hz), 5.09 (m, 2H), 5.46 (bs, 2H), 6.38 (t, 1H, J=7.5 Hz), 6.66 (d, 1H, J=8.3 Hz), 6.80 (s, 1H), 6.99-7.03 (m, 2H), 7.32-7.39 (m, 5H), 7.52 (s, 1H), 8.28 (d, 1H, J=9.2 Hz). MS APCI, m/z=521, 523. LC/MS: 2.71 min.

### c. Benzyl [(2,3-cis-)2-(4-bromo-2-thienyl)-4-oxo-2,3,4,5-tetrahydro-1,5-benzothiazepin-3-yl]carbamate (96c)

The title compound was prepared according to the method of Example 94c, employing methyl β-[(2-aminophenyl)thio]-N-[(benzyloxy)carbonyl]-3-(4-bromo-2-thienyl)alaninate (96b) (4.79 g, 9.19 mmol), p-toluenesulfonic acid hydrate (0.101 g, 0.532 mmol), and o-xylene (90 mL). The reaction was heated at 170° C. for 2 h, then at ambient temperature for 2 d. The resulting precipitate was filtered, washed with o-xylene and ether affording the title compound (3.99 g, 89%) as a white solid. ¹H NMR (300 MHz, d₆-DMSO) δ4.52-4.56 (m, 1H), 4.99 (s, 2H), 5.46 (d, 1H, J=6.6 Hz), 6.76 (d, 1H, J=7.4 Hz), 7.09 (s, 1H), 7.20-7.36 (m, 7H), 7.51 (t, 1H, J=7.6 Hz), 7.66 (d, 1H, J=7.5 Hz), 7.69 (s, 1H), 10.52 (s, 1H). MS APCI, m/z=489, 491. LC/MS: 2.76 min.

### d. Methyl 5-((2,3-cis)-3-{[(benzyloxy)carbonyl]amino}-4-oxo-2,3,4,5-tetrahydro-1,5-benzothiazepin-2-yl)thiophene-3-carboxylate (96d)

To a degassed solution of benzyl [(2,3-cis)-2-(4-bromo-2-thienyl)-4-oxo-2,3,4,5-tetrahydro-1,5-benzothiazepin-3-yl]carbamate (26c) (0.502 g, 1.02 mmol) and triethylamine (0.145 g, 1.43 mmol) in anhydrous 1:1 (v/v) methanol/DMSO (19 mL) was added 1,3-bis(diphenylphosphino)propane (42.4 mg, 0.103 mmol) and palladium (II) acetate (21.8 mg, 0.0972 mmol). The reaction mixture was purged with CO and heated at 75° C. under a CO atmosphere for 2 d. After cooling, methanol (20 mL) was added, and the reaction mixture was filtered through diatomaceous earth. The filtrate was partitioned between water (100 mL) and EtOAc (100 mL). The organic phase was washed with water, dried, filtered and evaporated to a reddish-brown oil. Purification by flash chromatography on silica gel (20-60% EtOAc-hexane solvent gradient) afforded the title compound (201 mg, 42%) as a white solid. ¹H NMR (300 MHz, d₆-DMSO) δ3.80 (s, 3H), 4.53-4.57 (m, 1H), 4.99 (s, 2H), 5.50 (d, 1H, J=6.6 Hz), 6.66 (d, 1H, J=7.0 Hz), 7.09-7.31 (m, 7H), 7.49-7.54 (m, 2H), 7.66 (d, 1H, J=7.4 Hz), 8.33 (s, 1H), 10.52 (s, 1H). MS APCI, m/z=469 (M+1). LC/MS: 2.58 min.

### e. Methyl 5-[(2,3-cis)-3-amino-4-oxo-2,3,4,5-tetrahydro-1,5-benzothiazepin-2-yl]thiophene-3-carboxylate hydrobromide (96e)

The title compound was prepared according to the method of Example 94d, employing methyl 5-((2,3-cis)-3-{[(benzyloxy)carbonyl]amino}-4-oxo-2,3,4,5-tetrahydro-1,5-benzothiazepin-2-yl)thiophene-3-carboxylate (96d) (245 mg, 0.523 mmol) and 4.1M HBr in acetic acid (5.0 mL). The title compound was isolated as the hydrobromide salt (200 mg, 0.481 mmol, 92%). ¹H NMR (300 MHz, d₆-DMSO) δ3.82 (s, 3H), 4.32 (d, 1H, J=6.1 Hz), 5.63 (d, 1H, J=6.2 Hz), 7.25-7.36 (m, 2H), 7.53-7.73 (m, 3H), 8.21 (bs, 2H), 8.44 (s, 1H), 10.92 (s, 1H). MS APCI, m/z=291 (M+1). LC/MS: 1.43 min.

### Example 97

### N¹-[(2R,3R)-4-Oxo-2-phenyl-2,3,4,5-tetrahydro-1,5-benzothiazepin-3-yl]-N²-(phenylacetyl)-L-alaninamide (97)

To a solution of N¹-[(2,3-cis)-4-oxo-2-phenyl-2,3,4,5-tetrahydro-1,5-benzothiazepin-3-yl]-L-alaninamide 97b (162 mg), HOBt (74 mg), phenylacetic acid (75 mg) and EDAC-HCl (10Smg) in DCM (10 mL) was added triethylamine (202 mg). The mixture was stirred at 25° C. under nitrogen for 16 h and purified by flash chromatography (1:1 hexane:EtOAc) to afford the title compound as a 1:1 mixture with the 2S,3R diastereomer, white solid (117 mg), m.p. 114-124° C. ¹H NMR (300 MHz, d6-DMSO) δ 0.913 (d, 1.5H, J=7 Hz), 1.01 (d, 1.5H, J=7 Hz), 4.12 (m, 1H), 4.66 (m, 1H), 5.13 (m, 1H), 7.15-7.52 (m, 14H), 8.08 (d, 0.5H, J=7 Hz), 8.22 (d, 0.5H, J=7 Hz), 10.53 (br, 1H). MS APCI, m/z=460 (M+1) LC/MS: 2.17 min.

The starting amine N¹-[(2S,3S)-4-oxo-2-phenyl-2,3,4,5-tetrahydro-1,5-benzothiazepin-3-yl]-L-alaninamide (97b) was prepared in the following manner:

### a. N²-[tert-Butoxycarbonyl]-N¹-[(2,3-cis)-4-oxo-2-phenyl-2,3,4,5-tetrahydro-1,5-benzothiazepin-3-yl]-L-alaninamide (97a)

To a solution of (2,3-cis)-3-amino-2-phenyl-2,3-dihydro-1,5-benzothiazepin-4(5H)-one hydrobromide (9d) (303 mg), HOBt (216 mg), N-(tert-butoxycarbonyl)-L-alanine (284 mg), 4-dimethylaminopyridine (2 mg) and EDAC-HCl (288 mg) in DCM (10 mL) was added triethylamine (404 mg). The mixture was stirred at 25° C. under nitrogen for 16 h and purified by flash chromatography (1:1 hexane-EtOAc) to afford the title compound (440 mg). ¹H NMR (300 MHz, d6-DMSO) δ0.88 (d, 1.5H, J=7 Hz), 0.95 (d, 1.5H, J=7 Hz), 1.30 (two peaks, 9H), 3.73 (t, 0.5H J=7 Hz), 3.90 (t, 0.5H, J=7 Hz), 4.66 (m, 1H), 5.14 (t, 1H, J=6 Hz), 6.61-7.53 (m, 10H), 7.7 (d, 1H, J=6 Hz), 10.55 (d, 1H, J=5 Hz). MS APCI, m/z=342 (M−99). LC/MS: 2.31 min.

### b. N¹-[(2,3-cis)-4-Oxo-2-phenyl-2,3,4,5-tetrahydro-1,5-benzothiazepin-3-yl]-L-alaninamide (97b)

A solution of N²-[tert-butoxycarbonyl]-N¹-[(2,3-cis)-4-oxo-2-phenyl-2,3,4,5-tetrahydro-1,5-benzothiazepin-3-yl]-L-alaninamide (97a) (440 mg) in trifluoroacetic acid (10 mL) was stirred for 10 min. and concentrated under reduced pressure. The resulting material was diluted with 15% solution of potassium carbonate (20 mL) and extracted twice with DCM (150 mL). The DCM layer was dried over anhydrous potassium carbonate and concentrated under reduce pressure to afford the desired material (325 mg). ¹H NMR (300 MHz, d6-DMSO) δ0.90 (d, 1.5H, J=7 Hz), 0.98 (d, 1.5H, J=7 Hz), 3.11 (h, 1H J=7 Hz), 4.66 (m, 1H), 5.15 (t, 1H, J=6 Hz), 7.13-7.76 (m, 11H), 10.5 (br., 1H). MS APCI, m/z=342 (M+1). LC/MS: 1.59 min.

### Example 98

### N¹-[(2R,3R)-4-Oxo-2-phenyl-2,3,4,5-tetrahydro-1,5-benzothiazepin-3-yl]-N²-(2-phenylethyl)-L-alaninamide (98)

A solution of N¹-[(2,3cis)-4-oxo-2-phenyl-2,3,4,5-tetrahydro-1,5-benzothiazepin-3-yl]-L-alaninamide (97b) (618 mg) in 20 mL of methanol was treated with phenylacetaldehyde (220 mg), acetic acid (0.6 mL) followed by sodium cyanoborohydride (200 mg) and the resulting reaction mixture was stirred at 25° C. for 16 h. At the end of this period, the reaction mixture was treated with 2M HCl (1 mL), stirred for 30 min., concentrated under reduced pressure and diluted with DCM. Upon washing with 20% sodium carbonate solution the DCM layer was dried over anhydrous potassium carbonated and concentrated under reduce pressure to afford the crude product that was purified by column chromatography on silica gel. Elution with 2:1 DCM:EtOAc afforded purified product which was dissolved in methanol (2 mL) and treated with HCl in ether. Upon diluting with ether (150 mL) and stirring for 1 h the solid was collected by filtration to afford the title compound hydrochloride as a 1:1 mixture with the 2S,3S diastereomer (435 mg), white solid, m.p. 184-195° C. ¹H NMR (300 MHz, d6-DMSO) δ 0.99 (d, 1.5H, J=7 Hz), 1.25 (d, 1.5H, J=7 Hz), 2.9 (m, 4H), 4.66 (m, 1H), 3.91 (m, 1H), 4.77 (m, 1H), 5.20 (m, 1H), 7.04-7.54 (m, 12H), 7.70 (m, 1H), 8.42 (m, 1H), 8.86 (br, 1H), 9.50 (br., 1H), 10.60 (m, 1H). MS APCI, m/z=446(M+1). LC/MS: 2.17 min.

### Example 99

### N²-[(3,5-Difluorophenyl)acetyl]-N¹-[(2S,3R)-4-oxo-2-(2-thienyl)-2,3,4,5-tetrahydro-1,5-benzothiazepin-3-yl]-L-alaninamide. (99)

A method similar to the one described for 97 was used except that (2,3-cis)-3-amino-2-(2-thienyl)-2,3-dihydro-1,5-benzothiazepin-4(5H)-one hydrobromide (99c) (52 mg) was used as the amine component and N-[(3,5-difluorophenyl)acetyl]-L-alanine (1e) was used as the acid component to afford the title compound as a 1:1 mixture with the (2R,3R) diastereomer, white solid (79 mg), m.p. 117-119° C. ¹H NMR (300 MHz, d6-DMSO) δ 1.087 (m, 3H), 3.42 (m, 2H), 4.24 (m, 1H), 4.66 (m, 1H), 5.44 (d, 1H, J=7 Hz), 6.89-7.22 (m, 8H), 7.45 (m, 2H), 7.65 (d, 1H, J=8 Hz), 8.22 (two d, 1H, J=7 Hz), 10.43 (m, 1H). MS APCI, m/z=524 (M+Na). LC/MS: 2.20 min.

The starting amine (2R,3S)-3-amino-2-(2-thienyl)-2,3-dihydro-1,5-benzothiazepin-4(5H)-one hydrobromide 9 was prepared in the following manner:

### a. Methyl (2Z)-2-{[(benzyloxy)carbonyl]amino}-3-(2-thienyl)acrylate (99a)

In a dry reaction vessel 2-thiophenecarboxaldehyde (336 mg) was treated with a solution of N-(benzyloxycarbonyl)-α-phosphonoglycine trimethylester (762 mg) in DCM (2 mL) followed by a solution of DBU (304 mg) in DCM (2 mL). The reaction mixture was stirred for 2 h and the desired product was purified by column chromatography over silica gel. Elution with 20:1 DCM-EtOAc afforded the desired product (530 mg): ¹H NMR (300 MHz, CDCl₃) δ 3.795 (s, 3H), 5.19 (s, 2H), 5.99 (br., 1H), 7.07 (m, 1H), 7.34 (m, 7H), 7.49 (d, 1H, J=5 Hz), 7.77 (s, 1H). LC/MS: 2.32 min.

### b. Benzyl [(2,3-cis)-4-oxo-2-(2-thienyl)-2,3,4,5-tetrahydro-1,5-benzothiazepin-3-yl]carbamate (99b)

A solution of triethylamine (101 mg) and 2-aminothiophenol (375 mg) in MeOH (2 mL) was added to a solution of methyl (2Z)-2-{[(benzyloxy)carbonyl]amino}-3-(2-thienyl)acrylate (99a) in MeOH (5 mL). The reaction mixture was heated to 60° C. for 16 h and the solid product was filtered and dissolved in xylene (10 mL). Upon treating pTSA (10 mg) the reaction mixture was heated to reflux for 2 h. At the end of this period the reaction mixture was cooled to 25° C. and the solid was filtered, washed with hexane to afford the title compound as a white solid (142 mg), m.p. 184-195° C. ¹H NMR (300 MHz, d6-DMSO) δ 4.518 (t, 1H, J=7 Hz), 4.99 (s, 2H), 5.52 (d, 1H, J=7 Hz), 6.89-7.29 (m, 9H), 7.53 (m, 2H), 6.65 (d, 1H, J=8 Hz), 10.49 (s, 1H). LC/MS: 2.47 min.

### c. (2,3-cis)-3-Amino-2-(2-thienyl)-2,3-dihydro-1,5-benzothiazepin-4(5H)-one hydrobromide (99c)

A solution of benzyl [(2R,3S)-4-oxo-2-(2-thienyl)-2,3,4,5-tetrahydro-1,5-benzothiazepin-3-yl]carbamate (99b) (142 mg) in 30% hydrobromic acid in acetic acid (1 mL) was stirred for 1 h and diluted with ether (35 mL). The precipitate was collected, washed with ether, suspended in 20% sodium carbonate solution and extracted with DCM and EtOAc. Drying the organic layers with anhydrous potassium carbonate and concentration under reduced pressure afforded the title compound (52 mg). This material was used without further characterization.

### Example 100

### N²-[(3,5-Difluorophenyl)acetyl]-N¹-[(2R,3R)-4-oxo-2-(3-thienyl)-2,3,4,5-tetrahydro-1,5-benzothiazepin-3-yl]-L-alaninamide. (100)

A method similar to the one described for 97 was used except that (2,3-cis)-3-amino-2-(3-thienyl)-2,3-dihydro-1,5-benzothiazepin-4(5H)-one hydrobromide (100c) (147 mg) was used as the amine component and N-[(3,5-difluorophenyl)acetyl]-L-alanine (1e) was used as the acid component to afford the title compound as a 1:1 mixture with the 2S,3S diastereomer, white solid (212 mg), m.p. 117-122° C. ¹H NMR (300 MHz, d6-DMSO) δ 1.03 (m, 3H), 3.40 (m, 2H), 4.20 (m, 1H), 4.61 (m, 1H), 5.26 (m, 1H), 6.90-7.36 (m, 7H), 7.52 (m, 4H), 7.65 (d, 1H, J=9 Hz), 8.28 (two d, 1H, J=7 Hz), 10.51 (d, 1H, J=12 Hz). MS APCI, m/z=501 (M+1). LC/MS: 2.20 min.

The starting amine (2,3-cis)-3-amino-2-(3-thienyl)-2,3-dihydro-1,5-benzothiazepin-4(5H)-one hydrobromide (100c) was prepared in the following manner:

### a. Methyl (2Z)-2-{[(benzyloxy)carbonyl]amino}-3-(3-thienyl)acrylate (100a)

A method similar to that used for the preparation of (99a) was used except that 3-thiophenecarboxaldehyde (762 mg) was used as the aldehyde component to afforded the desired product (549 mg). ¹H NMR (300 MHz, CDCl₃) δ 3.795 (s, 3H), 5.19 (s, 2H), 5.99 (br., 1H), 7.07 (m, 1H), 7.34 (m, 7H), 7.49 (d, 1H, J=5 Hz), 7.77 (s, 1H). LC/MS: 2.32 min.

### b. Benzyl [(2,3-cis)-4-oxo-2-(3-thienyl)-2,3,4,5-tetrahydro-1,5-benzothiazepin-3-yl]carbamate (100b)

A method similar to that used for the preparation of (99b) was used except that methyl (2Z)-2-{[(benzyloxy)carbonyl]amino}-3-(3-thienyl)acrylate (100a) (549 mg) was used as starting material to afford the title compound as a white solid. ¹H NMR (300 MHz, d6-DMSO) δ 4.5 (t, 1H, J=7 Hz), 4.96 (s, 2H), 5.33 (d, 1H, J=7 Hz), 6.10 (d, 1H, J=7 Hz), 7.14-7.30 (m, 8H), 7.50 (m, 3H), 6.65 (d, 1H, J=8 Hz), 10.47 (s, 1H). LC/MS: 2.56 min.

### c. (2,3-cis)-3-Amino-2-(3-thienyl)-2,3-dihydro-1,5-benzothiazepin-4(5H)-one hydrobromide (100c)

A method similar to that used for the preparation of (99c) was used except that benzyl [(2,3-cis)-4-oxo-2-(3-thienyl)-2,3,4,5-tetrahydro-1,5-benzothiazepin-3-yl]carbamate (100b) was used as starting material to afford the title compound (147 mg). This material was used without further characterization.

### Example 101

### N²-[(3,5-Difluorophenyl)acetyl]-N¹-[(2S,3R)-2-(2-furyl)-4-oxo-2,3,4,5-tetrahydro-1,5-benzothiazepin-3-yl]-L-alaninamide (101)

A method similar to the one described for (97) was used except that (2,3-cis)-3-amino-2-(2-furyl)-2,3-dihydro-1,5-benzothiazepin-4(5H)-one (101c) (56 mg) was used as the amine component and N-[(3,5-difluorophenyl)acetyl]-L-alanine (1e) was used as the acid component to afford the title compound as a 1:1 mixture with the 2R,3S diastereomer, white solid (44 mg), m.p. 125-130° C. ¹H NMR (300 MHz, d6-DMSO) δ 1.01 (m, 3H), 3.45 (m, 2H), 4.30 (m, 1H), 4.70 (m, 1H), 5.30 (m, 1H), 6.5 (m, 2H), 7.0 (m, 3H), 7.5 (m, 2H), 7.75 (m, 3H), 8.25 (two d, 1H, J=7 Hz), 10.45 (d, 1H, J=12 Hz). MS APCI, m/z=508 (M+Na). LC/MS: 2.20 min.

The starting amine (2,3-cis)-3-amino-2-(2-furyl)-2,3-dihydro-1,5-benzothiazepin-4(5H)-one hydrobromide (101c) was prepared in the following manner:

### a. Methyl (2Z)-2-{[(benzyloxy)carbonyl]amino}-3-(2-furyl)acrylate (101a)

A method similar to that used for the preparation of (99a) was used except that 2-furylcarboxaldehyde (288 mg) was used as the aldehyde component to afforded the desired product (530 mg). ¹H NMR (300 MHz, CDCl₃) δ 3.788 (s, 3H), 5.17 (s, 2H), 6.29 (m, 1H), 6.46 (m, 1H), 6.73 (br., 1H), 6.94 (s, 1H), 7.37 (m, 6H). LC/MS: 2.27 min.

### b. Benzyl [(2,3-cis)-4-oxo-2-(2-furyl)-2,3,4,5-tetrahydro-1,5-benzothiazepin-3-yl]carbamate 101b)

A method similar to that used for the preparation of (99b) was used except that methyl (2Z)-2-{[(benzyloxy)carbonyl]amino}-3-(2-furyl)acrylate 101a (530 mg) was used as starting material to afford the title compound as a white solid (94 mg). ¹H NMR (300 MHz, d6-DMSO) δ 4.51 (t, 1H, J=7 Hz), 4.98 (s, 2H), 5.32 (d, 1H, J=7 Hz), 6.52 (m, 2H), 6.90-7.66 (m, 11H), 10.43 (s, 1H). LC/MS: 2.31 min.

### c. (2,3-cis)-3-Amino-2-(2-furyl)-2,3-dihydro-1,5-benzothiazepin-4(5H)-one hydrobromide (101c)

A method similar to that used for the preparation of (99c) was used except that benzyl [(2,3-cis)-4-oxo-2-(2-furyl)-2,3,4,5-tetrahydro-1,5-benzothiazepin-3-yl]carbamate (101b) was used as starting material to afford the title compound (56 mg). This material was used in the next step without further characterization.

### Example 102

### N²-[(3,5-Difluorophenyl)acetyl]-N¹-[(2S,3R)-2-(3-furyl)-4-oxo-2,3,4,5-tetrahydro-1,5-benzothiazepin-3-yl]-L-alaninamide (102)

A method similar to the one described for 97 was used except that (2,3-cis)-3-amino-2-(3-furyl)-2,3-dihydro-1,5-benzothiazepin-4(5H)-one (102c) (80 mg) was used as the amine component and N-[(3,5-difluorophenyl)acetyl]-L-alanine (1e) was used as the acid component to afford the title compound as a 1:1 mixture with the 2R,3S diastereomer, white foam (144 mg), m.p. 115-120° C. ¹H NMR (300 MHz, d6-DMSO) δ 0.93 (m, 3H), 3.29 (m, 2H), 4.32 (m, 1H), 4.64 (m, 1H), 5.04 (m, 1H), 6.54 (m, 1H), 6.95 (m, 3H), 7.08 (m, 2H), 7.46 (m, 2H), 7.59 (m, 3H), 8.29 (two d, 1H, J=7 Hz), 10.42 (d, 1H, J=13 Hz). MS APCI, m/z=508 (M+Na) LC/MS: 2.31 min.

The starting amine (2,3-cis)-3-amino-2-(3-furyl)-2,3-dihydro-1,5-benzothiazepin-4(5H)-one hydrobromide (102c) was prepared in the following manner:

### a. Methyl (2Z)-2-{[(benzyloxy)carbonyl]amino}-3-(3-furyl)acrylate (102a)

A method similar to that used for the preparation of (99a) was used except that 3-furylcarboxaldehyde (288 mg) was used as the aldehyde component to afforded the desired product (531 mg). ¹H NMR (300 MHz, CDCl₃) δ 3.79 (s, 3H), 5.16 (s, 2H), 6.12 (br., 1H), 6.57 (s, 1H), 7.36 (m, 7H), 7.68 (s, 1H). LC/MS: 2.24 min.

### b. Benzyl [(2R,3S)-4-oxo-2-(3-furyl)-2,3,4,5-tetrahydro-1,5-benzothiazepin-3-yl]carbamate (102b)

A method similar to that used for the preparation of (99b) was used except that methyl (2Z)-2-{[(benzyloxy)carbonyl]amino}-3-(3-furyl)acrylate (102a) (530 mg) was used as starting material to afford the title compound as a white solid (235 mg). ¹H NMR (300 MHz, d6-DMSO) δ 4.66 (t, 1H, J=4 Hz), 4.97 (s, 2H), 5.20 (d, 1H, J=6 Hz), 6.38 (d, 2H, J=7 Hz), 6.5 (s, 1H), 7.07-7.75 (m, 1H), 10.44 (s, 1H). LC/MS: 2.32 min.

### c. (2,3-cis)-3-Amino-2-(3-furyl)-2,3-dihydro-1,5-benzothiazepin-4(5H-one hydrobromide (102c)

A method similar to that used for the preparation of (99c) was used except that benzyl [(2R,3S)-4-oxo-2-(3-furyl)-2,3,4,5-tetrahydro-1,5-benzothiazepin-3-yl]carbamate (102b) was used as starting material to afford the title compound (80 mg). This material was used without further characterization.

### Example 103

### N¹-[(2S,3R)-2-(5-Bromo-2-thienyl)-4-oxo-2,3,4,5-tetrahydro-1,5-benzothiazepin-3-yl]-N²-[(3,5-difluorophenyl)acetyl]-L-alaninamide (103)

A method similar to the one described for (97) was used except that (2,3-cis)-3-amino-2-(5-bromo-2-thienyl)-2,3-dihydro-1,5-benzothiazepin-4(5H)-one (103c) (460 mg) was used as the amine component and N-[(3,5-difluorophenyl)acetyl]-L-alanine (1e) was used as the acid component to afford the title compound as a 1:1 mixture with the 2R,3S diastereomer (357 mg), white solid, m.p. 139-140° C. ¹H NMR (300 MHz, d6-DMSO) δ 1.09 (m, 3H), 3.45 (m, 2H), 4.30 (m, 1H), 4.66 (m, 1H), 5.36 (m, 1H), 6.87-7.25 (m, 6H), 7.42 (t, 1H, J=11 Hz), 7.64-7.83 (m, 2H), 8.31 (two d, 2H, J=7 Hz), 10.55 (d, 1H, J=8 Hz). MS APCI, m/z=582 (M+1). LC/MS: 2.41 min.

The starting amine (2,3-cis)-3-amino-2-(5-bromo-2-thienyl)-2,3-dihydro-1,5-benzothiazepin-4(5H)-one hydrobromide (103c) was prepared in the following manner:

### a. Methyl (2Z)-2-{[(benzyloxy)carbonyl]amino}-3-(5-bromo-2-thienyl)acrylate (103a)

A method similar to that used for the preparation of (99a) was used except that the reaction product was passed through a Dowex ion exchange resin 50×2-200 and 5-bromo-2-thiphenecarboxaldehyde (955 mg) was used as the aldehyde component to afforded the desired product (1.80 g). ¹H NMR (300 MHz, CDCl₃) δ 3.79 (s, 3H), 5.20 (s, 2H), 5.93 (br., 1H), 6.95 (m, 1H), 7.36 (m, 5H), 7.70 (s, 1H). LC/MS: 2.36 min.

### b. Benzyl [(2,3-cis)-4-oxo-2-(5-bromo-2-thienyl)-2,3,4,5-tetrahydro-1,5-benzothiazepin-3-yl]carbamate (103b)

A method similar to that used for the preparation of (99b) was used except that methyl (2Z)-2-{[(benzyloxy)carbonyl]amino}-3-(5-bromo-2-thienyll)acrylate (103a) (1.8 g) was used as the starting material to afford the title compound (651 mg). ¹H NMR (300 MHz, d6-DMSO) δ 4.93 (m, 1H), 4.98 (s, 2H), 5.11 (br., 1H), 5.46 (d, 1H, J=6 Hz), 6.55 (d, 1H, J=7 Hz), 7.11 (d, 1H, J=4 Hz), 7.13-7.74 (m, 10H), 10.51 (s, 1H). LC/MS: 2.66 min.

### c. (2,3-cis)-3-Amino-2-(5-bromo-2-thienyl)-2,3-dihydro-1,5-benzothiazepin-4(5H)-one hydrobromide (103c)

A method similar to that used for the preparation of (99c) was used except that benzyl [(2,3-cis)-4-oxo-2-(5-bromo-2-thienyl)-2,3,4,5-tetrahydro-1,5-benzothiazepin-3-yl]carbamate (103b) was used as the starting material to afford the title compound (460 mg). This material was used without further purification.

### Example 104

### N¹-[(2S,3R)-2-(4-Bromo-2-thienyl)-4-oxo-2,3,4,5-tetrahydro-1,5-benzothiazepin-3-yl]-N²-[(3,5-difluorophenyl)acetyl]-L-alaninamide (104)

A method similar to the one described for (97) was used except that (2,3-cis)-3-amino-2-(4-bromo-2-thienyl)-2,3-dihydro-1,5-benzothiazepin-4(5H)-one (104c) (320 mg) was used as the amine component and N-[(3,5-difluorophenyl)acetyl]-L-alanine (1e) was used as the acid component to afford the title compound in 1:1 mixture with the 2R,3S diastereomer (357 mg), white solid, m.p. 145-155° C. ¹H NMR (300 MHz, d6-DMSO) δ 1.09 (m, 3H), 3.42 (m, 2H), 4.31 (m, 1H), 4.69 (m, 1H), 5.38 (m, 1H), 6.90-7.30 (m, 6H), 7.50 (t, 1H, J=7.5 Hz), 7.657 (m, 2H), 7.88 (d, 1H, J=7.5 Hz), 8.31 (two d, 2H, J=7 Hz), 10.55 (d, 1H, J=8 Hz). MS APCI, m/z=582 (M+1) LC/MS: 2.37 min.

The starting amine (2,3-cis)-3-amino-2-(4-bromo-2-thienyl)-2,3-dihydro-1,5-benzothiazepin-4(5H)-one hydrobromide (104c) was prepared in the following manner:

### a. Methyl (2Z)-2-{[(benzyloxy)carbonyl]amino}-3-(4-bromo-2-thienyl)acrylate (104a)

A method similar to that used for the preparation of (99a) was used except that the reaction product was passed through a Dowex ion exchange resin 50×2-200 and 4-bromo-2-thiphenecarboxaldehyde (955 mg) was used as the aldehyde component to afford the desired product (1.52 g). ¹H NMR (300 MHz, CDCl₃) δ 3.79 (s, 3H), 5.24 (s, 2H), 5.99 (br., 1H), 7.20-7.37 (m, 7H), 7.67 (s, 1H). LC/MS: 2.37 min.

### b. Benzyl [(2,3-cis)-4-oxo-2-(4-bromo-2-thienyl)-2,3,4,5-tetrahydro-1,5-benzothiazepin-3-yl]carbamate (104b)

A method similar to that used for the preparation of (99b) was used except that methyl (2Z)-2-{[(benzyloxy)carbonyl]amino}-3-(4-bromo-2-thienyl)acrylate (104a) (1.8 g) was used as starting material to afford the title compound (528 mg). ¹H NMR (300 MHz, d6-DMSO) δ 4.51 (m, 1H), 4.98 (s, 2H), 5.47 (d, 1H, J=6 Hz), 6.74 (d, 1H, J=7 Hz), 7.09 (s, 1H), 7.32 (m, 7H), 7.51 (t, 1H, J=5 Hz), 7.689 (m, 2H), 10.51 (s, 1H). LC/MS: 2.63 min.

### c. (2,3-cis)-3-Amino-2-(4-bromo-2-thienyl)-2,3-dihydro-1,5-benzothiazepin-4(5R)-one hydrobromide 104c

A method similar to that used for the preparation of (99c) was used except that benzyl [(2,3-cis)-4-oxo-2-(4-bromo-2-thienyl)-2,3,4,5-tetrahydro-1,5-benzothiazepin-3-yl]carbamate (104b) was used as starting material to afford the title compound (460 mg). This material was used without further purification.

### Example 105

### N-[(3,5-Difluorophenyl)acetyl]-N-[(2R,3R)-4-oxo-2-phenyl-2,3,4,5-tetrahydro-1,5-benzothiazepin-3-yl]-L-phenylalaninamide) (105)

A method similar to the one described for (97) was used except that N-[(2,3-cis)-4-oxo-2-phenyl-2,3,4,5-tetrahydro-1,5-benzothiazepin-3-yl]-L-phenylalaninamide (105a) was used as the amine component and 3,5-difluorophenylacetic acid was used as the acid component to afford the title compound as a 1:1 mixture with the 2S,3S diastereomer (58 mg), white solid, m.p. 115-120° C. ¹H NMR (300 MHz, d6-DMSO) δ 4.45 (m, 1H), 4.75 (m, 1H), 5.10 (m, 1H), 6.70 (m, 2H), 6.95-7.90 (m, 16H), 8.25 (two d, 1H, J=8 Hz), 10.5 (m, 1H). MS APCI, m/z=572 (M+1). LC/MS: 2.68 min.

The starting amine N-[(2,3-cis)-4-oxo-2-phenyl-2,3,4,5-tetrahydro-1,5-benzothiazepin-3-yl]-L-phenylalaninamide (105a) was prepared in the following manner:

### a. N-[(2S,3S)-4-Oxo-2-phenyl-2,3,4,5-tetrahydro-1,5-benzothiazepin-3-yl]-L-phenylalaninamide (105a)

To a solution of (2S,3S)-3-amino-2-phenyl-2,3-dihydro-1,5-benzothiazepin-4(5H)-one hydrobromide (9d) (91 mg) in 10 mL of THF was added Fmoc-Phe-Opfp (185 mg) followed by DIEA (58 μL). The reaction mixture was stirred for 30 min and treated with DBU (0.1 mL) and stirred for 10 min. The material thus produced was used in the next step without purification or characterization.

### Example 106

### N²-[(3,5-Difluorophenyl)acetyl]-N¹-[(2R,3R)-4-oxo-2-phenyl-2,3,4,5-tetrahydro-1,5-benzothiazepin-3-yl]glycinamide (106)

A method similar to the one described for (97) was used except that N-[(2,3-cis)-4-oxo-2-phenyl-2,3,4,5-tetrahydro-1,5-benzothiazepin-3-yl]glycinamide (106a) was used as the amine component and 3,5-difluorophenylacetic acid was used as the acid component to afford the title compound as a 1:1 mixture with the 2S,3S diastereomer (80 mg), white solid, m.p. 125-130° C. ¹H NMR (300 MHz, d6-DMSO) δ 4.76 (t, 1H, J=7 Hz), 5.12 (d, 1H, J=7 Hz), 6.91-7.49 (m, 12H), 7.67 (d, 1H, J=7 Hz), 8.25 (s, 1H), 10.49 (s, 1H). MS APCI, m/z=482(M+1). LC/MS: 2.27 min.

The starting amine N-[(2,3-cis)-4-oxo-2-phenyl-2,3,4,5-tetrahydro-1,5-benzothiazepin-3-yl]-L-phenylalaninamide (106a) was prepared in the following manner:

### a. N-[(2,3-cis)-4-Oxo-2-phenyl-2,3,4,5-tetrahydro-1,5-benzothiazepin-3-yl]glycinamide (106a)

A method similar to the preparation of (105a) was used except that Fmoc-Gly-Opfp (153 mg) was used in place of Fmoc-Phe-Opfp. The material thus produced was used in the next step without purification or characterization.

### Example 107

### N²-[(3,5-Difluorophenyl)acetyl]-N¹-[(2R,3R)-4-oxo-2-phenyl-2,3,4,5-tetrahydro-1,5-benzothiazepin-3-yl]-L-valinamide (107)

A method similar to the one described for (97) was used except that (2,3-cis)-3-amino-2-phenyl-2,3-dihydro-1,5-benzothiazepin-4(5 h)-one hydrobromide (9d) (105 mg) was used as the amine component and N-[(3,5-difluorophenyl)acetyl]-L-valine (81 mg) 107b was used as the acid component to afford the title compound in 1:1 mixture with the (2S,3S) diastereomer (91 mg), white solid, m.p. 110-115° C. ¹H NMR (300 MHz, d6-DMSO) δ 4.76 (t, 1H, J=7 Hz), 5.12 (d, 1H, J=7 Hz), 6.91-7.49 (m, 12H), 7.67 (d, 1H, J=7 Hz), 8.25 (s, 1H), 10.49 (s, 1H). MS APCI, m/z=524(M+1). LC/MS: 2.54 min.

The starting acid N-[(3,5-difluorophenyl)acetyl]-L-valine (107b) was prepared in the following manner:

### a. Methyl N-[(3,5-difluorophenyl)acetyl]-L-valinate (107a)

To a solution of methyl-L-valinate hydrochloride (168 mg) in DCM (6 mL) was added HOBt (135 mg), 3,5-difluorophenylacetic acid (172 mg), EDCI (191 mg) and DIEA (0.522 mL). The reaction mixture was stirred for 16 h, diluted with DCM (50 mL), washed with 5% hydrochloric acid (10 mL) and 10% potassium carbonate (10 mL). The organic layer after drying over anhydrous magnesium sulfate and concentration under reduced pressure afforded the title compound (272 mg). ¹H NMR (300 MHz, CDCl₃) δ 0.87 (d, 1.5H, J=7 Hz), 0.89 (d, 1.5H, J=7 Hz), 2.01 (m, 1H), 3.58 (s, 2H), 3.63 (s, 3H), 4.18 (d, d, 1H, J=6 Hz, J=8 Hz), 7.0 (m, 3H), 8.45 (d, 1H, J=8 Hz). MS APCI, m/z=244(M+1). LC/MS: 1.97 min.

### b. N-[(3,5-Difluorophenyl)acetyl]-L-valine (107b)

A solution of methyl N-[(3,5-difluorophenyl)acetyl]-L-valinate (107a) in methanol (10 mL) was treated with 1N sodium hydroxide solution (2 mL) and stirred for 2 h. At the end of this period the reaction mixture was acidified with 1N hydrochloric acid solution, diluted with saturated sodium chloride and extracted with DCM (100 mL). After drying over anhydrous magnesium sulfate and concentration under reduced pressure the organic layer afforded the title compound (210 mg). ¹H NMR (300 MHz, CDCl₃)) δ 0.87 (m, 3H), 2.05 (m, 1H), 3.58 (m, 2H), 4.15 (t, 1H, J=8 Hz), 7.07 (m, 3H), 8.30 (m, 1H), 12.61 (s, 1H). MS APCI, m/z=226(M+1) LC/MS: 1.84 min.

### Example 108

### N²-[(3,5-Difluorophenyl)acetyl]-N¹-[(2R,3R)-4-oxo-2-phenyl-2,3,4,5-tetrahydro-1,5-benzothiazepin-3-yl]-L-valinamide (108)

A method similar to the one described for (97) was used except that (2,3-cis)-3-amino-2-phenyl-2,3-dihydro-1,5-benzothiazepin-4(5H)-one hydrobromide (9d) (105 mg) was used as the amine component and N-[(3,5-difluorophenyl)acetyl]-L-leucine (108b) (85 mg) was used as the acid component to afford the title compound as a 1:1 mixture with the 2S,3S diastereomer (107 mg), white solid, m.p. 180-190° C. ¹H NMR (300 MHz, d6-DMSO) δ 4.76 (t, 1H, J=7 Hz), 5.12 (d, 1H, J=7 Hz), 6.91-7.49 (m, 12H), 7.67 (d, 1H, J=7 Hz), 8.25 (s, 1H), 10.49 (s, 1H). MS APCI, m/z=538(M+1). LC/MS: 2.65 min.

The starting acid N-[(3,5-difluorophenyl)acetyl]-L-leucine (108b) was prepared in the following manner:

### a. Methyl N-[(3,5-difluorophenyl)acetyl]-L-leucinate (108a)

A method similar to that used for the preparation of (107a) was used except that methyl-L-leucinate hydrochloride (182 mg) was used instead of methyl-L-valinate hydrochloride to afford the desired product (280 mg). ¹H NMR (300 MHz, CDCl₃) δ 0.69 (d, 3H, J=6 Hz), 0.81 (d, 3H, J=6 Hz), 1.48 (m, 3H), 3.47 (s, 2H), 3.61 (s, 3H), 4.26 (m, 1H), 7.07 (m, 3H), 8.54 (d, 1H, J=8 Hz). MS APCI, m/z=300(M+1). LC/MS: 2.15 min.

### b. N-[(3,5-Difluorophenyl)acetyl]-L-leucine (108b)

A method similar to that used for the preparation of (107b) was used except that methyl N-[(3,5-difluorophenyl)acetyl]-L-leucinate (108a) (280 mg) was used instead of methyl N-[(3,5-difluorophenyl)acetyl]-L-valinate to afford the desired product (216 mg). ¹H NMR (300 MHz, CDCl₃) δ 0.83 (d, 3, J=6 Hz), 0.89 (d, 3H, J=6 Hz), 1.51 (m, 3H), 3.52 (s, 2H), 4.22 (m, 1H), 7.07 (m, 3H), 8.41 (d, 11H, J=8 Hz), 12.54 (s, 11H).

### Example 109

### N²-[(3,5-Difluorophenyl)acetyl]-N¹-[(2R,3R)-4-oxo-2-phenyl-2,3,4,5-tetrahydro-1,5-benzothiazepin-3-yl]-L-methioninamide (109)

A method similar to the one described for (97) was used except that (2,3-cis)-3-amino-2-phenyl-2,3-dihydro-1,5-benzothiazepin-4(5H)-one hydrobromide (9d) (105 mg) was used as the amine component and N-[(3,5-difluorophenyl)acetyl]-L-methionine (109b) (91 mg) was used as the acid component to afford the title compound as a 1:1 mixture with the (2S,3S) diastereomer (63 mg), white solid, m.p. 85-90° C. ¹H NMR (300 MHz, d6-DMSO) δ 1.55 (m, 4H), 2.20 (s, 3H), 2.40 (m, 2H), 3.03 (m, 4H), 4.26 (m, 1H), 4.72 (m, 1H), 5.17 (d, 1H, J=7 Hz), 5.829 (d, 1H, j=6 Hz), 6.89-7.79 (m, 12H), 8.21 (m, 1H), 10.53 (m, 1H). MS APCI, m/z=556(M+1). LC/MS: 2.55 min.

The starting acid N-[(3,5-difluorophenyl)acetyl]-L-methionine (109b) was prepared in the following manner:

### a. Methyl N-[(3,5-difluorophenyl)acetyl]-L-methioninate (109a)

A method similar to that used for the preparation of (107a) was used except that methyl-L-methionine hydrochloride (214 mg) was used instead of methyl-L-valinate hydrochloride to afford the desired product (300 mg). ¹H NMR (300 MHz, CDCl₃) δ 1.178 (t, 3H, J=7 Hz), 1.93 (m, 2H), 2.02 (s, 3H), 2.51 (m, 2H), 3.53 (s, 2H), 4.38 (m, 1H), 7.07 (m, 3H), 8.54 (d, 1H, J=8 Hz). LC/MS: 2.28 min.

### b. N-[(3,5-Difluorophenyl)acetyl]-L-methionine (109b)

A method similar to that used for the preparation of (107b) was used except that methyl N-[(3,5-difluorophenyl)acetyl]-L-methioninate (109a) (300 mg) was used instead of methyl N-[(3,5-difluorophenyl)acetyl]-L-valinate to afford the desired product (237 mg). This material was not further characterized but used in the next step.

### Example 110

### N²-[(3,5-Difluorophenyl)acetyl]-3-(1H-indol-2-yl)-N¹-[(2R,3R)-4-oxo-2-phenyl-2,3,4,5-tetrahydro-1,5-benzothiazepin-3-yl]-L-alaninamide (110)

A method similar to the one described for (97) was used except that (2,3-cis S)-3-amino-2-phenyl-2,3-dihydro-1,5-benzothiazepin-4(5H)-one hydrobromide (9d) (105 mg) was used as the amine component and N-[(3,5-difluorophenyl)acetyl]-3-(1H-indol-2-yl)-L-alanine (110b) (119 mg) was used as the acid component to afford the title compound as a 1:1 mixture with the 2S,3S diastereomer (173 mg), white solid, m.p. 85-90° C. ¹H NMR (300 MHz, d6-DMSO) δ 2.40 (m, 2H), 3.03 (m, 4H), 4.26 (m, 1H), 4.72 (m, 1H), 5.17 (d, 1H, J=7 Hz), 5.82 (d, 1H, J=6 Hz), 6.89-7.79 (m, 12H), 8.21 (m, 1H), 10.53 (m, 1H). MS APCI, m/z=611(M+1). LC/MS: 2.66 min.

The starting acid N-[(3,5-difluorophenyl)acetyl]-3-(1H-indol-2-yl)-L-alanine (110b) was prepared in the following manner:

### a. Methyl N-[(3,5-difluorophenyl)acetyl]-3-(1H-indol-2-yl)-L-alaninate (110a)

A method similar to that used for the preparation of (107a) was used except that methyl-L-triptophane hydrochloride (214 mg) was used instead of methyl-L-valinate hydrochloride to afford the desired product (365 mg). ¹H NMR (300 MHz, CDCl₃) δ 3.17 (m, 2H), 3.43 (s, 2H), 4.90 (m, 1H), 5.94 (d, 1H, J=7 Hz), 6.62 (m, 2H), 6.71 (m, 1H), 7.12 (m, 1H), 7.17 (m, 1H), 7.36 (d, 1H, J=8 Hz), 7.39 (d, 1H, J=8 Hz), 8.09 (s, 1H). MS APCI, m/z=373(M+1). LC/MS: 2.28 min.

### b. N-[(3,5-Difluorophenyl)acetyl]-3-(1H-indol-2-yl)-L-alanine (110b)

A method similar to that used for the preparation of (107b) was used except that methyl N-[(3,5-difluorophenyl)acetyl]-L-methioninate (110a) (300 mg) was used instead of methyl N-[(3,5-difluorophenyl)acetyl]-L-valinate to afford the desired product (341 mg). This material was not further characterized but used in the next step.

### Example 111

### N²-[(3,5-Difluorophenyl)acetyl]-N¹-[(2R,3R)-4-oxo-2-phenyl-2,3,4,5-tetrahydro-1,5-benzothiazepin-3-yl]-L-α-aspartic acid (111)

A method similar to the one described for (97) was used except that (2,3-cis)-3-amino-2-phenyl-2,3-dihydro-1,5-benzothiazepin-4(5H)-one hydrobromide (9d) (105 mg) was used as the amine component and 4-tert-butyl N-[(3,5-difluorophenyl)acetyl]-L-aspartate (110b) (115 mg) was used as the acid component to afford a product which was treated with trifluoroacetic acid (2 mL) and stirred for 5 min. At the end of this period the reaction mixture was concentrated under reduced pressure, diluted with EtOAc and hexanes and the solid was filtered to afford the title compound as a 1:1 mixture with the (2S,3S) diastereomer (118 mg), white solid, m.p. 65-80° C. ¹H NMR (300 MHz, d6-DMSO) δ 4.44 (m, 1H), 4.72 (m, 1H), 5.13 (d, 1H, J=7 Hz), 6.03 (s, 1H), 6.87-7.73 (m, 11H), 8.41 (m, 1H), 9.38 (s, 1H), 10.51 (m, 1H). MS APCI, m/z=540(M+1). LC/MS: 2.66 min.

The starting acid 4-tert-butyl N-[(3,5-difluorophenyl)acetyl]-L-aspartate (110b) was prepared in the following manner:

### a. 4-tert-Butyl 1-methyl N-[(3,5-difluorophenyl)acetyl]-L-aspartate (111a)

A method similar to that used for the preparation of (107a) was used except that 4-tert-butyl 1-methyl-L-aspartate hydrochloride (239 mg) was used instead of methyl-L-valinate hydrochloride to afford the desired product (350 mg). ¹H NMR (300 MHz, CDCl₃) δ 2.69 (d, d, 1H, J=5 Hz, J=5 Hz), 2.95 (dd, 1H, J=5 Hz, J=5 Hz), 3.56 (s, 2H), 3.74 (s, 3H), 4.82 (m, 1H), 6.50 (d, 1H, J=7 Hz), 6.79 (m, 3H).

### b. 4-tert-Butyl N-[(3,5-difluorophenyl)acetyl]-L-aspartate (111b)

A method similar to that used for the preparation of (107b) was used except that 4-tert-butyl 1-methyl N-[(3,5-difluorophenyl)acetyl]-L-aspartate (111a) (350 mg) was used instead of methyl N-[(3,5-difluorophenyl)acetyl]-L-valinate to afford the desired product (292 mg). This material was not further characterized but used in the next step

### Example 112

### N²-[(3,5-Difluorophenyl)acetyl]-N¹-[(2R,3R)-4-oxo-2-phenyl-2,3,4,5-tetrahydro-1,5-benzothiazepin-3-yl]-L-α-glutamic acid (112c)

A method similar to the one described for (97) was used except that (2,3-cis)-3-amino-2-phenyl-2,3-dihydro-1,5-benzothiazepin-4(5H)-one hydrobromide (9d) (105 mg) was used as the amine component and 4-tert-butyl N-[(3,5-difluorophenyl)acetyl]-L-glutamate (112b) (119 mg) was used as the acid component to afford a product which was treated with trifluoroacetic acid (2 mL) and stirred for 5 min. At the end of this period the reaction mixture was concentrated under reduced pressure, diluted with EtOAc and hexanes and the solid was filtered to afford the title compound in 1:1 mixture with the (2S,3S) diastereomer (160 mg), white solid, m.p. 65-80° C. ¹H NMR (300 MHz, d6-DMSO) δ 4.20 (m, 1H), 4.72 (m, 1H), 5.15 (d, 1H, J=7 Hz), 6.03 (s, 1H), 6.89-7.73 (m, 11H), 8.41 (m, 1H), 9.39 (s, 1H), 10.52 (m, 1H). MS APCI, m/z=554(M+1). LC/MS: 2.27 min.

The starting acid 4-tert-butyl N-[(3,5-difluorophenyl)acetyl]-L-glutamate (112b) was prepared in the following manner:

### a. 4-tert-Butyl 1-methyl N-[(3,5-difluorophenyl)acetyl]-L-glutamate (112a)

A method similar to that used for the preparation of (107a) was used except that 4-tert-butyl 1-methyl-L-glutamate hydrochloride (253 mg) was used instead of methyl-L-valinate hydrochloride to afford the desired product (360 mg). ¹H NMR (300 MHz, CDCl₃) δ 1.95-2.34 (m, 4H), 3.47 (s, 2H), 3.79 (s, 3H), 4.59 (m, 1H), 6.3 (d, 1H, J=7 Hz), 6.79 (m, 3H).

### b. 4-tert-Butyl N-[(3,5-difluorophenyl)acetyl]-L-glutamate (112b)

A method similar to that used for the preparation of (107b) was used except that 4-tert-butyl 1-methyl N-[(3,5-difluorophenyl)acetyl]-L-glutamate (111a) (360 mg) was used instead of methyl N-[(3,5-difluorophenyl)acetyl]-L-valinate to afford the desired product (325 mg). This material was not further characterized but used in the next step

### Example 113

### N¹-[(2R,3S)-4-Oxo-2-phenyl-2,3,4,5-tetrahydro-1,5-benzoxazepin-3-yl]-N²-(phenylacetyl)-L-alaninamide (113)

To a solution of N¹-[(2,3-cis)-4-oxo-2-phenyl-2,3,4,5-tetrahydro-1,5-benzoxazepin-3-yl]-L-alaninamide (113a) (130 mg), HOBt (81 mg), phenylacetic acid (68 mg), and EDAC-HCl (115 mg) in DCM (10 mL) was added triethylamine (126 mg). The mixture was stirred at 25° C. under nitrogen for 16 h and purified by flash chromatography (2:1 DCM-EtOAc) to afford the title compound in 1:1 mixture with the 2S,3R diastereomer (135 mg), white solid, m.p. 122-136° C. ¹H NMR (300 MHz, CDCl₃) δ 0.97 (d, 1.5H, J=5 Hz), 1.05 (d, 1.5H, J=5 Hz), 3.47 (two peaks, 2H), 4.53 (p, 0.5H, J=7 Hz), 4.60 (p, 0.5H, J=7 Hz), 4.89 (t, 1H, J=6 Hz), 5.11(t, 1H, J=6 Hz), 5.78 (d, d, 1H, J=7 Hz, J=10 Hz), 6.08 (d, 0.5H, J=8 Hz), 6.2 (d, 0.5 Hz, J=8 Hz), 6.61 (d, 0.5H, J=6 Hz), 6.80 (d, 0.5H, J=7 Hz), 6.91-7.34 (m, 14H), 7.997 (s, 1H). MS APCI, m/z=466 (M+Na). LC/MS: 2.04 min.

The starting amine N¹-[(2,3-cis)-4-oxo-2-phenyl-2,3,4,5-tetrahydro-1,5-benzoxazepin-3-yl]-L-alaninamide (113a) was prepared in the following manner:

### a. N¹-[(2,3-cis)-4-Oxo-2-phenyl-2,3,4,5-tetrahydro-1,5-benzoxazepin-3-yl]-L-alaninamide (113a)

A solution of N²-[tert-Butoxycarbonyl]-N¹-[(2,3-cis)-4-oxo-2-phenyl-2,3,4,5-tetrahydro-1,5-benzoxazepin-3-yl]-L-alaninamide (6f) (1.94 g) in trifluoroacetic acid (10 mL) was stirred for 10 min. and concentrated under reduced pressure. The resulting material was diluted with 15% solution of sodium carbonate (50 mL) and extracted twice with DCM (100 mL). The DCM layer was dried over anhydrous potassium carbonate and concentrated under reduce pressure to afford the desired material (1.33 g). ¹H NMR (300 MHz, CDCl₃) δ 1.18 (m, 3H), 3.37 (m, 1H), 5.21 (m, 1H), 5.80 (d, 1H, J=7 Hz), 6.91-7.54 (m, 9H), 7.70 (s, 1H). MS APCI, m/z=326 (M+1). LC/MS: 1.24 min.

### Example 114

### N²-[(2-Fluorophenyl)acetyl]-N¹-[(2R,3S)-4-oxo-2-phenyl-2,3,4,5-tetrahydro-1,5-benzoxazepin-3-yl]-L-alaninamide (114)

A method similar to the one described for (113) was used except that 2-fluorophenylacetic acid (77 mg) was used instead of phenylacetic acid to afford the title compound as a 1:1 mixture with the 2S,3R diastereomer (150 mg), white solid, m.p. 115-135° C. ¹H NMR (300 MHz, CDCl₃) δ 0.97 (d, 1.5H, J=5 Hz), 1.25 (d, 1.5H, J=5 Hz), 3.49 (m, 2H), 4.63 (p, 0.5H, J=7 Hz), 4.75 (p, 0.5H, J=7 Hz), 4.85 (t, 1H, J=6 Hz), 5.11 (t, 1H, J=6 Hz), 5.80 (dd, 1H, J=7 Hz, J=10 Hz), 6.08 (d, 0.5H, J=8 Hz), 6.27 (d, 0.5 Hz, J=8 Hz), 6.38 (d, 0.5 h, J=6 Hz), 6.66 (d, 0.5H, J=7 Hz), 6.78-7.34 (m, 13H), 8.02 (s, 1H). MS APCI, m/z=484 (M+Na). LC/MS: 2.09 min.

### Example 115

### N²-[(3-Fluorophenyl)acetyl]-N¹-[(2R,3S)-4-oxo-2-phenyl-2,3,4,5-tetrahydro-1,5-benzoxazepin-3-yl]-L-alaninamide (115)

A method similar to the one described for (113) was used except that 3-fluorophenylacetic acid (77 mg) was used in place of phenylacetic acid to afford the title compound as a 1:1 mixture with the (2S,3R) diastereomer (140 mg), white solid, m.p. 130-140° C. ¹H NMR (300 MHz, CDCl₃) δ 0.97 (d, 1.5H, J=5 Hz), 1.27 (d, 1.5H, J=5 Hz), 3.45 (two peaks, 2H), 4.52 (m, 0.5H), 4.67 (m, 0.5H), 4.91 (t, 1H, J=6 Hz), 5.12 (t, 1H, J=6 Hz), 5.79 (dd, 1H, J=7 Hz, J=10 Hz), 6.15 (d, 0.5H, J=8 Hz), 6.28 (d, 0.5 Hz, J=8 Hz), 6.63 (d, 0.5 h, J=6 Hz), 6.70 (d, 0.5H, J=7 Hz), 6.86-7.70 (m, 13H), 7.96 (s, 1H). MS APCI, m/z=484 (M+Na). LC/MS: 2.09 mm.

### Example 116

### N²-[(4-Fluorophenyl)acetyl]-N¹-[(2R,3S)-4-oxo-2-phenyl-2,3,4,5-tetrahydro-1,5-benzoxazepin-3-yl]-L-alaninamide (116)

A method similar to the one described for (113) was used except that 4-fluorophenylacetic acid (77 mg) was used instead of phenylacetic acid to afford the title compound as a 1:1 mixture with the 2S,3R diastereomer (134 mg), white solid, m.p. 130-140° C. ¹H NMR (300 MHz, CDCl₃) δ 0.97 (d, 1.5H, J=5 Hz), 1.07 (d, 1.5H, J=5 Hz), 3.43 (two peaks, 2H), 4.52 (m, 0.5H), 4.57 (m, 0.5H), 4.93 (t, 1H, J=6 Hz), 5.11 (t, 1H, J=6 Hz), 5.78 (dd, 1H, J=7 Hz, J=10 Hz), 6.05 (d, 0.5H, J=8 Hz), 6.21 (d, 0.5 Hz, J=8 Hz), 6.33 (d, 0.5 h, J=6 Hz), 6.66 (d, 0.5H, J=7 Hz), 6.88-7.60 (m, 13H), 7.96 (s, 1H). MS APCI, m/z=484 (M+Na). LC/MS: 2.10 min.

### Example 117

### N¹-[(2R,3S,5aS,9aS)-5-(Cyclopropylmethyl)-4-oxo-2-phenyldecahydro-1,5-benzoxazepin-3-yl]-N²-[(3,5-difluorophenyl)acetyl]alaninamide (117)

Using a procedure similar to that described in Example 1, except using (2R,3S,5aS,9aS)-3-amino-5-(cyclopropylmethyl)-2-phenyloctahydro-1,5-benzoxazepin-4(5H)-one (117e) (16 mg, 0.05 mmol) as the amine component, the title compound (117) was obtained as a white solid (10 mg, 37%). ¹H NMR (300 MHz, CDCl₃) δ0.36 (m, 2H), 0.64 (m, 2H), 1.20 (m, 1H), 1.29 (d, 3H), 1.5 (m, 4H), 1.87 (m, 2H), 2.17 (m, 2H), 2.87 (dd, 1H, J=6.8, 14.0 Hz), 3.39 (t, 1H), 3.49, (s, 2H), 3.80 (dd, 1H), 4.23 (m, 1H), 4.30 (q, 1H), 5.26 (d, 1H, J=6.46), 5.43 (t, 1H, J=6.46), 6.01, (d, 1H), 6.47 (d, 1H), 6.72, (m, 1H), 6.83 (d, 2H), 7.26 (m, 5H). MS APCI, m/z=540(M+1). LC/MS: 2.71 min.

The amine component, (2R,3S,5aS,9aS)-3-amino-5-(cyclopropylmethyl)-2-phenyloctahydro-1,5-benzoxazepin-4(5H)-one (117e) was prepared in the following manner:

### a. (1,2-trans)-2-[(Cyclopropylmethyl)amino]cyclohexanol (117a)

To a stirred, cooled (5-10° C.) slurry of trans-2-aminocyclohexanol hydrochloride (9.86 g, 65 mmol) in toluene (30 mL) was added consecutively magnesium sulfate (0.79 g, 6.5 mmol), triethylamine (13.6 mL, 97.5 mmol) and cyclopropanecarboxaldehyde (4.9 g, 70 mmol) and the mixture stirred in the ice bath for ˜12 h, rising to 25° C. for ˜12 h as the ice melted. The mixture was filtered, the salt cake washed with a little toluene and the filtrate stripped in vacuo. The resulting residue, dissolved in methanol (30 mL), was cooled in an ice bath as sodium borohydride (2.70 g, 71.3 mmol) was added portionwise with stirring. The mixture was stirred in the bath as the ice melted for 2.5 h, re-cooled in an ice bath and treated cautiously with acetone (6.5 mL). After stirring for 15 min the solvent was stripped, the residue treated with ether, the solids filtered off, washed with ether and the filtrate stripped in vacuo. The resulting material was kugelrohred and the title compound was collected at 170-210° C. at 2 torr (bulb temperature), soft white solid (2.90 g, 26%). The material was used in the next step without further purification. ¹H NMR (300 MHz, CDCl₃) [symbol] 0.06-0.17 (m, 2H), 0.42-0.53 (m, 2H), 0.87-1.00 (m, 2H), 1.14-1.35 (m, 4H), 1.71-1.73 (m, 2H), 2.02-2.07 (m, 3H), 2.18-2.27 (m, 1H), 2.44-2.56 (m, 2H), 3.13-3.21 (m, 1H).

### b. (2R,3S)—N-(Cyclopropylmethyl)-N-[(1R,2R)-2-hydroxycyclohexyl]-3-phenyloxirane-2-carboxamide (117b)

To a stirred solution of (1,2-trans)-2-[(cyclopropylmethyl)amino]cyclohexanol (117a) (4.34 g, 25.6 mmol) in THF (100 mL) was added potassium (2R,3S)-3-phenyloxirane-2-carboxylate (64a) (5.06 g, 25.0 mmol), HOBt (4.20 g, 27.4 mmol), NMM (8.25 mL, 75.0 mmol) and EDAC-HCl (5.94 g, 31.0 mmol). The mixture was stirred at ambient temperature under nitrogen for ˜12 h. The solvent was evaporated and the residue was purified by flash chromatography on silica gel eluting with DCM-EtOAc (3:1 then 1:1) followed by 10% methanol-DCM to afford the title compound as a sticky white gum (5.22 g, 66%). The foam when dissolved in a little ether gave, on standing at 25° C. for ˜12 h, a white solid (1.65 g, 21%), mp 110-120° C. TLC R_{f}˜0.50 (4:1 hexane:ether). ¹H NMR (300 MHz, CDCl₃) δ0.19-0.41 (m, 2H), 0.53-0.59 (m, 2H), 0.80-1.37 (m, 4H), 1.45-1.60 (m, 1H), 1.65-1.86 (m, 3H), 2.03-2.18 (m, 2H), 3.10-3.82 (m, 4H), 3.99-4.13 (m, 1H), 7.36 (m, 5H). HPLC (Method B): 3.67 min. On standing in the HPLC vial the 3.67 min peak diminished and a peak at 4.57 min (117c) increased.

A sample of the title compound (˜2 mg) in an NMR tube containing CD₃CN (0.75 mL) was treated with TFA (2 μL) and the spectra showed complete conversion to 117c. ¹H NMR (300 MHz, CD₃CN) δ0.34-0.38 (m, 2H), 0.49-0.57 (m, 2H), 1.02-1.13 (m, 1H), 1.22-1.43 (m, 3H), 1.67-1.83 (m, 3H), 1.92-1.98 (m, 1H), 2.06-2.11 (m, 1H), 3.32 (dd 1H, J=7 Hz, J=14 Hz), 3.60-3.68 (m, 1H), 3.78 (dd, 1H, J=7 Hz, J=14 Hz), 3.75-3.88 (m, 1H), 4.33 (d, 1H, J=8.8 Hz), 4.64 (d, 1H, J=8.8 Hz), 7.27-7.40 (m, 5H).

### c. (2S,3R,5aR,9aR)-5-(Cyclopropylmethyl)-3-hydroxy-2-phenyloctahydro-1,5-benzoxazepin-4(5H)-one (117c)

A solution of (2R,3S)—N-(cyclopropylmethyl)-N-[(1R,2R)-2-hydroxycyclohexyl]-3-phenyloxirane-2-carboxamide (117b) (1.05 g, 3.33 mmol) in 125 mL acetonitrile/TFA (0.4%) was stirred for 6 h. After evaporation the crude oil was purified by chromatography (CHCl₃) to afford the title compound (850 mg, 81%). HPLC: 4.54 min (Method B). ¹H NMR (300 MHz, CDCl₃) δ0.37-0.44 (m, 2H), 0.49-0.57 (m, 2H), 0.85 (m, 1H), 1.03 (m, 1H), 1.14-1.50 (m, 2H), 1.66-1.80 (m, 2H), 1.83-2.02 (m, 1H), 2.03-2.16 (m, 2H), 3.32 (dd, 1H, J=7 Hz, J=14 Hz), 3.60 (m 1H), 3.74 (m, 1H), 3.85 (dd, 1H, J=7 Hz, J=14 Hz), 4.34 (m, 1H), 4.60 (m, 1H), 7.27-7.40 (m, 5H).

### d. (2R,3S,5aS,9aS)-3-Azido-5-(cyclopropylmethyl)-2-phenyloctahydro-1,5-benzoxazepin-4(5H)-one (117d)

Using a procedure similar to Example 31 part c, except using (2S,3R,5aR,9aR)-5-(cyclopropylmethyl)-3-hydroxy-2-phenyloctahydro-1,5-benzoxazepin-4(5H)-one (117c) (450 mg, 1.42 mmol), slightly impure title compound (30c) (112 mg, 23%) was obtained as an off white solid. ¹H NMR (300 MHz, CDCl₃) δ0.28-0.34 (m, 2H), 0.49-0.57 (m, 2H), 1.02-1.13 (m, 1H), 1.22-1.50 (m, 4H), 1.80 (m, 2H), 2.16 (m, 2H), 3.25 (dd, 1H, J=7 Hz, J=14 Hz), 3.40 (m 1H), 3.57 (dd, 1H, J=7 Hz, J=14 Hz), 4.06 (m, 1H), 4.38 (d, 1H, J=1.7 Hz), 5.41 (d, 1H, J=1.7 Hz), 7.23-7.36 (m, 3H), 7.42-7.50, (m, 2H).

### e. (2R,3S,5aS,9aS)-3-Amino-5-(cyclopropylmethyl)-2-Phenyloctahydro-1,5-benzoxazepin-4(5H)-one (117e)

Using a procedure similar to that described in example 30, part d, except using (2R,3S,5aS,9aS)-3-azido-5-(cyclopropylmethyl)-2-phenyloctahydro-1,5-benzoxazepin-4(5H)-one (117d) (110 mg, 0.325 mmol) as the azido component, the product was obtained as a slightly crude yellow solid (35 mg). The crude salt was partitioned between saturated sodium bicarbonate and EtOAc. The organic phase was separated and washed consecutively with water and brine, dried, filtered and evaporated and column chromatographed (5% methanol-CHCl₃) to yield the title compound (117e) (16 mg, 16%) as an off white solid. MS APCI, m/z=315(M+1). LC/MS: 1.80 min.

### Examples 118 and 119

### (2S)-2-(2-(3,5-difluorophenyl)acetamido)-N-((2R,3S)-5-cyclohexyl-2-(2,5-difluorophenyl)-2,3,4,5-tetrahydro-4-oxobenzo[b][1,4]thiazepin-3-yl)propanamide (118) and

### (2S)-2-(2-(3,5-difluorophenyl)acetamido)-N-((2S,3R)-5-cyclohexyl-2-(2,5-difluorophenyl)-2,3,4,5-tetrahydro-4-oxobenzo[b][1,4]thiazepin-3-yl)propanamide (119) (NOTE:Absolute stereochemistry of each not known only relative)

To a solution of racemic 2,3-trans-3-amino-5-cyclohexyl-2-(2,5-difluorophenyl)-2,3-dihydro-1,5-benzothiazepin-4(5H)-one (118f) (40 mg) in DCM (5 mL) at 0° C. under N₂ was added N-[(3,5-difluorophenyl)acetyl]-L-alanine (118e) (25 mg), HOBt-hydrate (35 mg), EDAC-HCl (30 mg) and N-methyl morpholine (18 uL). The reaction mixture was stirred 1 h at 0° C. then an additional 2 h at 25° C. The mixture was then concentrated in vacuo and partitioned between water and EtOAc. The organic phase was collected and consecutively washed with water, saturated aqueous sodium bicarbonate, and brine, dried, filtered and evaporated to yield a mixture of the title compounds. The mixture was separated by flash chromatography (30-50% gradient of EtOAc in hexanes) to afford the enantiomerically pure title compounds; early eluting trans-isomer 1 (23 mg) as an off-white solid, and the later eluting trans-isomer 2 (23 mg) also as an off-white solid.

Isomer 1 (118):

¹H NMR (300 MHz, CDCl₃) δ 0.97 (d, 3H, J=7 Hz), 1.1-1.5 (m, 4H), 1.6-1.7 (m, 4H), 1.84 (m, 1H), 2.13 (m, 1H), 3.42 (s, 3H), 4.32 (q, 1H, J=7 Hz), 4.4-4.5 (m, 1H), 4.57 (d, 1H, J=11Hz), 4.79 (a t, 1H, J=9 Hz), 5.94 (d, 1H, J=7 Hz), 6.6-6.8 (m, 4H), 6.9-7.0 (m, 2H), 7.3-7.5 (m, 2H), 7.54 (m, 1H), 7.67 (d, 1H, J=7 Hz) MS APCI, m/z=614(M+H)

Isomer 2 (119)

¹H NMR (300 MHz, CDCl₃) δ 1.0-1.3 (m, 2H), 1.20 (d, 3H, J=7 Hz), 1.3-1.5 (m, 2H), 1.6-1.8 (m, 4H), 1.83 (m, 1H), 2.12 (m, 1H), 3.38 (s, 3H), 4.24 (q, 1H, J=7 Hz), 4.4-4.6 (m, 1H), 4.57 (d, 1H, J=11 Hz), 4.75 (a t, 1H, J=9 Hz), 5.73 (d, 1H, J=7 Hz), 6.7-6.8 (m, 4H), 6.9-7.0 (m, 2H), 7.3-7.5 (m, 2H), 7.54 (a t, 1H, J=7 Hz), 7.64 (d, 1H, J=7 Hz) MS APCI, m/z=614(M+H)

The intermediate (118f) was prepared as follows:

### a. Methyl (2Z)-2-{[(benzyloxy)carbonyl]amino}-3-(2,5-difluorophenyl)prop-2-enoate (118a)

A stirred solution of N-(benzyloxycarbonyl)-α-phosphonoglycine trimethyl ester (6.1 g) and 2,5-difluorobenzaldehyde (2.0 g) in dry DCM (60 mL), was treated dropwise with a solution of DBU (2.5 mL) in DCM (20 mL). The mixture was stirred at 25° C. for 2 h, then was concentrated to approximately 20 mL and partitioned between EtOAc (150 mL) and 1N hydrochloric acid (50 mL). The organic extract was collected, consecutively washed with 1N hydrochloric acid, water, saturated aqueous sodium bicarbonate, and brine, dried (sodium sulfate), filtered and evaporated. The crude product (6.5 g) was purified by flash chromatography (20% EtOAc/hexanes) to yield the title compound (4.0 g, 82%). ¹H NMR (300 MHz, CDCl₃) δ 3.85 (s, 3H), 5.10, (s, 2H), 6.60 (bs, 1H), 6.9-7.1 (m, 2H), 7.21 (m, 1H), 7.2-7.3 (m, 6H). MS APCI, m/z=348(M⁺). LC/MS: 2.53 min (Method A).

### b. Methyl β-[(2-aminophenyl)thio]-N-[(benzyloxy)carbonyl]-2,5-difluorophenylalaninate (118b)

### Method A

To an ice-cooled solution of sodium methoxide (760 mg) in anhydrous methanol (20 mL) under N₂ (vacuum degassed 3× with nitrogen) was added 2-aminothiophenol (1.7 g). The reaction mixture stirred at 0° C. for 10 min and then a solution of methyl (2Z)-2-{[(benzyloxy)carbonyl]amino}-3-(2,5-difluorophenyl)prop-2-enoate (2.32 g) in methanol (10 mL) was added. The reaction mixture was heated to reflux for 2 h and then was cooled to 25° C. and stirred 12 h. The reaction mixture was concentrated to ca. 10 mL, then was partitioned between cold 1N hydrochloric acid (75 mL) and EtOAc (125 mL). The organic phase was separated and consecutively washed with 1N hydrochloric acid (4×), dilute aqueous sodium bicarbonate and brine, dried, filtered and evaporated. The title compound was isolated as the hydrochloride salt. (3.0 g, 88%, 2:1 Z:E). ¹H NMR (300 MHz, d6-DMSO) δ 3.4 (s, 2H), 3.7 (s, 1H), 4.6-5.1 (m, 7H), 6.3 (t, 0.67H), 6.4 (t, 0.33H), 6.7-7.4 (m, 10H), 8.1 (d, 0.33H), 8.4 (d, 0.67H). MS APCI, m/z=473(M⁺). LC/MS: 2.78 min.

### Method B

To an ice-cooled solution of 2-aminothiophenol (8.7 g) in anhydrous methanol under N₂ (vacuum degassed 3× with nitrogen) was added methyl (2Z)-2-{[(benzyloxy)carbonyl]amino}-3-(2,5-difluorophenyl)prop-2-enoate (3.46 g) followed by triethylamine (975 uL). The reaction mixture was stirred at 25° C. for 4 d, then was reduced in vacuo to near dryness. The mixture was partitioned between cold 1N hydrochloric acid (75 mL) and EtOAc (125 mL). The organic phase was separated and consecutively washed with 1N hydrochloric acid (4×), dilute aqueous sodium bicarbonate and brine, dried, filtered and evaporated to yield 5.8 g of an oil. Purification by flash chromatography (25% EtOAc/hexanes) afforded the title compound (4.3 g, 65%) Z:E ratio of 82:18. ¹H NMR (300 MHz, CDCl₃) δ 3.48 (s, 2.4H), 3.71 (s, 0.6H), 4.28 (s, 1.6H), 4.72 (s, 0.4H, 4.8-5.1 (m, 4H), 5.3 (d, 0.2H), 5.86 (d, 0.8H), 6.58 (t, 0.8H), 6.68 (d, 0.8H), 6.9-7.4 (m, 8H). MS APCI, m/z=473(M⁺). LC/MS: 2.78 min.

### c. Benzyl trans-2-(2,5-difluorophenyl)-4-oxo-2,3,4,5-tetrahydro-1,5-benzothiazepin-3-ylcarbamate (118c)

A suspension of methyl β-[(2-aminophenyl)thio]-N-[(benzyloxy)carbonyl]-2,5-difluorophenylalaninate (4:1, Z:E) (4.3 g) and p-toluenesulfonic acid (catalytic) in xylenes (100 mL) was heated to reflux for 2 h, using a Dean-Stark apparatus to remove water. The mixture was then cooled, resulting in precipitation of the crude product as a white solid (3.3 g, 4:1, cis:trans by NMR). Additional purification by flash chromatography on silica gel eluting with 25% Ethyl Acetate in Hexanes afforded the pure title compound as an off-white solid (600 mg). ¹H NMR (300 MHz, DMSO-d₆) δ 4.56 (d, 0.5H, J=9 Hz), 4.60 (d, 0.5H, J=9 Hz), 4.79 (d, 1H, J=11 Hz), 4.88 (s, 2H), 6.81 (m, 1H), 7.1-7.3 (m, 8H), 7.46 (d, 1H, J=7 Hz), 7.54 (t, 1H, J=7 Hz), 7.92 (d, 1H, J=9 Hz), 10.30 (s, 1H). MS APCI, m/z=441 (M+H).

### d. benzyl trans-5-(cyclohex-2-enyl)-2-(2,5-difluorophenyl)-2,3,4,5-tetrahydro-4-oxobenzo[b][1,4]thiazepin-3-ylcarbamate (118d)

To a solution of benzyl trans-2-(2,5-difluorophenyl)-4-oxo-2,3,4,5-tetrahydro-1,5-benzothiazepin-3-ylcarbamate (1c) (80 mg) in THF (5 mL) under N2 was added powdered potassium hydroxide (13 mg), tetrabutylammonium bromide (6 mg) and 1-bromo-2-cyclohexene (40 μl). The reaction mixture was stirred at 25° C. for ˜12 h, then was partitioned between water and EtOAc. The organic phase was separated and consecutively washed with water and brine, dried, filtered and evaporated to yield the title compound as a white solid (15 mg). This material was used without further purification. ¹H NMR (300 MHz, CDCl₃) δ 1.36 (m, 1H), 1.5-2.2 (m, 5H), 4.62 (s, 2H), 4.80 (m, 1H), 4.91 (a d, 1H), 5.24 (m, 1H), 5.68 (m, 2H), 5.90 (m, 1H), 6.88 (m, 3H), 7.2-7.5 (m, 8H), 7.61 (d, 1.0H, J=7 Hz). MS APCI, m/z=521 (M+H)

### e. N-[(3,5-Difluorophenyl)acetyl]-L-alanine (118e)

To a stirred solution of 3,5-difluorophenylacetic acid (6.02 g, 34.97 mmol), L-alanine methyl ester hydrochloride (4.88 g, 34.96 mmol) and HOBt (5.20 g, 38.48 mmol) in DCM (200 mL) under nitrogen at 0° C. was added NMM (8.84 g, 87.39 mmol) and EDAC-HCl (7.38 g, 38.49 mmol). The mixture was allowed to warm gradually to ambient temperature and stir 12 h. The reaction was diluted with EtOAc and extracted sequentially with aqueous sodium bicarbonate,
1N aqueous HCl and brine. The organic phase was dried, filtered and evaporated. The residue was purified by flash chromatography on silica gel eluting with 1:1 (v/v) hexanes:EtOAc to afford N-[(3,5-difluorophenyl)acetyl]-L-alanine methyl ester (7.91 g, 88% yield) as a white solid. ¹H NMR (300 MHz, CDCl₃) δ 1.39 (d, 3H, J=7.0 Hz), 3.54 (s, 2H), 3.75 (s, 3H), 4.59 (m, 1H), 6.02 (br 1H), 6.67-6.87 (m, 3H). MS APCI, m/z=258 (M+1). LC/MS: 1.68 min. Lithium hydroxide (1.40 g, 33.33 mmol) in water (60 mL) was added dropwise to a solution of N-[(3,5-difluorophenyl)acetyl]-L-alanine methyl ester (7.79 g, 30.28 mmol) in 1,4-dioxane (150 mL). After 2 h the solvent was evaporated. The residue was dissolved in water and the solution extracted with diethyl ether. The aqueous phase was acidified with 1N aqueous HCl and extracted with EtOAc three times. The combined EtOAc extracts were dried, filtered and evaporated to afford the title compound (7.16 g, 97% yield) as a white solid. ¹H NMR (300 MHz, d6-DMSO) δ 1.28 (d, 3H, J=7.4 Hz), 3.51 (s, 2H), 4.20 (m, 1H), 6.93-7.12 (m, 3H), 8.44 (d, 1H, J=7.0 Hz), 12.46 (br, 1H). HPLC Method A: 2.12 min.

### f. trans-3-amino-5-cyclohexyl-2-(2,5-difluorophenyl)-2,3-dihydrobenzo[b][1,4]thiazepin-4(5H)-one (118f)

A mixture of benzyl trans-5-(cyclohex-2-enyl)-2-(2,5-difluorophenyl)-2,3,4,5-tetrahydro-4-oxobenzo[b][1,4]thiazepin-3-ylcarbamate (1d) (90 mg) and 10% palladium on carbon (110 mg, DeGussa type 50% wt water) in glacial acetic acid (15 mL) was hydrogenated at 50 psi H2 for 18 h. The reaction mixture was filtered through Celite and concentrated in vacuo. The crude product was converted to its hydrochloride salt through treatment with a small excess of ethanolic HCl. The resulting solid after evaporation was triturated with diethylether to afford the title compound (32 mg) as a white solid. ¹H NMR (300 MHz, CDCl₃) δ 0.8-1.8 (m, 10H), 1.85 (d, 1H), 2.20 (d, 1H), 3.56 (d, 1H), 4.50 (d, 1H), 4.60 (m, 1H), 6.58 (m, 1H), 6.8-7.1 (m, 2H), 7.2-7.4 (m, 2H), 7.5-7.6 (m, 2H). MS APCI, m/z=389 (M+H). LC/MS 2.32 min.

### Example 120

### N²-[(3,5-difluorophenyl)acetyl]-N¹-[(2R,3R/2S,3S)-2-(3,4-dichlororophenyl)-4-oxo-2,3,4,5-tetrahydro-1,5-benzothiazepin-3-yl]-L-alaninamide (120)

Using a procedure similar to that described in Example 10 (WO2004/031154), except using racemic 2,3-trans-3-amino-2-(3,4-dichlorophenyl)-2,3-dihydro-1,5-benzothiazepin-4(5H)-one hydrobromide (120d) as the amine component, the title compound (120) was obtained as a 1:1 mixture of the (2R,3S):(2S,3R) diastereomers (90 mg, 63%) as a white solid. ¹H NMR (300 MHz, DMSO-d₆) δ 0.66 (d, 1.5H, J=7 Hz), δ 1.02 (d, 1.5H, J=7 Hz), 3.39 (s, 2H), 4.06 (q, 0.5H, J=7 Hz), 4.12 (q, 0.5H, J=7 Hz), 4.6-4.8 (m, 2H), 6.90 (d, 2H, J=7 Hz), 7.0-7.2 (m, 2H), 7.3-7.6 (m, 6H), 8.05 (m, 1H), 8.32 (d, 0.5H, J=7 Hz), 8.58 (d, 0.5H, J=9 Hz), 10.27 (s, 0.5H), 10.33 (s, 0.5H). MS APCI, m/z=585/587(M+Na). LC/MS 2.62 min.

The starting amine, (120d) was prepared as follows:

### d. (2,3-trans)-3-amino-2-(3,4-dichlorophen-1)-2,3-dihydro-1,5-benzothiazepin-4(5H)-one hydrobromide (120d)

Using a procedure similar to that described in Example 10d (WO2004/031154), except using racemic [(2,3-trans)-2-(3,4-dichlorophenyl)-2,3-dihydro-1,5-benzothiazepin-4(5H)-one (120e) (obtained as a secondary product from Example 10c) (Method D), the title compound (120d) was obtained as a 1:1 mixture of racemic trans diastereomers (214 mg, 96%) as a white solid. ¹H NMR (300 MHz, DMSO-d₆) δ 4.30 (m, 1H), 4.80 (d, 1H, J=11Hz), 7.2-7.4 (m, 3H), 7.50 (s, 1H), 7.55-7.70 (m, 3H), 8.24 (bs, 3H), 10.7 (s, 1H). MS APCI, m/z=363/365 (M+Na), LC/MS 1.9 min.

### Example 121

### N²-[(3,5-Difluorophenyl)acetyl]-N¹-[(3,4-trans)-8-fluoro-2-oxo-4-phenyl-2,3,4,5-tetrahydro-1H-1-benzazepin-3-yl]-L-alaninamide (121)

To a solution of (3,4-trans)-3-amino-8-fluoro-4-phenyl-1,3,4,5-tetrahydro-2H-1-benzazepin-2-one (121c) (30 mg) in DCM (12 mL) at 0° C. under N₂ was added N-[3,5-difluorophenyl)acetyl]-L-alanine (121e) (28 mg), HOBT-hydrate (30 mg), EDAC-HCl (32 mg) and N-methyl morpholine (28 μL). The reaction mixture was stirred for 1 h at 0° C. and then for 2 h at 25° C. The mixture was concentrated in vacuo and consecutively washed with water, saturated aqueous sodium bicarbonate, and brine, dried over sodium sulfate, filtered and evaporated to yield the title compound (30 mg, 54%) as an off-white solid. NMR studies revealed a mixture of 2 trans diastereomers. ¹H NMR (400 MHz, CD₃OD) δ 0.60 (d 1.5H), 1.12 (d, 1.5H), 2.6 (d, 1H), 3.23 (d, 1H), 3.56 (m, 1H), 4.16 (t, 1H), 4.58 (m, 1H), 6.88-7.44 (m, 8H), 8.0 (d, 1H), 8.1 (d, 1H), 8.2 (d, 1H), 9.90 (d, 1H). LC/MS: m/z=496, (M⁺+H) and 518 (M+Na)⁺, retention time=2.35 min.

The required intermediate compound (121c) was prepared as follows.

### a. Dimethyl [2-(4-fluoro-2-nitrophenyl)-1-phenylethyl]malonate (121a)

A solution of benzylidene malonate (14.2 g) in DMF (280 mL) was treated with sodium hydride (2.57 g, 95%). A solution of 4-fluoro-2-nitrotoluene in DMF (10 mL) was added over 1H, and the reaction mixture was stirred at 25° C. for 12 h and then quenched by the addition of glacial acetic acid (175 mL) at 0° C. A total of 500 mL of 70:30 water-methanol was added with stirring, and the organics were extracted with EtOAc. The organic extracts were combined and washed with saturated aqueous potassium carbonate solution and brine, dried over sodium sulfate, filtered, and concentrated in vacuo to give a dark brown oil. Flash chromatography on silica gel (75:25 hexane-EtOAc, then 50:50 hexane-EtOAc) provided 7.0 g (30%) of the title compound as a red-brown solid. ¹H NMR (300 MHz, CDCl₃) δ 3.72 (d, 2H), 3.73 (d, 1H), 4.32 (m, 1H), 7.09-7.29 (m, 7H), 7.71 (m, 1H). LC/MS: m/z=398 (M+Na)⁺, retention time=2.66 min.

### b. Dimethyl [2-(2-amino-4-fluorophenyl)-1-phenylethyl]malonate (121b)

To a solution of dimethyl [2-(4-fluoro-2-nitrophenyl)-1-phenylethyl]malonate (121a) (5.0 g) in methanol (20 mL) was added ammonium chloride (1.5 g) and zinc dust (11.0 g). The reaction mixture was then heated to reflux for 1 h. The reaction mixture was filtered through a Celite pad, and the organic solvents were removed in vacuo. The resulting yellow oil was dissolved in EtOAc (100 mL) and washed with saturated aqueous potassium carbonate solution, the organic layer was dried over sodium sulfate, filtered, and concentrated in vacuo to afford the title compound as a tan gum (4.1 g, 90%). LC/MS: m/z=346 (M+H)⁺, retention time=2.51 min.

### c. Methyl (3,4-trans)-8-fluoro-2-oxo-4-phenyl-2,3,4,5-tetrahydro-1H-1-benzazepine-3-carboxylate (121c)

A solution of dimethyl [2-(2-amino-4-fluorophenyl)-1-phenylethyl]malonate (121b) (4.3 g) in methanol (130 mL) was treated with sodium methoxide (1.73 g). The reaction mixture was heated to reflux for 5 h, cooled to 25° C., and acidified with 1N hydrochloric acid. The methanol was evaporated in vacuo, the residue was extracted with EtOAc, and the extract was washed with brine, 1N hydrochloric acid, and brine, dried over sodium sulfate, filtered, and concentrated in vacuo. The resulting crude product (4.0 g) was triturated with 1:1 diethyl ether-EtOAc to give the title compound as a colorless solid (3.87 g, 93%). ¹H NMR (300 MHz, CDCl₃) δ 2.4 (m, 2H), 3.23 (m, 1H), 3.72 (s, 3H), 3.77 (m, 1H), 6.71-7.50 (m, 7H), 8.95 (s, 1H). LC/MS: m/z=336 (M+Na)⁺, retention time=2.28 min. HPLC Method C.

### d. Methyl (3,4-cis)-8-fluoro-3-hydroxyl-2-oxo-4-phenyl-2,3,4,5-tetrahydro-1H-benzazepine-3-carboxylate (121d)

A solution of methyl (3,4-trans)-8-fluoro-2-oxo-4-phenyl-2,3,4,5-tetrahydro-1H-1-benzazepine-3-carboxylate (121c) (156 mg) in THF (10 mL) was cooled to −78° C. under nitrogen. Potassium hexamethyldisilazide (0.5 M in toluene, 4.0 mL, 4 equiv) was added, and the reaction mixture was stirred for 1 h at −78° C. Trimethyl phosphite (0.24 mL, 4 equiv) was added, and bubbling with oxygen gas through the solution was started. Bubbling with oxygen gas was continued while the temperature was allowed to warm to 0° C. over approximately 30 min. The reaction was quenched with acetic acid (7 mL), the solvents were partially removed in vacuo, EtOAc was added, and the organic layer was washed with 1N hydrochloric acid, saturated potassium carbonate, and brine, dried over sodium sulfate, filtered, and concentrated in vacuo to provide the title compound as a light yellow solid (130 mg, 80%). LC/MS: m/z=330 (M+H)⁺, retention time=2.22 min. HPLC Method C.

### e. (3,4-cis)-8-Fluoro-3-hydroxy)-4-phenyl-1,3,4,5-tetrahydro-2H-1-benzazepin-2-one (121e)

A solution of methyl (3,4-trans)-8-fluoro-3-hydroxyl-2-oxo-4-phenyl-2,3,4,5-tetrahydro-1H-1-benzazepine-3-carboxylate (8d) (1.4 g) and LiI (2.3 g, 4 equiv) in pyridine (35 mL) and water (0.35 mL) was heated to reflux for 3 h. Pyridine was removed in vacuo, EtOAc was added, and the EtOAc solution was washed with 1N hydrochloric acid, saturated aqueous potassium carbonate solution, and brine, dried over sodium sulfate, and filtered. A small amount of insoluble material was collected from the separatory funnel. This material was washed several times with water and ether, and then used to seed the EtOAc solution. Refrigeration and filtration provided the title compound as an off-white solid (700 mg, 60%). ¹H NMR (300 MHz, CDCl₃) δ 1.78-2.05 (m, 2H), 2.87 (m, 1H), 4.20 (m, 1H), 4.43 (d, 1H), 6.71-7.75 (m, 7H), 8.35 (s, 1H). LC/MS: m/z=272 (M+H)⁺, retention time=1.95 min. HPLC Method C

### f. (3,4-cis)-8-Fluoro-2-oxo-4-phenyl-2,3,4,5-tetrahydro-1H-1-benzazepin-3-yl 4-methylbenzensulfonate (121f)

To a solution of (3,4-cis)-8-fluoro-3-hydroxyl-4-phenyl-1,3,4,5-tetrahydro-2H-1-benzazepin-2-one (121e) (140.0 mg) in pyridine (4 mL) was added p-toluenesulfonyl chloride (170.0 mg, 2.3 equiv) at 0° C. The reaction mixture was stirred for 3 h at 0° C., then for 24 h at 25° C. It was then diluted with DCM (50 mL), and washed several times with water, saturated aqueous copper sulfate, and brine, dried over sodium sulfate, filtered and concentrated in-vacuo to afford the title compound as a brown oil (140.0 mg, 63.6%). LC/MS: m/z=426 (M+H)⁺, retention time=2.72 min.

### g. (3,4-trans)-3-Azido-8-fluoro-4-phenyl-1,3,4,5-tetrahydro-2H-1-benzazepin-2-one (121b)

To a solution of (3,4-cis)-8-fluoro-2-oxo-4-phenyl-2,3,4,5-tetrahydro-1H-1-benzazepin-3-yl 4-methylbenzensulfonate (121f) (220.0 mg) in DMF (4 mL) was added sodium azide (135.2 mg, 4.0 equiv) at 25° C. The reaction mixture was heated to 90° C. for 24 h, cooled, diluted with EtOAc (50 mL), and washed several times with water and brine, dried over sodium sulfate, filtered and concentrated in vacuo to provide a brown residue. Flash chromatography (hexane:EtOAc=4:1) afforded the title compound (70.0 mg, 47.2%). LC/MS: m/z=297 (M+H)⁺, retention time=2.44 min.

### h. (3,4-trans)-3-Amino-8-fluoro-4-phenyl-1,3,4,5-tetrahydro-2H-1-benzazepin-2-one (121h)

To a solution of (3,4-trans)-3-azide-8-fluoro-4-phenyl-1,3,4,5-tetrahydro-2H-1-benzazepin-2-one (121 g) (65.0 mg) in THF (5 mL) was added PS-triphenylphosphine (1.0 g, 1.2 mmol/g, 5.5 equiv) at 25° C. The reaction mixture was stirred at 25° C. for 24 h. The mixture was filtered, and the resin was extracted with THF (10 mL) and EtOAc (10 mL). The combined organic extracts were concentrated in vacuo to provide brown residue. Flash chromatography (hexane:EtOAc 1:1) provided the title compound (40.0 mg, 60.0%). ¹H NMR (300 MHz, CDCl₃) δ 1.42-1.52 (m, 2H), 1.98 (s, 2H), 2.94 (m, 1H), 3.85 (d, 1H), 6.62-7.74 (m, 8H). LC/MS: m/z=272 (M+H)⁺. NMR studies revealed a mixture of 2 trans diastereomers.

### Example 122

### 2-{[(3,5-Difluorophenyl)acetyl]amino}-N-(4-oxo-2-phenyl-2,3,4,5-tetrahydro-1,5-benzoxazepin-3-yl)propanamide

A solution of 4-oxo-2-phenyl-2,3,4,5-tetrahydro-1,5-benzoxazepin-3-aminium chloride (122d) (895 mg, 3.08 mmol), N-[(3,5-difluorophenyl)acetyl]alanine (122f) (861 mg, 3.53 mmol, 1.1 equiv), HOBt (624 mg, 4.62 mmol, 1.5 equiv) and NMM (1.4 mL, 12.32 mmol, 4.0 equiv) in DCM (10 mL) was treated with EDC (886 mg, 4.62 mmol, 1.5 equiv). The reaction mixture was stirred for 12 min at 25° C. under Ar. The reaction was then quenched with 10% HCl and extracted with EtOAc. The organics were washed with 10% NaOH and then dried with NaCl(sat) and Na₂SO₄(s). The solvents were removed under reduced pressure and the resulting residue was purified by column chromatography utilizing an ISCO system (hexane-EtOAc) to give the desired product as a mixture of trans diastereomers. ¹H NMR (300 MHz, d6-dmso) δ: 1.21-1.0 (m, 3H), 4.15 (m, 1H), 5.73 (d, 1H), 5.86 (m, 1H), 6.64 (m, 3H), 6.97 (m, 2H), 7.09 (ma, 1H), 7.23 (m, 2H), 7.31 (m, 3H), 8.56 (m, 1H), 10.45 (s, 1H); m/z 480.

### a. N-(Tert-butoxycarbonyl)-b-hydroxyphenylalanine (122a)

A solution of d,l-3-phenylserine hydrate (30.00 g, 0.166 mol) and (BOC)₂O (42 mL, 0.182 mol, 1.1 equiv) in THF (100 mL) was stirred for 12 h at 80° C. under Ar. The reaction was quenched with 10% HCl and extracted with EtOAc. The organics were dried with NaCl(sat) and then Na₂SO₄(s). The solvents were removed under reduced pressure to give a white solid; m/z 282.

### b. N-(Tert-butoxycarbonyl)-b-(2-nitrophenoxy)phenylalanine (122b)

A solution of N-(Tert-butoxycarbonyl)-b-hydroxyphenylalanine (122a) (10.05 g, 0.0357 mol) and 1-fluoro-2-nitrobenzene (3.77 mL, 0.0357 mol) in THF (100 mL) was treated with KHMDS (15.68 g, 0.0790 mol, 2.2 equiv). The reaction stirred for 12 h at 25° C. under Ar. The reaction was quenched with 10% HCl and extracted with EtOAc. The organics were dried with NaCl(sat) and then Na₂SO₄(s). The solvents were removed under reduced pressure to give the desired product; m/z 403.

### c. Tert-butyl (4-oxo-2-phenyl-2,3,4,5-tetrahydro-1,5-benzoxazepin-3-yl)carbamate (122c)

A solution of N-(Tert-butoxycarbonyl)-b-(2-nitrophenoxy)phenylalanine (122b) (14.37 g, 0.0357 mol) and Pd/C 30% (200 mg) in MeOH (200 mL) was treated with H₂. The reaction stirred for 12 h. The reaction was filtered through celite and. the solvents were removed under reduced pressure to give the crude product. Ethyl ether was then added to the residue and a white precipitate formed which was collected by vacuum filtration to provide the desired product as a mixture of the trans diastereomers (2.00 g, 16%); m/z 403.

### d. 4-Oxo-2-phenyl-2,3,4,5-tetrahydro-1,5-benzoxazepin-3-aminium chloride (122d)

A solution of Tert-butyl (4-oxo-2-phenyl-2,3,4,5-tetrahydro-1,5-benzoxazepin-3-yl)carbamate (122c) (1.15 g, 3.23 mmol) was treated with 4.0 M HCl in dioxane (10 mL). The reaction stirred for 30 min. Ethyl ether was then added to the solution and the white precipitate was collected by vacuum filtration to provide the desired product as a mixture of the trans diastereomers (940 mg g, 99%); m/z 255.

### e. Methyl N-[(3,5-difluorophenyl)acetyl]alaninate (122e)

A solution of 3,5-difluorophenylacetic acid (1.00 g, 5.81 mmol), d,l-alanine hydrochloride (811 mg, 5.81 mmol) and iPr₂NEt (3.1 mL, 17.43 mmol, 3.0 equiv) in DCM (20 mL) was treated with DCC (1.80 g, 8.72 mmol, 1.5 equiv). The reaction mixture was stirred for 12 h at 25° C. under Ar. The resulting dicyclohexylurea was removed by filtration and the solvents were removed under reduced pressure to give the desired product utilized directly without further purification.

### f. N-[(3,5-Difluorophenyl)acetyl]alanine (122f)

A solution of methyl N-[(3,5-difluorophenyl)acetyl]alaninate (122e) (1.72 g, 5.81 mmol) in THF (15 mL) was treated with 2N LiOH (4.4 mL, 8.72 mmol, 1.5 equiv). The reaction mixture was stirred for 12 h at 25° C. under Ar. Water was added to the reaction mixture and extracted with EtOAc. The aqueous layer was acidified with 10% HCl and extracted with EtOAc. The organics were dried with NaCl(sat) and Na₂SO₄(s). The solvents were removed under reduced pressure to give the desired product.

### Example 123

### Determination of the Presence of Notch 1 in Cancerous Cell Lines and Tissues

The expression of Notch 1 immunoreactive protein is determined by Western analysis of protein lysates from several human tumor cell lines (NCI-H460, A549, MCF-7, MDA-MB-468, MDA-MB-231, PC-3, DU-145, MiaPaCa-2, HCT116 and SUP-T1 (ATCC, Manassas, Va. or ECACC, Porton Down, UK) grown in vitro as described above. Protein lysates are obtained by conventional methods from human tumour xenografts (MDA-MB-231, MDA-MB-468, MCF-7, SKOV3, OVCAR-5, Colo 205, MDAH2774, BT474, MiaPaCa2, Calu-6 and A2780) (ATCC, Manassas, Va.) generated in nude mice by standard methods (see, e.g., Anticancer Drug Development Guide Preclinical Screening, Clinical Trials and Approval, Ed: Beverly A. Teicher, Humana Press, 1997). Protein lysates from 5 human breast tumours are also analyzed (Asterand, Detroit, Mich.). These tumors (Stage IIIb to IV) are obtained as frozen samples and homogenized in a buffer comprising 20 mM Tris-HCl, 500 mM sodium chloride, 1% NP-40, 1× protease inhibitor cocktail (Roche, Indianapolis, Ind.) and 1× phosphatase inhibitors (Sigma Chemical Co. St. Louis, Mo.) at 4° C. The lysates are then centrifuged at 10,000 g and the cell debris pellet is removed. The supernatants are assayed for protein content before SDS-PAGE is performed using conventional methods.

The presence of active Notch 1 protein (Notch 1 intracellular domain) is determined by Western analysis. Immunoblotting is performed using a primary antibody to cleaved Notch 1 (Val1744; Cell Signaling Technology, Danvers, Mass.) and a goat anti-rabbit horseradish peroxidase conjugated secondary antibody (Santa Cruz Biotechnology, Santa Cruz, Calif.). For Western blotting, equal amounts of protein extracted from each sample (20 μg) are loaded into pre-cast 4-12% NuPAGE Tris-Glycine gels (Invitrogen, Carlsbad, Calif.). Proteins are separated by SDS-PAGE and transferred electrophoretically to polyvinylidene diflouride membranes (Invitrogen, Carlsbad, Calif.) using a semi-dry blotter. The membrane is washed briefly in Tris-buffered saline (TBS) (Bio-Rad Laboratories, Hercules, Calif.) and blocked for 1 h in TBS supplemented with 0.05% (v/v) Tween 20 and 5% (w/v) dried milk, before being incubated for ˜12 h with primary antibody in TBS supplemented with 0.05% (v/v) Tween 20 (TBST) and 5% (w/v) dried milk. Following washing in TBST, the membrane is incubated with secondary antibody as indicated prior to visualization via enhanced chemiluminescent detection using DuraWest reagent (Pierce, Rockford, Ill.) and detection with Amersham Hyperfilm MP (Amersham, Piscataway, N.J.) according to the manufacturer's instructions. The level of protein loading is confirmed using an antibody against Hsp60 and detection with a donkey anti-goat secondary antibody (Santa Cruz Biotechnology, Santa Cruz, Calif.) using the method described above.

Data indicate that the cleaved Notch 1 antibody detects a band of approximately 100 kDa in the panel of cell lines tested, with the exception of DU-145 cells, suggesting that the Notch pathway is active in cancerous cell lines derived from multiple sources, including breast, lung and pancreas. These results are confirmed in protein extracts derived from the xenograft panel. Furthermore, cleaved Notch 1 receptor immunoreactivity is detected in protein samples derived from the five different human breast tumors assayed. Additional forms of Notch 1, as well as other Notch family members, including Notch 3 and 4 receptors, as well as additional xenograft tumor samples, may also be analyzed in analogous fashion.

### Example 124

### Determination of the Ability of Compounds of the Invention to Block Notch Signaling

T lymphoblast SUP-T1 cells (CRL-1942, ATCC, Manassas, Va.) are routinely cultured in culture medium comprising RPMI-1640 (Invitrogen, Carlsbad, Calif.) supplemented with 10% Fetal Bovine serum (Sigma Chemical Co., St. Louis, Mo.) and 2 mM glutamine (Invitrogen, Carlsbad, Calif.) and grown at 37° C. at 95%/5% (v/v) air/CO₂ in 95% relative humidity (standard cell culture conditions). Cells in culture medium are plated into flat-bottomed tissue culture plates (Corning Costar, Acton, Mass.) Subsequently, a compound of Formula (1) or vehicle (e.g., 0.1% (v/v) DMSO) may be added and plates are incubated for a further 12-16 h under standard cell culture conditions. Subsequently, the presence of active Notch 1 protein (Notch 1 intracellular domain) is determined by Western analysis according to conventional methods. Immunoblotting is performed using a primary antibody to cleaved Notch 1 (Val 1744; Cell Signaling Technology, Danvers, Mass.) and a goat anti-rabbit horseradish peroxidase conjugated secondary antibody (Santa Cruz Biotechnology, Santa Cruz, Calif.). For Western blotting, equal amounts of protein extracted from each sample (20 μg) are loaded into pre-cast 4-12% NuPAGE Tris-Glycine gels (Invitrogen, Carlsbad, Calif.). Proteins are separated by SDS-PAGE and transferred electrophoretically to polyvinylidene diflouride membranes (Invitrogen, Carlsbad, Calif.) using a semi-dry blotter. The membrane is washed briefly in Tris-buffered saline (TBS) (Bio-Rad Laboratories, Hercules, Calif.) and blocked for 1 h in TBS supplemented with 0.05% (v/v) Tween 20 (TBST) and 5% (w/v) dried milk, before being incubated for ˜12 h with primary antibody in TBS supplemented with 0.05% (v/v) Tween 20 (TBST) and 5% (w/v) dried milk. Following washing in TBST, the membrane is incubated with secondary antibody as indicated prior to visualization via enhanced chemiluminescent detection using DuraWest reagent (Pierce Biotechnology, Rockford, Ill.) and is detected with Amersham Hyperfilm MP according to the manufacturer's instructions. The level of protein loading is confirmed using an antibody against Hsp60 and detection with a donkey anti-goat secondary antibody using the method described above.

Results of experiments with compounds of Formula (1), including ‘N2-[(3,5-difluorophenyl)acetyl]-N1-[(2R,3S)-7-fluoro-4-oxo-2-phenyl-2,3,4,5-tetrahydro-1,5-benzoxazepin-3-yl]-L-alaninamide, N2-[(3,5-difluorophenyl)acetyl]-N1-[(2R,3R)-4-oxo-2-(3-thienyl)-2,3,4,5-tetrahydro-1,5-benzothiazepin-3-yl]-L-alaninamide and N2-[(2-fluorophenyl)acetyl]-N1-[(2R,3S)-4-oxo-2-phenyl-2,3,4,5-tetrahydro-1,5-benzoxazepin-3-yl]-L-alaninamide, indicate that formation of the Notch 1 intracellular domain that is generated following the cleavage of the Notch 1 protein by gamma-secretase can be inhibited by agents that block gamma-secretase enzyme activity. The Notch 1 antibody detects a band of approximately 100 kDa, consistent with the expected molecular weight of the Notch 1 intracellular domain and as described by the manufacturer (Cell Signaling Technology, Danvers, Mass.). Despite loading similar protein concentrations (as determined by the presence of equivalent amounts of Hsp60), the appearance of this band is reduced in a concentration-dependent manner by the compounds of above. The estimated IC50 for these inhibitor is as follows: 3.98×10⁻⁹ M for N2-[(3,5-difluorophenyl)acetyl]-N1-[(2R,3S)-7-fluoro-4-oxo-2-phenyl-2,3,4,5-tetrahydro-1,5-benzoxazepin-3-yl]-L-alaninamide; 5.01×10⁻⁸ M for N2-[(3,5-difluorophenyl)acetyl]-N1-[(2R,3R)-4-oxo-2-(3-thienyl)-2,3,4,5-tetrahydro-1,5-benzothiazepin-3-yl]-L-alaninamide, and 1.66×10⁻⁸ M for ‘N2-[(2-fluorophenyl)acetyl]-N1-[(2R,3S)-4-oxo-2-phenyl-2,3,4,5-tetrahydro-1,5-benzoxazepin-3-yl]-L-alaninamide. These results suggest that the compounds are potent inhibitors of Notch 1 activation and signaling.

### Example 125

### Effects of Compounds of the Present Invention on Other Notch Receptors

The effects of compounds disclosed herein on other Notch receptors (e.g., Notch 3 and Notch 4) both in SUP-T1 cells and other cell lines, including breast and lung cancer cell lines, may be demonstrated using methods analogous to those described in Examples 123 and 124.

1. A method for the treatment of disorders associated with activation of the Notch signal transduction pathway comprising administering a therapeutically effective amount of a compound of Formula (I): 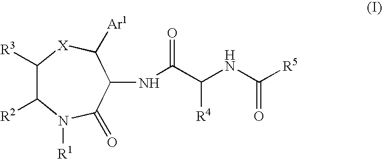
wherein:
X is CH₂, O, NR¹, SO₂ or S;
Ar¹ is a 5- or 6-membered ring optionally substituted with 0, 1, 2, or 3 R^{e} moieties, said ring having 0, 1, 2 or 3 nitrogen, oxygen or sulfur atoms, but no more than 2 oxygen atoms or 2 sulfur atoms or 1 oxygen and 1 sulfur atom;
R¹ is H, —C₁₋₃alkylC₃₋₆cycloalkyl, C₁₋₆alkyl, C₃₋₆alkenyl, C₃₋₆alkynyl, C₃₋₆cycloalkyl, C₂₋₄alkylNR^{a}R^{b}, C₁₋₄alkylC(═O)R^{d}; or C₁₋₃alkylphenyl substituted with 0, 1, 2 or 3 R^{e};
R^{a} and R^{b} are at each occurrence independently selected from H, C₁₋₄alkyl or C₃₋₆cycloalkyl, or R^{a} and R^{b} and the N to which they are attached in combination form a 5 or 6-membered N-linked heterocycle having 2 nitrogen atoms, wherein the non-linked nitrogen is substituted with R^{c} or 1 nitrogen and 1 oxygen, ring atoms wherein there is no non-linked nitrogen;
R^{c} is, at each occurrence independently selected from H, C₁₋₃alkyl, or phenyl substituted with 0, 1, 2, or 3 R^{e};
R^{d} is, at each occurrence independently selected from C₁₋₃alkyl, hydroxy, C₁₋₃alkoxy, or NR^{a}R^{b};
R^{e} is, at each occurrence independently selected from H, OH, F, Cl, Br, I, CN, NO₂, CF₃, C₁₋₆alkyl, or C₁₋₆alkoxy;
R² and R³ are at each occurrence independently selected from H, C₁₋₆alkyl, C₄₋₆cycloalkyl, aryl, or heteroaryl, or R² and R³ in combination form a fused phenyl or cyclohexyl moiety that may be substituted with 0, 1 or 2 R^{f} moieties,
R^{f} is NO₂, F, Cl, Br, I, CF₃, CN, C₁₋₆alkyl, or C₁₋₆alkoxy;
R⁴ is H, CHR⁷R⁸, 5- or 6-membered cycloalkyl, 5- or 6-membered ring optionally substituted with 0, 1, or 2 R^{f} moieties, said ring having 0, 1, 2 or 3 nitrogen, oxygen or sulfur atoms, but no more than 2 oxygen atoms or 2 sulfur atoms or 1 oxygen and 1 sulfur atom;
R⁵ is C₁₋₃alkylR⁹ or CH(OH)R¹⁰;
R⁷ and R³ are, at each occurrence are independently selected from H, C₁₋₄alkyl, OH, SH, CH₂SCH₃, CONH₂, CH₂CONH₂, CO₂H, CH₂CO₂H, (CH₂)₃NHCH(NH₂)₂, C₁₋₄alkylamino, indolyl, imidazolyl, phenyl or hydroxyphenyl or R⁷ and R³ in combination form a 6-membered ring optionally substituted with 0, 1 or 2 R^{f} moieties said heterocyclic ring having 0, 1, 2 or 3 nitrogen, oxygen or sulfur atoms, but no more than 2 oxygen atoms or 2 sulfur atoms or 1 oxygen and 1 sulfur atom;
R⁹ is phenyl substituted with 0, 1, 2 or 3 R^{e};
R¹⁰ is C₁₋₆alkyl or R⁹;
in free form or in a pharmaceutically acceptable salt form or in the form of a pharmaceutically acceptable solvate of the compound or the salt, to a human or animal patient in need thereof.

2. The method of claim 1, wherein X is S, O, or CH₂.

3. The method of claim 1 wherein Ar¹ is a 6-membered aromatic ring optionally substituted with 1, 2 R^{e} moieties wherein R^{e} is F or Cl, C₁₋₆alkyl, or C₁₋₆alkoxy, or Ar¹ is a 5-membered heterocyclic ring optionally substituted with 1 R^{e} moiety wherein R^{e} is F, Cl, Br, C₁₋₆alkyl.

4. The method of claim 1 wherein R¹ is H, C₂₋₄alkylNR^{a}R^{b}, C₁₋₆alkyl, C₃₋₆cycloalkyl, C₃₋₆alkynyl, —C₁₋₄alkylC(═O)C₁₋₃alkoxy, —C₁₋₃alkylC₃₋₆cycloalkyl, or C₁₋₃alkylphenyl substituted with C₁₋₆alkoxy.

5. The method of claim 1 wherein R² and R³ are each H, or combined to form a fused phenyl moiety substituted with F, Cl, or C₁₋₆alkoxy, or combined to form a cyclohexyl.

6. The method of any of claim 1 wherein R⁴ is H, CHR⁷R³ wherein R⁷ and R³ is H, C₁₋₄alkyl, CH₂CH₂SCH₃, CO₂H, or CH₂CO₂H, OH, or a 6-membered aromatic ring optionally substituted with 1 F, or a 6-membered cycloalkyl.

7. The method of claim 1 wherein R⁵ is C₁₋₃alkylR⁹ wherein R⁹ is a phenyl substituted with 2 F or is CH(OH)R¹⁰ wherein R¹⁰ is C₄alkyl or R⁹ wherein R⁹ is phenyl optionally substituted with 0, 1, or 2 F.

8. The method of claim 1 wherein:
X is S, O, or CH₂;
Ar¹ is a phenyl optionally substituted with 1, 2 R^{e} moieties wherein R^{e} is F or Cl, methyl, or methoxy, or Ar¹ is a furyl, thienyl optionally substituted with 1 R^{e} moiety wherein R^{e} is F, Cl, Br, methyl;
R¹ is H, dimethylaminoethyl, 2-morpholinoethyl, methyl or isopropyl, cyclohexyl, 2-propyn-1-yl, methoxycarbonylmethyl, carboxymethyl, cyclopropylmethyl, or 4-methoxybenzyl;
R² and R³ are each H, or combined to form a fused phenyl moiety substituted with F, Cl, or methoxy, or combined to form a cyclohexyl;
R⁴ is H, methyl, benzyl, isopropyl, isopropylmethyl, indol-2ylmethyl, CH₂CH₂SCH₃, or CH₂CO₂H, CH₂CH₂CO₂H, CH₂OH, or phenyl optionally substituted with 1 F, or cyclohexyl; and
R⁵ is benzyl, 1-hydroxy-3-methylbutyl, o-hydroxy-3,5-difluorobenzyl, 3,5-difluorobenzyl or 3-5-difluorophenyl, 2-fluorobenzyl, 3-fluorobenzyl, or 4-fluorobenzyl.

9. The method according to claim 1 wherein the disorder to be treated is cancer.

10. The method according to claim 1 wherein the disorder is selected from the group consisting of hematologic, genitourinary, gynecologic, digestive, gastrointestinal, neurologic, breast, lung and mucoepidermoid cancers.

11. The method according to claim 1 wherein the disorder is selected from the group consisting of leukemia, multiple myeloma, extramedullary plasmacytoma, renal cell carcinoma and ovarian, endometrial, cervical, colon, prostate, or pancreatic cancer.

12. The method according to claim 1 wherein the disorder is T cell acute lymphocytic leukemia.
